(19) United States
(12) Patent Application Publication
(10) Pub. No.: US 2020/0360431 A1
(43) Pub. Date: Nov. 19, 2020

(54) BCMA-TARGETING CHIMERIC ANTIGEN RECEPTOR, CD19-TARGETING CHIMERIC ANTIGEN RECEPTOR, AND COMBINATION THERAPIES

(71) Applicants: Novartis AG, Basel (CH); The Trustees of the University of Pennsylvania, Philadelphia, PA (US)

(72) Inventors: Alfred Garfall, Wallingford, PA (US); Adam David Cohen, Bala Cynwyd, PA (US); Michael C. Milone, Cherry Hill, NJ (US); Gabriela Plesa, Blue Bell, PA (US); Edward A. Stadtmauer, Bryn Mawr, PA (US); Carl H. June, Merion Station, PA (US)

(21) Appl. No.: 16/764,459

(22) PCT Filed: Nov. 15, 2018
(86) PCT No.: PCT/US2018/061239

§ 371 Date: May 15, 2020

Related U.S. Application Data
(60) Provisional application No. 62/588,836, filed on Nov. 20, 2017.
(60) Provisional application No. 62/586,834, filed on Nov. 15, 2017.

(51) Int. Cl. A61K 35/17; C07K 16/28; A61K 31/675; A61K 31/7076; A61K 31/454

(52) U.S. Cl. CPC A61K 35/17; C07K 16/2878; A61K 31/454; A61K 31/675; A61K 31/7076; C07K 16/2803

(57) ABSTRACT

The invention provides compositions and methods for treating diseases associated with expression of BCMA. The invention also relates to a method of administering a BCMA-targeting chimeric antigen receptor (CAR) therapy and an additional therapeutic agent.

### RELATED APPLICATIONS

This application claims priority to U.S. Ser. No. 62/586,834 filed Nov. 15, 2017, and U.S. Ser. No. 62/588,836 filed Nov. 20, 2017, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 8, 2018, is named N2067-7145WO_SL.txt and is 676,612 bytes in size.

### FIELD OF THE INVENTION

The present invention relates generally to the use of cells engineered to express a chimeric antigen receptor targeting B-cell maturation antigen protein (BCMA), optionally in combination with an additional therapeutic agent, to treat a disease associated with the expression of BCMA. The invention further describes prognostic biomarkers for BCMA-targeted therapies.

### BACKGROUND OF THE INVENTION

BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma (MM), Hodgkin's and non-Hodgkin's lymphoma, diffuse large B-cell lymphoma (DLBCL), various leukemias (e.g., chronic lymphocytic leukaemia (CLL)), and glioblastoma.

Given the ongoing need for improved strategies for targeting diseases such as cancer, new compositions and methods for improving therapeutic agents that target BCMA, e.g., anti-BCMA chimeric antigen receptor (CAR) therapies, are highly desirable.

### SUMMARY OF THE INVENTION

The disclosure features, at least in part, a method of treating a disease or disorder associated with expression of B-cell maturation antigen (BCMA), comprising administering to the subject a BCMA CAR-expressing cell therapy.

In one aspect, disclosed herein is a method of treating a subject comprising administering to the subject a BCMA CAR-expressing cell therapy, wherein the subject has stage III high-risk multiple myeloma (e.g., stage III high-risk multiple myeloma based on Revised International Staging System), thereby treating the subject.

In one embodiment, the subject has received first-line therapy (e.g., induction therapy, e.g., induction therapy comprising one, two, or all of: lenalidomide, bortezomib, or dexamethasone) before the administration of the BCMA CAR-expressing cell therapy. In one embodiment, the subject has responded or is responding to the first line therapy (e.g., induction therapy, e.g., induction therapy comprising one, two, or all of: lenalidomide, bortezomib, or dexamethasone). In one embodiment, the subject has shown complete response, very good partial response, or partial response after receiving the first line therapy (e.g., induction therapy, e.g., induction therapy comprising one, two, or all of: lenalidomide, bortezomib, or dexamethasone).

In one aspect, disclosed herein is a method of treating a subject having a disease associated with the expression of BCMA (e.g., stage III high-risk multiple myeloma, e.g., stage III high-risk multiple myeloma based on Revised International Staging System), comprising administering to the subject a BCMA CAR-expressing cell therapy, wherein the BCMA CAR-expressing cell therapy is administered after first-line therapy (e.g., induction therapy, e.g., induction therapy comprising one, two, or all of: lenalidomide, bortezomib, or dexamethasone), wherein the subject has responded or is responding to the first line therapy (e.g., induction therapy, e.g., induction therapy comprising one, two, or all of: lenalidomide, bortezomib, or dexamethasone), e.g., the subject has shown complete response, very good partial response, or partial response after receiving the first line therapy (e.g., induction therapy, e.g., induction therapy comprising one, two, or all of: lenalidomide, bortezomib, or dexamethasone).

In some embodiments, the method disclosed herein further comprises administering to the subject a CD19 CAR-expressing cell therapy. Without wishing to be bound by theory, multiple myeloma may be mediated, at least in part, by a minor subset of multiple myeloma cells with cancer stem cell properties, which resemble B lymphocytes and express CD19. A CD19 CAR-expressing cell therapy may increase the efficacy of a BCMA CAR-expressing cell therapy by targeting early lineage cancer cells, e.g., cancer stem cells, modulating the immune response, depleting regulatory B cells, and/or improving the tumor microenvironment. In some embodiments, the CD19 CAR-expressing cell therapy is administered prior to, concurrently with, or after the administration of the BCMA CAR-expressing cell therapy. In some embodiments, the CD19 CAR-expressing cell therapy is administered concurrently with the administration of the BCMA CAR-expressing cell therapy.

In some embodiments, the BCMA CAR-expressing cell therapy is administered in a single infusion or a split-dose infusion. In some embodiments, the BCMA CAR-expressing cell therapy is administered in a single infusion. In some embodiments, the BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ cells (e.g., viable CAR-expressing cells), e.g., about 5×10⁸ cells (e.g., viable CAR-expressing cells), e.g., about 5×10⁸ cells (e.g., viable CAR-expressing cells) in a single infusion.

In some embodiments, the CD19 CAR-expressing cell therapy is administered in a single infusion or a split-dose infusion. In some embodiments, the CD19 CAR-expressing cell therapy is administered in a single infusion. In some embodiments, the CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ cells (e.g., viable CAR-expressing cells), e.g., about 5×10⁸ cells (e.g., viable CAR-expressing cells), e.g., about 5×10⁸ cells (e.g., viable CAR-expressing cells) in a single infusion.

In some embodiments, the method disclosed herein further comprises administering to the subject a conditioning agent (e.g., a lymphodepletion agent, e.g., a lymphodepleting chemotherapy, e.g., cyclophosphamide and/or fludarabine) before administering the BCMA CAR-expressing cell therapy or the CD19 CAR-expressing cell therapy. In some embodiments, the BCMA CAR-expressing cell therapy and/or the CD19 CAR-expressing cell therapy are administered 2, 3, or 4 days, e.g., 3 days after the administration of the conditioning agent is completed (e.g., after the administration of a last dose of the lymphodepletion agent, e.g., a last dose of the lymphodepleting chemotherapy, e.g., a last dose of cyclophosphamide and/or fludarabine). In some embodiments, the method disclosed herein further comprises, prior to the administration of the conditioning agent (e.g., the lymphodepletion agent, e.g., the lymphodepleting chemotherapy, e.g., cyclophosphamide and/or fludarabine), obtaining a sample (e.g., an apheresis sample) from the subject and manufacturing the BCMA CAR-expressing cell therapy and/or the CD19 CAR-expressing cell therapy using the sample.

In some embodiments, the method disclosed herein further comprises administering a maintenance agent (e.g., lenalidomide) after the administration of the BCMA CAR-expressing cell therapy and/or the CD19 CAR-expressing cell therapy, e.g., 28, 29, 30, 31, or 32 days after the administration of the BCMA CAR-expressing cell therapy and/or the CD19 CAR-expressing cell therapy.

In one aspect, disclosed herein is a method of evaluating the effectiveness of a CAR-expressing cell therapy in a subject having a disease associated with the expression of BCMA (e.g., stage III high-risk multiple myeloma, e.g., stage III high-risk multiple myeloma based on Revised International Staging System), wherein the subject has received or is receiving the CAR-expressing cell therapy, and wherein the CAR-expressing cell therapy comprises a combination of a BCMA CAR-expressing cell therapy and a CD19 CAR-expressing cell therapy, comprising:

(i) acquiring a first value of the level of anti-SOX2 immune response (e.g., anti-SOX2 antibody response or T cell response) in the subject, e.g., in a sample from the subject, and/or

(ii) acquiring a second value of SOX2 level or activity (e.g., SOX2 expression level) in the subject, e.g., in a sample from the subject, at at least one time point after the subject began receiving the CAR-expressing cell therapy, wherein:

(1) an increase in the first value, as compared to a first reference value, and/or a decrease in the second value, as compared to a second reference value, indicates that the CAR-expressing cell therapy is effective in the subject (e.g., the subject responds to the CAR-expressing cell therapy); and

(2) a decrease in the first value, as compared to a first reference value, and/or an increase in the second value, as compared to a second reference value, indicates that the CAR-expressing cell therapy is ineffective or minimally effective in the subject (e.g., the subject does not respond or only minimally responds to the CAR-expressing cell therapy); wherein:

(i) the first reference value is:

the level of anti-SOX2 immune response (e.g., anti-SOX2 antibody response or T cell response) in the subject prior to the at least one time point (e.g., before the subject began receiving the CAR-expressing cell therapy, or after the subject began receiving the CAR-expressing cell therapy but prior to the at least one time point);

the level of anti-SOX2 immune response (e.g., anti-SOX2 antibody response or T cell response) in a different subject having the disease associated with the expression of BCMA; or

an average level of anti-SOX2 immune response (e.g., anti-SOX2 antibody response or T cell response) in a population of subjects having the disease associated with the expression of BCMA; and

(ii) the second reference value is:

the SOX2 level or activity (e.g., SOX2 expression level) in the subject prior to the at least one time point (e.g., before the subject began receiving the CAR-expressing cell therapy, or after the subject began receiving the CAR-expressing cell therapy but prior to the at least one time point);

the SOX2 level or activity (e.g., SOX2 expression level) in a different subject having the disease associated with the expression of BCMA; or

an average SOX2 level or activity (e.g., SOX2 expression level) in a population of subjects having the disease associated with the expression of BCMA;

thereby evaluating the effectiveness of a CAR-expressing cell therapy.

In one aspect, disclosed herein is a method of treating a subject having a disease associated with the expression of BCMA (e.g., stage III high-risk multiple myeloma, e.g., stage III high-risk multiple myeloma based on Revised International Staging System), comprising:

in response to a determination that the subject, after being administered a CAR-expressing cell therapy comprising a combination of a BCMA CAR-expressing cell therapy and a CD19 CAR-expressing cell therapy, has not achieved, or has not been identified as having achieved, an increase in the level of anti-SOX2 immune response (e.g., anti-SOX2 antibody response or T cell response) and/or a decrease in SOX2 level or activity (e.g., SOX2 expression level) in the subject, e.g., in a sample from the subject, administering a second therapy or procedure to the subject,

thereby treating the subject.

In some embodiments, the second therapy or procedure is chosen from one or more of chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy.

In certain embodiments of the foregoing methods, the BCMA CAR-expressing cell therapy comprises a cell (e.g., a population of cells) expressing a BCAM CAR, wherein the BCMA CAR comprises one or more of (e.g., all three of) heavy chain complementary determining region 1 (HCDR1), HCDR2, and HCDR3 of any BCMA scFv domain amino acid sequence listed in Table 2 or 3 and/or one or more of (e.g., all three of) light chain complementary determining region 1 (LCDR1), LCDR2, and LCDR3 of any BCMA scFv domain amino acid sequence listed in Table 2 or 3, or a sequence with 95-99% identity thereof.

In certain embodiments of the foregoing methods, the BCMA CAR-expressing cell therapy comprises a cell (e.g., a population of cells) expressing a BCAM CAR, wherein the BCMA CAR comprises a heavy chain variable region (VH) listed in Table 2 or 3 and/or a light chain variable region (VL) listed in Table 2 or 3, or a sequence with 95-99% identity thereof.

In certain embodiments of the foregoing methods, the BCMA CAR-expressing cell therapy comprises a cell (e.g., a population of cells) expressing a BCAM CAR, wherein the BCMA CAR comprises a BCMA scFv domain amino acid sequence listed in Table 2 or 3 (e.g., SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, and SEQ ID NO: 149), or a sequence with 95-99% identity thereof.

In certain embodiments of the foregoing methods, the BCMA CAR-expressing cell therapy comprises a cell (e.g., a population of cells) expressing a BCAM CAR, wherein the BCMA CAR comprises a full-length BCMA CAR amino acid sequence listed in Table 2 or 3 (e.g., the amino acid sequence of the immature BCMA CAR comprises the amino acid sequence of SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, and SEQ ID NO: 233), or a sequence with 95-99% identity thereof.

In certain embodiments of the foregoing methods, the BCMA CAR-expressing cell therapy comprises a cell (e.g., a population of cells) expressing a BCAM CAR, wherein the BCMA CAR is encoded by a nucleic acid sequence listed in Table 2 or 3 (e.g., SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170), or a sequence with 95-99% identify thereof.

In certain embodiments of the foregoing methods, the CD19 CAR-expressing cell therapy comprises a cell (e.g., a population of cells) expressing a CD19 CAR, wherein the CD19 CAR comprises one or more of (e.g., all three of) heavy chain complementary determining region 1 (HCDR1), HCDR2, and HCDR3 listed in Table 6 or 7 and/or one or more of (e.g., all three of) light chain complementary determining region 1 (LCDR1), LCDR2, and LCDR3 listed in Table 6 or 8, or a sequence with 95-99% identity thereof.

In certain embodiments of the foregoing methods, the CD19 CAR-expressing cell therapy comprises a cell (e.g., a population of cells) expressing a CD19 CAR, wherein the CD19 CAR comprises a heavy chain variable region (VH) of any CD19 scFv domain amino acid sequence listed in Table 6 and/or a light chain variable region (VL) of any CD19 scFv domain amino acid sequence listed in Table 6, or a sequence with 95-99% identify thereof.

In certain embodiments of the foregoing methods, the CD19 CAR-expressing cell therapy comprises a cell (e.g., a population of cells) expressing a CD19 CAR, wherein the CD19 CAR comprises a CD19 scFv domain amino acid sequence listed in Table 6, or a sequence with 95-99% identity thereof.

In certain embodiments of the foregoing methods, the CD19 CAR-expressing cell therapy comprises a cell (e.g., a population of cells) expressing a CD19 CAR, wherein the CD19 CAR comprises a full-length CD19 CAR amino acid sequence listed in Table 6, or a sequence with 95-99% identity thereof.

In certain embodiments of the foregoing methods, the CD19 CAR-expressing cell therapy comprises a cell (e.g., a population of cells) expressing a CD19 CAR, wherein the CD19 CAR is encoded by a nucleic acid sequence listed in Table 6, or a sequence with 95-99% identity thereof.

In certain embodiments of the foregoing methods, the subject is a human patient.

In one aspect, disclosed herein is a method of treating a subject comprising administering to the subject a first BCMA CAR-expressing cell therapy, wherein the subject has multiple myeloma, wherein:

(i) the subject has stage III high-risk multiple myeloma, e.g., stage III high-risk multiple myeloma based on Revised International Staging System,

(ii) the subject shows beta-2-microglobulin ≥5.5 mg/L and high-risk FISH features: deletion 17p, t(14;16), t(14;20), t(4;14);

(iii) the subject shows beta-2-microglobulin ≥5.5 mg/L and LDH greater than upper limit of normal;

(iv) the subject shows metaphase karyotype with >3 structural abnormalities except hyperdiploidy;

(v) the subject has plasma cell leukemia, e.g., the subject shows >20% plasma cells in peripheral blood;

(vi) the subject fails to achieve a partial response or better (e.g., based on IMWG 2016 criteria, e.g., as described in Table 5) to an Imid/PI combination (thalidomide, lenalidomide, or pomalidomide in combination with bortezomib, ixazomib, or carfilzomib); or

(vii) the subject progresses on first-line therapy with an Imid/PI combination within six months of starting therapy,

thereby treating the subject.

In some embodiments, the first BCMA CAR-expressing cell therapy is administered after first-line therapy (e.g., induction therapy, e.g., induction therapy comprising one, two, or all of: lenalidomide, bortezomib, or dexamethasone) or second-line therapy, e.g., at least three cycles of the first-line therapy or second-line therapy, wherein the subject has responded or is responding to the first-line therapy or second-line therapy, e.g., the subject has shown at least a minimal response, e.g., the subject has shown a complete response, a very good partial response, a partial response, or a minimal response after receiving the first-line therapy or second-line therapy, e.g., based on IMWG 2016 criteria, e.g., as described in Table 5.

In one aspect, disclosed herein is a method of treating a subject having a disease associated with the expression of BCMA, comprising administering to the subject a first BCMA CAR-expressing cell therapy, wherein the first BCMA CAR-expressing cell therapy is administered after first-line therapy (e.g., induction therapy, e.g., induction therapy comprising one, two, or all of: lenalidomide, bortezomib, or dexamethasone) or second-line therapy, e.g., at least three cycles of the first-line therapy or second-line therapy, wherein the subject has responded or is responding to the first-line therapy or second-line therapy, e.g., the subject has shown at least a minimal response, e.g., the subject has shown a complete response, a very good partial response, a partial response, or a minimal response after receiving the first-line therapy or second-line therapy, e.g., based on IMWG 2016 criteria, e.g., as described in Table 5.

In some embodiments, the subject has not shown or is not showing a complete response or a stringent complete response to the first-line therapy or second-line therapy, e.g., based on IMWG 2016 criteria, e.g., as described in Table 5.

In some embodiments, the subject has shown or is showing a complete response or a stringent complete response to the first-line therapy or second-line therapy, wherein the subject has shown or is showing minimal residual disease, e.g., as measured by bone marrow flow cytometry, e.g., clonal plasma cells are detectable in bone marrow by flow cytometry, e.g., based on IMWG 2016 criteria, e.g., as described in Table 5.

In some embodiments, the first BCMA CAR-expressing cell therapy is administered after the second-line therapy, wherein the subject advanced to the second-line therapy due to disease progression during first-line therapy, wherein the disease progression occurred within six months of beginning the first-line therapy.

In some embodiments, the subject has not received high-dose melphalan or autologous or allogeneic stem cell transplantation.

In some embodiments, the method further comprises administering to the subject a first CD19 CAR-expressing cell therapy.

In some embodiments, the first CD19 CAR-expressing cell therapy is administered prior to, concurrently with, or after the administration of the first BCMA CAR-expressing cell therapy.

In some embodiments, the first CD19 CAR-expressing cell therapy is administered on the same day as the first BCMA CAR-expressing cell therapy, optionally wherein the first CD19 CAR-expressing cell therapy is administered at least one hour after the completion of the administration of the first BCMA CAR-expressing cell therapy.

In some embodiments, the first CD19 CAR-expressing cell therapy is administered after the first BCMA CAR-expressing cell therapy, wherein if the subject develops acute infusion reaction after the administration of the first BCMA CAR-expressing cell therapy, the first CD19 CAR-expressing cell therapy is administered up to 48 hours (e.g., 24, 36, or 48 hours) after the administration of the first BCMA CAR-expressing cell therapy.

In some embodiments, the first BCMA CAR-expressing cell therapy is administered in a single infusion or a split-dose infusion. In some embodiments, the first BCMA CAR-expressing cell therapy is administered in a single infusion. In some embodiments, the first BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the first CD19 CAR-expressing cell therapy is administered in a single infusion or a split-dose infusion. In some embodiments, the first CD19 CAR-expressing cell therapy is administered in a single infusion. In some embodiments, the first CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the method further comprises administering to the subject a first conditioning agent (e.g., a lymphodepletion agent, e.g., a lymphodepleting chemotherapy, e.g., cyclophosphamide and/or fludarabine) before administering the first BCMA CAR-expressing cell therapy and/or the first CD19 CAR-expressing cell therapy. In some embodiments, the method comprises administering to the subject cyclophosphamide and fludarabine before administering the first BCMA CAR-expressing cell therapy and/or the first CD19 CAR-expressing cell therapy, optionally wherein:

(i) cyclophosphamide is administered at 300 mg/m² intravenously daily for three days; and

(ii) fludarabine is administered at 30 mg/m² intravenously daily for three days.

In some embodiments, the first BCMA CAR-expressing cell therapy and/or the first CD19 CAR-expressing cell therapy are administered 2, 3, or 4 days, e.g., 3 days, after the administration of the first conditioning agent is completed (e.g., after the administration of a last dose of the lymphodepletion agent, e.g., a last dose of the lymphodepleting chemotherapy, e.g., a last dose of cyclophosphamide and/or fludarabine).

In some embodiments, the method further comprises, prior to the administration of the first conditioning agent (e.g., the lymphodepletion agent, e.g., the lymphodepleting chemotherapy, e.g., cyclophosphamide and/or fludarabine), obtaining a first sample (e.g., an apheresis sample) from the subject and manufacturing the first BCMA CAR-expressing cell therapy and/or the first CD19 CAR-expressing cell therapy using the sample.

In some embodiments, the method further comprises, prior to the administration of the first conditioning agent (e.g., the lymphodepletion agent, e.g., the lymphodepleting chemotherapy, e.g., cyclophosphamide and/or fludarabine) and after obtaining the first sample, obtaining a second sample (e.g., stem cells) from the subject for preparing autologous stem cell transplantation.

In some embodiments, the method further comprises administering to the subject a maintenance agent (e.g., lenalidomide) after the administration of the first BCMA CAR-expressing cell therapy and/or the first CD19 CAR-expressing cell therapy, e.g., at the later of:

(i) 26, 27, 28, 29, 30, 31, or 32 days, e.g., 28 days, after the administration of the first BCMA CAR-expressing cell therapy and/or the first CD19 CAR-expressing cell therapy; or

(ii) resolution of grade ≤2 of treatment-related toxicity.

In some embodiments, the method further comprises administering to the subject a second BCMA CAR-expressing cell therapy after the administration of the maintenance agent, wherein:

(i) about 80-100 (e.g., about 90 days) have elapsed since the administration of the first BCMA CAR-expressing cell therapy;

(ii) the subject's multiple myeloma has progressed after the administration of the first BCMA CAR-expressing cell therapy; or

(iii) the subject has exhibited or is exhibiting objective evidence of residual multiple myeloma after the administration of the first BCMA CAR-expressing cell therapy.

In some embodiments, the method further comprises administering to the subject a second CD19 CAR-expressing cell therapy after the administration of the maintenance agent, wherein >3% peripheral blood lymphocytes of the subject are CD19+ after the administration of the first CD19 CAR-expressing cell therapy, e.g., 7-28 days after the administration of the first CD19 CAR-expressing cell therapy.

In some embodiments, the second CD19 CAR-expressing cell therapy is administered prior to, concurrently with, or after the administration of the second BCMA CAR-expressing cell therapy.

In some embodiments, the second CD19 CAR-expressing cell therapy is administered on the same day as the second BCMA CAR-expressing cell therapy, optionally wherein the second CD19 CAR-expressing cell therapy is administered at least one hour after the completion of the administration of the second BCMA CAR-expressing cell therapy.

In some embodiments, the second CD19 CAR-expressing cell therapy is administered after the second BCMA CAR-expressing cell therapy, wherein if the subject develops acute infusion reaction after the administration of the second BCMA CAR-expressing cell therapy, the second CD19 CAR-expressing cell therapy is administered up to 48 hours (e.g., 24, 36, or 48 hours) after the administration of the second BCMA CAR-expressing cell therapy.

In some embodiments, the second BCMA CAR-expressing cell therapy is administered in a single infusion or a split-dose infusion. In some embodiments, the second BCMA CAR-expressing cell therapy is administered in a single infusion. In some embodiments, the second BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the second CD19 CAR-expressing cell therapy is administered in a single infusion or a split-dose infusion. In some embodiments, the second CD19 CAR-expressing cell therapy is administered in a single infusion. In some embodiments, the second CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the second BCMA CAR-expressing cell therapy is the same as the first BCMA CAR-expressing cell therapy.

In some embodiments, the second CD19 CAR-expressing cell therapy is the same as the first CD19 CAR-expressing cell therapy.

In some embodiments, the method further comprises administering to the subject a second conditioning agent (e.g., a lymphodepletion agent, e.g., a lymphodepleting chemotherapy, e.g., cyclophosphamide and/or fludarabine) before administering the second BCMA CAR-expressing cell therapy and/or the second CD19 CAR-expressing cell therapy. N In some embodiments, the method comprises administering to the subject cyclophosphamide and fludarabine before administering the second BCMA CAR-expressing cell therapy and/or the second CD19 CAR-expressing cell therapy, optionally wherein:

(i) cyclophosphamide is administered at 300 mg/m² intravenously daily for three days; and

(ii) fludarabine is administered at 30 mg/m² intravenously daily for three days. In some embodiments, the method comprises administering to the subject cyclophosphamide, e.g., at 1.5 g/m², before administering the second BCMA CAR-expressing cell therapy and/or the second CD19 CAR-expressing cell therapy.

In one aspect, disclosed herein is a method of treating a subject having a disease associated with the expression of BCMA, comprising administering to the subject a first BCMA CAR-expressing cell therapy, wherein the subject has high-risk multiple myeloma, e.g., stage III high-risk multiple myeloma based on Revised International Staging System. In one aspect, disclosed herein is a method of treating a subject having a disease associated with the expression of BCMA, comprising administering to the subject a first BCMA CAR-expressing cell therapy, wherein the subject is receiving or has received a first-line therapy (e.g., induction therapy, e.g., induction therapy comprising one, two, or all of: lenalidomide, bortezomib, or dexamethasone) or a second-line therapy, e.g., at least three cycles of the first-line therapy or second-line therapy, e.g., based on IMWG 2016 criteria, e.g., as described in Table 5, and the subject has not progressed from the first-line or second-line therapy. In one aspect, disclosed herein is a method of treating a subject having a disease associated with the expression of BCMA, comprising administering to the subject a first BCMA CAR-expressing cell therapy, wherein the subject has shown at least a minimal response, e.g., the subject has shown a very good partial response, a partial response, or a minimal response, to a most recent therapy received by the subject (e.g., the first-line therapy or second-line therapy), e.g., based on IMWG 2016 criteria, e.g., as described in Table 5.

In one aspect, disclosed herein is a method of treating a subject having a disease associated with the expression of BCMA, comprising administering to the subject a first BCMA CAR-expressing cell therapy, wherein: (i) the subject has high-risk multiple myeloma, e.g., stage III high-risk multiple myeloma based on Revised International Staging System; (ii) the subject is receiving or has received a first-line therapy (e.g., induction therapy, e.g., induction therapy comprising one, two, or all of: lenalidomide, bortezomib, or dexamethasone) or a second-line therapy, e.g., at least three cycles of the first-line therapy or second-line therapy, e.g., based on IMWG 2016 criteria, e.g., as described in Table 5, and the subject has not progressed from the first-line or second-line therapy; and (iii) the subject has shown at least a minimal response, e.g., the subject has shown a very good partial response, a partial response, or a minimal response, to a most recent therapy received by the subject (e.g., the first-line therapy or second-line therapy), e.g., based on IMWG 2016 criteria, e.g., as described in Table 5, thereby treating the subject.

In some embodiments, the subject is receiving or has received a first-line therapy and has not received a second-line therapy. In some embodiments, the subject has not progressed from the first-line therapy. In some embodiments, the subject is receiving or has received a second-line therapy and has not received a third-line therapy, wherein the subject advanced to the second-line therapy due to disease progression during or after receiving a first-line therapy, wherein the disease progression occurred within one year of beginning the first-line therapy or within six months of completing the first-line therapy. In some embodiments, the subject has not progressed from the second-line therapy.

In some embodiments, the subject has not shown or is not showing a complete response or a stringent complete response to the most recent therapy received by the subject (e.g., the first-line therapy or second-line therapy), e.g., based on IMWG 2016 criteria, e.g., as described in Table 5.

In some embodiments, the subject has not received cytotoxic chemotherapy (e.g., doxorubicin, cyclophosphamide, etoposide, or cisplatin) with the following exceptions: (a) the subject has received low-dose weekly cyclophosphamide (e.g., ≤500 mg/m²/week), or (b) the subject has received a single cycle of continuous infusion of cyclophosphamide. In some embodiments, T cells are isolated from the subject to manufacture the first BCMA CAR-expressing cell therapy before the subject receives cytotoxic chemotherapy. In some embodiments, the subject has not received autologous or allogeneic stem cell transplantation. In some embodiments, the subject has initiated systemic therapy for multiple myeloma within one year.

In some embodiments, the subject shows beta-2-microglobulin ≥5.5 mg/L and high-risk FISH features: deletion 17p, t(14;16), t(14;20), t(4;14). In some embodiments, the subject shows beta-2-microglobulin ≥5.5 mg/L and LDH greater than upper limit of normal. In some embodiments, the subject shows metaphase karyotype with >3 structural abnormalities except hyperdiploidy. In some embodiments, the subject has plasma cell leukemia, e.g., the subject shows >20% plasma cells in peripheral blood. In some embodiments, the subject fails to achieve a partial response or better (e.g., based on IMWG 2016 criteria, e.g., as described in Table 5) to an Imid/PI combination (thalidomide, lenalidomide, or pomalidomide in combination with bortezomib, ixazomib, or carfilzomib). In some embodiments, the subject progresses on a first-line therapy with an Imid/PI combination within one year (e.g., within six months) of starting the first-line therapy; or within six months of completing the first-line therapy.

In one aspect, disclosed herein is a method of treating a subject having a disease associated with the expression of BCMA, comprising administering to the subject a first BCMA CAR-expressing cell therapy, wherein the subject has high-risk multiple myeloma. In one aspect, disclosed herein is a method of treating a subject having a disease associated with the expression of BCMA, comprising administering to the subject a first BCMA CAR-expressing cell therapy, wherein the subject's multiple myeloma has relapsed after or has been refractory to at least two regimens, e.g., a proteasome inhibitor and/or thalidomide or its analog (e.g., thalidomide, lenalidomide, or pomalidomide). In one aspect, disclosed herein is a method of treating a subject having a disease associated with the expression of BCMA, comprising administering to the subject a first BCMA CAR-expressing cell therapy, wherein the subject has shown at least a minimal response, e.g., the subject has shown a very good partial response, a partial response, or a minimal response, to a most recent therapy received by the subject (e.g., a third-line therapy, e.g., a salvage therapy, e.g., a standard salvage therapy), e.g., based on IMWG 2016 criteria, e.g., as described in Table 5.

In one aspect, disclosed herein is a method of treating a subject having a disease associated with the expression of BCMA, comprising administering to the subject a first BCMA CAR-expressing cell therapy, wherein: (i) the subject has high-risk multiple myeloma, (ii) the subject's multiple myeloma has relapsed after or has been refractory to at least two regimens, e.g., a proteasome inhibitor and/or thalidomide or its analog (e.g., thalidomide, lenalidomide, or pomalidomide), and (iii) the subject has shown at least a minimal response, e.g., the subject has shown a very good partial response, a partial response, or a minimal response, to a most recent therapy received by the subject (e.g., a third-line therapy, e.g., a salvage therapy, e.g., a standard salvage therapy), e.g., based on IMWG 2016 criteria, e.g., as described in Table 5, thereby treating the subject.

In some embodiments, the subject has not shown or is not showing a complete response or a stringent complete response to the most recent therapy received by the subject (e.g., a third-line therapy, e.g., a salvage therapy, e.g., a standard salvage therapy), e.g., based on IMWG 2016 criteria, e.g., as described in Table 5. In some embodiments, the subject shows detectable residual disease after receiving the most recent therapy (e.g., a third-line therapy, e.g., a salvage therapy, e.g., a standard salvage therapy).

In some embodiments, the subject has not received an anti-BCMA cell therapy, e.g., a BCMA CAR-expressing cell therapy. In some embodiments, the subject progressed within one year of receiving melphalan and stem cell transplantation (e.g., autologous stem cell transplantation).

In some embodiments, the method further comprises administering to the subject a first CD19 CAR-expressing cell therapy. In some embodiments, the first CD19 CAR-expressing cell therapy is administered prior to, concurrently with, or after the administration of the first BCMA CAR-expressing cell therapy. In some embodiments, the first CD19 CAR-expressing cell therapy is administered on the same day as the first BCMA CAR-expressing cell therapy, optionally wherein the first CD19 CAR-expressing cell therapy is administered at least one hour after the completion of the administration of the first BCMA CAR-expressing cell therapy. In some embodiments, the first CD19 CAR-expressing cell therapy is administered after the first BCMA CAR-expressing cell therapy, wherein if the subject develops acute infusion reaction after the administration of the first BCMA CAR-expressing cell therapy, the first CD19 CAR-expressing cell therapy is administered up to 48 hours (e.g., 24, 36, or 48 hours) after the administration of the first BCMA CAR-expressing cell therapy.

In some embodiments, the first BCMA CAR-expressing cell therapy is administered in a single infusion or a split-dose infusion. In some embodiments, the first BCMA CAR-expressing cell therapy is administered in a single infusion. In some embodiments, the first BCMA CAR-expressing cell therapy is administered in a split-dose infusion, e.g., wherein the subject receives about 10% of a total dose on a first infusion date, about 30% of a total dose on a second infusion date, and about 60% of a total dose on a third infusion date. In some embodiments, the first BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells, e.g., in a single infusion, e.g., intravenously.

In some embodiments, the first CD19 CAR-expressing cell therapy is administered in a single infusion or a split-dose infusion. In some embodiments, the first CD19 CAR-expressing cell therapy is administered in a single infusion. In some embodiments, the first CD19 CAR-expressing cell therapy is administered in a split-dose infusion, e.g., wherein the subject receives about 10% of a total dose on a first infusion date, about 30% of a total dose on a second infusion date, and about 60% of a total dose on a third infusion date. In some embodiments, the first CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells, e.g., in a single infusion, e.g., intravenously.

In some embodiments, the method further comprises administering to the subject a first conditioning agent (e.g., a lymphodepletion agent, e.g., a lymphodepleting chemotherapy, e.g., cyclophosphamide and/or fludarabine) before administering the first BCMA CAR-expressing cell therapy and/or the first CD19 CAR-expressing cell therapy. In some embodiments, the method comprises administering to the subject cyclophosphamide and fludarabine before administering the first BCMA CAR-expressing cell therapy and/or the first CD19 CAR-expressing cell therapy. In some embodiments, cyclophosphamide is administered at 300 mg/m² intravenously daily for three days. In some embodiments, fludarabine is administered at 30 mg/m² intravenously daily for three days. In some embodiments, the first BCMA CAR-expressing cell therapy and/or the first CD19 CAR-expressing cell therapy are administered 2, 3, or 4 days, e.g., 3 days, after the administration of the first conditioning agent is completed (e.g., after the administration of a last dose of the lymphodepletion agent, e.g., a last dose of the lymphodepleting chemotherapy, e.g., a last dose of cyclophosphamide and/or fludarabine). In some embodiments, the method further comprises, prior to the administration of the first conditioning agent (e.g., the lymphodepletion agent, e.g., the lymphodepleting chemotherapy, e.g., cyclophosphamide and/or fludarabine), obtaining a first sample (e.g., an apheresis sample) from the subject and manufacturing the first BCMA CAR-expressing cell therapy and/or the first CD19 CAR-expressing cell therapy using the sample. In some embodiments, the method further comprises, prior to the administration of the first conditioning agent (e.g., the lymphodepletion agent, e.g., the lymphodepleting chemotherapy, e.g., cyclophosphamide and/or fludarabine) and after obtaining the first sample, obtaining a second sample (e.g., stem cells) from the subject for preparing autologous stem cell transplantation.

In some embodiments, the method further comprises administering to the subject a maintenance agent (e.g., lenalidomide) after the administration of the first BCMA CAR-expressing cell therapy and/or the first CD19 CAR-expressing cell therapy, e.g., at the later of: (i) 26, 27, 28, 29, 30, 31, or 32 days, e.g., 28 days, after the administration of the first BCMA CAR-expressing cell therapy and/or the first CD19 CAR-expressing cell therapy; or (ii) resolution of grade ≤2 of treatment-related toxicity.

In some embodiments, the method further comprises administering to the subject a second BCMA CAR-expressing cell therapy after the administration of the maintenance agent, wherein 80-100 days (e.g., 90 days) have elapsed since the administration of the first BCMA CAR-expressing cell therapy. In some embodiments, the method further comprises administering to the subject a second BCMA CAR-expressing cell therapy after the administration of the maintenance agent, wherein the subject's multiple myeloma has progressed after the administration of the first BCMA CAR-expressing cell therapy. In some embodiments, the method further comprises administering to the subject a second BCMA CAR-expressing cell therapy after the administration of the maintenance agent, wherein the subject has exhibited or is exhibiting objective evidence of residual multiple myeloma after the administration of the first BCMA CAR-expressing cell therapy.

In some embodiments, the method further comprises administering to the subject a second CD19 CAR-expressing cell therapy after the administration of the maintenance agent, wherein >3% peripheral blood lymphocytes of the subject are CD19+ after the administration of the first CD19 CAR-expressing cell therapy, e.g., 7-28 days after the administration of the first CD19 CAR-expressing cell therapy. In some embodiments, the second CD19 CAR-expressing cell therapy is administered prior to, concurrently with, or after the administration of the second BCMA CAR-expressing cell therapy. In some embodiments, the second CD19 CAR-expressing cell therapy is administered on the same day as the second BCMA CAR-expressing cell therapy, optionally wherein the second CD19 CAR-expressing cell therapy is administered at least one hour after the completion of the administration of the second BCMA CAR-expressing cell therapy. In some embodiments, the second CD19 CAR-expressing cell therapy is administered after the second BCMA CAR-expressing cell therapy, wherein if the subject develops acute infusion reaction after the administration of the second BCMA CAR-expressing cell therapy, the second CD19 CAR-expressing cell therapy is administered up to 48 hours (e.g., 24, 36, or 48 hours) after the administration of the second BCMA CAR-expressing cell therapy.

In some embodiments, the second BCMA CAR-expressing cell therapy is administered in a single infusion or a split-dose infusion. In some embodiments, the second BCMA CAR-expressing cell therapy is administered in a single infusion. In some embodiments, the second BCMA CAR-expressing cell therapy is administered in a split-dose infusion, e.g., wherein the subject receives about 10% of a total dose on a first infusion date, about 30% of a total dose on a second infusion date, and about 60% of a total dose on a third infusion date. In some embodiments, the second BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the second CD19 CAR-expressing cell therapy is administered in a single infusion or a split-dose infusion. In some embodiments, the second CD19 CAR-expressing cell therapy is administered in a single infusion. In some embodiments, the second CD19 CAR-expressing cell therapy is administered in a split-dose infusion, e.g., wherein the subject receives about 10% of a total dose on a first infusion date, about 30% of a total dose on a second infusion date, and about 60% of a total dose on a third infusion date. In some embodiments, the second CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

In some embodiments, the second BCMA CAR-expressing cell therapy is the same as the first BCMA CAR-expressing cell therapy. In some embodiments, the second CD19 CAR-expressing cell therapy is the same as the first CD19 CAR-expressing cell therapy.

In some embodiments, the method further comprises administering to the subject a second conditioning agent (e.g., a lymphodepletion agent, e.g., a lymphodepleting chemotherapy, e.g., cyclophosphamide and/or fludarabine) before administering the second BCMA CAR-expressing cell therapy and/or the second CD19 CAR-expressing cell therapy. In some embodiments, the method comprises administering to the subject cyclophosphamide and fludarabine before administering the second BCMA CAR-expressing cell therapy and/or the second CD19 CAR-expressing cell therapy. In some embodiments, cyclophosphamide is administered at 300 mg/m² intravenously daily for three days. In some embodiments, fludarabine is administered at 30 mg/m² intravenously daily for three days. In some embodiments, the method comprises administering to the subject cyclophosphamide, e.g., at 1.5 g/m², before administering the second BCMA CAR-expressing cell therapy and/or the second CD19 CAR-expressing cell therapy.

In some embodiments of the aforementioned methods, the first or second BCMA CAR-expressing cell therapy comprises a cell (e.g., a population of cells) expressing a BCAM CAR, wherein:

(i) the BCMA CAR comprises one or more of (e.g., all three of) heavy chain complementary determining region 1 (HCDR1), HCDR2, and HCDR3 of any BCMA scFv domain amino acid sequence listed in Table 2 or 3 and/or one or more of (e.g., all three of) light chain complementary determining region 1 (LCDR1), LCDR2, and LCDR3 of any BCMA scFv domain amino acid sequence listed in Table 2 or 3, or a sequence with 95-99% identity thereof;

(ii) the BCMA CAR comprises a heavy chain variable region (VH) listed in Table 2 or 3 and/or a light chain variable region (VL) listed in Table 2 or 3, or a sequence with 95-99% identity thereof;

(iii) the BCMA CAR comprises a BCMA scFv domain amino acid sequence listed in Table 2 or 3 (e.g., SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, and SEQ ID NO: 149), or a sequence with 95-99% identity thereof;

(iv) the BCMA CAR comprises a full-length BCMA CAR amino acid sequence listed in Table 2 or 3 (e.g., the amino acid sequence of the immature BCMA CAR comprises the amino acid sequence of SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, and SEQ ID NO: 233), or a sequence with 95-99% identity thereof; or

(v) the BCMA CAR is encoded by a nucleic acid sequence listed in Table 2 or 3 (e.g., SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170), or a sequence with 95-99% identity thereof.

In some embodiments of the aforementioned methods, the first or second CD19 CAR-expressing cell therapy comprises a cell (e.g., a population of cells) expressing a CD19 CAR, wherein:

(i) the CD19 CAR comprises one or more of (e.g., all three of) heavy chain complementary determining region 1 (HCDR1), HCDR2, and HCDR3 listed in Table 6 or 7 and/or one or more of (e.g., all three of) light chain complementary determining region 1 (LCDR1), LCDR2, and LCDR3 listed in Table 6 or 8, or a sequence with 95-99% identity thereof;

(ii) the CD19 CAR comprises a heavy chain variable region (VH) of any CD19 scFv domain amino acid sequence listed in Table 6 and/or a light chain variable region (VL) of any CD19 scFv domain amino acid sequence listed in Table 6, or a sequence with 95-99% identity thereof;

(iii) the CD19 CAR comprises a CD19 scFv domain amino acid sequence listed in Table 6, or a sequence with 95-99% identity thereof;

(iv) the CD19 CAR comprises a full-length CD19 CAR amino acid sequence listed in Table 6, or a sequence with 95-99% identity thereof; or

(v) the CD19 CAR is encoded by a nucleic acid sequence listed in Table 6, or a sequence with 95-99% identity thereof.

In some embodiments of the aforementioned methods, the subject is a human patient.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

Other features, objects, and advantages of the invention will be apparent from the description and drawings, and from the claims.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a clinical trial schematic. PR=partial response. Cy/Flu=cyclophosphamide+fludarabine.

### DESCRIPTION

### Definitions

Unless defined otherwise, all technical and scientific terms used herein have the same meaning as commonly understood by one of ordinary skill in the art to which the invention pertains.

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for mataining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). A second antisense transcript derived from the BCMA locus has been described, which may play a role in regulating BCMA expression. (Laabi Y. et al., Nucleic Acids Res., 1994, 22:1147-1154). Additional transcript variants have been described with unknown significance (Smirnova A S et al. Mol Immunol., 2008, 45(4): 1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. Other cells with express CD19 are provided below in the definition of “disease associated with expression of CD19.” It is also an early marker of B cell progenitors. See, e.g., Nicholson et al. Mol. Immun 34 (16-17): 1157-1165 (1997). In one aspect the antigen-binding portion of the CART recognizes and binds an antigen within the extracellular domain of the CD19 protein. In one aspect, the CD19 protein is expressed on a cancer cell.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4 1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMACAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et at, 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises an scFv.

As used herein, the term “binding domain” or “antibody molecule” (also referred to herein as “anti-target (e.g., BCMA) binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival. The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration”or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of BCMA” includes, but is not limited to, a disease associated with a cell which expresses BCMA (e.g., wild-type or mutant BCMA) or condition associated with a cell which expresses BCMA (e.g., wild-type or mutant BCMA) including, e.g., proliferative diseases such as a cancer or malignancy or a precancerous condition such as a myelodysplasia, a myelodysplastic syndrome or a preleukemia; or a noncancer related indication associated with a cell which expresses BCMA (e.g., wild-type or mutant BCMA). For the avoidance of doubt, a disease associated with expression of BCMA may include a condition associated with a cell which does not presently express BCMA, e.g., because BCMA expression has been downregulated, e.g., due to treatment with a molecule targeting BCMA, e.g., a BCMA inhibitor described herein, but which at one time expressed BCMA. In one aspect, a cancer associated with expression of BCMA (e.g., wild-type or mutant BCMA) is a hematological cancer. In one aspect, the hematological cancer is a leukemia or a lymphoma. In one aspect, a cancer associated with expression of BCMA (e.g., wild-type or mutant BCMA) is a malignancy of differentiated plasma B cells. In one aspect, a cancer associated with expression of BCMA(e.g., wild-type or mutant BCMA) includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of BMCA (e.g., wild-type or mutant BCMA) comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma. In embodiments, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). Further diseases associated with expression of BCMA (e.g., wild-type or mutant BCMA) expression include, but not limited to, e.g., atypical and/or non-classical cancers, malignancies, precancerous conditions or proliferative diseases associated with expression of BCMA (e.g., wild-type or mutant BCMA), e.g., a cancer described herein, e.g., a prostate cancer (e.g., castrate-resistant or therapy-resistant prostate cancer, or metastatic prostate cancer), pancreatic cancer, or lung cancer.

Non-cancer related conditions that are associated with BCMA (e.g., wild-type or mutant BCMA) include viral infections; e.g., HIV, fungal infections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important. In embodiments, a non-cancer related indication associated with expression of BCMA includes but is not limited to, e.g., autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, e.g., a primary signaling sequence of CD3-zeta.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In one embodiment, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In one embodiment, the “zeta stimulatory domain” or a “CD3-zeta stimulatory domain” is the sequence provided as SEQ ID NO: 18 or 20 or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In one embodiment, the “4-1BB costimulatory domain” is the sequence provided as SEQ ID NO: 14 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10 (SEQ ID NO: 28). In one embodiment, the flexible polypeptide linkers include, but are not limited to, (Gly4 Ser)4 (SEQ ID NO: 29) or (Gly4 Ser)3 (SEQ ID NO: 30). In another embodiment, the linkers include multiple repeats of (Gly2Ser), (GlySer) or (Gly3Ser) (SEQ ID NO: 31). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 32), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjuction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative immune effector cells (e.g., T cells or NK cells)/PD-1 positive immune effector cells (e.g., T cells or NK cells).

In an embodiment, the low, immune enhancing, dose of mTOR inhibitor results in an increase in the number of naive T cells. In an embodiment, the low, immune enhancing, dose of mTOR inhibitor results in one or more of the following:

an increase in the expression of one or more of the following markers: CD62Lhigh, CD127high, CD27+, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62Lhigh, increased CD127high, increased CD27+, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3¹ Edition, Cambridge University Press, Cambridge, 1987.

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂ alkyl, C₁-C₁₀ alkyl, and C₁-C₆ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” as used herein refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH═CH₂.

The term “aryl” as used herein refers to a monocyclic, bicyclic or polycyclic hydrocarbon ring system, wherein at least one ring is aromatic. Representative aryl groups include fully aromatic ring systems, such as phenyl (e.g., (C₆) aryl), naphthyl (e.g., (C₁₀) aryl), and anthracenyl (e.g., (C₁₄) aryl), and ring systems where an aromatic carbon ring is fused to one or more non-aromatic carbon rings, such as indanyl, phthalimidyl, naphthimidyl, or tetrahydronaphthyl, and the like.

The term “carbocyclyl” as used herein refers to monocyclic, or fused, spiro-fused, and/or bridged bicyclic or polycyclic hydrocarbon ring system containing 3-18 carbon atoms, wherein each ring is either completely saturated or contains one or more units of unsaturation, but where no ring is aromatic. Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like).

The term “carbonyl” as used herein refers to —C═O.

The term “cyano” as used herein refers to —CN.

The terms “halo” or “halogen” as used herein refer to fluorine (fluoro, —F), chlorine (chloro, —Cl), bromine (bromo, —Br), or iodine (iodo, —I).

The term “heteroalkyl” as used herein refers to a monovalent saturated straight or branched alkyl chain wherein at least one carbon atom in the chain is replaced with a heteroatom, such as O, S, or N, provided that upon substitution, the chain comprises at least one carbon atom. In some embodiments, a heteroalkyl group may comprise, e.g., 1-12, 1-10, or 1-6 carbon atoms, referred to herein as C₁-C₁₂ heteroalkyl, C₁-C₁₀ heteroalkyl, and C₁-C₆heteroalkyl. In certain instances, a heteroalkyl group comprises 1, 2, 3, or 4 independently selected heteroatoms in place of 1, 2, 3, or 4 individual carbon atoms in the alkyl chain. Representative heteroalkyl groups include —CH₂NHC(O)CH₃, —CH₂CH₂OCH₃, —CH₂CH₂NHCH₃, —CH₂CH₂N(CH₃)CH₃, and the like.

The term “heteroaryl” as used herein refers to a monocyclic, bicyclic or polycyclic ring system wherein at least one ring is both aromatic and comprises a heteroatom; and wherein no other rings are heterocyclyl (as defined below). Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, thiazolo-[4,5-c]-pyridinyl, 4,5,6,7-tetrahydrothieno[2,3-c]pyridinyl, 5,6-dihydro-4H-thieno[2,3-c]pyrrolyl, 4,5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

The term “heterocyclyl” as used herein refers to a monocyclic, or fused, spiro-fused, and/or bridged bicyclic and polycyclic ring systems where at least one ring is saturated or partially unsaturated (but not aromatic) and comprises a heteroatom. A heterocyclyl can be attached to its pendant group at any heteroatom or carbon atom that results in a stable structure and any of the ring atoms can be optionally substituted. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl; and (iii) at least one ring is non-aromatic and comprises a heteroatom and at least one other ring is aromatic and comprises a heteroatom, e.g., 3,4-dihydro-1H-pyrano[4,3-c]pyridinyl, and 1,2,3,4-tetrahydro-2,6-naphthyridinyl. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “oxo” as used herein refers to ═O.

The term “thiocarbonyl” as used herein refers to C═S.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), Formula (I-a), and/or Formula (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

It is to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “tautomers” refer to compounds that are interchangeable forms of a particular compound structure, and that vary in the displacement of hydrogen atoms and electrons. Thus, two structures may be in equilibrium through the movement of 7t electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E.L. Eliel, Ed., Univ. of Notre Dame Press, Notre Dame, Ind. 1972).

Various aspects of the compositions and methods herein are described in further detail below. Additional definitions are set out throughout the specification.

### DETAILED DESCRIPTION

The present invention provides, at least in part, a method of treating a subject having a disease associated with BCMA expression, comprising administering to the subject an effective amount of a cell (e.g., a population of cells) that expresses a CAR molecule that binds BCMA (a “BCMA CAR-expressing cell”). In some embodiments, the disease associated with expression of BCMA is a hematologic cancer, e.g., ALL, CLL, DLBCL, or multiple myeloma. In some embodiments, the subject has stage III high-risk multiple myeloma (e.g., stage III high-risk multiple myeloma based on Revised International Staging System), thereby treating the subject. In some embodiments, the BCMA CAR-expressing cell therapy is administered based on the acquisition of a level of a biomarker from a patient sample. In some embodiments, the BCMA CAR-expressing cell therapy is administered to the subject in combination with a second therapy. In some embodiments, the BCMA CAR-expressing cell therapy and the second therapy are administered simultaneously or sequentially. In some embodiments, the second therapy is a CD19 CAR-expressing cell therapy.

### Chimeric Antigen Receptor (CAR)

In one aspect, disclosed herein are methods using a cell (e.g., a population of cells) that expresses a CAR molecule. In one aspect, an exemplary CAR construct comprises an optional leader sequence (e.g., a leader sequence described herein), an antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

Sequences of non-limiting examples of various components that can be part of a CAR molecule described herein, are listed in Table 1, where “aa” stands for amino acids, and “na” stands for nucleic acids that encode the corresponding peptide.

TABLE 1
Sequences of various components of CAR (aa-amino acid sequence, na-nucleic acid sequence).

| SEQ ID | | |
|---|---|---|
| NO: | description | Sequence |
| SEQ ID | EF-1 promoter | CGTGAGGCTCCGGTGCCCGTCAGTGGGCAGAGCGCACATCGCCCA |
| NO: 1 | | CAGTCCCCGAGAAGTTGGGGGGAGGGGTCGGCAATTGAACCGGT |
| | | GCCTAGAGAAGGTGGCGCGGGGTAAACTGGGAAAGTGATGTCGT |
| | | GTACTGGCTCCGCCTTTTTCCCGAGGGTGGGGGAGAACCGTATAT |
| | | AAGTGCAGTAGTCGCCGTGAACGTTCTTTTTCGCAACGGGTTTGCC |
| | | GCCAGAACACAGGTAAGTGCCGTGTGTGGTTCCCGCGGGCCTGGC |
| | | CTCTTTACGGGTTATGGCCCTTGCGTGCCTTGAATTACTTCCACCT |
| | | GGCTGCAGTACGTGATTCTTGATCCCGAGCTTCGGGTTGGAAGTG |
| | | GGTGGGAGAGTTCGAGGCCTTGCGCTTAAGGAGCCCCTTCGCCTC |
| | | GTGCTTGAGTTGAGGCCTGGCCTGGGCGCTGGGGCCGCCGCGTGC |
| | | GAATCTGGTGGCACCTTCGCGCCTGTCTCGCTGCTTTCGATAAGTC |
| | | TCTAGCCATTTAAAATTTTTGATGACCTGCTGCGACGCTTTTTTTCT |
| | | GGCAAGATAGTCTTGTAAATGCGGGCCAAGATCTGCACACTGGTA |
| | | TTTCGGTTTTTGGGGCCGCGGGCGGCGACGGGGCCCGTGCGTCCC |
| | | AGCGCACATGTTCGGCGAGGCGGGGCCTGCGAGCGCGGCCACCG |
| | | AGAATCGGACGGGGGTAGTCTCAAGCTGGCCGGCCTGCTCTGGTG |
| | | CCTGGCCTCGCGCCGCCGTGTATCGCCCCGCCCTGGGCGGCAAGG |
| | | CTGGCCCGGTCGGCACCAGTTGCGTGAGCGGAAAGATGGCCGCTT |
| | | CCCGGCCCTGCTGCAGGGAGCTCAAAATGGAGGACGCGGCGCTCG |
| | | GGAGAGCGGGCGGGTGAGTCACCCACACAAAGGAAAAGGGCCTT |
| | | TCCGTCCTCAGCCGTCGCTTCATGTGACTCCACGGAGTACCGGGCG |
| | | CCGTCCAGGCACCTCGATTAGTTCTCGAGCTTTTGGAGTACGTCGT |
| | | CTTTAGGTTGGGGGGAGGGGTTTTATGCGATGGAGTTTCCCCACA |
| | | CTGAGTGGGTGGAGACTGAAGTTAGGCCAGCTTGGCACTTGATGT |
| | | AATTCTCCTTGGAATTTGCCCTTTTTGAGTTTGGATCTTGGTTCATT |
| | | CTCAAGCCTCAGACAGTGGTTCAAAGTTTTTTTCTTCCATTTCAGG |
| | | TGTCGTGA |
| SEQ ID | Leader (aa) | MALPVTALLLPLALLLHAARP |
| NO: 2 | | |
| SEQ ID | Leader (na) | ATGGCCCTGCCTGTGACAGCCCTGCTGCTGCCTCTGGCTCTGCTGC |
| NO: 3 | | TGCATGCCGCTAGACCC |
| SEQ ID | Leader (na) | ATGGCCCTCCCTGTCACCGCCCTGCTGCTTCCGCTGGCTCTTCTGC |
| NO: 33 | | TCCACGCCGCTCGGCCC |
| SEQ ID | CD 8 hinge | TTTPAPRPPTPAPTIASQPLSLRPEACRPAAGGAVHTRGLDFACD |
| NO: 4 | (aa) | |
| SEQ ID | CD8 hinge | ACCACGACGCCAGCGCCGCGACCACCAACACCGGCGCCCACCATC |
| NO: 5 | (na) | GCGTCGCAGCCCCTGTCCCTGCGCCCAGAGGCGTGCCGGCCAGCG |
| | | GCGGGGGGCGCAGTGCACACGAGGGGGCTGGACTTCGCCTGTGAT |
| SEQ ID | Ig4 hinge (aa) | ESKYGPPCPPCPAPEFLGGPSVFLFPPKPKDTLMISRTPEVTCVVVDVS |
| NO: 6 | | QEDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFL |
| | | YSRLTVDKSRWQEGNVFSCSVMHEALHNHYTQKSLSLSLGKM |
| SEQ ID | Ig4 hinge (na) | GAGAGCAAGTACGGCCCTCCCTGCCCCCCTTGCCCTGCCCCCGAG |
| NO: 7 | | TTCCTGGGCGGACCCAGCGTGTTCCTGTTCCCCCCCAAGCCCAAGG |
| | | ACACCCTGATGATCAGCCGGACCCCCGAGGTGACCTGTGTGGTGG |
| | | TGGACGTGTCCCAGGAGGACCCCGAGGTCCAGTTCAACTGGTACG |
| | | TGGACGGCGTGGAGGTGCACAACGCCAAGACCAAGCCCCGGGAG |
| | | GAGCAGTTCAATAGCACCTACCGGGTGGTGTCCGTGCTGACCGTG |
| | | CTGCACCAGGACTGGCTGAACGGCAAGGAATACAAGTGTAAGGT |
| | | GTCCAACAAGGGCCTGCCCAGCAGCATCGAGAAAACCATCAGCA |
| | | AGGCCAAGGGCCAGCCTCGGGAGCCCCAGGTGTACACCCTGCCCC |
| | | CTAGCCAAGAGGAGATGACCAAGAACCAGGTGTCCCTGACCTGCC |
| | | TGGTGAAGGGCTTCTACCCCAGCGACATCGCCGTGGAGTGGGAGA |
| | | GCAACGGCCAGCCCGAGAACAACTACAAGACCACCCCCCCTGTGC |
| | | TGGACAGCGACGGCAGCTTCTTCCTGTACAGCCGGCTGACCGTGG |
| | | ACAAGAGCCGGTGGCAGGAGGGCAACGTCTTTAGCTGCTCCGTGA |
| | | TGCACGAGGCCCTGCACAACCACTACACCCAGAAGAGCCTGAGCC |
| | | TGTCCCTGGGCAAGATG |
| SEQ ID | IgD hinge (aa) | RWPESPKAQASSVPTAQPQAEGSLAKATTAPATTRNTGRGGEEKKKE |
| NO: 8 | | KEKEEQEERETKTPECPSHTQPLGVYLLTPAVQDLWLRDKATFTCFV |
| | | VGSDLKDAHLTWEVAGKVPTGGVEEGLLERHSNGSQSQHSRLTLPR |
| | | SLWNAGTSVTCTLNHPSLPPQRLMALREPAAQAPVKLSLNLLASSDP |
| | | PEAASWLLCEVSGFSPPNILLMWLEDQREVNTSGFAPARPPPQPGSTT |
| | | FWAWSVLRVPAPPSPQPATYTCVVSHEDSRTLLNASRSLEVSYVTDH |
| SEQ ID | IgD hinge (na) | AGGTGGCCCGAAAGTCCCAAGGCCCAGGCATCTAGTGTTCCTACT |
| NO: 9 | | GCACAGCCCCAGGCAGAAGGCAGCCTAGCCAAAGCTACTACTGCA |
| | | CCTGCCACTACGCGCAATACTGGCCGTGGCGGGGAGGAGAAGAA |
| | | AAAGGAGAAAGAGAAAGAAGAACAGGAAGAGAGGGAGACCAAG |
| | | ACCCCTGAATGTCCATCCCATACCCAGCCGCTGGGCGTCTATCTCT |
| | | TGACTCCCGCAGTACAGGACTTGTGGCTTAGAGATAAGGCCACCT |
| | | TTACATGTTTCGTCGTGGGCTCTGACCTGAAGGATGCCCATTTGAC |
| | | TTGGGAGGTTGCCGGAAAGGTACCCACAGGGGGGGTTGAGGAAG |
| | | GGTTGCTGGAGCGCCATTCCAATGGCTCTCAGAGCCAGCACTCAA |
| | | GACTCACCCTTCCGAGATCCCTGTGGAACGCCGGGACCTCTGTCA |
| | | CATGTACTCTAAATCATCCTAGCCTGCCCCCACAGCGTCTGATGGC |
| | | CCTTAGAGAGCCAGCCGCCCAGGCACCAGTTAAGCTTAGCCTGAA |
| | | TCTGCTCGCCAGTAGTGATCCCCCAGAGGCCGCCAGCTGGCTCTTA |
| | | TGCGAAGTGTCCGGCTTTAGCCCGCCCAACATCTTGCTCATGTGGC |
| | | TGGAGGACCAGCGAGAAGTGAACACCAGCGGCTTCGCTCCAGCCC |
| | | GGCCCCCACCCCAGCCGGGTTCTACCACATTCTGGGCCTGGAGTG |
| | | TCTTAAGGGTCCCAGCACCACCTAGCCCCCAGCCAGCCACATACA |
| | | CCTGTGTTGTGTCCCATGAAGATAGCAGGACCCTGCTAAATGCTTC |
| | | TAGGAGTCTGGAGGTTTCCTACGTGACTGACCATT |
| SEQ ID | GS | GGGGSGGGGS |
| NO: 10 | hinge/linker | |
| | (aa) | |
| SEQ ID | GS | GGTGGCGGAGGTTCTGGAGGTGGAGGTTCC |
| NO: 11 | hinge/linker | |
| | (na) | |
| SEQ ID | CD8TM (aa) | IYIWAPLAGTCGVLLLSLVITLYC |
| NO: 12 | | |
| SEQ ID | CD8 TM (na) | ATCTACATCTGGGCGCCCTTGGCCGGGACTTGTGGGGTCCTTCTCC |
| NO: 13 | | TGTCACTGGTTATCACCCTTTACTGC |
| SEQ ID | CD8 TM (na) | ATCTACATTTGGGCCCCTCTGGCTGGTACTTGCGGGGTCCTGCTGC |
| NO: 34 | | TTTCACTCGTGATCACTCTTTACTGT |
| SEQ ID | 4-1BB | KRGRKKLLYIFKQPFMRPVQTTQEEDGCSCRFPEEEEGGCEL |
| NO: 14 | intracellular | |
| | domain (aa) | |
| SEQ ID | 4-1BB | AAACGGGGCAGAAAGAAACTCCTGTATATATTCAAACAACCATTT |
| NO: 15 | intracellular | ATGAGACCAGTACAAACTACTCAAGAGGAAGATGGCTGTAGCTGC |
| | domain (na) | CGATTTCCAGAAGAAGAAGAAGGAGGATGTGAACTG |
| SEQ ID | 4-1BB | AAGCGCGGTCGGAAGAAGCTGCTGTACATCTTTAAGCAACCCTTC |
| NO: 35 | intracellular | ATGAGGCCTGTGCAGACTACTCAAGAGGAGGACGGCTGTTCATGC |
| | domain (na) | CGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPEPACSP |
| NO: 16 | | |
| SEQ ID | CD27 (na) | AGGAGTAAGAGGAGCAGGCTCCTGCACAGTGACTACATGAACAT |
| NO: 17 | | GACTCCCCGCCGCCCCGGGCCCACCCGCAAGCATTACCAGCCCTA |
| | | TGCCCCACCACGCGACTTCGCAGCCTATCGCTCC |
| SEQ ID | CD3-zeta (aa) | RVKFSRSADAPAYKQGQNQLYNELNLGRREEYDVLDKRRGRDPEM |
| NO: 18 | | GGKPRRKNPQEGLYNELQKDKMAEAYSEIGMKGERRRGKGHDGLY |
| | | QGLSTATKDTYDALHMQALPPR |
| SEQ ID | CD3-zeta (na) | AGAGTGAAGTTCAGCAGGAGCGCAGACGCCCCCGCGTACAAGCA |
| NO: 19 | | GGGCCAGAACCAGCTCTATAACGAGCTCAATCTAGGACGAAGAG |
| | | AGGAGTACGATGTTTTGGACAAGAGACGTGGCCGGGACCCTGAGA |
| | | TGGGGGGAAAGCCGAGAAGGAAGAACCCTCAGGAAGGCCTGTAC |
| | | AATGAACTGCAGAAAGATAAGATGGCGGAGGCCTACAGTGAGAT |
| | | TGGGATGAAAGGCGAGCGCCGGAGGGGCAAGGGGCACGATGGCC |
| | | TTTACCAGGGTCTCAGTACAGCCACCAAGGACACCTACGACGCCC |
| | | TTCACATGCAGGCCCTGCCCCCTCGC |
| SEQ ID | CD3-zeta (na) | CGCGTGAAATTCAGCCGCAGCGCAGATGCTCCAGCCTACAAGCAG |
| NO: 36 | | GGGCAGAACCAGCTCTACAACGAACTCAATCTTGGTCGGAGAGAG |
| | | GAGTACGACGTGCTGGACAAGCGGAGAGGACGGGACCCAGAAAT |
| | | GGGCGGGAAGCCGCGCAGAAAGAATCCCCAAGAGGGCCTGTACA |
| | | ACGAGCTCCAAAAGGATAAGATGGCAGAAGCCTATAGCGAGATT |
| | | GGTATGAAAGGGGAACGCAGAAGAGGCAAAGGCCACGACGGACT |
| | | GTACCAGGGACTCAGCACCGCCACCAAGGACACCTATGACGCTCT |
| | | TCACATGCAGGCCCTGCCGCCTCGG |
| SEQ ID | CD3-zeta (aa) | RVKFSRSADAPAYQQGQNQLYNELNLGRREEYDVLDKRRGRDPEM |
| NO: 20 | | GGKPRRKNPQEGLYNELQKDKMAEAYSEIGMKGERRRGKGHDGLY |
| | | QGLSTATKDTYDALHMQALPPR |
| SEQ ID | CD3-zeta (na) | AGAGTGAAGTTCAGCAGGAGCGCAGACGCCCCCGCGTACCAGCA |
| NO: 21 | | GGGCCAGAACCAGCTCTATAACGAGCTCAATCTAGGACGAAGAG |
| | | AGGAGTACGATGTTTTGGACAAGAGACGTGGCCGGGACCCTGAGA |
| | | TGGGGGGAAAGCCGAGAAGGAAGAACCCTCAGGAAGGCCTGTAC |
| | | AATGAACTGCAGAAAGATAAGATGGCGGAGGCCTACAGTGAGAT |
| | | TGGGATGAAAGGCGAGCGCCGGAGGGGCAAGGGGCACGATGGCC |
| | | TTTACCAGGGTCTCAGTACAGCCACCAAGGACACCTACGACGCCC |
| | | TTCACATGCAGGCCCTGCCCCCTCGC |
| SEQ ID | linker | GGGGS |
| NO: 22 | | |
| SEQ ID | linker | GGTGGCGGAGGTTCTGGAGGTGGAGGTTCC |
| NO: 23 | | |
| SEQ ID | PD-1 | Pgwfldspdrpwnpptfspallvvtegdnatftcsfsntsesfvlnwyrmspsnqtdklaafpedrsqpgq |
| NO: 24 | extracellular | dcrfrvtqlpngrdfhmsvvrarrndsgtylcgaislapkaqikeslraelrvterraevptahpspsprpag |
| | domain (aa) | qfqtlv |
| SEQ ID | PD-1 | Cccggatggtttctggactctccggatcgcccgtggaatcccccaaccttctcaccggcactcttggttgtgact |
| NO: 25 | extracellular | gagggcgataatgcgaccttcacgtgctcgttctccaacacctccgaatcattcgtgctgaactggtaccgcat |
| | domain (na) | gagcccgtcaaaccagaccgacaagctcgccgcgtttccggaagatcggtcgcaaccgggacaggattgtc |
| | | ggttccgcgtgactcaactgccgaatggcagagacttccacatgagcgtggtccgcgctaggcgaaacgact |
| | | ccgggacctacctgtgcggagccatctcgctggcgcctaaggcccaaatcaaagagagcttgagggccgaa |
| | | ctgagagtgaccgagcgcagagctgaggtgccaactgcacatccatccccatcgcctcggcctgcggggca |
| | | gtttcagaccctggtc |
| SEQ ID | PD-1 CAR | Malpvtalllplalllhaarppgwfldspdrpwnpptfspallvvtegdnatftcsfsntsesfvlnwyrmsp |
| NO: 26 | (aa) with | snqtdklaafpedrsqpgqdcrfrvtqlpngrdfhmsvvrarrndsgtylcgaislapkaqikeslraelrvt |
| | signal | erraevptahpspsprpagqfqtlvtttpaprpptpaptiasqplslrpeacrpaaggavhtrgldfacdiyiw |
| | | aplagtcgvlllslvitlyckrgrkkllyifkqpfmrpvqttqeedgcscrfpeeeeggcelrvkfsrsadapa |
| | | ykqgqnqlynelnlgrreeydvldlargrdpemggkprrknpqeglynelqkdkmaeayseigmkge |
| | | rrrgkghdglyqglstatkdtydalhmqalppr |
| SEQ ID | PD-1 CAR | Atggccctccctgtcactgccctgcttctccccctcgcactcctgctccacgccgctagaccacccggatggtt |
| NO: 27 | (na) | tctggactctccggatcgcccgtggaatcccccaaccttctcaccggcactcttggttgtgactgagggcgata |
| | | atgcgaccttcacgtgctcgttctccaacacctccgaatcattcgtgctgaactggtaccgcatgagcccgtcaa |
| | | accagaccgacaagctcgccgcgtttccggaagatcggtcgcaaccgggacaggattgtcggttccgcgtg |
| | | actcaactgccgaatggcagagacttccacatgagcgtggtccgcgctaggcgaaacgactccgggaccta |
| | | cctgtgcggagccatctcgctggcgcctaaggcccaaatcaaagagagcttgagggccgaactgagagtga |
| | | ccgagcgcagagctgaggtgccaactgcacatccatccccatcgcctcggcctgcggggcagtttcagacc |
| | | ctggtcacgaccactccggcgccgcgcccaccgactccggccccaactatcgcgagccagcccctgtcgct |
| | | gaggccggaagcatgccgccctgccgccggaggtgctgtgcatacccggggattggacttcgcatgcgaca |
| | | tctacatttgggctcctctcgccggaacttgtggcgtgctccttctgtccctggtcatcaccctgtactgcaagcg |
| | | gggtcggaaaaagcttctgtacattttcaagcagcccttcatgaggcccgtgcaaaccacccaggaggagga |
| | | cggttgctcctgccggttccccgaagaggaagaaggaggttgcgagctgcgcgtgaagttctcccggagcg |
| | | ccgacgcccccgcctataagcagggccagaaccagctgtacaacgaactgaacctgggacggcgggaag |
| | | agtacgatgtgctggacaagcggcgcggccgggaccccgaaatgggcgggaagcctagaagaaagaacc |
| | | ctcaggaaggcctgtataacgagctgcagaaggacaagatggccgaggcctactccgaaattgggatgaag |
| | | ggagagcggcggaggggaaaggggcacgacggcctgtaccaaggactgtccaccgccaccaaggacac |
| | | atacgatgccctgcacatgcaggcccttccccctcgc |
| SEQ ID | linker | (Gly-Gly-Gly-Ser)n, where n = 1-10 |
| NO: 28 | | |
| SEQ ID | linker | (Gly4 Ser)4 |
| NO: 29 | | |
| SEQ ID | linker | (Gly4 Ser)3 |
| NO: 30 | | |
| SEQ ID | linker | (Gly3Ser) |
| NO: 31 | | |
| SEQ ID | polyA | [a]₅₀₋₅₀₀₀ |
| NO: 32 | | |
| SEQ ID | PD1 CAR (aa) | Pgwfldspdrpwnpptfspallvvtegdnatftcsfsntsesfvlnwyrmspsnqtdklaafpedrsqpgq |
| NO: 37 | | dcrfrvtqlpngrdfhmsvvrarrndsgtylcgaislapkaqikeslraelrvterraevptahpspsprpag |
| | | qfqtlvtttpaprpptpaptiasqplslrpeacrpaaggavhtrgldfacdiyiwaplagtcgvlllslvitlyck |
| | | rgrkkllyifkqpfmrpvqttqeedgcscrfpeeeeggcelrvkfsrsadapaykqgqnqlynelnlgrree |
| | | ydvldkrrgrdpemggkprrknpqeglynelqkdkmaeayseigmkgerrrgkghdglyqglstatkd |
| | | tydalhmqalppr |
| SEQ ID | ICOS | TKKKYSSSVHDPNGEYMFMRAVNTAKKSRLTDVTL |
| NO: 38 | intracellular | |
| | domain (aa) | |
| SEQ ID | ICOS | ACAAAAAAGAAGTATTCATCCAGTGTGCACGACCCTAACGGTGAA |
| NO: 271 | intracellular | TACATGTTCATGAGAGCAGTGAACACAGCCAAAAAATCCAGACTC |
| | domain (na) | ACAGATGTGACCCTA |
| SEQ ID | ICOS TM | TTTPAPRPPTPAPTIASQPLSLRPEACRPAAGGAVHTRGLDFACDFWL |
| NO: 272 | domain (aa) | PIGCAAFVVVCILGCILICWL |
| SEQ ID | ICOS TM | ACCACGACGCCAGCGCCGCGACCACCAACACCGGCGCCCACCATC |
| NO: 273 | domain (na) | GCGTCGCAGCCCCTGTCCCTGCGCCCAGAGGCGTGCCGGCCAGCG |
| | | GCGGGGGGCGCAGTGCACACGAGGGGGCTGGACTTCGCCTGTGAT |
| | | TTCTGGTTACCCATAGGATGTGCAGCCTTTGTTGTAGTCTGCATTT |
| | | TGGGATGCATACTTATTTGTTGGCTT |
| SEQ ID | CD28 | RSKRSRLLHSDYMNMTPRRPGPTRKHYQPYAPPRDFAAYRS |
| NO: 274 | intracellular | |
| | domain (aa) | |
| SEQ ID | CD28 | AGGAGTAAGAGGAGCAGGCTCCTGCACAGTGACTACATGAACAT |
| NO: 275 | intracellular | GACTCCCCGCCGCCCCGGGCCCACCCGCAAGCATTACCAGCCCTA |
| | domain (na) | TGCCCCACCACGCGACTTCGCAGCCTATCGCTCC |

### CAR Antigen Binding Domain

In one aspect, the portion of the CAR comprising the antigen binding domain comprises an antigen binding domain that targets a tumor antigen, e.g., a tumor antigen described herein. In some embodiments, the antigen binding domain binds to: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

The antigen binding domain can be any domain that binds to an antigen, including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CAR Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO: 4. In one aspect, the transmembrane domain comprises (e.g., consists of) a transmembrane domain of SEQ ID NO: 12.

In one aspect, the hinge or spacer comprises an IgG4 hinge. For example, in one embodiment, the hinge or spacer comprises a hinge of the amino acid sequence of SEQ ID NO: 6. In some embodiments, the hinge or spacer comprises a hinge encoded by a nucleotide sequence of SEQ ID NO: 7.

In one aspect, the hinge or spacer comprises an IgD hinge. For example, in one embodiment, the hinge or spacer comprises a hinge of the amino acid sequence of SEQ ID NO: 8. In some embodiments, the hinge or spacer comprises a hinge encoded by a nucleotide sequence of SEQ ID NO: 9.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of SEQ ID NO: 10. In some embodiments, the linker is encoded by a nucleotide sequence of SEQ ID NO: 11.

In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 14. In one aspect, the signaling domain of CD3-zeta is a signaling domain of SEQ ID NO: 18.

In one aspect, the intracellular signaling domain is designed to comprise the signaling domain of CD3-zeta and the signaling domain of CD27. In one aspect, the signaling domain of CD27 comprises an amino acid sequence of SEQ ID NO: 16. In one aspect, the signalling domain of CD27 is encoded by a nucleic acid sequence of SEQ ID NO: 17.

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs. For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell. As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGFbeta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

### BCMA CAR

In one aspect, the CAR disclosed herein binds to BCMA. Exemplary BCMA CARs can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In certain embodiments, the domains are contiguous and in the same reading frame to form a single fusion protein. In other embodiments, the domain are in separate polypeptides, e.g., as in an RCAR molecule as described herein.

The sequences of exemplary BCMA CAR molecules or fragments thereof are disclosed in Tables 2 and 3. In certain embodiments, the full length BCMA CAR molecule includes one or more CDRs, VH, VL, scFv, or full-length sequences of, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, as disclosed in Tables 2 and 3, or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

TABLE 2
Amino Acid and Nucleic Acid Sequences of exemplary anti-BCMA scFv domains and
BCMA CAR molecules. The amino acid sequences variable heavy chain and variable
light chain sequences for each scFv is also provided.

| | | |
|---|---|---|
| | SEQ | |
| Name/ | ID | |
| Description | NO: | Sequence |

139109

| | | |
|---|---|---|
| 139109-aa | 49 | EVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIQLTQSPSSLSASVGDRVTITCRASQSISSYLNWYQQKPGK |
| | | APKLLIYAASSLQSGVPSRFSGSGSGTDFTLTISSLQPEDFATYYCQQSYSTPYTFGQ |
| | | GTKVEIK |
| 139109-nt | 64 | GAAGTGCAATTGGTGGAATCAGGGGGAGGACTTGTGCAGCCTGGAGGATCGCTGAGAC |
| ScFv domain | | TGTCATGTGCCGTGTCCGGCTTTGCCCTGTCCAACCACGGGATGTCCTGGGTCCGCCG |
| | | CGCGCCTGGAAAGGGCCTCGAATGGGTGTCGGGTATTGTGTACAGCGGTAGCACCTAC |
| | | TATGCCGCATCCGTGAAGGGGAGATTCACCATCAGCCGGGACAACTCCAGGAACACTC |
| | | TGTACCTCCAAATGAATTCGCTGAGGCCAGAGGACACTGCCATCTACTACTGCTCCGC |
| | | GCATGGCGGAGAGTCCGACGTCTGGGGACAGGGGACCACCGTGACCGTGTCTAGCGCG |
| | | TCCGGCGGAGGCGGCAGCGGGGGTCGGGCATCAGGGGGCGGCGGATCGGACATCCAGC |
| | | TCACCCAGTCCCCGAGCTCGCTGTCCGCCTCCGTGGGAGATCGGGTCACCATCACGTG |
| | | CCGCGCCAGCCAGTCGATTTCCTCCTACCTGAACTGGTACCAACAGAAGCCCGGAAAA |
| | | GCCCCGAAGCTTCTCATCTACGCCGCCTCGAGCCTGCAGTCAGGAGTGCCCTCACGGT |
| | | TCTCCGGCTCCGGTTCCGGTACTGATTTCACCCTGACCATTTCCTCCCTGCAACCGGA |
| | | GGACTTCGCTACTTACTACTGCCAGCAGTCGTACTCCACCCCCTACACTTTCGGACAA |
| | | GGCACCAAGGTCGAAATCAAG |
| 139109-aa | 79 | EVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139109-aa | 94 | DIQLTQSPSSLSASVGDRVTITCRASQSISSYLNWYQQKPGKAPKLLIYAASSLQSGV |
| VL | | PSRFSGSGSGTDFTLTISSLQPEDFATYYCQQSYSTPYTFGQGTKVEIK |
| 139109-aa | 109 | MALPVTALLLPLALLLHAARPEVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIQLTQSPSSLSASVGDRVTI |
| | | TCRASQSISSYLNWYQQKPGKAPKLLIYAASSLQSGVPSRFSGSGSGTDFTLTISSLQ |
| | | PEDFATYYCQQSYSTPYTFGQGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139109-nt | 124 | ATGGCCCTCCCTGTCACCGCCCTGCTGCTTCCGCTGGCTCTTCTGCTCCACGCCGCTC |
| Full CAR | | GGCCCGAAGTGCAATTGGTGGAATCAGGGGGAGGACTTGTGCAGCCTGGAGGATCGCT |
| | | GAGACTGTCATGTGCCGTGTCCGGCTTTGCCCTGTCCAACCACGGGATGTCCTGGGTC |
| | | CGCCGCGCGCCTGGAAAGGGCCTCGAATGGGTGTCGGGTATTGTGTACAGCGGTAGCA |
| | | CCTACTATGCCGCATCCGTGAAGGGGAGATTCACCATCAGCCGGGACAACTCCAGGAA |
| | | CACTCTGTACCTCCAAATGAATTCGCTGAGGCCAGAGGACACTGCCATCTACTACTGC |
| | | TCCGCGCATGGCGGAGAGTCCGACGTCTGGGGACAGGGGACCACCGTGACCGTGTCTA |
| | | GCGCGTCCGGCGGAGGCGGCAGCGGGGGTCGGGCATCAGGGGGCGGCGGATCGGACAT |
| | | CCAGCTCACCCAGTCCCCGAGCTCGCTGTCCGCCTCCGTGGGAGATCGGGTCACCATC |
| | | ACGTGCCGCGCCAGCCAGTCGATTTCCTCCTACCTGAACTGGTACCAACAGAAGCCCG |
| | | GAAAAGCCCCGAAGCTTCTCATCTACGCCGCCTCGAGCCTGCAGTCAGGAGTGCCCTC |
| | | ACGGTTCTCCGGCTCCGGTTCCGGTACTGATTTCACCCTGACCATTTCCTCCCTGCAA |
| | | CCGGAGGACTTCGCTACTTACTACTGCCAGCAGTCGTACTCCACCCCCTACACTTTCG |
| | | GACAAGGCACCAAGGTCGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |

139103

| | | |
|---|---|---|
| 139103-aa | 39 | QVQLVESGGGLVQPGRSLRLSCAASGFTFSNYAMSWVRQAPGKGLGWVSGISRSGENT |
| ScFv domain | | YYADSVKGRFTISRDNSKNTLYLQMNSLRDEDTAVYYCARSPAHYYGGMDVWGQGTTV |
| | | TVSSASGGGGSGGRASGGGGSDIVLTQSPGTLSLSPGERATLSCRASQSISSSFLAWY |
| | | QQKPGQAPRLLIYGASRRATGIPDRFSGSGSGTDFTLTISRLEPEDSAVYYCQQYHSS |
| | | PSWTFGQGTKLEIK |
| 139103-nt | 54 | CAAGTGCAACTCGTGGAATCTGGTGGAGGACTCGTGCAACCCGGAAGATCGCTTAGAC |
| ScFv domain | | TGTCGTGTGCCGCCAGCGGGTTCACTTTCTCGAACTACGCGATGTCCTGGGTCCGCCA |
| | | GGCACCCGGAAAGGGACTCGGTTGGGTGTCCGGCATTTCCCGGTCCGGCGAAAATACC |
| | | TACTACGCCGACTCCGTGAAGGGCCGCTTCACCATCTCAAGGGACAACAGCAAAAACA |
| | | CCCTGTACTTGCAAATGAACTCCCTGCGGGATGAAGATACAGCCGTGTACTATTGCGC |
| | | CCGGTCGCCTGCCCATTACTACGGCGGAATGGACGTCTGGGGACAGGGAACCACTGTG |
| | | ACTGTCAGCAGCGCGTCGGGTGGCGGCGGCTCAGGGGGTCGGGCCTCCGGGGGGGGAG |
| | | GGTCCGACATCGTGCTGACCCAGTCCCCGGGAACCCTGAGCCTGAGCCCGGGAGAGCG |
| | | CGCGACCCTGTCATGCCGGGCATCCCAGAGCATTAGCTCCTCCTTTCTCGCCTGGTAT |
| | | CAGCAGAAGCCCGGACAGGCCCCGAGGCTGCTGATCTACGGCGCTAGCAGAAGGGCTA |
| | | CCGGAATCCCAGACCGGTTCTCCGGCTCCGGTTCCGGGACCGATTTCACCCTTACTAT |
| | | CTCGCGCCTGGAACCTGAGGACTCCGCCGTCTACTACTGCCAGCAGTACCACTCATCC |
| | | CCGTCGTGGACGTTCGGACAGGGCACCAAGCTGGAGATTAAG |
| 139103-aa | 69 | QVQLVESGGGLVQPGRSLRLSCAASGFTFSNYAMSWVRQAPGKGLGWVSGISRSGENT |
| VH | | YYADSVKGRFTISRDNSKNTLYLQMNSLRDEDTAVYYCARSPAHYYGGMDVWGQGTTV |
| | | TVSS |
| 139103-aa | 84 | DIVLTQSPGTLSLSPGERATLSCRASQSISSSFLAWYQQKPGQAPRLLIYGASRRATG |
| VL | | IPDRFSGSGSGTDFTLTISRLEPEDSAVYYCQQYHSSPSWTFGQGTKLEIK |
| 139103-aa | 99 | MALPVTALLLPLALLLHAARPQVQLVESGGGLVQPGRSLRLSCAASGFTFSNYAMSWV |
| Full CAR | | RQAPGKGLGWVSGISRSGENTYYADSVKGRFTISRDNSKNTLYLQMNSLRDEDTAVYY |
| | | CARSPAHYYGGMDVWGQGTTVTVSSASGGGGSGGRASGGGGSDIVLTQSPGTLSLSPG |
| | | ERATLSCRASQSISSSFLAWYQQKPGQAPRLLIYGASRRATGIPDRFSGSGSGTDFTL |
| | | TISRLEPEDSAVYYCQQYHSSPSWTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139103-nt | 114 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGCAACCCGGAAGATCGCT |
| | | TAGACTGTCGTGTGCCGCCAGCGGGTTCACTTTCTCGAACTACGCGATGTCCTGGGTC |
| | | CGCCAGGCACCCGGAAAGGGACTCGGTTGGGTGTCCGGCATTTCCCGGTCCGGCGAAA |
| | | ATACCTACTACGCCGACTCCGTGAAGGGCCGCTTCACCATCTCAAGGGACAACAGCAA |
| | | AAACACCCTGTACTTGCAAATGAACTCCCTGCGGGATGAAGATACAGCCGTGTACTAT |
| | | TGCGCCCGGTCGCCTGCCCATTACTACGGCGGAATGGACGTCTGGGGACAGGGAACCA |
| | | CTGTGACTGTCAGCAGCGCGTCGGGTGGCGGCGGCTCAGGGGGTCGGGCCTCCGGGGG |
| | | GGGAGGGTCCGACATCGTGCTGACCCAGTCCCCGGGAACCCTGAGCCTGAGCCCGGGA |
| | | GAGCGCGCGACCCTGTCATGCCGGGCATCCCAGAGCATTAGCTCCTCCTTTCTCGCCT |
| | | GGTATCAGCAGAAGCCCGGACAGGCCCCGAGGCTGCTGATCTACGGCGCTAGCAGAAG |
| | | GGCTACCGGAATCCCAGACCGGTTCTCCGGCTCCGGTTCCGGGACCGATTTCACCCTT |
| | | ACTATCTCGCGCCTGGAACCTGAGGACTCCGCCGTCTACTACTGCCAGCAGTACCACT |
| | | CATCCCCGTCGTGGACGTTCGGACAGGGCACCAAGCTGGAGATTAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

139105

| | | |
|---|---|---|
| 139105-aa | 40 | QVQLVESGGGLVQPGRSLRLSCAASGFTFDDYAMHWVRQAPGKGLEWVSGISWNSGSI |
| ScFv domain | | GYADSVKGRFTISRDNAKNSLYLQMNSLRAEDTALYYCSVHSFLAYWGQGTLVTVSSA |
| | | SGGGGSGGRASGGGGSDIVMTQTPLSLPVTPGEPASISCRSSQSLLHSNGYNYLDWYL |
| | | QKPGQSPQLLIYLGSNRASGVPDRFSGSGSGTDFTLKISRVEAEDVGVYYCMQALQTP |
| | | YTFGQGTKVEIK |
| 139105-nt | 55 | CAAGTGCAACTCGTCGAATCCGGTGGAGGTCTGGTCCAACCTGGTAGAAGCCTGAGAC |
| ScFv domain | | TGTCGTGTGCGGCCAGCGGATTCACCTTTGATGACTATGCTATGCACTGGGTGCGGCA |
| | | GGCCCCAGGAAAGGGCCTGGAATGGGTGTCGGGAATTAGCTGGAACTCCGGGTCCATT |
| | | GGCTACGCCGACTCCGTGAAGGGCCGCTTCACCATCTCCCGCGACAACGCAAAGAACT |
| | | CCCTGTACTTGCAAATGAACTCGCTCAGGGCTGAGGATACCGCGCTGTACTACTGCTC |
| | | CGTGCATTCCTTCCTGGCCTACTGGGGACAGGGAACTCTGGTCACCGTGTCGAGCGCC |
| | | TCCGGCGGCGGGGGCTCGGGTGGACGGGCCTCGGGCGGAGGGGGGTCCGACATCGTGA |
| | | TGACCCAGACCCCGCTGAGCTTGCCCGTGACTCCCGGAGAGCCTGCATCCATCTCCTG |
| | | CCGGTCATCCCAGTCCCTTCTCCACTCCAACGGATACAACTACCTCGACTGGTACCTC |
| | | CAGAAGCCGGGACAGAGCCCTCAGCTTCTGATCTACCTGGGGTCAAATAGAGCCTCAG |
| | | GAGTGCCGGATCGGTTCAGCGGATCTGGTTCGGGAACTGATTTCACTCTGAAGATTTC |
| | | CCGCGTGGAAGCCGAGGACGTGGGCGTCTACTACTGTATGCAGGCGCTGCAGACCCCC |
| | | TATACCTTCGGCCAAGGGACGAAAGTGGAGATCAAG |
| 139105-aa | 70 | QVQLVESGGGLVQPGRSLRLSCAASGFTFDDYAMHWVRQAPGKGLEWVSGISWNSGSI |
| VH | | GYADSVKGRFTISRDNAKNSLYLQMNSLRAEDTALYYCSVHSFLAYWGQGTLVTVSS |
| 139105-aa | 85 | DIVMTQTPLSLPVTPGEPASISCRSSQSLLHSNGYNYLDWYLQKPGQSPQLLIYLGSN |
| VL | | RASGVPDRFSGSGSGTDFTLKISRVEAEDVGVYYCMQALQTPYTFGQGTKVEIK |
| 139105-aa | 100 | MALPVTALLLPLALLLHAARPQVQLVESGGGLVQPGRSLRLSCAASGFTFDDYAMHWV |
| Full CAR | | RQAPGKGLEWVSGISWNSGSIGYADSVKGRFTISRDNAKNSLYLQMNSLRAEDTALYY |
| | | CSVHSFLAYWGQGTLVTVSSASGGGGSGGRASGGGGSDIVMTQTPLSLPVTPGEPASI |
| | | SCRSSQSLLHSNGYNYLDWYLQKPGQSPQLLIYLGSNRASGVPDRFSGSGSGTDFTLK |
| | | ISRVEAEDVGVYYCMQALQTPYTFGQGTKVEIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| 139105-nt | 115 | ATGGCCCTCCCTGTCACCGCCCTGCTGCTTCCGCTGGCTCTTCTGCTCCACGCCGCTC |
| Full CAR | | GGCCCCAAGTGCAACTCGTCGAATCCGGTGGAGGTCTGGTCCAACCTGGTAGAAGCCT |
| | | GAGACTGTCGTGTGCGGCCAGCGGATTCACCTTTGATGACTATGCTATGCACTGGGTG |
| | | CGGCAGGCCCCAGGAAAGGGCCTGGAATGGGTGTCGGGAATTAGCTGGAACTCCGGGT |
| | | CCATTGGCTACGCCGACTCCGTGAAGGGCCGCTTCACCATCTCCCGCGACAACGCAAA |
| | | GAACTCCCTGTACTTGCAAATGAACTCGCTCAGGGCTGAGGATACCGCGCTGTACTAC |
| | | TGCTCCGTGCATTCCTTCCTGGCCTACTGGGGACAGGGAACTCTGGTCACCGTGTCGA |
| | | GCGCCTCCGGCGGCGGGGGCTCGGGTGGACGGGCCTCGGGCGGAGGGGGGTCCGACAT |
| | | CGTGATGACCCAGACCCCGCTGAGCTTGCCCGTGACTCCCGGAGAGCCTGCATCCATC |
| | | TCCTGCCGGTCATCCCAGTCCCTTCTCCACTCCAACGGATACAACTACCTCGACTGGT |
| | | ACCTCCAGAAGCCGGGACAGAGCCCTCAGCTTCTGATCTACCTGGGGTCAAATAGAGC |
| | | CTCAGGAGTGCCGGATCGGTTCAGCGGATCTGGTTCGGGAACTGATTTCACTCTGAAG |
| | | ATTTCCCGCGTGGAAGCCGAGGACGTGGGCGTCTACTACTGTATGCAGGCGCTGCAGA |
| | | CCCCCTATACCTTCGGCCAAGGGACGAAAGTGGAGATCAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |

139111

| | | |
|---|---|---|
| 139111-aa | 41 | EVQLLESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIVMTQTPLSLSVTPGQPASISCKSSQSLLRNDGKTPLYWYL |
| | | QKAGQPPQLLIYEVSNRFSGVPDRFSGSGSGTDFTLKISRVEAEDVGAYYCMQNIQFP |
| | | SFGGGTKLEIK |
| 139111-nt | 56 | GAAGTGCAATTGTTGGAATCTGGAGGAGGACTTGTGCAGCCTGGAGGATCACTGAGAC |
| ScFv domain | | TTTCGTGTGCGGTGTCAGGCTTCGCCCTGAGCAACCACGGCATGAGCTGGGTGCGGAG |
| | | AGCCCCGGGGAAGGGTCTGGAATGGGTGTCCGGGATCGTCTACTCCGGTTCAACTTAC |
| | | TACGCCGCAAGCGTGAAGGGTCGCTTCACCATTTCCCGCGATAACTCCCGGAACACCC |
| | | TGTACCTCCAAATGAACTCCCTGCGGCCCGAGGACACCGCCATCTACTACTGTTCCGC |
| | | GCATGGAGGAGAGTCCGATGTCTGGGGACAGGGCACTACCGTGACCGTGTCGAGCGCC |
| | | TCGGGGGGAGGAGGCTCCGGCGGTCGCGCCTCCGGGGGGGGTGGCAGCGACATTGTGA |
| | | TGACGCAGACTCCACTCTCGCTGTCCGTGACCCCGGGACAGCCCGCGTCCATCTCGTG |
| | | CAAGAGCTCCCAGAGCCTGCTGAGGAACGACGGAAAGACTCCTCTGTATTGGTACCTC |
| | | CAGAAGGCTGGACAGCCCCCGCAACTGCTCATCTACGAAGTGTCAAATCGCTTCTCCG |
| | | GGGTGCCGGATCGGTTTTCCGGCTCGGGATCGGGCACCGACTTCACCCTGAAAATCTC |
| | | CAGGGTCGAGGCCGAGGACGTGGGAGCCTACTACTGCATGCAAAACATCCAGTTCCCT |
| | | TCCTTCGGCGGCGGCACAAAGCTGGAGATTAAG |
| 139111-aa | 71 | EVQLLESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139111-aa | 86 | DIVMTQTPLSLSVTPGQPASISCKSSQSLLRNDGKTPLYWYLQKAGQPPQLLIYEVSN |
| VL | | RFSGVPDRFSGSGSGTDFTLKISRVEAEDVGAYYCMQNIQFPSFGGGTKLEIK |
| 139111-aa | 101 | MALPVTALLLPLALLLHAARPEVQLLESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIVMTQTPLSLSVTPGQPASI |
| | | SCKSSQSLLRNDGKTPLYWYLQKAGQPPQLLIYEVSNRFSGVPDRFSGSGSGTDFTLK |
| | | ISRVEAEDVGAYYCMQNIQFPSFGGGTKLEIKTTTPAPRPPTPAPTIASQPLSLRPEA |
| | | CRPAAGGAVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERRRGKGHDGL |
| | | YQGLSTATKDTYDALHMQALPPR |
| 139111-nt | 116 | ATGGCCCTCCCTGTCACCGCCCTGCTGCTTCCGCTGGCTCTTCTGCTCCACGCCGCTC |
| Full CAR | | GGCCCGAAGTGCAATTGTTGGAATCTGGAGGAGGACTTGTGCAGCCTGGAGGATCACT |
| | | GAGACTTTCGTGTGCGGTGTCAGGCTTCGCCCTGAGCAACCACGGCATGAGCTGGGTG |
| | | CGGAGAGCCCCGGGGAAGGGTCTGGAATGGGTGTCCGGGATCGTCTACTCCGGTTCAA |
| | | CTTACTACGCCGCAAGCGTGAAGGGTCGCTTCACCATTTCCCGCGATAACTCCCGGAA |
| | | CACCCTGTACCTCCAAATGAACTCCCTGCGGCCCGAGGACACCGCCATCTACTACTGT |
| | | TCCGCGCATGGAGGAGAGTCCGATGTCTGGGGACAGGGCACTACCGTGACCGTGTCGA |
| | | GCGCCTCGGGGGGAGGAGGCTCCGGCGGTCGCGCCTCCGGGGGGGGTGGCAGCGACAT |
| | | TGTGATGACGCAGACTCCACTCTCGCTGTCCGTGACCCCGGGACAGCCCGCGTCCATC |
| | | TCGTGCAAGAGCTCCCAGAGCCTGCTGAGGAACGACGGAAAGACTCCTCTGTATTGGT |
| | | ACCTCCAGAAGGCTGGACAGCCCCCGCAACTGCTCATCTACGAAGTGTCAAATCGCTT |
| | | CTCCGGGGTGCCGGATCGGTTTTCCGGCTCGGGATCGGGCACCGACTTCACCCTGAAA |
| | | ATCTCCAGGGTCGAGGCCGAGGACGTGGGAGCCTACTACTGCATGCAAAACATCCAGT |
| | | TCCCTTCCTTCGGCGGCGGCACAAAGCTGGAGATTAAGACCACTACCCCAGCACCGAG |
| | | GCCACCCACCCCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCAGATGCTCCAGC |
| | | CTACAAGCAGGGGCAGAACCAGCTCTACAACGAACTCAATCTTGGTCGGAGAGAGGAG |
| | | TACGACGTGCTGGACAAGCGGAGAGGACGGGACCCAGAAATGGGCGGGAAGCCGCGCA |
| | | GAAAGAATCCCCAAGAGGGCCTGTACAACGAGCTCCAAAAGGATAAGATGGCAGAAGC |
| | | CTATAGCGAGATTGGTATGAAAGGGGAACGCAGAAGAGGCAAAGGCCACGACGGACTG |
| | | TACCAGGGACTCAGCACCGCCACCAAGGACACCTATGACGCTCTTCACATGCAGGCCC |
| | | TGCCGCCTCGG |

139100

| | | |
|---|---|---|
| 139100-aa | 42 | QVQLVQSGAEVRKTGASVKVSCKASGYIFDNFGINWVRQAPGQGLEWMGWINPKNNNT |
| ScFv domain | | NYAQKFQGRVTITADESTNTAYMEVSSLRSEDTAVYYCARGPYYYQSYMDVWGQGTMV |
| | | TVSSASGGGGSGGRASGGGGSDIVMTQTPLSLPVTPGEPASISCRSSQSLLHSNGYNY |
| | | LNWYLQKPGQSPQLLIYLGSKRASGVPDRFSGSGSGTDFTLHITRVGAEDVGVYYCMQ |
| | | ALQTPYTFGQGTKLEIK |
| 139100-nt | 57 | CAAGTCCAACTCGTCCAGTCCGGCGCAGAAGTCAGAAAAACCGGTGCTAGCGTGAAAG |
| ScFv domain | | TGTCCTGCAAGGCCTCCGGCTACATTTTCGATAACTTCGGAATCAACTGGGTCAGACA |
| | | GGCCCCGGGCCAGGGGCTGGAATGGATGGGATGGATCAACCCCAAGAACAACAACACC |
| | | AACTACGCACAGAAGTTCCAGGGCCGCGTGACTATCACCGCCGATGAATCGACCAATA |
| | | CCGCCTACATGGAGGTGTCCTCCCTGCGGTCGGAGGACACTGCCGTGTATTACTGCGC |
| | | GAGGGGCCCATACTACTACCAAAGCTACATGGACGTCTGGGGACAGGGAACCATGGTG |
| | | ACCGTGTCATCCGCCTCCGGTGGTGGAGGCTCCGGGGGGCGGGCTTCAGGAGGCGGAG |
| | | GAAGCGATATTGTGATGACCCAGACTCCGCTTAGCCTGCCCGTGACTCCTGGAGAACC |
| | | GGCCTCCATTTCCTGCCGGTCCTCGCAATCACTCCTGCATTCCAACGGTTACAACTAC |
| | | CTGAATTGGTACCTCCAGAAGCCTGGCCAGTCGCCCCAGTTGCTGATCTATCTGGGCT |
| | | CGAAGCGCGCCTCCGGGGTGCCTGACCGGTTTAGCGGATCTGGGAGCGGCACGGACTT |
| | | CACTCTCCACATCACCCGCGTGGGAGCGGAGGACGTGGGAGTGTACTACTGTATGCAG |
| | | GCGCTGCAGACTCCGTACACATTCGGACAGGGCACCAAGCTGGAGATCAAG |
| 139100-aa | 72 | QVQLVQSGAEVRKTGASVKVSCKASGYIFDNFGINWVRQAPGQGLEWMGWINPKNNNT |
| VH | | NYAQKFQGRVTITADESTNTAYMEVSSLRSEDTAVYYCARGPYYYQSYMDVWGQGTMV |
| | | TVSS |
| 139100-aa | 87 | DIVMTQTPLSLPVTPGEPASISCRSSQSLLHSNGYNYLNWYLQKPGQSPQLLIYLGSK |
| VL | | RASGVPDRFSGSGSGTDFTLHITRVGAEDVGVYYCMQALQTPYTFGQGTKLEIK |
| 139100-aa | 102 | MALPVTALLLPLALLLHAARPQVQLVQSGAEVRKTGASVKVSCKASGYIFDNFGINWV |
| Full CAR | | RQAPGQGLEWMGWINPKNNNTNYAQKFQGRVTITADESTNTAYMEVSSLRSEDTAVYY |
| | | CARGPYYYQSYMDVWGQGTMVTVSSASGGGGSGGRASGGGGSDIVMTQTPLSLPVTPG |
| | | EPASISCRSSQSLLHSNGYNYLNWYLQKPGQSPQLLIYLGSKRASGVPDRFSGSGSGT |
| | | DFTLHITRVGAEDVGVYYCMQALQTPYTFGQGTKLEIKTTTPAPRPPTPAPTIASQPL |
| | | SLRPEACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQGQNQLYNELN |
| | | LGRREEYDVLDKRRGRDPEMGGKPRRKNPQEGLYNELQKDKMAEAYSEIGMKGERRRG |
| | | KGHDGLYQGLSTATKDTYDALHMQALPPR |
| 139100-nt | 117 | ATGGCCCTCCCTGTCACCGCCCTGCTGCTTCCGCTGGCTCTTCTGCTCCACGCCGCTC |
| Full CAR | | GGCCCCAAGTCCAACTCGTCCAGTCCGGCGCAGAAGTCAGAAAAACCGGTGCTAGCGT |
| | | GAAAGTGTCCTGCAAGGCCTCCGGCTACATTTTCGATAACTTCGGAATCAACTGGGTC |
| | | AGACAGGCCCCGGGCCAGGGGCTGGAATGGATGGGATGGATCAACCCCAAGAACAACA |
| | | ACACCAACTACGCACAGAAGTTCCAGGGCCGCGTGACTATCACCGCCGATGAATCGAC |
| | | CAATACCGCCTACATGGAGGTGTCCTCCCTGCGGTCGGAGGACACTGCCGTGTATTAC |
| | | TGCGCGAGGGGCCCATACTACTACCAAAGCTACATGGACGTCTGGGGACAGGGAACCA |
| | | TGGTGACCGTGTCATCCGCCTCCGGTGGTGGAGGCTCCGGGGGGCGGGCTTCAGGAGG |
| | | CGGAGGAAGCGATATTGTGATGACCCAGACTCCGCTTAGCCTGCCCGTGACTCCTGGA |
| | | GAACCGGCCTCCATTTCCTGCCGGTCCTCGCAATCACTCCTGCATTCCAACGGTTACA |
| | | ACTACCTGAATTGGTACCTCCAGAAGCCTGGCCAGTCGCCCCAGTTGCTGATCTATCT |
| | | GGGCTCGAAGCGCGCCTCCGGGGTGCCTGACCGGTTTAGCGGATCTGGGAGCGGCACG |
| | | GACTTCACTCTCCACATCACCCGCGTGGGAGCGGAGGACGTGGGAGTGTACTACTGTA |
| | | TGCAGGCGCTGCAGACTCCGTACACATTCGGACAGGGCACCAAGCTGGAGATCAAGAC |
| | | CACTACCCCAGCACCGAGGCCACCCACCCCGGCTCCTACCATCGCCTCCCAGCCTCTG |
| | | TCCCTGCGTCCGGAGGCATGTAGACCCGCAGCTGGTGGGGCCGTGCATACCCGGGGTC |
| | | TTGACTTCGCCTGCGATATCTACATTTGGGCCCCTCTGGCTGGTACTTGCGGGGTCCT |
| | | GCTGCTTTCACTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAAATTCAGCCG |
| | | CAGCGCAGATGCTCCAGCCTACAAGCAGGGGCAGAACCAGCTCTACAACGAACTCAAT |
| | | CTTGGTCGGAGAGAGGAGTACGACGTGCTGGACAAGCGGAGAGGACGGGACCCAGAAA |
| | | TGGGCGGGAAGCCGCGCAGAAAGAATCCCCAAGAGGGCCTGTACAACGAGCTCCAAAA |
| | | GGATAAGATGGCAGAAGCCTATAGCGAGATTGGTATGAAAGGGGAACGCAGAAGAGGC |
| | | AAAGGCCACGACGGACTGTACCAGGGACTCAGCACCGCCACCAAGGACACCTATGACG |
| | | CTCTTCACATGCAGGCCCTGCCGCCTCGG |

139101

| | | |
|---|---|---|
| 139101-aa | 43 | QVQLQESGGGLVQPGGSLRLSCAASGFTFSSDAMTWVRQAPGKGLEWVSVISGSGGTT |
| ScFv domain | | YYADSVKGRFTISRDNSKNTLYLQMNSLRAEDTAVYYCAKLDSSGYYYARGPRYWGQG |
| | | TLVTVSSASGGGGSGGRASGGGGSDIQLTQSPSSLSASVGDRVTITCRASQSISSYLN |
| | | WYQQKPGKAPKLLIYGASTLASGVPARFSGSGSGTHFTLTINSLQSEDSATYYCQQSY |
| | | KRASFGQGTKVEIK |
| 139101-nt | 58 | CAAGTGCAACTTCAAGAATCAGGCGGAGGACTCGTGCAGCCCGGAGGATCATTGCGGC |
| ScFv domain | | TCTCGTGCGCCGCCTCGGGCTTCACCTTCTCGAGCGACGCCATGACCTGGGTCCGCCA |
| | | GGCCCCGGGGAAGGGGCTGGAATGGGTGTCTGTGATTTCCGGCTCCGGGGGAACTACG |
| | | TACTACGCCGATTCCGTGAAAGGTCGCTTCACTATCTCCCGGGACAACAGCAAGAACA |
| | | CCCTTTATCTGCAAATGAATTCCCTCCGCGCCGAGGACACCGCCGTGTACTACTGCGC |
| | | CAAGCTGGACTCCTCGGGCTACTACTATGCCCGGGGTCCGAGATACTGGGGACAGGGA |
| | | ACCCTCGTGACCGTGTCCTCCGCGTCCGGCGGAGGAGGGTCGGGAGGGCGGGCCTCCG |
| | | GCGGCGGCGGTTCGGACATCCAGCTGACCCAGTCCCCATCCTCACTGAGCGCAAGCGT |
| | | GGGCGACAGAGTCACCATTACATGCAGGGCGTCCCAGAGCATCAGCTCCTACCTGAAC |
| | | TGGTACCAACAGAAGCCTGGAAAGGCTCCTAAGCTGTTGATCTACGGGGCTTCGACCC |
| | | TGGCATCCGGGGTGCCCGCGAGGTTTAGCGGAAGCGGTAGCGGCACTCACTTCACTCT |
| | | GACCATTAACAGCCTCCAGTCCGAGGATTCAGCCACTTACTACTGTCAGCAGTCCTAC |
| | | AAGCGGGCCAGCTTCGGACAGGGCACTAAGGTCGAGATCAAG |
| 139101-aa | 73 | QVQLQESGGGLVQPGGSLRLSCAASGFTFSSDAMTWVRQAPGKGLEWVSVISGSGGTT |
| VH | | YYADSVKGRFTISRDNSKNTLYLQMNSLRAEDTAVYYCAKLDSSGYYYARGPRYWGQG |
| | | TLVTVSS |
| 139101-aa | 88 | DIQLTQSPSSLSASVGDRVTITCRASQSISSYLNWYQQKPGKAPKLLIYGASTLASGV |
| VL | | PARESGSGSGTHFTLTINSLQSEDSATYYCQQSYKRASFGQGTKVEIK |
| 139101-aa | 103 | MALPVTALLLPLALLLHAARPQVQLQESGGGLVQPGGSLRLSCAASGFTFSSDAMTWV |
| Full CAR | | RQAPGKGLEWVSVISGSGGTTYYADSVKGRFTISRDNSKNTLYLQMNSLRAEDTAVYY |
| | | CAKLDSSGYYYARGPRYWGQGTLVTVSSASGGGGSGGRASGGGGSDIQLTQSPSSLSA |
| | | SVGDRVTITCRASQSISSYLNWYQQKPGKAPKLLIYGASTLASGVPARFSGSGSGTHF |
| | | TLTINSLQSEDSATYYCQQSYKRASFGQGTKVEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139101-nt | 118 | ATGGCCCTCCCTGTCACCGCCCTGCTGCTTCCGCTGGCTCTTCTGCTCCACGCCGCTC |
| Full CAR | | GGCCCCAAGTGCAACTTCAAGAATCAGGCGGAGGACTCGTGCAGCCCGGAGGATCATT |
| | | GCGGCTCTCGTGCGCCGCCTCGGGCTTCACCTTCTCGAGCGACGCCATGACCTGGGTC |
| | | CGCCAGGCCCCGGGGAAGGGGCTGGAATGGGTGTCTGTGATTTCCGGCTCCGGGGGAA |
| | | CTACGTACTACGCCGATTCCGTGAAAGGTCGCTTCACTATCTCCCGGGACAACAGCAA |
| | | GAACACCCTTTATCTGCAAATGAATTCCCTCCGCGCCGAGGACACCGCCGTGTACTAC |
| | | TGCGCCAAGCTGGACTCCTCGGGCTACTACTATGCCCGGGGTCCGAGATACTGGGGAC |
| | | AGGGAACCCTCGTGACCGTGTCCTCCGCGTCCGGCGGAGGAGGGTCGGGAGGGCGGGC |
| | | CTCCGGCGGCGGCGGTTCGGACATCCAGCTGACCCAGTCCCCATCCTCACTGAGCGCA |
| | | AGCGTGGGCGACAGAGTCACCATTACATGCAGGGCGTCCCAGAGCATCAGCTCCTACC |
| | | TGAACTGGTACCAACAGAAGCCTGGAAAGGCTCCTAAGCTGTTGATCTACGGGGCTTC |
| | | GACCCTGGCATCCGGGGTGCCCGCGAGGTTTAGCGGAAGCGGTAGCGGCACTCACTTC |
| | | ACTCTGACCATTAACAGCCTCCAGTCCGAGGATTCAGCCACTTACTACTGTCAGCAGT |
| | | CCTACAAGCGGGCCAGCTTCGGACAGGGCACTAAGGTCGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

139102

| | | |
|---|---|---|
| 139102-aa | 44 | QVQLVQSGAEVKKPGASVKVSCKASGYTFSNYGITWVRQAPGQGLEWMGWISAYNGNT |
| ScFv domain | | NYAQKFQGRVTMTRNTSISTAYMELSSLRSEDTAVYYCARGPYYYYMDVWGKGTMVTV |
| | | SSASGGGGSGGRASGGGGSEIVMTQSPLSLPVTPGEPASISCRSSQSLLYSNGYNYVD |
| | | WYLQKPGQSPQLLIYLGSNRASGVPDRFSGSGSGTDFKLQISRVEAEDVGIYYCMQGR |
| | | QFPYSFGQGTKVEIK |
| 139102-nt | 59 | CAAGTCCAACTGGTCCAGAGCGGTGCAGAAGTGAAGAAGCCCGGAGCGAGCGTGAAAG |
| ScFv domain | | TGTCCTGCAAGGCTTCCGGGTACACCTTCTCCAACTACGGCATCACTTGGGTGCGCCA |
| | | GGCCCCGGGACAGGGCCTGGAATGGATGGGGTGGATTTCCGCGTACAACGGCAATACG |
| | | AACTACGCTCAGAAGTTCCAGGGTAGAGTGACCATGACTAGGAACACCTCCATTTCCA |
| | | CCGCCTACATGGAACTGTCCTCCCTGCGGAGCGAGGACACCGCCGTGTACTATTGCGC |
| | | CCGGGGACCATACTACTACTACATGGATGTCTGGGGGAAGGGGACTATGGTCACCGTG |
| | | TCATCCGCCTCGGGAGGCGGCGGATCAGGAGGACGCGCCTCTGGTGGTGGAGGATCGG |
| | | AGATCGTGATGACCCAGAGCCCTCTCTCCTTGCCCGTGACTCCTGGGGAGCCCGCATC |
| | | CATTTCATGCCGGAGCTCCCAGTCACTTCTCTACTCCAACGGCTATAACTACGTGGAT |
| | | TGGTACCTCCAAAAGCCGGGCCAGAGCCCGCAGCTGCTGATCTACCTGGGCTCGAACA |
| | | GGGCCAGCGGAGTGCCTGACCGGTTCTCCGGGTCGGGAAGCGGGACCGACTTCAAGCT |
| | | GCAAATCTCGAGAGTGGAGGCCGAGGACGTGGGAATCTACTACTGTATGCAGGGCCGC |
| | | CAGTTTCCGTACTCGTTCGGACAGGGCACCAAAGTGGAAATCAAG |
| 139102-aa | 74 | QVQLVQSGAEVKKPGASVKVSCKASGYTFSNYGITWVRQAPGQGLEWMGWISAYNGNT |
| VH | | NYAQKFQGRVTMTRNTSISTAYMELSSLRSEDTAVYYCARGPYYYYMDVWGKGTMVTV |
| | | SS |
| 139102-aa | 89 | EIVMTQSPLSLPVTPGEPASISCRSSQSLLYSNGYNYVDWYLQKPGQSPQLLIYLGSN |
| VL | | RASGVPDRFSGSGSGTDFKLQISRVEAEDVGIYYCMQGRQFPYSFGQGTKVEIK |
| 139102-aa | 104 | MALPVTALLLPLALLLHAARPQVQLVQSGAEVKKPGASVKVSCKASGYTFSNYGITWV |
| Full CAR | | RQAPGQGLEWMGWISAYNGNTNYAQKFQGRVTMTRNTSISTAYMELSSLRSEDTAVYY |
| | | CARGPYYYYMDVWGKGTMVTVSSASGGGGSGGRASGGGGSEIVMTQSPLSLPVTPGEP |
| | | ASISCRSSQSLLYSNGYNYVDWYLQKPGQSPQLLIYLGSNRASGVPDRFSGSGSGTDF |
| | | KLQISRVEAEDVGIYYCMQGRQFPYSFGQGTKVEIKTTTPAPRPPTPAPTIASQPLSL |
| | | RPEACRPAAGGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGMKGERRRGKG |
| | | HDGLYQGLSTATKDTYDALHMQALPPR |
| 139102-nt | 119 | ATGGCCCTCCCTGTCACCGCCCTGCTGCTTCCGCTGGCTCTTCTGCTCCACGCCGCTC |
| Full CAR | | GGCCCCAAGTCCAACTGGTCCAGAGCGGTGCAGAAGTGAAGAAGCCCGGAGCGAGCGT |
| | | GAAAGTGTCCTGCAAGGCTTCCGGGTACACCTTCTCCAACTACGGCATCACTTGGGTG |
| | | CGCCAGGCCCCGGGACAGGGCCTGGAATGGATGGGGTGGATTTCCGCGTACAACGGCA |
| | | ATACGAACTACGCTCAGAAGTTCCAGGGTAGAGTGACCATGACTAGGAACACCTCCAT |
| | | TTCCACCGCCTACATGGAACTGTCCTCCCTGCGGAGCGAGGACACCGCCGTGTACTAT |
| | | TGCGCCCGGGGACCATACTACTACTACATGGATGTCTGGGGGAAGGGGACTATGGTCA |
| | | CCGTGTCATCCGCCTCGGGAGGCGGCGGATCAGGAGGACGCGCCTCTGGTGGTGGAGG |
| | | ATCGGAGATCGTGATGACCCAGAGCCCTCTCTCCTTGCCCGTGACTCCTGGGGAGCCC |
| | | GCATCCATTTCATGCCGGAGCTCCCAGTCACTTCTCTACTCCAACGGCTATAACTACG |
| | | TGGATTGGTACCTCCAAAAGCCGGGCCAGAGCCCGCAGCTGCTGATCTACCTGGGCTC |
| | | GAACAGGGCCAGCGGAGTGCCTGACCGGTTCTCCGGGTCGGGAAGCGGGACCGACTTC |
| | | AAGCTGCAAATCTCGAGAGTGGAGGCCGAGGACGTGGGAATCTACTACTGTATGCAGG |
| | | GCCGCCAGTTTCCGTACTCGTTCGGACAGGGCACCAAAGTGGAAATCAAGACCACTAC |
| | | CCCAGCACCGAGGCCACCCACCCCGGCTCCTACCATCGCCTCCCAGCCTCTGTCCCTG |
| | | CGTCCGGAGGCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGTCCTGCTGCT |
| | | TTCACTCGTGATCACTCTTTACTGTAAGCGCGGTCGGAAGAAGCTGCTGTACATCTTT |
| | | AAGCAACCCTTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAGCCGCAGCGC |
| | | AGATGCTCCAGCCTACAAGCAGGGGCAGAACCAGCTCTACAACGAACTCAATCTTGGT |
| | | CGGAGAGAGGAGTACGACGTGCTGGACAAGCGGAGAGGACGGGACCCAGAAATGGGCG |
| | | GGAAGCCGCGCAGAAAGAATCCCCAAGAGGGCCTGTACAACGAGCTCCAAAAGGATAA |
| | | GATGGCAGAAGCCTATAGCGAGATTGGTATGAAAGGGGAACGCAGAAGAGGCAAAGGC |
| | | CACGACGGACTGTACCAGGGACTCAGCACCGCCACCAAGGACACCTATGACGCTCTTC |
| | | ACATGCAGGCCCTGCCGCCTCGG |

139104

| | | |
|---|---|---|
| 139104-aa | 45 | EVQLLETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPATLSVSPGESATLSCRASQSVSSNLAWYQQKPGQ |
| | | APRLLIYGASTRASGIPDRFSGSGSGTDFTLTISSLQAEDVAVYYCQQYGSSLTFGGG |
| | | TKVEIK |
| 139104-nt | 60 | GAAGTGCAATTGCTCGAAACTGGAGGAGGTCTGGTGCAACCTGGAGGATCACTTCGCC |
| ScFv domain | | TGTCCTGCGCCGTGTCGGGCTTTGCCCTGTCCAACCATGGAATGAGCTGGGTCCGCCG |
| | | CGCGCCGGGGAAGGGCCTCGAATGGGTGTCCGGCATCGTCTACTCCGGCTCCACCTAC |
| | | TACGCCGCGTCCGTGAAGGGCCGGTTCACGATTTCACGGGACAACTCGCGGAACACCC |
| | | TGTACCTCCAAATGAATTCCCTTCGGCCGGAGGATACTGCCATCTACTACTGCTCCGC |
| | | CCACGGTGGCGAATCCGACGTCTGGGGCCAGGGAACCACCGTGACCGTGTCCAGCGCG |
| | | TCCGGGGGAGGAGGAAGCGGGGGTAGAGCATCGGGTGGAGGCGGATCAGAGATCGTGC |
| | | TGACCCAGTCCCCCGCCACCTTGAGCGTGTCACCAGGAGAGTCCGCCACCCTGTCATG |
| | | CCGCGCCAGCCAGTCCGTGTCCTCCAACCTGGCTTGGTACCAGCAGAAGCCGGGGCAG |
| | | GCCCCTAGACTCCTGATCTATGGGGCGTCGACCCGGGCATCTGGAATTCCCGATAGGT |
| | | TCAGCGGATCGGGCTCGGGCACTGACTTCACTCTGACCATCTCCTCGCTGCAAGCCGA |
| | | GGACGTGGCTGTGTACTACTGTCAGCAGTACGGAAGCTCCCTGACTTTCGGTGGCGGG |
| | | ACCAAAGTCGAGATTAAG |
| 139104-aa | 75 | EVQLLETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139104-aa | 90 | EIVLTQSPATLSVSPGESATLSCRASQSVSSNLAWYQQKPGQAPRLLIYGASTRASGI |
| VL | | PDRFSGSGSGTDFTLTISSLQAEDVAVYYCQQYGSSLTFGGGTKVEIK |
| 139104-aa | 105 | MALPVTALLLPLALLLHAARPEVQLLETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPATLSVSPGESATL |
| | | SCRASQSVSSNLAWYQQKPGQAPRLLIYGASTRASGIPDRFSGSGSGTDFTLTISSLQ |
| | | AEDVAVYYCQQYGSSLTFGGGTKVEIKTTTPAPRPPTPAPTIASQPLSLRPEACRPAA |
| | | GGAVHTRGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRRGKGHDGLYQGLS |
| | | TATKDTYDALHMQALPPR |
| 139104-nt | 120 | ATGGCCCTCCCTGTCACCGCCCTGCTGCTTCCGCTGGCTCTTCTGCTCCACGCCGCTC |
| Full CAR | | GGCCCGAAGTGCAATTGCTCGAAACTGGAGGAGGTCTGGTGCAACCTGGAGGATCACT |
| | | TCGCCTGTCCTGCGCCGTGTCGGGCTTTGCCCTGTCCAACCATGGAATGAGCTGGGTC |
| | | CGCCGCGCGCCGGGGAAGGGCCTCGAATGGGTGTCCGGCATCGTCTACTCCGGCTCCA |
| | | CCTACTACGCCGCGTCCGTGAAGGGCCGGTTCACGATTTCACGGGACAACTCGCGGAA |
| | | CACCCTGTACCTCCAAATGAATTCCCTTCGGCCGGAGGATACTGCCATCTACTACTGC |
| | | TCCGCCCACGGTGGCGAATCCGACGTCTGGGGCCAGGGAACCACCGTGACCGTGTCCA |
| | | GCGCGTCCGGGGGAGGAGGAAGCGGGGGTAGAGCATCGGGTGGAGGCGGATCAGAGAT |
| | | CGTGCTGACCCAGTCCCCCGCCACCTTGAGCGTGTCACCAGGAGAGTCCGCCACCCTG |
| | | TCATGCCGCGCCAGCCAGTCCGTGTCCTCCAACCTGGCTTGGTACCAGCAGAAGCCGG |
| | | GGCAGGCCCCTAGACTCCTGATCTATGGGGCGTCGACCCGGGCATCTGGAATTCCCGA |
| | | TAGGTTCAGCGGATCGGGCTCGGGCACTGACTTCACTCTGACCATCTCCTCGCTGCAA |
| | | GCCGAGGACGTGGCTGTGTACTACTGTCAGCAGTACGGAAGCTCCCTGACTTTCGGTG |
| | | GCGGGACCAAAGTCGAGATTAAGACCACTACCCCAGCACCGAGGCCACCCACCCCGGC |
| | | TCCTACCATCGCCTCCCAGCCTCTGTCCCTGCGTCCGGAGGCATGTAGACCCGCAGCT |
| | | GGTGGGGCCGTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCTTTACTGTAA |
| | | GCGCGGTCGGAAGAAGCTGCTGTACATCTTTAAGCAACCCTTCATGAGGCCTGTGCAG |
| | | ACTACTCAAGAGGAGGACGGCTGTTCATGCCGGTTCCCAGAGGAGGAGGAAGGCGGCT |
| | | GCGAACTGCGCGTGAAATTCAGCCGCAGCGCAGATGCTCCAGCCTACAAGCAGGGGCA |
| | | GAACCAGCTCTACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAGAATCCCCAAG |
| | | AGGGCCTGTACAACGAGCTCCAAAAGGATAAGATGGCAGAAGCCTATAGCGAGATTGG |
| | | TATGAAAGGGGAACGCAGAAGAGGCAAAGGCCACGACGGACTGTACCAGGGACTCAGC |
| | | ACCGCCACCAAGGACACCTATGACGCTCTTCACATGCAGGCCCTGCCGCCTCGG |

139106

| | | |
|---|---|---|
| 139106-aa | 46 | EVQLVETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVMTQSPATLSVSPGERATLSCRASQSVSSKLAWYQQKPGQ |
| | | APRLLMYGASIRATGIPDRFSGSGSGTEFTLTISSLEPEDFAVYYCQQYGSSSWTFGQ |
| | | GTKVEIK |
| 139106-nt | 61 | GAAGTGCAATTGGTGGAAACTGGAGGAGGACTTGTGCAACCTGGAGGATCATTGAGAC |
| ScFv domain | | TGAGCTGCGCAGTGTCGGGATTCGCCCTGAGCAACCATGGAATGTCCTGGGTCAGAAG |
| | | GGCCCCTGGAAAAGGCCTCGAATGGGTGTCAGGGATCGTGTACTCCGGTTCCACTTAC |
| | | TACGCCGCCTCCGTGAAGGGGCGCTTCACTATCTCACGGGATAACTCCCGCAATACCC |
| | | TGTACCTCCAAATGAACAGCCTGCGGCCGGAGGATACCGCCATCTACTACTGTTCCGC |
| | | CCACGGTGGAGAGTCTGACGTCTGGGGCCAGGGAACTACCGTGACCGTGTCCTCCGCG |
| | | TCCGGCGGTGGAGGGAGCGGCGGCCGCGCCAGCGGCGGCGGAGGCTCCGAGATCGTGA |
| | | TGACCCAGAGCCCCGCTACTCTGTCGGTGTCGCCCGGAGAAAGGGCGACCCTGTCCTG |
| | | CCGGGCGTCGCAGTCCGTGAGCAGCAAGCTGGCTTGGTACCAGCAGAAGCCGGGCCAG |
| | | GCACCACGCCTGCTTATGTACGGTGCCTCCATTCGGGCCACCGGAATCCCGGACCGGT |
| | | TCTCGGGGTCGGGGTCCGGTACCGAGTTCACACTGACCATTTCCTCGCTCGAGCCCGA |
| | | GGACTTTGCCGTCTATTACTGCCAGCAGTACGGCTCCTCCTCATGGACGTTCGGCCAG |
| | | GGGACCAAGGTCGAAATCAAG |
| 139106-aa | 76 | EVQLVETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139106-aa | 91 | EIVMTQSPATLSVSPGERATLSCRASQSVSSKLAWYQQKPGQAPRLLMYGASIRATGI |
| VL | | PDRFSGSGSGTEFTLTISSLEPEDFAVYYCQQYGSSSWTFGQGTKVEIK |
| 139106-aa | 106 | MALPVTALLLPLALLLHAARPEVQLVETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVMTQSPATLSVSPGERATL |
| | | SCRASQSVSSKLAWYQQKPGQAPRLLMYGASIRATGIPDRFSGSGSGTEFTLTISSLE |
| | | PEDFAVYYCQQYGSSSWTFGQGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139106-nt | 121 | ATGGCCCTCCCTGTCACCGCCCTGCTGCTTCCGCTGGCTCTTCTGCTCCACGCCGCTC |
| Full CAR | | GGCCCGAAGTGCAATTGGTGGAAACTGGAGGAGGACTTGTGCAACCTGGAGGATCATT |
| | | GAGACTGAGCTGCGCAGTGTCGGGATTCGCCCTGAGCAACCATGGAATGTCCTGGGTC |
| | | AGAAGGGCCCCTGGAAAAGGCCTCGAATGGGTGTCAGGGATCGTGTACTCCGGTTCCA |
| | | CTTACTACGCCGCCTCCGTGAAGGGGCGCTTCACTATCTCACGGGATAACTCCCGCAA |
| | | TACCCTGTACCTCCAAATGAACAGCCTGCGGCCGGAGGATACCGCCATCTACTACTGT |
| | | TCCGCCCACGGTGGAGAGTCTGACGTCTGGGGCCAGGGAACTACCGTGACCGTGTCCT |
| | | CCGCGTCCGGCGGTGGAGGGAGCGGCGGCCGCGCCAGCGGCGGCGGAGGCTCCGAGAT |
| | | CGTGATGACCCAGAGCCCCGCTACTCTGTCGGTGTCGCCCGGAGAAAGGGCGACCCTG |
| | | TCCTGCCGGGCGTCGCAGTCCGTGAGCAGCAAGCTGGCTTGGTACCAGCAGAAGCCGG |
| | | GCCAGGCACCACGCCTGCTTATGTACGGTGCCTCCATTCGGGCCACCGGAATCCCGGA |
| | | CCGGTTCTCGGGGTCGGGGTCCGGTACCGAGTTCACACTGACCATTTCCTCGCTCGAG |
| | | CCCGAGGACTTTGCCGTCTATTACTGCCAGCAGTACGGCTCCTCCTCATGGACGTTCG |
| | | GCCAGGGGACCAAGGTCGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |

139107

| | | |
|---|---|---|
| 139107-aa | 47 | EVQLVETGGGVVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPGTLSLSPGERATLSCRASQSVGSTNLAWYQQKPG |
| | | QAPRLLIYDASNRATGIPDRFSGGGSGTDFTLTISRLEPEDFAVYYCQQYGSSPPWTF |
| | | GQGTKVEIK |
| 139107-nt | 62 | GAAGTGCAATTGGTGGAGACTGGAGGAGGAGTGGTGCAACCTGGAGGAAGCCTGAGAC |
| ScFv domain | | TGTCATGCGCGGTGTCGGGCTTCGCCCTCTCCAACCACGGAATGTCCTGGGTCCGCCG |
| | | GGCCCCTGGGAAAGGACTTGAATGGGTGTCCGGCATCGTGTACTCGGGTTCCACCTAC |
| | | TACGCGGCCTCAGTGAAGGGCCGGTTTACTATTAGCCGCGACAACTCCAGAAACACAC |
| | | TGTACCTCCAAATGAACTCGCTGCGGCCGGAAGATACCGCTATCTACTACTGCTCCGC |
| | | CCATGGGGGAGAGTCGGACGTCTGGGGACAGGGCACCACTGTCACTGTGTCCAGCGCT |
| | | TCCGGCGGTGGTGGAAGCGGGGGACGGGCCTCAGGAGGCGGTGGCAGCGAGATTGTGC |
| | | TGACCCAGTCCCCCGGGACCCTGAGCCTGTCCCCGGGAGAAAGGGCCACCCTCTCCTG |
| | | TCGGGCATCCCAGTCCGTGGGGTCTACTAACCTTGCATGGTACCAGCAGAAGCCCGGC |
| | | CAGGCCCCTCGCCTGCTGATCTACGACGCGTCCAATAGAGCCACCGGCATCCCGGATC |
| | | GCTTCAGCGGAGGCGGATCGGGCACCGACTTCACCCTCACCATTTCAAGGCTGGAACC |
| | | GGAGGACTTCGCCGTGTACTACTGCCAGCAGTATGGTTCGTCCCCACCCTGGACGTTC |
| | | GGCCAGGGGACTAAGGTCGAGATCAAG |
| 139107-aa | 77 | EVQLVETGGGVVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139107-aa | 92 | EIVLTQSPGTLSLSPGERATLSCRASQSVGSTNLAWYQQKPGQAPRLLIYDASNRATG |
| VL | | IPDRFSGGGSGTDFTLTISRLEPEDFAVYYCQQYGSSPPWTFGQGTKVEIK |
| 139107-aa | 107 | MALPVTALLLPLALLLHAARPEVQLVETGGGVVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPGTLSLSPGERATL |
| | | SCRASQSVGSTNLAWYQQKPGQAPRLLIYDASNRATGIPDRFSGGGSGTDFTLTISRL |
| | | EPEDFAVYYCQQYGSSPPWTFGQGTKVEIKTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 139107-nt | 122 | ATGGCCCTCCCTGTCACCGCCCTGCTGCTTCCGCTGGCTCTTCTGCTCCACGCCGCTC |
| Full | CAR | GGCCCGAAGTGCAATTGGTGGAGACTGGAGGAGGAGTGGTGCAACCTGGAGGAAGCCT |
| | | GAGACTGTCATGCGCGGTGTCGGGCTTCGCCCTCTCCAACCACGGAATGTCCTGGGTC |
| | | CGCCGGGCCCCTGGGAAAGGACTTGAATGGGTGTCCGGCATCGTGTACTCGGGTTCCA |
| | | CCTACTACGCGGCCTCAGTGAAGGGCCGGTTTACTATTAGCCGCGACAACTCCAGAAA |
| | | CACACTGTACCTCCAAATGAACTCGCTGCGGCCGGAAGATACCGCTATCTACTACTGC |
| | | TCCGCCCATGGGGGAGAGTCGGACGTCTGGGGACAGGGCACCACTGTCACTGTGTCCA |
| | | GCGCTTCCGGCGGTGGTGGAAGCGGGGGACGGGCCTCAGGAGGCGGTGGCAGCGAGAT |
| | | TGTGCTGACCCAGTCCCCCGGGACCCTGAGCCTGTCCCCGGGAGAAAGGGCCACCCTC |
| | | TCCTGTCGGGCATCCCAGTCCGTGGGGTCTACTAACCTTGCATGGTACCAGCAGAAGC |
| | | CCGGCCAGGCCCCTCGCCTGCTGATCTACGACGCGTCCAATAGAGCCACCGGCATCCC |
| | | GGATCGCTTCAGCGGAGGCGGATCGGGCACCGACTTCACCCTCACCATTTCAAGGCTG |
| | | GAACCGGAGGACTTCGCCGTGTACTACTGCCAGCAGTATGGTTCGTCCCCACCCTGGA |
| | | CGTTCGGCCAGGGGACTAAGGTCGAGATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |

139108

| | | |
|---|---|---|
| 139108-aa | 48 | QVQLVESGGGLVKPGGSLRLSCAASGFTFSDYYMSWIRQAPGKGLEWVSYISSSGSTI |
| ScFv domain | | YYADSVKGRFTISRDNAKNSLYLQMNSLRAEDTAVYYCARESGDGMDVWGQGTTVTVS |
| | | SASGGGGSGGRASGGGGSDIQMTQSPSSLSASVGDRVTITCRASQSISSYLNWYQQKP |
| | | GKAPKLLIYAASSLQSGVPSRFSGSGSGTDFTLTISSLQPEDFATYYCQQSYTLAFGQ |
| | | GTKVDIK |
| 139108-nt | 63 | CAAGTGCAACTCGTGGAATCTGGTGGAGGACTCGTGAAACCTGGAGGATCATTGAGAC |
| ScFv domain | | TGTCATGCGCGGCCTCGGGATTCACGTTCTCCGATTACTACATGAGCTGGATTCGCCA |
| | | GGCTCCGGGGAAGGGACTGGAATGGGTGTCCTACATTTCCTCATCCGGCTCCACCATC |
| | | TACTACGCGGACTCCGTGAAGGGGAGATTCACCATTAGCCGCGATAACGCCAAGAACA |
| | | GCCTGTACCTTCAGATGAACTCCCTGCGGGCTGAAGATACTGCCGTCTACTACTGCGC |
| | | AAGGGAGAGCGGAGATGGGATGGACGTCTGGGGACAGGGTACCACTGTGACCGTGTCG |
| | | TCGGCCTCCGGCGGAGGGGGTTCGGGTGGAAGGGCCAGCGGCGGCGGAGGCAGCGACA |
| | | TCCAGATGACCCAGTCCCCCTCATCGCTGTCCGCCTCCGTGGGCGACCGCGTCACCAT |
| | | CACATGCCGGGCCTCACAGTCGATCTCCTCCTACCTCAATTGGTATCAGCAGAAGCCC |
| | | GGAAAGGCCCCTAAGCTTCTGATCTACGCAGCGTCCTCCCTGCAATCCGGGGTCCCAT |
| | | CTCGGTTCTCCGGCTCGGGCAGCGGTACCGACTTCACTCTGACCATCTCGAGCCTGCA |
| | | GCCGGAGGACTTCGCCACTTACTACTGTCAGCAAAGCTACACCCTCGCGTTTGGCCAG |
| | | GGCACCAAAGTGGACATCAAG |
| 139108-aa | 78 | QVQLVESGGGLVKPGGSLRLSCAASGFTFSDYYMSWIRQAPGKGLEWVSYISSSGSTI |
| VH | | YYADSVKGRFTISRDNAKNSLYLQMNSLRAEDTAVYYCARESGDGMDVWGQGTTVTVS |
| | | S |
| 139108-aa | 93 | DIQMTQSPSSLSASVGDRVTITCRASQSISSYLNWYQQKPGKAPKLLIYAASSLQSGV |
| VL | | PSRFSGSGSGTDFTLTISSLQPEDFATYYCQQSYTLAFGQGTKVDIK |
| 139108-aa | 108 | MALPVTALLLPLALLLHAARPQVQLVESGGGLVKPGGSLRLSCAASGFTFSDYYMSWI |
| Full CAR | | RQAPGKGLEWVSYISSSGSTIYYADSVKGRFTISRDNAKNSLYLQMNSLRAEDTAVYY |
| | | CARESGDGMDVWGQGTTVTVSSASGGGGSGGRASGGGGSDIQMTQSPSSLSASVGDRV |
| | | TITCRASQSISSYLNWYQQKPGKAPKLLIYAASSLQSGVPSRFSGSGSGTDFTLTISS |
| | | LQPEDFATYYCQQSYTLAFGQGTKVDIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139108-nt | 123 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGAAACCTGGAGGATCATT |
| | | GAGACTGTCATGCGCGGCCTCGGGATTCACGTTCTCCGATTACTACATGAGCTGGATT |
| | | CGCCAGGCTCCGGGGAAGGGACTGGAATGGGTGTCCTACATTTCCTCATCCGGCTCCA |
| | | CCATCTACTACGCGGACTCCGTGAAGGGGAGATTCACCATTAGCCGCGATAACGCCAA |
| | | GAACAGCCTGTACCTTCAGATGAACTCCCTGCGGGCTGAAGATACTGCCGTCTACTAC |
| | | TGCGCAAGGGAGAGCGGAGATGGGATGGACGTCTGGGGACAGGGTACCACTGTGACCG |
| | | TGTCGTCGGCCTCCGGCGGAGGGGGTTCGGGTGGAAGGGCCAGCGGCGGCGGAGGCAG |
| | | CGACATCCAGATGACCCAGTCCCCCTCATCGCTGTCCGCCTCCGTGGGCGACCGCGTC |
| | | ACCATCACATGCCGGGCCTCACAGTCGATCTCCTCCTACCTCAATTGGTATCAGCAGA |
| | | AGCCCGGAAAGGCCCCTAAGCTTCTGATCTACGCAGCGTCCTCCCTGCAATCCGGGGT |
| | | CCCATCTCGGTTCTCCGGCTCGGGCAGCGGTACCGACTTCACTCTGACCATCTCGAGC |
| | | CTGCAGCCGGAGGACTTCGCCACTTACTACTGTCAGCAAAGCTACACCCTCGCGTTTG |
| | | GCCAGGGCACCAAAGTGGACATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |

139110

| | | |
|---|---|---|
| 139110-aa | 50 | QVQLVQSGGGLVKPGGSLRLSCAASGFTFSDYYMSWIRQAPGKGLEWVSYISSSGNTI |
| ScFv domain | | YYADSVKGRFTISRDNAKNSLYLQMNSLRAEDTAVYYCARSTMVREDYWGQGTLVTVS |
| | | SASGGGGSGGRASGGGGSDIVLTQSPLSLPVTLGQPASISCKSSESLVHNSGKTYLNW |
| | | FHQRPGQSPRRLIYEVSNRDSGVPDRFTGSGSGTDFTLKISRVEAEDVGVYYCMQGTH |
| | | WPGTFGQGTKLEIK |
| 139110-nt | 65 | CAAGTGCAACTGGTGCAAAGCGGAGGAGGATTGGTCAAACCCGGAGGAAGCCTGAGAC |
| ScFv domain | | TGTCATGCGCGGCCTCTGGATTCACCTTCTCCGATTACTACATGTCATGGATCAGACA |
| | | GGCCCCGGGGAAGGGCCTCGAATGGGTGTCCTACATCTCGTCCTCCGGGAACACCATC |
| | | TACTACGCCGACAGCGTGAAGGGCCGCTTTACCATTTCCCGCGACAACGCAAAGAACT |
| | | CGCTGTACCTTCAGATGAATTCCCTGCGGGCTGAAGATACCGCGGTGTACTATTGCGC |
| | | CCGGTCCACTATGGTCCGGGAGGACTACTGGGGACAGGGCACACTCGTGACCGTGTCC |
| | | AGCGCGAGCGGGGGTGGAGGCAGCGGTGGACGCGCCTCCGGCGGCGGCGGTTCAGACA |
| | | TCGTGCTGACTCAGTCGCCCCTGTCGCTGCCGGTCACCCTGGGCCAACCGGCCTCAAT |
| | | TAGCTGCAAGTCCTCGGAGAGCCTGGTGCACAACTCAGGAAAGACTTACCTGAACTGG |
| | | TTCCATCAGCGGCCTGGACAGTCCCCACGGAGGCTCATCTATGAAGTGTCCAACAGGG |
| | | ATTCGGGGGTGCCCGACCGCTTCACTGGCTCCGGGTCCGGCACCGACTTCACCTTGAA |
| | | AATCTCCAGAGTGGAAGCCGAGGACGTGGGCGTGTACTACTGTATGCAGGGTACCCAC |
| | | TGGCCTGGAACCTTTGGACAAGGAACTAAGCTCGAGATTAAG |
| 139110-aa | 80 | QVQLVQSGGGLVKPGGSLRLSCAASGFTFSDYYMSWIRQAPGKGLEWVSYISSSGNTI |
| VH | | YYADSVKGRFTISRDNAKNSLYLQMNSLRAEDTAVYYCARSTMVREDYWGQGTLVTVS |
| | | S |
| 139110-aa | 95 | DIVLTQSPLSLPVTLGQPASISCKSSESLVHNSGKTYLNWFHQRPGQSPRRLIYEVSN |
| VL | | RDSGVPDRFTGSGSGTDFTLKISRVEAEDVGVYYCMQGTHWPGTFGQGTKLEIK |
| 139110-aa | 110 | MALPVTALLLPLALLLHAARPQVQLVQSGGGLVKPGGSLRLSCAASGFTFSDYYMSWI |
| Full CAR | | RQAPGKGLEWVSYISSSGNTIYYADSVKGRFTISRDNAKNSLYLQMNSLRAEDTAVYY |
| | | CARSTMVREDYWGQGTLVTVSSASGGGGSGGRASGGGGSDIVLTQSPLSLPVTLGQPA |
| | | SISCKSSESLVHNSGKTYLNWFHQRPGQSPRRLIYEVSNRDSGVPDRFTGSGSGTDFT |
| | | LKISRVEAEDVGVYYCMQGTHWPGTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139110-nt | 125 | ATGGCCCTCCCTGTCACCGCCCTGCTGCTTCCGCTGGCTCTTCTGCTCCACGCCGCTC |
| Full CAR | | GGCCCCAAGTGCAACTGGTGCAAAGCGGAGGAGGATTGGTCAAACCCGGAGGAAGCCT |
| | | GAGACTGTCATGCGCGGCCTCTGGATTCACCTTCTCCGATTACTACATGTCATGGATC |
| | | AGACAGGCCCCGGGGAAGGGCCTCGAATGGGTGTCCTACATCTCGTCCTCCGGGAACA |
| | | CCATCTACTACGCCGACAGCGTGAAGGGCCGCTTTACCATTTCCCGCGACAACGCAAA |
| | | GAACTCGCTGTACCTTCAGATGAATTCCCTGCGGGCTGAAGATACCGCGGTGTACTAT |
| | | TGCGCCCGGTCCACTATGGTCCGGGAGGACTACTGGGGACAGGGCACACTCGTGACCG |
| | | TGTCCAGCGCGAGCGGGGGTGGAGGCAGCGGTGGACGCGCCTCCGGCGGCGGCGGTTC |
| | | AGACATCGTGCTGACTCAGTCGCCCCTGTCGCTGCCGGTCACCCTGGGCCAACCGGCC |
| | | TCAATTAGCTGCAAGTCCTCGGAGAGCCTGGTGCACAACTCAGGAAAGACTTACCTGA |
| | | ACTGGTTCCATCAGCGGCCTGGACAGTCCCCACGGAGGCTCATCTATGAAGTGTCCAA |
| | | CAGGGATTCGGGGGTGCCCGACCGCTTCACTGGCTCCGGGTCCGGCACCGACTTCACC |
| | | TTGAAAATCTCCAGAGTGGAAGCCGAGGACGTGGGCGTGTACTACTGTATGCAGGGTA |
| | | CCCACTGGCCTGGAACCTTTGGACAAGGAACTAAGCTCGAGATTAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

139112

| | | |
|---|---|---|
| 139112-aa | 51 | QVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIRLTQSPSPLSASVGDRVTITCQASEDINKFLNWYHQTPGK |
| | | APKLLIYDASTLQTGVPSRFSGSGSGTDFTLTINSLQPEDIGTYYCQQYESLPLTFGG |
| | | GTKVEIK |
| 139112-nt | 66 | CAAGTGCAACTCGTGGAATCTGGTGGAGGACTCGTGCAACCCGGTGGAAGCCTTAGGC |
| ScFv domain | | TGTCGTGCGCCGTCAGCGGGTTTGCTCTGAGCAACCATGGAATGTCCTGGGTCCGCCG |
| | | GGCACCGGGAAAAGGGCTGGAATGGGTGTCCGGCATCGTGTACAGCGGGTCAACCTAT |
| | | TACGCCGCGTCCGTGAAGGGCAGATTCACTATCTCAAGAGACAACAGCCGGAACACCC |
| | | TGTACTTGCAAATGAATTCCCTGCGCCCCGAGGACACCGCCATCTACTACTGCTCCGC |
| | | CCACGGAGGAGAGTCGGACGTGTGGGGCCAGGGAACGACTGTGACTGTGTCCAGCGCA |
| | | TCAGGAGGGGGTGGTTCGGGCGGCCGGGCCTCGGGGGGAGGAGGTTCCGACATTCGGC |
| | | TGACCCAGTCCCCGTCCCCACTGTCGGCCTCCGTCGGCGACCGCGTGACCATCACTTG |
| | | TCAGGCGTCCGAGGACATTAACAAGTTCCTGAACTGGTACCACCAGACCCCTGGAAAG |
| | | GCCCCCAAGCTGCTGATCTACGATGCCTCGACCCTTCAAACTGGAGTGCCTAGCCGGT |
| | | TCTCCGGGTCCGGCTCCGGCACTGATTTCACTCTGACCATCAACTCATTGCAGCCGGA |
| | | AGATATCGGGACCTACTATTGCCAGCAGTACGAATCCCTCCCGCTCACATTCGGCGGG |
| | | GGAACCAAGGTCGAGATTAAG |
| 139112-aa | 81 | QVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139112-aa | 96 | DIRLTQSPSPLSASVGDRVTITCQASEDINKFLNWYHQTPGKAPKLLIYDASTLQTGV |
| VL | | PSRFSGSGSGTDFTLTINSLQPEDIGTYYCQQYESLPLTFGGGTKVEIK |
| 139112-aa | 111 | MALPVTALLLPLALLLHAARPQVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIRLTQSPSPLSASVGDRVTI |
| | | TCQASEDINKFLNWYHQTPGKAPKLLIYDASTLQTGVPSRFSGSGSGTDFTLTINSLQ |
| | | PEDIGTYYCQQYESLPLTFGGGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139112-nt | 126 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGCAACCCGGTGGAAGCCT |
| | | TAGGCTGTCGTGCGCCGTCAGCGGGTTTGCTCTGAGCAACCATGGAATGTCCTGGGTC |
| | | CGCCGGGCACCGGGAAAAGGGCTGGAATGGGTGTCCGGCATCGTGTACAGCGGGTCAA |
| | | CCTATTACGCCGCGTCCGTGAAGGGCAGATTCACTATCTCAAGAGACAACAGCCGGAA |
| | | CACCCTGTACTTGCAAATGAATTCCCTGCGCCCCGAGGACACCGCCATCTACTACTGC |
| | | TCCGCCCACGGAGGAGAGTCGGACGTGTGGGGCCAGGGAACGACTGTGACTGTGTCCA |
| | | GCGCATCAGGAGGGGGTGGTTCGGGCGGCCGGGCCTCGGGGGGAGGAGGTTCCGACAT |
| | | TCGGCTGACCCAGTCCCCGTCCCCACTGTCGGCCTCCGTCGGCGACCGCGTGACCATC |
| | | ACTTGTCAGGCGTCCGAGGACATTAACAAGTTCCTGAACTGGTACCACCAGACCCCTG |
| | | GAAAGGCCCCCAAGCTGCTGATCTACGATGCCTCGACCCTTCAAACTGGAGTGCCTAG |
| | | CCGGTTCTCCGGGTCCGGCTCCGGCACTGATTTCACTCTGACCATCAACTCATTGCAG |
| | | CCGGAAGATATCGGGACCTACTATTGCCAGCAGTACGAATCCCTCCCGCTCACATTCG |
| | | GCGGGGGAACCAAGGTCGAGATTAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |

139113

| | | |
|---|---|---|
| 139113-aa | 52 | EVQLVETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSETTLTQSPATLSVSPGERATLSCRASQSVGSNLAWYQQKPGQ |
| | | GPRLLIYGASTRATGIPARFSGSGSGTEFTLTISSLQPEDFAVYYCQQYNDWLPVTFG |
| | | QGTKVEIK |
| 139113-nt | 67 | GAAGTGCAATTGGTGGAAACTGGAGGAGGACTTGTGCAACCTGGAGGATCATTGCGGC |
| ScFv domain | | TCTCATGCGCTGTCTCCGGCTTCGCCCTGTCAAATCACGGGATGTCGTGGGTCAGACG |
| | | GGCCCCGGGAAAGGGTCTGGAATGGGTGTCGGGGATTGTGTACAGCGGCTCCACCTAC |
| | | TACGCCGCTTCGGTCAAGGGCCGCTTCACTATTTCACGGGACAACAGCCGCAACACCC |
| | | TCTATCTGCAAATGAACTCTCTCCGCCCGGAGGATACCGCCATCTACTACTGCTCCGC |
| | | ACACGGCGGCGAATCCGACGTGTGGGGACAGGGAACCACTGTCACCGTGTCGTCCGCA |
| | | TCCGGTGGCGGAGGATCGGGTGGCCGGGCCTCCGGGGGCGGCGGCAGCGAGACTACCC |
| | | TGACCCAGTCCCCTGCCACTCTGTCCGTGAGCCCGGGAGAGAGAGCCACCCTTAGCTG |
| | | CCGGGCCAGCCAGAGCGTGGGCTCCAACCTGGCCTGGTACCAGCAGAAGCCAGGACAG |
| | | GGTCCCAGGCTGCTGATCTACGGAGCCTCCACTCGCGCGACCGGCATCCCCGCGAGGT |
| | | TCTCCGGGTCGGGTTCCGGGACCGAGTTCACCCTGACCATCTCCTCCCTCCAACCGGA |
| | | GGACTTCGCGGTGTACTACTGTCAGCAGTACAACGATTGGCTGCCCGTGACATTTGGA |
| | | CAGGGGACGAAGGTGGAAATCAAA |
| 139113-aa | 82 | EVQLVETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139113-aa | 97 | ETTLTQSPATLSVSPGERATLSCRASQSVGSNLAWYQQKPGQGPRLLIYGASTRATGI |
| VL | | PARFSGSGSGTEFTLTISSLQPEDFAVYYCQQYNDWLPVTFGQGTKVEIK |
| 139113-aa | 112 | MALPVTALLLPLALLLHAARPEVQLVETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSETTLTQSPATLSVSPGERATL |
| | | SCRASQSVGSNLAWYQQKPGQGPRLLIYGASTRATGIPARFSGSGSGTEFTLTISSLQ |
| | | PEDFAVYYCQQYNDWLPVTFGQGTKVEIKTTTPAPRPPTPAPTIASQPLSLRPEACRP |
| | | AAGGAVHTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGKGHDGLYQG |
| | | LSTATKDTYDALHMQALPPR |
| 139113-nt | 127 | ATGGCCCTCCCTGTCACCGCCCTGCTGCTTCCGCTGGCTCTTCTGCTCCACGCCGCTC |
| Full CAR | | GGCCCGAAGTGCAATTGGTGGAAACTGGAGGAGGACTTGTGCAACCTGGAGGATCATT |
| | | GCGGCTCTCATGCGCTGTCTCCGGCTTCGCCCTGTCAAATCACGGGATGTCGTGGGTC |
| | | AGACGGGCCCCGGGAAAGGGTCTGGAATGGGTGTCGGGGATTGTGTACAGCGGCTCCA |
| | | CCTACTACGCCGCTTCGGTCAAGGGCCGCTTCACTATTTCACGGGACAACAGCCGCAA |
| | | CACCCTCTATCTGCAAATGAACTCTCTCCGCCCGGAGGATACCGCCATCTACTACTGC |
| | | TCCGCACACGGCGGCGAATCCGACGTGTGGGGACAGGGAACCACTGTCACCGTGTCGT |
| | | CCGCATCCGGTGGCGGAGGATCGGGTGGCCGGGCCTCCGGGGGCGGCGGCAGCGAGAC |
| | | TACCCTGACCCAGTCCCCTGCCACTCTGTCCGTGAGCCCGGGAGAGAGAGCCACCCTT |
| | | AGCTGCCGGGCCAGCCAGAGCGTGGGCTCCAACCTGGCCTGGTACCAGCAGAAGCCAG |
| | | GACAGGGTCCCAGGCTGCTGATCTACGGAGCCTCCACTCGCGCGACCGGCATCCCCGC |
| | | GAGGTTCTCCGGGTCGGGTTCCGGGACCGAGTTCACCCTGACCATCTCCTCCCTCCAA |
| | | CCGGAGGACTTCGCGGTGTACTACTGTCAGCAGTACAACGATTGGCTGCCCGTGACAT |
| | | TTGGACAGGGGACGAAGGTGGAAATCAAAACCACTACCCCAGCACCGAGGCCACCCAC |
| | | CCCGGCTCCTACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAGGAGGAGGAAG |
| | | GCGGCTGCGAACTGCGCGTGAAATTCAGCCGCAGCGCAGATGCTCCAGCCTACAAGCA |
| | | GGGGCAGAACCAGCTCTACAACGAACTCAATCTTGGTCGGAGAGAGGAGTACGACGTG |
| | | CTGGACAAGCGGAGAGGACGGGACCCAGAAATGGGCGGGAAGCCGCGCAGAAAGAATC |
| | | CCCAAGAGGGCCTGTACAACGAGCTCCAAAAGGATAAGATGGCAGAAGCCTATAGCGA |
| | | GATTGGTATGAAAGGGGAACGCAGAAGAGGCAAAGGCCACGACGGACTGTACCAGGGA |
| | | CTCAGCACCGCCACCAAGGACACCTATGACGCTCTTCACATGCAGGCCCTGCCGCCTC |
| | | GG |

139114

| | | |
|---|---|---|
| 139114-aa | 53 | EVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPGTLSLSPGERATLSCRASQSIGSSSLAWYQQKPG |
| | | QAPRLLMYGASSRASGIPDRFSGSGSGTDFTLTISRLEPEDFAVYYCQQYAGSPPFTF |
| | | GQGTKVEIK |
| 139114-nt | 68 | GAAGTGCAATTGGTGGAATCTGGTGGAGGACTTGTGCAACCTGGAGGATCACTGAGAC |
| ScFv domain | | TGTCATGCGCGGTGTCCGGTTTTGCCCTGAGCAATCATGGGATGTCGTGGGTCCGGCG |
| | | CGCCCCCGGAAAGGGTCTGGAATGGGTGTCGGGTATCGTCTACTCCGGGAGCACTTAC |
| | | TACGCCGCGAGCGTGAAGGGCCGCTTCACCATTTCCCGCGATAACTCCCGCAACACCC |
| | | TGTACTTGCAAATGAACTCGCTCCGGCCTGAGGACACTGCCATCTACTACTGCTCCGC |
| | | ACACGGAGGAGAATCCGACGTGTGGGGCCAGGGAACTACCGTGACCGTCAGCAGCGCC |
| | | TCCGGCGGCGGGGGCTCAGGCGGACGGGCTAGCGGCGGCGGTGGCTCCGAGATCGTGC |
| | | TGACCCAGTCGCCTGGCACTCTCTCGCTGAGCCCCGGGGAAAGGGCAACCCTGTCCTG |
| | | TCGGGCCAGCCAGTCCATTGGATCATCCTCCCTCGCCTGGTATCAGCAGAAACCGGGA |
| | | CAGGCTCCGCGGCTGCTTATGTATGGGGCCAGCTCAAGAGCCTCCGGCATTCCCGACC |
| | | GGTTCTCCGGGTCCGGTTCCGGCACCGATTTCACCCTGACTATCTCGAGGCTGGAGCC |
| | | AGAGGACTTCGCCGTGTACTACTGCCAGCAGTACGCGGGGTCCCCGCCGTTCACGTTC |
| | | GGACAGGGAACCAAGGTCGAGATCAAG |
| 139114-aa | 83 | EVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139114-aa | 98 | EIVLTQSPGTLSLSPGERATLSCRASQSIGSSSLAWYQQKPGQAPRLLMYGASSRASG |
| VL | | IPDRFSGSGSGTDFTLTISRLEPEDFAVYYCQQYAGSPPFTFGQGTKVEIK |
| 139114-aa | 113 | MALPVTALLLPLALLLHAARPEVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPGTLSLSPGERATL |
| | | SCRASQSIGSSSLAWYQQKPGQAPRLLMYGASSRASGIPDRFSGSGSGTDFTLTISRL |
| | | EPEDFAVYYCQQYAGSPPFTFGQGTKVEIKTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 139114-nt | 128 | ATGGCCCTCCCTGTCACCGCCCTGCTGCTTCCGCTGGCTCTTCTGCTCCACGCCGCTC |
| Full CAR | | GGCCCGAAGTGCAATTGGTGGAATCTGGTGGAGGACTTGTGCAACCTGGAGGATCACT |
| | | GAGACTGTCATGCGCGGTGTCCGGTTTTGCCCTGAGCAATCATGGGATGTCGTGGGTC |
| | | CGGCGCGCCCCCGGAAAGGGTCTGGAATGGGTGTCGGGTATCGTCTACTCCGGGAGCA |
| | | CTTACTACGCCGCGAGCGTGAAGGGCCGCTTCACCATTTCCCGCGATAACTCCCGCAA |
| | | CACCCTGTACTTGCAAATGAACTCGCTCCGGCCTGAGGACACTGCCATCTACTACTGC |
| | | TCCGCACACGGAGGAGAATCCGACGTGTGGGGCCAGGGAACTACCGTGACCGTCAGCA |
| | | GCGCCTCCGGCGGCGGGGGCTCAGGCGGACGGGCTAGCGGCGGCGGTGGCTCCGAGAT |
| | | CGTGCTGACCCAGTCGCCTGGCACTCTCTCGCTGAGCCCCGGGGAAAGGGCAACCCTG |
| | | TCCTGTCGGGCCAGCCAGTCCATTGGATCATCCTCCCTCGCCTGGTATCAGCAGAAAC |
| | | CGGGACAGGCTCCGCGGCTGCTTATGTATGGGGCCAGCTCAAGAGCCTCCGGCATTCC |
| | | CGACCGGTTCTCCGGGTCCGGTTCCGGCACCGATTTCACCCTGACTATCTCGAGGCTG |
| | | GAGCCAGAGGACTTCGCCGTGTACTACTGCCAGCAGTACGCGGGGTCCCCGCCGTTCA |
| | | CGTTCGGACAGGGAACCAAGGTCGAGATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |

149362

| | | |
|---|---|---|
| 149362-aa | 129 | QVQLQESGPGLVKPSETLSLTCTVSGGSISSSYYYWGWIRQPPGKGLEWIGSIYYSGS |
| ScFv domain | | AYYNPSLKSRVTISVDTSKNQFSLRLSSVTAADTAVYYCARHWQEWPDAFDIWGQGTM |
| | | VTVSSGGGGSGGGGSGGGGSETTLTQSPAFMSATPGDKVIISCKASQDIDDAMNWYQQ |
| | | KPGEAPLFIIQSATSPVPGIPPRFSGSGFGTDFSLTINNIESEDAAYYFCLQHDNFPL |
| | | TFGQGTKLEIK |
| 149362-nt ScFv | 150 | CAAGTGCAGCTTCAGGAAAGCGGACCGGGCCTGGTCAAGCCATCCGAAACTCTCTCCC |
| domain | | TGACTTGCACTGTGTCTGGCGGTTCCATCTCATCGTCGTACTACTACTGGGGCTGGAT |
| | | TAGGCAGCCGCCCGGAAAGGGACTGGAGTGGATCGGAAGCATCTACTATTCCGGCTCG |
| | | GCGTACTACAACCCTAGCCTCAAGTCGAGAGTGACCATCTCCGTGGATACCTCCAAGA |
| | | ACCAGTTTTCCCTGCGCCTGAGCTCCGTGACCGCCGCTGACACCGCCGTGTACTACTG |
| | | TGCTCGGCATTGGCAGGAATGGCCCGATGCCTTCGACATTTGGGGCCAGGGCACTATG |
| | | GTCACTGTGTCATCCGGGGGTGGAGGCAGCGGGGGAGGAGGGTCCGGGGGGGGAGGTT |
| | | CAGAGACAACCTTGACCCAGTCACCCGCATTCATGTCCGCCACTCCGGGAGACAAGGT |
| | | CATCATCTCGTGCAAAGCGTCCCAGGATATCGACGATGCCATGAATTGGTACCAGCAG |
| | | AAGCCTGGCGAAGCGCCGCTGTTCATTATCCAATCCGCAACCTCGCCCGTGCCTGGAA |
| | | TCCCACCGCGGTTCAGCGGCAGCGGTTTCGGAACCGACTTTTCCCTGACCATTAACAA |
| | | CATTGAGTCCGAGGACGCCGCCTACTACTTCTGCCTGCAACACGACAACTTCCCTCTC |
| | | ACGTTCGGCCAGGGAACCAAGCTGGAAATCAAG |
| 149362-aa | 171 | QVQLQESGPGLVKPSETLSLTCTVSGGSISSSYYYWGWIRQPPGKGLEWIGSIYYSGS |
| VH | | AYYNPSLKSRVTISVDTSKNQFSLRLSSVTAADTAVYYCARHWQEWPDAFDIWGQGTM |
| | | VTVSS |
| 149362-aa | 192 | ETTLTQSPAFMSATPGDKVIISCKASQDIDDAMNWYQQKPGEAPLFIIQSATSPVPGI |
| VL | | PPRFSGSGFGTDFSLTINNIESEDAAYYFCLQHDNFPLTFGQGTKLEIK |
| 149362-aa Full | 213 | MALPVTALLLPLALLLHAARPQVQLQESGPGLVKPSETLSLTCTVSGGSISSSYYYWG |
| CAR | | WIRQPPGKGLEWIGSIYYSGSAYYNPSLKSRVTISVDTSKNQFSLRLSSVTAADTAVY |
| | | YCARHWQEWPDAFDIWGQGTMVTVSSGGGGSGGGGSGGGGSETTLTQSPAFMSATPGD |
| | | KVIISCKASQDIDDAMNWYQQKPGEAPLFIIQSATSPVPGIPPRFSGSGFGTDFSLTI |
| | | NNIESEDAAYYFCLQHDNFPLTFGQGTKLEIKTTTPAPRPPTPAPTIASQPLSLRPEA |
| | | CRPAAGGAVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERRRGKGHDGL |
| | | YQGLSTATKDTYDALHMQALPPR |
| 149362-nt | 234 | ATGGCCCTCCCTGTCACCGCCCTGCTGCTTCCGCTGGCTCTTCTGCTCCACGCCGCTC |
| Full CAR | | GGCCCCAAGTGCAGCTTCAGGAAAGCGGACCGGGCCTGGTCAAGCCATCCGAAACTCT |
| | | CTCCCTGACTTGCACTGTGTCTGGCGGTTCCATCTCATCGTCGTACTACTACTGGGGC |
| | | TGGATTAGGCAGCCGCCCGGAAAGGGACTGGAGTGGATCGGAAGCATCTACTATTCCG |
| | | GCTCGGCGTACTACAACCCTAGCCTCAAGTCGAGAGTGACCATCTCCGTGGATACCTC |
| | | CAAGAACCAGTTTTCCCTGCGCCTGAGCTCCGTGACCGCCGCTGACACCGCCGTGTAC |
| | | TACTGTGCTCGGCATTGGCAGGAATGGCCCGATGCCTTCGACATTTGGGGCCAGGGCA |
| | | CTATGGTCACTGTGTCATCCGGGGGTGGAGGCAGCGGGGGAGGAGGGTCCGGGGGGGG |
| | | AGGTTCAGAGACAACCTTGACCCAGTCACCCGCATTCATGTCCGCCACTCCGGGAGAC |
| | | AAGGTCATCATCTCGTGCAAAGCGTCCCAGGATATCGACGATGCCATGAATTGGTACC |
| | | AGCAGAAGCCTGGCGAAGCGCCGCTGTTCATTATCCAATCCGCAACCTCGCCCGTGCC |
| | | TGGAATCCCACCGCGGTTCAGCGGCAGCGGTTTCGGAACCGACTTTTCCCTGACCATT |
| | | AACAACATTGAGTCCGAGGACGCCGCCTACTACTTCTGCCTGCAACACGACAACTTCC |
| | | CTCTCACGTTCGGCCAGGGAACCAAGCTGGAAATCAAGACCACTACCCCAGCACCGAG |
| | | GCCACCCACCCCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCAGATGCTCCAGC |
| | | CTACAAGCAGGGGCAGAACCAGCTCTACAACGAACTCAATCTTGGTCGGAGAGAGGAG |
| | | TACGACGTGCTGGACAAGCGGAGAGGACGGGACCCAGAAATGGGCGGGAAGCCGCGCA |
| | | GAAAGAATCCCCAAGAGGGCCTGTACAACGAGCTCCAAAAGGATAAGATGGCAGAAGC |
| | | CTATAGCGAGATTGGTATGAAAGGGGAACGCAGAAGAGGCAAAGGCCACGACGGACTG |
| | | TACCAGGGACTCAGCACCGCCACCAAGGACACCTATGACGCTCTTCACATGCAGGCCC |
| | | TGCCGCCTCGG |

149363

| | | |
|---|---|---|
| 149363-aa | 130 | VNLRESGPALVKPTQTLTLTCTFSGFSLRTSGMCVSWIRQPPGKALEWLARIDWDEDK |
| ScFv domain | | FYSTSLKTRLTISKDTSDNQVVLRMTNMDPADTATYYCARSGAGGTSATAFDIWGPGT |
| | | MVTVSSGGGGSGGGGSGGGGSDIQMTQSPSSLSASVGDRVTITCRASQDIYNNLAWFQ |
| | | LKPGSAPRSLMYAANKSQSGVPSRFSGSASGTDFTLTISSLQPEDFATYYCQHYYRFP |
| | | YSFGQGTKLEIK |
| 149363-nt ScFv | 151 | CAAGTCAATCTGCGCGAATCCGGCCCCGCCTTGGTCAAGCCTACCCAGACCCTCACTC |
| domain | | TGACCTGTACTTTCTCCGGCTTCTCCCTGCGGACTTCCGGGATGTGCGTGTCCTGGAT |
| | | CAGACAGCCTCCGGGAAAGGCCCTGGAGTGGCTCGCTCGCATTGACTGGGATGAGGAC |
| | | AAGTTCTACTCCACCTCACTCAAGACCAGGCTGACCATCAGCAAAGATACCTCTGACA |
| | | ACCAAGTGGTGCTCCGCATGACCAACATGGACCCAGCCGACACTGCCACTTACTACTG |
| | | CGCGAGGAGCGGAGCGGGCGGAACCTCCGCCACCGCCTTCGATATTTGGGGCCCGGGT |
| | | ACCATGGTCACCGTGTCAAGCGGAGGAGGGGGGTCCGGGGGCGGCGGTTCCGGGGGAG |
| | | GCGGATCGGACATTCAGATGACTCAGTCACCATCGTCCCTGAGCGCTAGCGTGGGCGA |
| | | CAGAGTGACAATCACTTGCCGGGCATCCCAGGACATCTATAACAACCTTGCGTGGTTC |
| | | CAGCTGAAGCCTGGTTCCGCACCGCGGTCACTTATGTACGCCGCCAACAAGAGCCAGT |
| | | CGGGAGTGCCGTCCCGGTTTTCCGGTTCGGCCTCGGGAACTGACTTCACCCTGACGAT |
| | | CTCCAGCCTGCAACCCGAGGATTTCGCCACCTACTACTGCCAGCACTACTACCGCTTT |
| | | CCCTACTCGTTCGGACAGGGAACCAAGCTGGAAATCAAG |
| 149363-aa | 172 | QVNLRESGPALVKPTQTLTLTCTFSGFSLRTSGMCVSWIRQPPGKALEWLARIDWDED |
| VH | | KFYSTSLKTRLTISKDTSDNQVVLRMTNMDPADTATYYCARSGAGGTSATAFDIWGPG |
| | | TMVTVSS |
| 149363-aa | 193 | DIQMTQSPSSLSASVGDRVTITCRASQDIYNNLAWFQLKPGSAPRSLMYAANKSQSGV |
| VL | | PSRFSGSASGTDFTLTISSLQPEDFATYYCQHYYRFPYSFGQGTKLEIK |
| 149363-aa Full | 214 | MALPVTALLLPLALLLHAARPQVNLRESGPALVKPTQTLTLTCTFSGFSLRTSGMCVS |
| CAR | | WIRQPPGKALEWLARIDWDEDKFYSTSLKTRLTISKDTSDNQVVLRMTNMDPADTATY |
| | | YCARSGAGGTSATAFDIWGPGTMVTVSSGGGGSGGGGSGGGGSDIQMTQSPSSLSASV |
| | | GDRVTITCRASQDIYNNLAWFQLKPGSAPRSLMYAANKSQSGVPSRFSGSASGTDFTL |
| | | TISSLQPEDFATYYCQHYYRFPYSFGQGTKLEIKTTTPAPRPPTPAPTIASQPLSLRP |
| | | EACRPAAGGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGERRRGKGHD |
| | | GLYQGLSTATKDTYDALHMQALPPR |
| 149363-nt | 235 | ATGGCCCTCCCTGTCACCGCCCTGCTGCTTCCGCTGGCTCTTCTGCTCCACGCCGCTC |
| Full CAR | | GGCCCCAAGTCAATCTGCGCGAATCCGGCCCCGCCTTGGTCAAGCCTACCCAGACCCT |
| | | CACTCTGACCTGTACTTTCTCCGGCTTCTCCCTGCGGACTTCCGGGATGTGCGTGTCC |
| | | TGGATCAGACAGCCTCCGGGAAAGGCCCTGGAGTGGCTCGCTCGCATTGACTGGGATG |
| | | AGGACAAGTTCTACTCCACCTCACTCAAGACCAGGCTGACCATCAGCAAAGATACCTC |
| | | TGACAACCAAGTGGTGCTCCGCATGACCAACATGGACCCAGCCGACACTGCCACTTAC |
| | | TACTGCGCGAGGAGCGGAGCGGGCGGAACCTCCGCCACCGCCTTCGATATTTGGGGCC |
| | | CGGGTACCATGGTCACCGTGTCAAGCGGAGGAGGGGGGTCCGGGGGCGGCGGTTCCGG |
| | | GGGAGGCGGATCGGACATTCAGATGACTCAGTCACCATCGTCCCTGAGCGCTAGCGTG |
| | | GGCGACAGAGTGACAATCACTTGCCGGGCATCCCAGGACATCTATAACAACCTTGCGT |
| | | GGTTCCAGCTGAAGCCTGGTTCCGCACCGCGGTCACTTATGTACGCCGCCAACAAGAG |
| | | CCAGTCGGGAGTGCCGTCCCGGTTTTCCGGTTCGGCCTCGGGAACTGACTTCACCCTG |
| | | ACGATCTCCAGCCTGCAACCCGAGGATTTCGCCACCTACTACTGCCAGCACTACTACC |
| | | GCTTTCCCTACTCGTTCGGACAGGGAACCAAGCTGGAAATCAAGACCACTACCCCAGC |
| | | ACCGAGGCCACCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCATGCCGGTTCC |
| | | CAGAGGAGGAGGAAGGCGGCTGCGAACTGCGCGTGAAATTCAGCCGCAGCGCAGATGC |
| | | TCCAGCCTACAAGCAGGGGCAGAACCAGCTCTACAACGAACTCAATCTTGGTCGGAGA |
| | | GAGGAGTACGACGTGCTGGACAAGCGGAGAGGACGGGACCCAGAAATGGGCGGGAAGC |
| | | CGCGCAGAAAGAATCCCCAAGAGGGCCTGTACAACGAGCTCCAAAAGGATAAGATGGC |
| | | AGAAGCCTATAGCGAGATTGGTATGAAAGGGGAACGCAGAAGAGGCAAAGGCCACGAC |
| | | GGACTGTACCAGGGACTCAGCACCGCCACCAAGGACACCTATGACGCTCTTCACATGC |
| | | AGGCCCTGCCGCCTCGG |

149364

| | | |
|---|---|---|
| 149364-aa | 131 | EVQLVESGGGLVKPGGSLRLSCAASGFTFSSYSMNWVRQAPGKGLEWVSSISSSSSYI |
| ScFv domain | | YYADSVKGRFTISRDNAKNSLYLQMNSLRAEDTAVYYCAKTIAAVYAFDIWGQGTTVT |
| | | VSSGGGGSGGGGSGGGGSEIVLTQSPLSLPVTPEEPASISCRSSQSLLHSNGYNYLDW |
| | | YLQKPGQSPQLLIYLGSNRASGVPDRFSGSGSGTDFTLKISRVEAEDVGVYYCMQALQ |
| | | TPYTFGQGTKLEIK |
| 149364-nt ScFv | 152 | GAAGTGCAGCTTGTCGAATCCGGGGGGGGACTGGTCAAGCCGGGCGGATCACTGAGAC |
| domain | | TGTCCTGCGCCGCGAGCGGCTTCACGTTCTCCTCCTACTCCATGAACTGGGTCCGCCA |
| | | AGCCCCCGGGAAGGGACTGGAATGGGTGTCCTCTATCTCCTCGTCGTCGTCCTACATC |
| | | TACTACGCCGACTCCGTGAAGGGAAGATTCACCATTTCCCGCGACAACGCAAAGAACT |
| | | CACTGTACTTGCAAATGAACTCACTCCGGGCCGAAGATACTGCTGTGTACTATTGCGC |
| | | CAAGACTATTGCCGCCGTCTACGCTTTCGACATCTGGGGCCAGGGAACCACCGTGACT |
| | | GTGTCGTCCGGTGGTGGTGGCTCGGGCGGAGGAGGAAGCGGCGGCGGGGGGTCCGAGA |
| | | TTGTGCTGACCCAGTCGCCACTGAGCCTCCCTGTGACCCCCGAGGAACCCGCCAGCAT |
| | | CAGCTGCCGGTCCAGCCAGTCCCTGCTCCACTCCAACGGATACAATTACCTCGATTGG |
| | | TACCTTCAGAAGCCTGGACAAAGCCCGCAGCTGCTCATCTACTTGGGATCAAACCGCG |
| | | CGTCAGGAGTGCCTGACCGGTTCTCCGGCTCGGGCAGCGGTACCGATTTCACCCTGAA |
| | | AATCTCCAGGGTGGAGGCAGAGGACGTGGGAGTGTATTACTGTATGCAGGCGCTGCAG |
| | | ACTCCGTACACATTTGGGCAGGGCACCAAGCTGGAGATCAAG |
| 149364-aa VH | 173 | EVQLVESGGGLVKPGGSLRLSCAASGFTFSSYSMNWVRQAPGKGLEWVSSISSSSSYI |
| | | YYADSVKGRFTISRDNAKNSLYLQMNSLRAEDTAVYYCAKTIAAVYAFDIWGQGTTVT |
| | | VSS |
| 149364-aa VL | 194 | EIVLTQSPLSLPVTPEEPASISCRSSQSLLHSNGYNYLDWYLQKPGQSPQLLIYLGSN |
| | | RASGVPDRFSGSGSGTDFTLKISRVEAEDVGVYYCMQALQTPYTFGQGTKLEIK |
| 149364-aa Full | 215 | MALPVTALLLPLALLLHAARPEVQLVESGGGLVKPGGSLRLSCAASGFTFSSYSMNWV |
| CAR | | RQAPGKGLEWVSSISSSSSYIYYADSVKGRFTISRDNAKNSLYLQMNSLRAEDTAVYY |
| | | CAKTIAAVYAFDIWGQGTTVTVSSGGGGSGGGGSGGGGSEIVLTQSPLSLPVTPEEPA |
| | | SISCRSSQSLLHSNGYNYLDWYLQKPGQSPQLLIYLGSNRASGVPDRFSGSGSGTDFT |
| | | LKISRVEAEDVGVYYCMQALQTPYTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 149364-nt | 236 | ATGGCCCTCCCTGTCACCGCCCTGCTGCTTCCGCTGGCTCTTCTGCTCCACGCCGCTC |
| Full CAR | | GGCCCGAAGTGCAGCTTGTCGAATCCGGGGGGGGACTGGTCAAGCCGGGCGGATCACT |
| | | GAGACTGTCCTGCGCCGCGAGCGGCTTCACGTTCTCCTCCTACTCCATGAACTGGGTC |
| | | CGCCAAGCCCCCGGGAAGGGACTGGAATGGGTGTCCTCTATCTCCTCGTCGTCGTCCT |
| | | ACATCTACTACGCCGACTCCGTGAAGGGAAGATTCACCATTTCCCGCGACAACGCAAA |
| | | GAACTCACTGTACTTGCAAATGAACTCACTCCGGGCCGAAGATACTGCTGTGTACTAT |
| | | TGCGCCAAGACTATTGCCGCCGTCTACGCTTTCGACATCTGGGGCCAGGGAACCACCG |
| | | TGACTGTGTCGTCCGGTGGTGGTGGCTCGGGCGGAGGAGGAAGCGGCGGCGGGGGGTC |
| | | CGAGATTGTGCTGACCCAGTCGCCACTGAGCCTCCCTGTGACCCCCGAGGAACCCGCC |
| | | AGCATCAGCTGCCGGTCCAGCCAGTCCCTGCTCCACTCCAACGGATACAATTACCTCG |
| | | ATTGGTACCTTCAGAAGCCTGGACAAAGCCCGCAGCTGCTCATCTACTTGGGATCAAA |
| | | CCGCGCGTCAGGAGTGCCTGACCGGTTCTCCGGCTCGGGCAGCGGTACCGATTTCACC |
| | | CTGAAAATCTCCAGGGTGGAGGCAGAGGACGTGGGAGTGTATTACTGTATGCAGGCGC |
| | | TGCAGACTCCGTACACATTTGGGCAGGGCACCAAGCTGGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

149365

| | | |
|---|---|---|
| 149365-aa | 132 | EVQLVESGGGLVKPGGSLRLSCAASGFTFSDYYMSWIRQAPGKGLEWVSYISSSGSTI |
| ScFv domain | | YYADSVKGRFTISRDNAKNSLYLQMNSLRAEDTAVYYCARDLRGAFDIWGQGTMVTVS |
| | | SGGGGSGGGGSGGGGSSYVLTQSPSVSAAPGYTATISCGGNNIGTKSVHWYQQKPGQA |
| | | PLLVIRDDSVRPSKIPGRFSGSNSGNMATLTISGVQAGDEADFYCQVWDSDSEHVVFG |
| | | GGTKLTVL |
| 149365-nt ScFv | 153 | GAAGTCCAGCTCGTGGAGTCCGGCGGAGGCCTTGTGAAGCCTGGAGGTTCGCTGAGAC |
| domain | | TGTCCTGCGCCGCCTCCGGCTTCACCTTCTCCGACTACTACATGTCCTGGATCAGACA |
| | | GGCCCCGGGAAAGGGCCTGGAATGGGTGTCCTACATCTCGTCATCGGGCAGCACTATC |
| | | TACTACGCGGACTCAGTGAAGGGGCGGTTCACCATTTCCCGGGATAACGCGAAGAACT |
| | | CGCTGTATCTGCAAATGAACTCACTGAGGGCCGAGGACACCGCCGTGTACTACTGCGC |
| | | CCGCGATCTCCGCGGGGCATTTGACATCTGGGGACAGGGAACCATGGTCACAGTGTCC |
| | | AGCGGAGGGGGAGGATCGGGTGGCGGAGGTTCCGGGGGTGGAGGCTCCTCCTACGTGC |
| | | TGACTCAGAGCCCAAGCGTCAGCGCTGCGCCCGGTTACACGGCAACCATCTCCTGTGG |
| | | CGGAAACAACATTGGGACCAAGTCTGTGCACTGGTATCAGCAGAAGCCGGGCCAAGCT |
| | | CCCCTGTTGGTGATCCGCGATGACTCCGTGCGGCCTAGCAAAATTCCGGGACGGTTCT |
| | | CCGGCTCCAACAGCGGCAATATGGCCACTCTCACCATCTCGGGAGTGCAGGCCGGAGA |
| | | TGAAGCCGACTTCTACTGCCAAGTCTGGGACTCAGACTCCGAGCATGTGGTGTTCGGG |
| | | GGCGGAACCAAGCTGACTGTGCTC |
| 149365-aa VH | 174 | EVQLVESGGGLVKPGGSLRLSCAASGFTFSDYYMSWIRQAPGKGLEWVSYISSSGSTI |
| | | YYADSVKGRFTISRDNAKNSLYLQMNSLRAEDTAVYYCARDLRGAFDIWGQGTMVTVS |
| | | S |
| 149365-aa VL | 195 | SYVLTQSPSVSAAPGYTATISCGGNNIGTKSVHWYQQKPGQAPLLVIRDDSVRPSKIP |
| | | GRFSGSNSGNMATLTISGVQAGDEADFYCQVWDSDSEHVVFGGGTKLTVL |
| 149365-aa Full | 216 | MALPVTALLLPLALLLHAARPEVQLVESGGGLVKPGGSLRLSCAASGFTFSDYYMSWI |
| CAR | | RQAPGKGLEWVSYISSSGSTIYYADSVKGRFTISRDNAKNSLYLQMNSLRAEDTAVYY |
| | | CARDLRGAFDIWGQGTMVTVSSGGGGSGGGGSGGGGSSYVLTQSPSVSAAPGYTATIS |
| | | CGGNNIGTKSVHWYQQKPGQAPLLVIRDDSVRPSKIPGRFSGSNSGNMATLTISGVQA |
| | | GDEADFYCQVWDSDSEHVVFGGGTKLTVLTTTPAPRPPTPAPTIASQPLSLRPEACRP |
| | | AAGGAVHTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGKGHDGLYQG |
| | | LSTATKDTYDALHMQALPPR |
| 149365-nt | 237 | ATGGCCCTCCCTGTCACCGCCCTGCTGCTTCCGCTGGCTCTTCTGCTCCACGCCGCTC |
| Full CAR | | GGCCCGAAGTCCAGCTCGTGGAGTCCGGCGGAGGCCTTGTGAAGCCTGGAGGTTCGCT |
| | | GAGACTGTCCTGCGCCGCCTCCGGCTTCACCTTCTCCGACTACTACATGTCCTGGATC |
| | | AGACAGGCCCCGGGAAAGGGCCTGGAATGGGTGTCCTACATCTCGTCATCGGGCAGCA |
| | | CTATCTACTACGCGGACTCAGTGAAGGGGCGGTTCACCATTTCCCGGGATAACGCGAA |
| | | GAACTCGCTGTATCTGCAAATGAACTCACTGAGGGCCGAGGACACCGCCGTGTACTAC |
| | | TGCGCCCGCGATCTCCGCGGGGCATTTGACATCTGGGGACAGGGAACCATGGTCACAG |
| | | TGTCCAGCGGAGGGGGAGGATCGGGTGGCGGAGGTTCCGGGGGTGGAGGCTCCTCCTA |
| | | CGTGCTGACTCAGAGCCCAAGCGTCAGCGCTGCGCCCGGTTACACGGCAACCATCTCC |
| | | TGTGGCGGAAACAACATTGGGACCAAGTCTGTGCACTGGTATCAGCAGAAGCCGGGCC |
| | | AAGCTCCCCTGTTGGTGATCCGCGATGACTCCGTGCGGCCTAGCAAAATTCCGGGACG |
| | | GTTCTCCGGCTCCAACAGCGGCAATATGGCCACTCTCACCATCTCGGGAGTGCAGGCC |
| | | GGAGATGAAGCCGACTTCTACTGCCAAGTCTGGGACTCAGACTCCGAGCATGTGGTGT |
| | | TCGGGGGCGGAACCAAGCTGACTGTGCTCACCACTACCCCAGCACCGAGGCCACCCAC |
| | | CCCGGCTCCTACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAGGAGGAGGAAG |
| | | GCGGCTGCGAACTGCGCGTGAAATTCAGCCGCAGCGCAGATGCTCCAGCCTACAAGCA |
| | | GGGGCAGAACCAGCTCTACAACGAACTCAATCTTGGTCGGAGAGAGGAGTACGACGTG |
| | | CTGGACAAGCGGAGAGGACGGGACCCAGAAATGGGCGGGAAGCCGCGCAGAAAGAATC |
| | | CCCAAGAGGGCCTGTACAACGAGCTCCAAAAGGATAAGATGGCAGAAGCCTATAGCGA |
| | | GATTGGTATGAAAGGGGAACGCAGAAGAGGCAAAGGCCACGACGGACTGTACCAGGGA |
| | | CTCAGCACCGCCACCAAGGACACCTATGACGCTCTTCACATGCAGGCCCTGCCGCCTC |
| | | GG |

149366

| | | |
|---|---|---|
| 149366-aa | 133 | QVQLVQSGAEVKKPGASVKVSCKPSGYTVTSHYIHWVRRAPGQGLEWMGMINPSGGVT |
| ScFv domain | | AYSQTLQGRVTMTSDTSSSTVYMELSSLRSEDTAMYYCAREGSGSGWYFDFWGRGTLV |
| | | TVSSGGGGSGGGGSGGGGSSYVLTQPPSVSVSPGQTASITCSGDGLSKKYVSWYQQKA |
| | | GQSPVVLISRDKERPSGIPDRFSGSNSADTATLTISGTQAMDEADYYCQAWDDTTVVF |
| | | GGGTKLTVL |
| 149366-nt ScFv | 154 | CAAGTGCAGCTGGTGCAGAGCGGGGCCGAAGTCAAGAAGCCGGGAGCCTCCGTGAAAG |
| domain | | TGTCCTGCAAGCCTTCGGGATACACCGTGACCTCCCACTACATTCATTGGGTCCGCCG |
| | | CGCCCCCGGCCAAGGACTCGAGTGGATGGGCATGATCAACCCTAGCGGCGGAGTGACC |
| | | GCGTACAGCCAGACGCTGCAGGGACGCGTGACTATGACCTCGGATACCTCCTCCTCCA |
| | | CCGTCTATATGGAACTGTCCAGCCTGCGGTCCGAGGATACCGCCATGTACTACTGCGC |
| | | CCGGGAAGGATCAGGCTCCGGGTGGTATTTCGACTTCTGGGGAAGAGGCACCCTCGTG |
| | | ACTGTGTCATCTGGGGGAGGGGGTTCCGGTGGTGGCGGATCGGGAGGAGGCGGTTCAT |
| | | CCTACGTGCTGACCCAGCCACCCTCCGTGTCCGTGAGCCCCGGCCAGACTGCATCGAT |
| | | TACATGTAGCGGCGACGGCCTCTCCAAGAAATACGTGTCGTGGTACCAGCAGAAGGCC |
| | | GGACAGAGCCCGGTGGTGCTGATCTCAAGAGATAAGGAGCGGCCTAGCGGAATCCCGG |
| | | ACAGGTTCTCGGGTTCCAACTCCGCGGACACTGCTACTCTGACCATCTCGGGGACCCA |
| | | GGCTATGGACGAAGCCGATTACTACTGCCAAGCCTGGGACGACACTACTGTCGTGTTT |
| | | GGAGGGGGCACCAAGTTGACCGTCCTT |
| 149366-aa VH | 175 | QVQLVQSGAEVKKPGASVKVSCKPSGYTVTSHYIHWVRRAPGQGLEWMGMINPSGGVT |
| | | AYSQTLQGRVTMTSDTSSSTVYMELSSLRSEDTAMYYCAREGSGSGWYFDFWGRGTLV |
| | | TVSS |
| 149366-aa VL | 196 | SYVLTQPPSVSVSPGQTASITCSGDGLSKKYVSWYQQKAGQSPVVLISRDKERPSGIP |
| | | DRFSGSNSADTATLTISGTQAMDEADYYCQAWDDTTVVFGGGTKLTVL |
| 149366-aa Full | 217 | MALPVTALLLPLALLLHAARPQVQLVQSGAEVKKPGASVKVSCKPSGYTVTSHYIHWV |
| CAR | | RRAPGQGLEWMGMINPSGGVTAYSQTLQGRVTMTSDTSSSTVYMELSSLRSEDTAMYY |
| | | CAREGSGSGWYFDFWGRGTLVTVSSGGGGSGGGGSGGGGSSYVLTQPPSVSVSPGQTA |
| | | SITCSGDGLSKKYVSWYQQKAGQSPVVLISRDKERPSGIPDRFSGSNSADTATLTISG |
| | | TQAMDEADYYCQAWDDTTVVFGGGTKLTVLTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 149366-nt | 238 | ATGGCCCTCCCTGTCACCGCCCTGCTGCTTCCGCTGGCTCTTCTGCTCCACGCCGCTC |
| Full CAR | | GGCCCCAAGTGCAGCTGGTGCAGAGCGGGGCCGAAGTCAAGAAGCCGGGAGCCTCCGT |
| | | GAAAGTGTCCTGCAAGCCTTCGGGATACACCGTGACCTCCCACTACATTCATTGGGTC |
| | | CGCCGCGCCCCCGGCCAAGGACTCGAGTGGATGGGCATGATCAACCCTAGCGGCGGAG |
| | | TGACCGCGTACAGCCAGACGCTGCAGGGACGCGTGACTATGACCTCGGATACCTCCTC |
| | | CTCCACCGTCTATATGGAACTGTCCAGCCTGCGGTCCGAGGATACCGCCATGTACTAC |
| | | TGCGCCCGGGAAGGATCAGGCTCCGGGTGGTATTTCGACTTCTGGGGAAGAGGCACCC |
| | | TCGTGACTGTGTCATCTGGGGGAGGGGGTTCCGGTGGTGGCGGATCGGGAGGAGGCGG |
| | | TTCATCCTACGTGCTGACCCAGCCACCCTCCGTGTCCGTGAGCCCCGGCCAGACTGCA |
| | | TCGATTACATGTAGCGGCGACGGCCTCTCCAAGAAATACGTGTCGTGGTACCAGCAGA |
| | | AGGCCGGACAGAGCCCGGTGGTGCTGATCTCAAGAGATAAGGAGCGGCCTAGCGGAAT |
| | | CCCGGACAGGTTCTCGGGTTCCAACTCCGCGGACACTGCTACTCTGACCATCTCGGGG |
| | | ACCCAGGCTATGGACGAAGCCGATTACTACTGCCAAGCCTGGGACGACACTACTGTCG |
| | | TGTTTGGAGGGGGCACCAAGTTGACCGTCCTTACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |

149367

| | | |
|---|---|---|
| 149367-aa | 134 | QVQLQESGPGLVKPSQTLSLTCTVSGGSISSGGYYWSWIRQHPGKGLEWIGYIYYSGS |
| ScFv domain | | TYYNPSLKSRVTISVDTSKNQFSLKLSSVTAADTAVYYCARAGIAARLRGAFDIWGQG |
| | | TMVTVSSGGGGSGGGGSGGGGSDIVMTQSPSSVSASVGDRVIITCRASQGIRNWLAWY |
| | | QQKPGKAPNLLIYAASNLQSGVPSRFSGSGSGADFTLTISSLQPEDVATYYCQKYNSA |
| | | PFTFGPGTKVDIK |
| 149367-nt ScFv | 155 | CAAGTGCAGCTTCAGGAGAGCGGCCCGGGACTCGTGAAGCCGTCCCAGACCCTGTCCC |
| domain | | TGACTTGCACCGTGTCGGGAGGAAGCATCTCGAGCGGAGGCTACTATTGGTCGTGGAT |
| | | TCGGCAGCACCCTGGAAAGGGCCTGGAATGGATCGGCTACATCTACTACTCCGGCTCG |
| | | ACCTACTACAACCCATCGCTGAAGTCCAGAGTGACAATCTCAGTGGACACGTCCAAGA |
| | | ATCAGTTCAGCCTGAAGCTCTCTTCCGTGACTGCGGCCGACACCGCCGTGTACTACTG |
| | | CGCACGCGCTGGAATTGCCGCCCGGCTGAGGGGTGCCTTCGACATTTGGGGACAGGGC |
| | | ACCATGGTCACCGTGTCCTCCGGCGGCGGAGGTTCCGGGGGTGGAGGCTCAGGAGGAG |
| | | GGGGGTCCGACATCGTCATGACTCAGTCGCCCTCAAGCGTCAGCGCGTCCGTCGGGGA |
| | | CAGAGTGATCATCACCTGTCGGGCGTCCCAGGGAATTCGCAACTGGCTGGCCTGGTAT |
| | | CAGCAGAAGCCCGGAAAGGCCCCCAACCTGTTGATCTACGCCGCCTCAAACCTCCAAT |
| | | CCGGGGTGCCGAGCCGCTTCAGCGGCTCCGGTTCGGGTGCCGATTTCACTCTGACCAT |
| | | CTCCTCCCTGCAACCTGAAGATGTGGCTACCTACTACTGCCAAAAGTACAACTCCGCA |
| | | CCTTTTACTTTCGGACCGGGGACCAAAGTGGACATTAAG |
| 149367-aa VH | 176 | QVQLQESGPGLVKPSQTLSLTCTVSGGSISSGGYYWSWIRQHPGKGLEWIGYIYYSGS |
| | | TYYNPSLKSRVTISVDTSKNQFSLKLSSVTAADTAVYYCARAGIAARLRGAFDIWGQG |
| | | TMVTVSS |
| 149367-aa VL | 197 | DIVMTQSPSSVSASVGDRVIITCRASQGIRNWLAWYQQKPGKAPNLLIYAASNLQSGV |
| | | PSRFSGSGSGADFTLTISSLQPEDVATYYCQKYNSAPFTFGPGTKVDIK |
| 149367-aa Full | 218 | MALPVTALLLPLALLLHAARPQVQLQESGPGLVKPSQTLSLTCTVSGGSISSGGYYWS |
| CAR | | WIRQHPGKGLEWIGYIYYSGSTYYNPSLKSRVTISVDTSKNQFSLKLSSVTAADTAVY |
| | | YCARAGIAARLRGAFDIWGQGTMVTVSSGGGGSGGGGSGGGGSDIVMTQSPSSVSASV |
| | | GDRVIITCRASQGIRNWLAWYQQKPGKAPNLLIYAASNLQSGVPSRFSGSGSGADFTL |
| | | TISSLQPEDVATYYCQKYNSAPFTFGPGTKVDIKTTTPAPRPPTPAPTIASQPLSLRP |
| | | EACRPAAGGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGERRRGKGHD |
| | | GLYQGLSTATKDTYDALHMQALPPR |
| 149367-nt | 239 | ATGGCCCTCCCTGTCACCGCCCTGCTGCTTCCGCTGGCTCTTCTGCTCCACGCCGCTC |
| Full CAR | | GGCCCCAAGTGCAGCTTCAGGAGAGCGGCCCGGGACTCGTGAAGCCGTCCCAGACCCT |
| | | GTCCCTGACTTGCACCGTGTCGGGAGGAAGCATCTCGAGCGGAGGCTACTATTGGTCG |
| | | TGGATTCGGCAGCACCCTGGAAAGGGCCTGGAATGGATCGGCTACATCTACTACTCCG |
| | | GCTCGACCTACTACAACCCATCGCTGAAGTCCAGAGTGACAATCTCAGTGGACACGTC |
| | | CAAGAATCAGTTCAGCCTGAAGCTCTCTTCCGTGACTGCGGCCGACACCGCCGTGTAC |
| | | TACTGCGCACGCGCTGGAATTGCCGCCCGGCTGAGGGGTGCCTTCGACATTTGGGGAC |
| | | AGGGCACCATGGTCACCGTGTCCTCCGGCGGCGGAGGTTCCGGGGGTGGAGGCTCAGG |
| | | AGGAGGGGGGTCCGACATCGTCATGACTCAGTCGCCCTCAAGCGTCAGCGCGTCCGTC |
| | | GGGGACAGAGTGATCATCACCTGTCGGGCGTCCCAGGGAATTCGCAACTGGCTGGCCT |
| | | GGTATCAGCAGAAGCCCGGAAAGGCCCCCAACCTGTTGATCTACGCCGCCTCAAACCT |
| | | CCAATCCGGGGTGCCGAGCCGCTTCAGCGGCTCCGGTTCGGGTGCCGATTTCACTCTG |
| | | ACCATCTCCTCCCTGCAACCTGAAGATGTGGCTACCTACTACTGCCAAAAGTACAACT |
| | | CCGCACCTTTTACTTTCGGACCGGGGACCAAAGTGGACATTAAGACCACTACCCCAGC |
| | | ACCGAGGCCACCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCATGCCGGTTCC |
| | | CAGAGGAGGAGGAAGGCGGCTGCGAACTGCGCGTGAAATTCAGCCGCAGCGCAGATGC |
| | | TCCAGCCTACAAGCAGGGGCAGAACCAGCTCTACAACGAACTCAATCTTGGTCGGAGA |
| | | GAGGAGTACGACGTGCTGGACAAGCGGAGAGGACGGGACCCAGAAATGGGCGGGAAGC |
| | | CGCGCAGAAAGAATCCCCAAGAGGGCCTGTACAACGAGCTCCAAAAGGATAAGATGGC |
| | | AGAAGCCTATAGCGAGATTGGTATGAAAGGGGAACGCAGAAGAGGCAAAGGCCACGAC |
| | | GGACTGTACCAGGGACTCAGCACCGCCACCAAGGACACCTATGACGCTCTTCACATGC |
| | | AGGCCCTGCCGCCTCGG |

149368

| | | |
|---|---|---|
| 149368-aa | 135 | QVQLVQSGAEVKKPGSSVKVSCKASGGTFSSYAISWVRQAPGQGLEWMGGIIPIFGTA |
| ScFv domain | | NYAQKFQGRVTITADESTSTAYMELSSLRSEDTAVYYCARRGGYQLLRWDVGLLRSAF |
| | | DIWGQGTMVTVSSGGGGSGGGGSGGGGSSYVLTQPPSVSVAPGQTARITCGGNNIGSK |
| | | SVHWYQQKPGQAPVLVLYGKNNRPSGVPDRFSGSRSGTTASLTITGAQAEDEADYYCS |
| | | SRDSSGDHLRVFGTGTKVTVL |
| 149368-nt ScFv | 156 | CAAGTGCAGCTGGTCCAGTCGGGCGCCGAGGTCAAGAAGCCCGGGAGCTCTGTGAAAG |
| domain | | TGTCCTGCAAGGCCTCCGGGGGCACCTTTAGCTCCTACGCCATCTCCTGGGTCCGCCA |
| | | AGCACCGGGTCAAGGCCTGGAGTGGATGGGGGGAATTATCCCTATCTTCGGCACTGCC |
| | | AACTACGCCCAGAAGTTCCAGGGACGCGTGACCATTACCGCGGACGAATCCACCTCCA |
| | | CCGCTTATATGGAGCTGTCCAGCTTGCGCTCGGAAGATACCGCCGTGTACTACTGCGC |
| | | CCGGAGGGGTGGATACCAGCTGCTGAGATGGGACGTGGGCCTCCTGCGGTCGGCGTTC |
| | | GACATCTGGGGCCAGGGCACTATGGTCACTGTGTCCAGCGGAGGAGGCGGATCGGGAG |
| | | GCGGCGGATCAGGGGGAGGCGGTTCCAGCTACGTGCTTACTCAACCCCCTTCGGTGTC |
| | | CGTGGCCCCGGGACAGACCGCCAGAATCACTTGCGGAGGAAACAACATTGGGTCCAAG |
| | | AGCGTGCATTGGTACCAGCAGAAGCCAGGACAGGCCCCTGTGCTGGTGCTCTACGGGA |
| | | AGAACAATCGGCCCAGCGGAGTGCCGGACAGGTTCTCGGGTTCACGCTCCGGTACAAC |
| | | CGCTTCACTGACTATCACCGGGGCCCAGGCAGAGGATGAAGCGGACTACTACTGTTCC |
| | | TCCCGGGATTCATCCGGCGACCACCTCCGGGTGTTCGGAACCGGAACGAAGGTCACCG |
| | | TGCTG |
| 149368-aa VH | 177 | QVQLVQSGAEVKKPGSSVKVSCKASGGTFSSYAISWVRQAPGQGLEWMGGIIPIFGTA |
| | | NYAQKFQGRVTITADESTSTAYMELSSLRSEDTAVYYCARRGGYQLLRWDVGLLRSAF |
| | | DIWGQGTMVTVSS |
| 149368-aa VL | 198 | SYVLTQPPSVSVAPGQTARITCGGNNIGSKSVHWYQQKPGQAPVLVLYGKNNRPSGVP |
| | | DRFSGSRSGTTASLTITGAQAEDEADYYCSSRDSSGDHLRVFGTGTKVTVL |
| 149368-aa Full | 219 | MALPVTALLLPLALLLHAARPQVQLVQSGAEVKKPGSSVKVSCKASGGTFSSYAISWV |
| CAR | | RQAPGQGLEWMGGIIPIFGTANYAQKFQGRVTITADESTSTAYMELSSLRSEDTAVYY |
| | | CARRGGYQLLRWDVGLLRSAFDIWGQGTMVTVSSGGGGSGGGGSGGGGSSYVLTQPPS |
| | | VSVAPGQTARITCGGNNIGSKSVHWYQQKPGQAPVLVLYGKNNRPSGVPDRFSGSRSG |
| | | TTASLTITGAQAEDEADYYCSSRDSSGDHLRVFGTGTKVTVLTTTPAPRPPTPAPTIA |
| | | SQPLSLRPEACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 149368-nt | 240 | ATGGCCCTCCCTGTCACCGCCCTGCTGCTTCCGCTGGCTCTTCTGCTCCACGCCGCTC |
| Full CAR | | GGCCCCAAGTGCAGCTGGTCCAGTCGGGCGCCGAGGTCAAGAAGCCCGGGAGCTCTGT |
| | | GAAAGTGTCCTGCAAGGCCTCCGGGGGCACCTTTAGCTCCTACGCCATCTCCTGGGTC |
| | | CGCCAAGCACCGGGTCAAGGCCTGGAGTGGATGGGGGGAATTATCCCTATCTTCGGCA |
| | | CTGCCAACTACGCCCAGAAGTTCCAGGGACGCGTGACCATTACCGCGGACGAATCCAC |
| | | CTCCACCGCTTATATGGAGCTGTCCAGCTTGCGCTCGGAAGATACCGCCGTGTACTAC |
| | | TGCGCCCGGAGGGGTGGATACCAGCTGCTGAGATGGGACGTGGGCCTCCTGCGGTCGG |
| | | CGTTCGACATCTGGGGCCAGGGCACTATGGTCACTGTGTCCAGCGGAGGAGGCGGATC |
| | | GGGAGGCGGCGGATCAGGGGGAGGCGGTTCCAGCTACGTGCTTACTCAACCCCCTTCG |
| | | GTGTCCGTGGCCCCGGGACAGACCGCCAGAATCACTTGCGGAGGAAACAACATTGGGT |
| | | CCAAGAGCGTGCATTGGTACCAGCAGAAGCCAGGACAGGCCCCTGTGCTGGTGCTCTA |
| | | CGGGAAGAACAATCGGCCCAGCGGAGTGCCGGACAGGTTCTCGGGTTCACGCTCCGGT |
| | | ACAACCGCTTCACTGACTATCACCGGGGCCCAGGCAGAGGATGAAGCGGACTACTACT |
| | | GTTCCTCCCGGGATTCATCCGGCGACCACCTCCGGGTGTTCGGAACCGGAACGAAGGT |
| | | CACCGTGCTGACCACTACCCCAGCACCGAGGCCACCCACCCCGGCTCCTACCATCGCC |
| | | TCCCAGCCTCTGTCCCTGCGTCCGGAGGCATGTAGACCCGCAGCTGGTGGGGCCGTGC |
| | | ATACCCGGGGTCTTGACTTCGCCTGCGATATCTACATTTGGGCCCCTCTGGCTGGTAC |
| | | TTGCGGGGTCCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTACTCAAGAGG |
| | | AGGACGGCTGTTCATGCCGGTTCCCAGAGGAGGAGGAAGGCGGCTGCGAACTGCGCGT |
| | | GAAATTCAGCCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGCGGAGAGGAC |
| | | GGGACCCAGAAATGGGCGGGAAGCCGCGCAGAAAGAATCCCCAAGAGGGCCTGTACAA |
| | | CGAGCTCCAAAAGGATAAGATGGCAGAAGCCTATAGCGAGATTGGTATGAAAGGGGAA |
| | | CGCAGAAGAGGCAAAGGCCACGACGGACTGTACCAGGGACTCAGCACCGCCACCAAGG |
| | | ACACCTATGACGCTCTTCACATGCAGGCCCTGCCGCCTCGG |

149369

| | | |
|---|---|---|
| 149369-aa | 136 | EVQLQQSGPGLVKPSQTLSLTCAISGDSVSSNSAAWNWIRQSPSRGLEWLGRTYYRSK |
| ScFv domain | | WYSFYAISLKSRIIINPDTSKNQFSLQLKSVTPEDTAVYYCARSSPEGLFLYWFDPWG |
| | | QGTLVTVSSGGDGSGGGGSGGGGSSSELTQDPAVSVALGQTIRITCQGDSLGNYYATW |
| | | YQQKPGQAPVLVIYGTNNRPSGIPDRFSASSSGNTASLTITGAQAEDEADYYCNSRDS |
| | | SGHHLLFGTGTKVTVL |
| 149369-nt ScFv | 157 | GAAGTGCAGCTCCAACAGTCAGGACCGGGGCTCGTGAAGCCATCCCAGACCCTGTCCC |
| domain | | TGACTTGTGCCATCTCGGGAGATAGCGTGTCATCGAACTCCGCCGCCTGGAACTGGAT |
| | | TCGGCAGAGCCCGTCCCGCGGACTGGAGTGGCTTGGAAGGACCTACTACCGGTCCAAG |
| | | TGGTACTCTTTCTACGCGATCTCGCTGAAGTCCCGCATTATCATTAACCCTGATACCT |
| | | CCAAGAATCAGTTCTCCCTCCAACTGAAATCCGTCACCCCCGAGGACACAGCAGTGTA |
| | | TTACTGCGCACGGAGCAGCCCCGAAGGACTGTTCCTGTATTGGTTTGACCCCTGGGGC |
| | | CAGGGGACTCTTGTGACCGTGTCGAGCGGCGGAGATGGGTCCGGTGGCGGTGGTTCGG |
| | | GGGGCGGCGGATCATCATCCGAACTGACCCAGGACCCGGCTGTGTCCGTGGCGCTGGG |
| | | ACAAACCATCCGCATTACGTGCCAGGGAGACTCCCTGGGCAACTACTACGCCACTTGG |
| | | TACCAGCAGAAGCCGGGCCAAGCCCCTGTGTTGGTCATCTACGGGACCAACAACAGAC |
| | | CTTCCGGCATCCCCGACCGGTTCAGCGCTTCGTCCTCCGGCAACACTGCCAGCCTGAC |
| | | CATCACTGGAGCGCAGGCCGAAGATGAGGCCGACTACTACTGCAACAGCAGAGACTCC |
| | | TCGGGTCATCACCTCTTGTTCGGAACTGGAACCAAGGTCACCGTGCTG |
| 149369-aa VH | 178 | EVQLQQSGPGLVKPSQTLSLTCAISGDSVSSNSAAWNWIRQSPSRGLEWLGRTYYRSK |
| | | WYSFYAISLKSRIIINPDTSKNQFSLQLKSVTPEDTAVYYCARSSPEGLFLYWFDPWG |
| | | QGTLVTVSS |
| 149369-aa VL | 199 | SSELTQDPAVSVALGQTIRITCQGDSLGNYYATWYQQKPGQAPVLVIYGTNNRPSGIP |
| | | DRFSASSSGNTASLTITGAQAEDEADYYCNSRDSSGHHLLFGTGTKVTVL |
| 149369-aa Full | 220 | MALPVTALLLPLALLLHAARPEVQLQQSGPGLVKPSQTLSLTCAISGDSVSSNSAAWN |
| CAR | | WIRQSPSRGLEWLGRTYYRSKWYSFYAISLKSRIIINPDTSKNQFSLQLKSVTPEDTA |
| | | VYYCARSSPEGLFLYWFDPWGQGTLVTVSSGGDGSGGGGSGGGGSSSELTQDPAVSVA |
| | | LGQTIRITCQGDSLGNYYATWYQQKPGQAPVLVIYGTNNRPSGIPDRFSASSSGNTAS |
| | | LTITGAQAEDEADYYCNSRDSSGHHLLFGTGTKVTVLTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| 149369-nt | 241 | ATGGCCCTCCCTGTCACCGCCCTGCTGCTTCCGCTGGCTCTTCTGCTCCACGCCGCTC |
| Full CAR | | GGCCCGAAGTGCAGCTCCAACAGTCAGGACCGGGGCTCGTGAAGCCATCCCAGACCCT |
| | | GTCCCTGACTTGTGCCATCTCGGGAGATAGCGTGTCATCGAACTCCGCCGCCTGGAAC |
| | | TGGATTCGGCAGAGCCCGTCCCGCGGACTGGAGTGGCTTGGAAGGACCTACTACCGGT |
| | | CCAAGTGGTACTCTTTCTACGCGATCTCGCTGAAGTCCCGCATTATCATTAACCCTGA |
| | | TACCTCCAAGAATCAGTTCTCCCTCCAACTGAAATCCGTCACCCCCGAGGACACAGCA |
| | | GTGTATTACTGCGCACGGAGCAGCCCCGAAGGACTGTTCCTGTATTGGTTTGACCCCT |
| | | GGGGCCAGGGGACTCTTGTGACCGTGTCGAGCGGCGGAGATGGGTCCGGTGGCGGTGG |
| | | TTCGGGGGGCGGCGGATCATCATCCGAACTGACCCAGGACCCGGCTGTGTCCGTGGCG |
| | | CTGGGACAAACCATCCGCATTACGTGCCAGGGAGACTCCCTGGGCAACTACTACGCCA |
| | | CTTGGTACCAGCAGAAGCCGGGCCAAGCCCCTGTGTTGGTCATCTACGGGACCAACAA |
| | | CAGACCTTCCGGCATCCCCGACCGGTTCAGCGCTTCGTCCTCCGGCAACACTGCCAGC |
| | | CTGACCATCACTGGAGCGCAGGCCGAAGATGAGGCCGACTACTACTGCAACAGCAGAG |
| | | ACTCCTCGGGTCATCACCTCTTGTTCGGAACTGGAACCAAGGTCACCGTGCTGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |

BCMA_EBB-C1978-A4

| | | |
|---|---|---|
| BCMA_EBB- | 137 | EVQLVESGGGLVQPGGSLRLSCAASGFTFSSYAMSWVRQAPGKGLEWVSAISGSGGST |
| C1978-A4-aa | | YYADSVKGRFTISRDNSKNTLYLQMNSLRAEDTAVYYCAKVEGSGSLDYWGQGTLVTV |
| ScFv domain | | SSGGGGSGGGGSGGGGSEIVMTQSPGTLSLSPGERATLSCRASQSVSSAYLAWYQQKP |
| | | GQPPRLLISGASTRATGIPDRFGGSGSGTDFTLTISRLEPEDFAVYYCQHYGSSFNGS |
| | | SLFTFGQGTRLEIK |
| BCMA_EBB- | 158 | GAAGTGCAGCTCGTGGAGTCAGGAGGCGGCCTGGTCCAGCCGGGAGGGTCCCTTAGAC |
| C1978-A4-nt | | TGTCATGCGCCGCAAGCGGATTCACTTTCTCCTCCTATGCCATGAGCTGGGTCCGCCA |
| ScFv domain | | AGCCCCCGGAAAGGGACTGGAATGGGTGTCCGCCATCTCGGGGTCTGGAGGCTCAACT |
| | | TACTACGCTGACTCCGTGAAGGGACGGTTCACCATTAGCCGCGACAACTCCAAGAACA |
| | | CCCTCTACCTCCAAATGAACTCCCTGCGGGCCGAGGATACCGCCGTCTACTACTGCGC |
| | | CAAAGTGGAAGGTTCAGGATCGCTGGACTACTGGGGACAGGGTACTCTCGTGACCGTG |
| | | TCATCGGGCGGAGGAGGTTCCGGCGGTGGCGGCTCCGGCGGCGGAGGGTCGGAGATCG |
| | | TGATGACCCAGAGCCCTGGTACTCTGAGCCTTTCGCCGGGAGAAAGGGCCACCCTGTC |
| | | CTGCCGCGCTTCCCAATCCGTGTCCTCCGCGTACTTGGCGTGGTACCAGCAGAAGCCG |
| | | GGACAGCCCCCTCGGCTGCTGATCAGCGGGGCCAGCACCCGGGCAACCGGAATCCCAG |
| | | ACAGATTCGGGGGTTCCGGCAGCGGCACAGATTTCACCCTGACTATTTCGAGGTTGGA |
| | | GCCCGAGGACTTTGCGGTGTATTACTGTCAGCACTACGGGTCGTCCTTTAATGGCTCC |
| | | AGCCTGTTCACGTTCGGACAGGGGACCCGCCTGGAAATCAAG |
| BCMA_EBB- | 179 | EVQLVESGGGLVQPGGSLRLSCAASGFTFSSYAMSWVRQAPGKGLEWVSAISGSGGST |
| C1978-A4-aa | | YYADSVKGRFTISRDNSKNTLYLQMNSLRAEDTAVYYCAKVEGSGSLDYWGQGTLVTV |
| VH | | SS |
| BCMA_EBB- | 200 | EIVMTQSPGTLSLSPGERATLSCRASQSVSSAYLAWYQQKPGQPPRLLISGASTRATG |
| C1978-A4-aa | | IPDRFGGSGSGTDFTLTISRLEPEDFAVYYCQHYGSSFNGSSLFTFGQGTRLEIK |
| VL | | |
| BCMA_EBB- | 221 | MALPVTALLLPLALLLHAARPEVQLVESGGGLVQPGGSLRLSCAASGFTFSSYAMSWV |
| C1978-A4-aa | | RQAPGKGLEWVSAISGSGGSTYYADSVKGRFTISRDNSKNTLYLQMNSLRAEDTAVYY |
| Full CART | | CAKVEGSGSLDYWGQGTLVTVSSGGGGSGGGGSGGGGSEIVMTQSPGTLSLSPGERAT |
| | | LSCRASQSVSSAYLAWYQQKPGQPPRLLISGASTRATGIPDRFGGSGSGTDFTLTISR |
| | | LEPEDFAVYYCQHYGSSFNGSSLFTFGQGTRLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 242 | ATGGCCCTCCCTGTCACCGCCCTGCTGCTTCCGCTGGCTCTTCTGCTCCACGCCGCTC |
| C1978-A4-nt | | GGCCCGAAGTGCAGCTCGTGGAGTCAGGAGGCGGCCTGGTCCAGCCGGGAGGGTCCCT |
| Full CART | | TAGACTGTCATGCGCCGCAAGCGGATTCACTTTCTCCTCCTATGCCATGAGCTGGGTC |
| | | CGCCAAGCCCCCGGAAAGGGACTGGAATGGGTGTCCGCCATCTCGGGGTCTGGAGGCT |
| | | CAACTTACTACGCTGACTCCGTGAAGGGACGGTTCACCATTAGCCGCGACAACTCCAA |
| | | GAACACCCTCTACCTCCAAATGAACTCCCTGCGGGCCGAGGATACCGCCGTCTACTAC |
| | | TGCGCCAAAGTGGAAGGTTCAGGATCGCTGGACTACTGGGGACAGGGTACTCTCGTGA |
| | | CCGTGTCATCGGGCGGAGGAGGTTCCGGCGGTGGCGGCTCCGGCGGCGGAGGGTCGGA |
| | | GATCGTGATGACCCAGAGCCCTGGTACTCTGAGCCTTTCGCCGGGAGAAAGGGCCACC |
| | | CTGTCCTGCCGCGCTTCCCAATCCGTGTCCTCCGCGTACTTGGCGTGGTACCAGCAGA |
| | | AGCCGGGACAGCCCCCTCGGCTGCTGATCAGCGGGGCCAGCACCCGGGCAACCGGAAT |
| | | CCCAGACAGATTCGGGGGTTCCGGCAGCGGCACAGATTTCACCCTGACTATTTCGAGG |
| | | TTGGAGCCCGAGGACTTTGCGGTGTATTACTGTCAGCACTACGGGTCGTCCTTTAATG |
| | | GCTCCAGCCTGTTCACGTTCGGACAGGGGACCCGCCTGGAAATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

BCMA_EBB-C1978-G1

| | | |
|---|---|---|
| BCMA_EBB- | 138 | EVQLVETGGGLVQPGGSLRLSCAASGITFSRYPMSWVRQAPGKGLEWVSGISD |
| C1978-G1-aa | | SGVSTYYADSAKGRFTISRDNSKNTLFLQMSSLRDEDTAVYYCVTRAGSEASD |
| ScFv domain | | IWGQGTMVTVSSGGGGSGGGGSGGGGSEIVLTQSPATLSLSPGERATLSCRAS |
| | | QSVSNSLAWYQQKPGQAPRLLIYDASSRATGIPDRFSGSGSGTDFTLTISRLEPE |
| | | DFAIYYCQQFGTSSGLTFGGGTKLEIK |
| BCMA_EBB- | 159 | GAAGTGCAACTGGTGGAAACCGGTGGCGGCCTGGTGCAGCCTGGAGGATCATTGAGGC |
| C1978-G1-nt | | TGTCATGCGCGGCCAGCGGTATTACCTTCTCCCGGTACCCCATGTCCTGGGTCAGACA |
| ScFv domain | | GGCCCCGGGGAAAGGGCTTGAATGGGTGTCCGGGATCTCGGACTCCGGTGTCAGCACT |
| | | TACTACGCCGACTCCGCCAAGGGACGCTTCACCATTTCCCGGGACAACTCGAAGAACA |
| | | CCCTGTTCCTCCAAATGAGCTCCCTCCGGGACGAGGATACTGCAGTGTACTACTGCGT |
| | | GACCCGCGCCGGGTCCGAGGCGTCTGACATTTGGGGACAGGGCACTATGGTCACCGTG |
| | | TCGTCCGGCGGAGGGGGCTCGGGAGGCGGTGGCAGCGGAGGAGGAGGGTCCGAGATCG |
| | | TGCTGACCCAATCCCCGGCCACCCTCTCGCTGAGCCCTGGAGAAAGGGCAACCTTGTC |
| | | CTGTCGCGCGAGCCAGTCCGTGAGCAACTCCCTGGCCTGGTACCAGCAGAAGCCCGGA |
| | | CAGGCTCCGAGACTTCTGATCTACGACGCTTCGAGCCGGGCCACTGGAATCCCCGACC |
| | | GCTTTTCGGGGTCCGGCTCAGGAACCGATTTCACCCTGACAATCTCACGGCTGGAGCC |
| | | AGAGGATTTCGCCATCTATTACTGCCAGCAGTTCGGTACTTCCTCCGGCCTGACTTTC |
| | | GGAGGCGGCACGAAGCTCGAAATCAAG |
| BCMA_EBB- | 180 | EVQLVETGGGLVQPGGSLRLSCAASGITFSRYPMSWVRQAPGKGLEWVSGISDSGVST |
| C1978-G1-aa | | YYADSAKGRFTISRDNSKNTLFLQMSSLRDEDTAVYYCVTRAGSEASDIWGQGTMVTV |
| VH | | SS |
| BCMA_EBB- | 201 | EIVLTQSPATLSLSPGERATLSCRASQSVSNSLAWYQQKPGQAPRLLIYDASSRATGI |
| C1978-G1-aa | | PDRFSGSGSGTDFTLTISRLEPEDFAIYYCQQFGTSSGLTFGGGTKLEIK |
| VL | | |
| BCMA_EBB- | 222 | MALPVTALLLPLALLLHAARPEVQLVETGGGLVQPGGSLRLSCAASGITFSRYP |
| C1978-G1-aa | | MSWVRQAPGKGLEWVSGISDSGVSTYYADSAKGRFTISRDNSKNTLFLQMSS |
| Full CART | | LRDEDTAVYYCVTRAGSEASDIWGQGTMVTVSSGGGGSGGGGSGGGGSEIVL |
| | | TQSPATLSLSPGERATLSCRASQSVSNSLAWYQQKPGQAPRLLIYDASSRATGI |
| | | PDRFSGSGSGTDFTLTISRLEPEDFAIYYCQQFGTSSGLTFGGGTKLEIKTTTPAP |
| | | RPPTPAPTIASQPLSLRPEACRPAAGGAVHTRGLDFACDIYIWAPLAGTCGVLL |
| | | LSLVITLYCKRGRKKLLYIFKQPFMRPVQTTQLEDGCSCRFPEELEGGCELRVK |
| | | FSRSADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYDALH |
| | | MQALPPR |
| BCMA_EBB- | 243 | ATGGCCCTCCCTGTCACCGCCCTGCTGCTTCCGCTGGCTCTTCTGCTCCACGCCGCTC |
| C1978-G1-nt | | GGCCCGAAGTGCAACTGGTGGAAACCGGTGGCGGCCTGGTGCAGCCTGGAGGATCATT |
| Full CART | | GAGGCTGTCATGCGCGGCCAGCGGTATTACCTTCTCCCGGTACCCCATGTCCTGGGTC |
| | | AGACAGGCCCCGGGGAAAGGGCTTGAATGGGTGTCCGGGATCTCGGACTCCGGTGTCA |
| | | GCACTTACTACGCCGACTCCGCCAAGGGACGCTTCACCATTTCCCGGGACAACTCGAA |
| | | GAACACCCTGTTCCTCCAAATGAGCTCCCTCCGGGACGAGGATACTGCAGTGTACTAC |
| | | TGCGTGACCCGCGCCGGGTCCGAGGCGTCTGACATTTGGGGACAGGGCACTATGGTCA |
| | | CCGTGTCGTCCGGCGGAGGGGGCTCGGGAGGCGGTGGCAGCGGAGGAGGAGGGTCCGA |
| | | GATCGTGCTGACCCAATCCCCGGCCACCCTCTCGCTGAGCCCTGGAGAAAGGGCAACC |
| | | TTGTCCTGTCGCGCGAGCCAGTCCGTGAGCAACTCCCTGGCCTGGTACCAGCAGAAGC |
| | | CCGGACAGGCTCCGAGACTTCTGATCTACGACGCTTCGAGCCGGGCCACTGGAATCCC |
| | | CGACCGCTTTTCGGGGTCCGGCTCAGGAACCGATTTCACCCTGACAATCTCACGGCTG |
| | | GAGCCAGAGGATTTCGCCATCTATTACTGCCAGCAGTTCGGTACTTCCTCCGGCCTGA |
| | | CTTTCGGAGGCGGCACGAAGCTCGAAATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |

BCMA_EBB-C1979-C1

| | | |
|---|---|---|
| BCMA_EBB- | 139 | QVQLVESGGGLVQPGGSLRLSCAASGFTFSSYAMSWVRQAPGKGLEWVSAISGSGGST |
| C1979-C1-aa | | YYADSVKGRFTISRDNAKNSLYLQMNSLRAEDTAIYYCARATYKRELRYYYGMDVWGQ |
| ScFv domain | | GTMVTVSSGGGGSGGGGSGGGGSEIVMTQSPGTVSLSPGERATLSCRASQSVSSSFLA |
| | | WYQQKPGQAPRLLIYGASSRATGIPDRFSGSGSGTDFTLTISRLEPEDSAVYYCQQYH |
| | | SSPSWTFGQGTRLEIK |
| BCMA_EBB- | 160 | CAAGTGCAGCTCGTGGAATCGGGTGGCGGACTGGTGCAGCCGGGGGGCTCACTTAGAC |
| C1979-C1-nt | | TGTCCTGCGCGGCCAGCGGATTCACTTTCTCCTCCTACGCCATGTCCTGGGTCAGACA |
| ScFv domain | | GGCCCCTGGAAAGGGCCTGGAATGGGTGTCCGCAATCAGCGGCAGCGGCGGCTCGACC |
| | | TATTACGCGGATTCAGTGAAGGGCAGATTCACCATTTCCCGGGACAACGCCAAGAACT |
| | | CCTTGTACCTTCAAATGAACTCCCTCCGCGCGGAAGATACCGCAATCTACTACTGCGC |
| | | TCGGGCCACTTACAAGAGGGAACTGCGCTACTACTACGGGATGGACGTCTGGGGCCAG |
| | | GGAACCATGGTCACCGTGTCCAGCGGAGGAGGAGGATCGGGAGGAGGCGGTAGCGGGG |
| | | GTGGAGGGTCGGAGATCGTGATGACCCAGTCCCCCGGCACTGTGTCGCTGTCCCCCGG |
| | | CGAACGGGCCACCCTGTCATGTCGGGCCAGCCAGTCAGTGTCGTCAAGCTTCCTCGCC |
| | | TGGTACCAGCAGAAACCGGGACAAGCTCCCCGCCTGCTGATCTACGGAGCCAGCAGCC |
| | | GGGCCACCGGTATTCCTGACCGGTTCTCCGGTTCGGGGTCCGGGACCGACTTTACTCT |
| | | GACTATCTCTCGCCTCGAGCCAGAGGACTCCGCCGTGTATTACTGCCAGCAGTACCAC |
| | | TCCTCCCCGTCCTGGACGTTCGGACAGGGCACAAGGCTGGAGATTAAG |
| BCMA_EBB- | 181 | QVQLVESGGGLVQPGGSLRLSCAASGFTFSSYAMSWVRQAPGKGLEWVSAISGSGGST |
| C1979-C1-aa | | YYADSVKGRFTISRDNAKNSLYLQMNSLRAEDTAIYYCARATYKRELRYYYGMDVWGQ |
| VH | | GTMVTVSS |
| BCMA_EBB- | 202 | EIVMTQSPGTVSLSPGERATLSCRASQSVSSSFLAWYQQKPGQAPRLLIYGASSRATG |
| C1979-C1-aa | | IPDRFSGSGSGTDFTLTISRLEPEDSAVYYCQQYHSSPSWTFGQGTRLEIK |
| VL | | |
| BCMA_EBB- | 223 | MALPVTALLLPLALLLHAARPQVQLVESGGGLVQPGGSLRLSCAASGFTFSSYAMSWV |
| C1979-C1-aa | | RQAPGKGLEWVSAISGSGGSTYYADSVKGRFTISRDNAKNSLYLQMNSLRAEDTAIYY |
| Full CART | | CARATYKRELRYYYGMDVWGQGTMVTVSSGGGGSGGGGSGGGGSEIVMTQSPGTVSLS |
| | | PGERATLSCRASQSVSSSFLAWYQQKPGQAPRLLIYGASSRATGIPDRFSGSGSGTDF |
| | | TLTISRLEPEDSAVYYCQQYHSSPSWTFGQGTRLEIKTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| BCMA_EBB- | 244 | ATGGCCCTCCCTGTCACCGCCCTGCTGCTTCCGCTGGCTCTTCTGCTCCACGCCGCTC |
| C1979-C1-nt | | GGCCCCAAGTGCAGCTCGTGGAATCGGGTGGCGGACTGGTGCAGCCGGGGGGCTCACT |
| Full CART | | TAGACTGTCCTGCGCGGCCAGCGGATTCACTTTCTCCTCCTACGCCATGTCCTGGGTC |
| | | AGACAGGCCCCTGGAAAGGGCCTGGAATGGGTGTCCGCAATCAGCGGCAGCGGCGGCT |
| | | CGACCTATTACGCGGATTCAGTGAAGGGCAGATTCACCATTTCCCGGGACAACGCCAA |
| | | GAACTCCTTGTACCTTCAAATGAACTCCCTCCGCGCGGAAGATACCGCAATCTACTAC |
| | | TGCGCTCGGGCCACTTACAAGAGGGAACTGCGCTACTACTACGGGATGGACGTCTGGG |
| | | GCCAGGGAACCATGGTCACCGTGTCCAGCGGAGGAGGAGGATCGGGAGGAGGCGGTAG |
| | | CGGGGGTGGAGGGTCGGAGATCGTGATGACCCAGTCCCCCGGCACTGTGTCGCTGTCC |
| | | CCCGGCGAACGGGCCACCCTGTCATGTCGGGCCAGCCAGTCAGTGTCGTCAAGCTTCC |
| | | TCGCCTGGTACCAGCAGAAACCGGGACAAGCTCCCCGCCTGCTGATCTACGGAGCCAG |
| | | CAGCCGGGCCACCGGTATTCCTGACCGGTTCTCCGGTTCGGGGTCCGGGACCGACTTT |
| | | ACTCTGACTATCTCTCGCCTCGAGCCAGAGGACTCCGCCGTGTATTACTGCCAGCAGT |
| | | ACCACTCCTCCCCGTCCTGGACGTTCGGACAGGGCACAAGGCTGGAGATTAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |

BCMA_EBB-C1978-C7

| | | |
|---|---|---|
| BCMA_EBB- | 140 | EVQLVETGGGLVQPGGSLRLSCAASGFTFSSYAMSWVRQAPGKGLEWVSAISGSGGST |
| C1978-C7-aa | | YYADSVKGRFTISRDNSKNTLYLQMNTLKAEDTAVYYCARATYKRELRYYYGMDVWGQ |
| ScFv domain | | GTTVTVSSGGGGSGGGGSGGGGSEIVLTQSPSTLSLSPGESATLSCRASQSVSTTFLA |
| | | WYQQKPGQAPRLLIYGSSNRATGIPDRFSGSGSGTDFTLTIRRLEPEDFAVYYCQQYH |
| | | SSPSWTFGQGTKVEIK |
| BCMA_EBB- | 161 | GAGGTGCAGCTTGTGGAAACCGGTGGCGGACTGGTGCAGCCCGGAGGAAGCCTCAGGC |
| C1978-C7-nt | | TGTCCTGCGCCGCGTCCGGCTTCACCTTCTCCTCGTACGCCATGTCCTGGGTCCGCCA |
| ScFv domain | | GGCCCCCGGAAAGGGCCTGGAATGGGTGTCCGCCATCTCTGGAAGCGGAGGTTCCACG |
| | | TACTACGCGGACAGCGTCAAGGGAAGGTTCACAATCTCCCGCGATAATTCGAAGAACA |
| | | CTCTGTACCTTCAAATGAACACCCTGAAGGCCGAGGACACTGCTGTGTACTACTGCGC |
| | | ACGGGCCACCTACAAGAGAGAGCTCCGGTACTACTACGGAATGGACGTCTGGGGCCAG |
| | | GGAACTACTGTGACCGTGTCCTCGGGAGGGGGTGGCTCCGGGGGGGGCGGCTCCGGCG |
| | | GAGGCGGTTCCGAGATTGTGCTGACCCAGTCACCTTCAACTCTGTCGCTGTCCCCGGG |
| | | AGAGAGCGCTACTCTGAGCTGCCGGGCCAGCCAGTCCGTGTCCACCACCTTCCTCGCC |
| | | TGGTATCAGCAGAAGCCGGGGCAGGCACCACGGCTCTTGATCTACGGGTCAAGCAACA |
| | | GAGCGACCGGAATTCCTGACCGCTTCTCGGGGAGCGGTTCAGGCACCGACTTCACCCT |
| | | GACTATCCGGCGCCTGGAACCCGAAGATTTCGCCGTGTATTACTGTCAACAGTACCAC |
| | | TCCTCGCCGTCCTGGACCTTTGGCCAAGGAACCAAAGTGGAAATCAAG |
| BCMA_EBB- | 182 | EVQLVETGGGLVQPGGSLRLSCAASGFTFSSYAMSWVRQAPGKGLEWVSAISGSGGST |
| C1978-C7-aa | | YYADSVKGRFTISRDNSKNTLYLQMNTLKAEDTAVYYCARATYKRELRYYYGMDVWGQ |
| VH | | GTTVTVSS |
| BCMA_EBB- | 203 | EIVLTQSPSTLSLSPGESATLSCRASQSVSTTFLAWYQQKPGQAPRLLIYGSSNRATG |
| C1978-C7-aa | | IPDRFSGSGSGTDFTLTIRRLEPEDFAVYYCQQYHSSPSWTFGQGTKVEIK |
| VL | | |
| BCMA_EBB- | 224 | MALPVTALLLPLALLLHAARPEVQLVETGGGLVQPGGSLRLSCAASGFTFSSYAMSWV |
| C1978-C7-aa | | RQAPGKGLEWVSAISGSGGSTYYADSVKGRFTISRDNSKNTLYLQMNTLKAEDTAVYY |
| Full CART | | CARATYKRELRYYYGMDVWGQGTTVTVSSGGGGSGGGGSGGGGSEIVLTQSPSTLSLS |
| | | PGESATLSCRASQSVSTTFLAWYQQKPGQAPRLLIYGSSNRATGIPDRFSGSGSGTDF |
| | | TLTIRRLEPEDFAVYYCQQYHSSPSWTFGQGTKVEIKTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| BCMA_EBB- | 245 | ATGGCCCTCCCTGTCACCGCCCTGCTGCTTCCGCTGGCTCTTCTGCTCCACGCCGCTC |
| C1978-C7-nt | | GGCCCGAGGTGCAGCTTGTGGAAACCGGTGGCGGACTGGTGCAGCCCGGAGGAAGCCT |
| Full CART | | CAGGCTGTCCTGCGCCGCGTCCGGCTTCACCTTCTCCTCGTACGCCATGTCCTGGGTC |
| | | CGCCAGGCCCCCGGAAAGGGCCTGGAATGGGTGTCCGCCATCTCTGGAAGCGGAGGTT |
| | | CCACGTACTACGCGGACAGCGTCAAGGGAAGGTTCACAATCTCCCGCGATAATTCGAA |
| | | GAACACTCTGTACCTTCAAATGAACACCCTGAAGGCCGAGGACACTGCTGTGTACTAC |
| | | TGCGCACGGGCCACCTACAAGAGAGAGCTCCGGTACTACTACGGAATGGACGTCTGGG |
| | | GCCAGGGAACTACTGTGACCGTGTCCTCGGGAGGGGGTGGCTCCGGGGGGGGCGGCTC |
| | | CGGCGGAGGCGGTTCCGAGATTGTGCTGACCCAGTCACCTTCAACTCTGTCGCTGTCC |
| | | CCGGGAGAGAGCGCTACTCTGAGCTGCCGGGCCAGCCAGTCCGTGTCCACCACCTTCC |
| | | TCGCCTGGTATCAGCAGAAGCCGGGGCAGGCACCACGGCTCTTGATCTACGGGTCAAG |
| | | CAACAGAGCGACCGGAATTCCTGACCGCTTCTCGGGGAGCGGTTCAGGCACCGACTTC |
| | | ACCCTGACTATCCGGCGCCTGGAACCCGAAGATTTCGCCGTGTATTACTGTCAACAGT |
| | | ACCACTCCTCGCCGTCCTGGACCTTTGGCCAAGGAACCAAAGTGGAAATCAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |

BCMA_EBB-C1978-D10

| | | |
|---|---|---|
| BCMA_EBB- | 141 | EVQLVETGGGLVQPGRSLRLSCAASGFTFDDYAMHWVRQAPGKGLEWVSGISWNSGSI |
| C1978-D10-aa | | GYADSVKGRFTISRDNAKNSLYLQMNSLRDEDTAVYYCARVGKAVPDVWGQGTTVTVS |
| ScFv domain | | SGGGGSGGGGSGGGGSDIVMTQTPSSLSASVGDRVTITCRASQSISSYLNWYQQKPGK |
| | | APKLLIYAASSLQSGVPSRFSGSGSGTDFTLTISSLQPEDFATYYCQQSYSTPYSFGQ |
| | | GTRLEIK |
| BCMA_EBB- | 162 | GAAGTGCAGCTCGTGGAAACTGGAGGTGGACTCGTGCAGCCTGGACGGTCGCTGCGGC |
| C1978-D10-nt | | TGAGCTGCGCTGCATCCGGCTTCACCTTCGACGATTATGCCATGCACTGGGTCAGACA |
| ScFv domain | | GGCGCCAGGGAAGGGACTTGAGTGGGTGTCCGGTATCAGCTGGAATAGCGGCTCAATC |
| | | GGATACGCGGACTCCGTGAAGGGAAGGTTCACCATTTCCCGCGACAACGCCAAGAACT |
| | | CCCTGTACTTGCAAATGAACAGCCTCCGGGATGAGGACACTGCCGTGTACTACTGCGC |
| | | CCGCGTCGGAAAAGCTGTGCCCGACGTCTGGGGCCAGGGAACCACTGTGACCGTGTCC |
| | | AGCGGCGGGGGTGGATCGGGCGGTGGAGGGTCCGGTGGAGGGGGCTCAGATATTGTGA |
| | | TGACCCAGACCCCCTCGTCCCTGTCCGCCTCGGTCGGCGACCGCGTGACTATCACATG |
| | | TAGAGCCTCGCAGAGCATCTCCAGCTACCTGAACTGGTATCAGCAGAAGCCGGGGAAG |
| | | GCCCCGAAGCTCCTGATCTACGCGGCATCATCACTGCAATCGGGAGTGCCGAGCCGGT |
| | | TTTCCGGGTCCGGCTCCGGCACCGACTTCACGCTGACCATTTCTTCCCTGCAACCCGA |
| | | GGACTTCGCCACTTACTACTGCCAGCAGTCCTACTCCACCCCTTACTCCTTCGGCCAA |
| | | GGAACCAGGCTGGAAATCAAG |
| BCMA_EBB- | 183 | EVQLVETGGGLVQPGRSLRLSCAASGFTFDDYAMHWVRQAPGKGLEWVSGISWNSGSI |
| C1978-D10-aa | | GYADSVKGRFTISRDNAKNSLYLQMNSLRDEDTAVYYCARVGKAVPDVWGQGTTVTVS |
| VH | | S |
| BCMA_EBB- | 204 | DIVMTQTPSSLSASVGDRVTITCRASQSISSYLNWYQQKPGKAPKLLIYAASSLQSGV |
| C1978-D10-aa | | PSRFSGSGSGTDFTLTISSLQPEDFATYYCQQSYSTPYSFGQGTRLEIK |
| VL | | |
| BCMA_EBB- | 225 | MALPVTALLLPLALLLHAARPEVQLVETGGGLVQPGRSLRLSCAASGFTFDDYAMHWV |
| C1978-D10-aa | | RQAPGKGLEWVSGISWNSGSIGYADSVKGRFTISRDNAKNSLYLQMNSLRDEDTAVYY |
| Full CART | | CARVGKAVPDVWGQGTTVTVSSGGGGSGGGGSGGGGSDIVMTQTPSSLSASVGDRVTI |
| | | TCRASQSISSYLNWYQQKPGKAPKLLIYAASSLQSGVPSRFSGSGSGTDFTLTISSLQ |
| | | PEDFATYYCQQSYSTPYSFGQGTRLEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| BCMA_EBB- | 246 | ATGGCCCTCCCTGTCACCGCCCTGCTGCTTCCGCTGGCTCTTCTGCTCCACGCCGCTC |
| C1978-D10-nt | | GGCCCGAAGTGCAGCTCGTGGAAACTGGAGGTGGACTCGTGCAGCCTGGACGGTCGCT |
| Full CART | | GCGGCTGAGCTGCGCTGCATCCGGCTTCACCTTCGACGATTATGCCATGCACTGGGTC |
| | | AGACAGGCGCCAGGGAAGGGACTTGAGTGGGTGTCCGGTATCAGCTGGAATAGCGGCT |
| | | CAATCGGATACGCGGACTCCGTGAAGGGAAGGTTCACCATTTCCCGCGACAACGCCAA |
| | | GAACTCCCTGTACTTGCAAATGAACAGCCTCCGGGATGAGGACACTGCCGTGTACTAC |
| | | TGCGCCCGCGTCGGAAAAGCTGTGCCCGACGTCTGGGGCCAGGGAACCACTGTGACCG |
| | | TGTCCAGCGGCGGGGGTGGATCGGGCGGTGGAGGGTCCGGTGGAGGGGGCTCAGATAT |
| | | TGTGATGACCCAGACCCCCTCGTCCCTGTCCGCCTCGGTCGGCGACCGCGTGACTATC |
| | | ACATGTAGAGCCTCGCAGAGCATCTCCAGCTACCTGAACTGGTATCAGCAGAAGCCGG |
| | | GGAAGGCCCCGAAGCTCCTGATCTACGCGGCATCATCACTGCAATCGGGAGTGCCGAG |
| | | CCGGTTTTCCGGGTCCGGCTCCGGCACCGACTTCACGCTGACCATTTCTTCCCTGCAA |
| | | CCCGAGGACTTCGCCACTTACTACTGCCAGCAGTCCTACTCCACCCCTTACTCCTTCG |
| | | GCCAAGGAACCAGGCTGGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |

BCMA_EBB-C1979-C12

| | | |
|---|---|---|
| BCMA_EBB- | 142 | EVQLVESGGGLVQPGRSLRLSCTASGFTFDDYAMHWVRQRPGKGLEWVASINWKGNSL |
| C1979-C12-aa | | AYGDSVKGRFAISRDNAKNTVFLQMNSLRTEDTAVYYCASHQGVAYYNYAMDVWGRGT |
| ScFv domain | | LVTVSSGGGGSGGGGSGGGGSEIVLTQSPGTLSLSPGERATLSCRATQSIGSSFLAWY |
| | | QQRPGQAPRLLIYGASQRATGIPDRFSGRGSGTDFTLTISRVEPEDSAVYYCQHYESS |
| | | PSWTFGQGTKVEIK |
| BCMA_EBB- | 163 | GAAGTGCAGCTCGTGGAGAGCGGGGGAGGATTGGTGCAGCCCGGAAGGTCCCTGCGGC |
| C1979-C12-nt | | TCTCCTGCACTGCGTCTGGCTTCACCTTCGACGACTACGCGATGCACTGGGTCAGACA |
| ScFv domain | | GCGCCCGGGAAAGGGCCTGGAATGGGTCGCCTCAATCAACTGGAAGGGAAACTCCCTG |
| | | GCCTATGGCGACAGCGTGAAGGGCCGCTTCGCCATTTCGCGCGACAACGCCAAGAACA |
| | | CCGTGTTTCTGCAAATGAATTCCCTGCGGACCGAGGATACCGCTGTGTACTACTGCGC |
| | | CAGCCACCAGGGCGTGGCATACTATAACTACGCCATGGACGTGTGGGGAAGAGGGACG |
| | | CTCGTCACCGTGTCCTCCGGGGGCGGTGGATCGGGTGGAGGAGGAAGCGGTGGCGGGG |
| | | GCAGCGAAATCGTGCTGACTCAGAGCCCGGGAACTCTTTCACTGTCCCCGGGAGAACG |
| | | GGCCACTCTCTCGTGCCGGGCCACCCAGTCCATCGGCTCCTCCTTCCTTGCCTGGTAC |
| | | CAGCAGAGGCCAGGACAGGCGCCCCGCCTGCTGATCTACGGTGCTTCCCAACGCGCCA |
| | | CTGGCATTCCTGACCGGTTCAGCGGCAGAGGGTCGGGAACCGATTTCACACTGACCAT |
| | | TTCCCGGGTGGAGCCCGAAGATTCGGCAGTCTACTACTGTCAGCATTACGAGTCCTCC |
| | | CCTTCATGGACCTTCGGTCAAGGGACCAAAGTGGAGATCAAG |
| BCMA_EBB- | 184 | EVQLVESGGGLVQPGRSLRLSCTASGFTFDDYAMHWVRQRPGKGLEWVASINWKGNSL |
| C1979-C12-aa | | AYGDSVKGRFAISRDNAKNTVFLQMNSLRTEDTAVYYCASHQGVAYYNYAMDVWGRGT |
| VH | | LVTVSS |
| BCMA_EBB- | 205 | EIVLTQSPGTLSLSPGERATLSCRATQSIGSSFLAWYQQRPGQAPRLLIYGASQRATG |
| C1979-C12-aa | | IPDRFSGRGSGTDFTLTISRVEPEDSAVYYCQHYESSPSWTFGQGTKVEIK |
| VL | | |
| BCMA_EBB- | 226 | MALPVTALLLPLALLLHAARPEVQLVESGGGLVQPGRSLRLSCTASGFTFDDYAMHWV |
| C1979-C12-aa | | RQRPGKGLEWVASINWKGNSLAYGDSVKGRFAISRDNAKNTVFLQMNSLRTEDTAVYY |
| Full CART | | CASHQGVAYYNYAMDVWGRGTLVTVSSGGGGSGGGGSGGGGSEIVLTQSPGTLSLSPG |
| | | ERATLSCRATQSIGSSFLAWYQQRPGQAPRLLIYGASQRATGIPDRFSGRGSGTDFTL |
| | | TISRVEPEDSAVYYCQHYESSPSWTFGQGTKVEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 247 | ATGGCCCTCCCTGTCACCGCCCTGCTGCTTCCGCTGGCTCTTCTGCTCCACGCCGCTC |
| C1979-C12-nt | | GGCCCGAAGTGCAGCTCGTGGAGAGCGGGGGAGGATTGGTGCAGCCCGGAAGGTCCCT |
| Full CART | | GCGGCTCTCCTGCACTGCGTCTGGCTTCACCTTCGACGACTACGCGATGCACTGGGTC |
| | | AGACAGCGCCCGGGAAAGGGCCTGGAATGGGTCGCCTCAATCAACTGGAAGGGAAACT |
| | | CCCTGGCCTATGGCGACAGCGTGAAGGGCCGCTTCGCCATTTCGCGCGACAACGCCAA |
| | | GAACACCGTGTTTCTGCAAATGAATTCCCTGCGGACCGAGGATACCGCTGTGTACTAC |
| | | TGCGCCAGCCACCAGGGCGTGGCATACTATAACTACGCCATGGACGTGTGGGGAAGAG |
| | | GGACGCTCGTCACCGTGTCCTCCGGGGGCGGTGGATCGGGTGGAGGAGGAAGCGGTGG |
| | | CGGGGGCAGCGAAATCGTGCTGACTCAGAGCCCGGGAACTCTTTCACTGTCCCCGGGA |
| | | GAACGGGCCACTCTCTCGTGCCGGGCCACCCAGTCCATCGGCTCCTCCTTCCTTGCCT |
| | | GGTACCAGCAGAGGCCAGGACAGGCGCCCCGCCTGCTGATCTACGGTGCTTCCCAACG |
| | | CGCCACTGGCATTCCTGACCGGTTCAGCGGCAGAGGGTCGGGAACCGATTTCACACTG |
| | | ACCATTTCCCGGGTGGAGCCCGAAGATTCGGCAGTCTACTACTGTCAGCATTACGAGT |
| | | CCTCCCCTTCATGGACCTTCGGTCAAGGGACCAAAGTGGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

BCMA_EBB-C1980-G4

| | | |
|---|---|---|
| BCMA_EBB- | 143 | EVQLVESGGGLVQPGGSLRLSCAASGFTFSSYAMSWVRQAPGKGLEWVSAIS |
| C1980-G4-aa | | GSGGSTYYADSVKGRFTISRDNSKNTLYLQMNSLRAEDTAVYYCAKVVRDG |
| ScFv domain | | MDVWGQGTTVTVSSGGGGSGGGGSGGGGSEIVLTQSPATLSLSPGERATLSCR |
| | | ASQSVSSSYLAWYQQKPGQAPRLLIYGASSRATGIPDRFSGNGSGTDFTLTISR |
| | | LEPEDFAVYYCQQYGSPPRFTFGPGTKVDIK |
| BCMA_EBB- | 164 | GAGGTGCAGTTGGTCGAAAGCGGGGGCGGGCTTGTGCAGCCTGGCGGATCACTGCGGC |
| C1980-G4-nt | | TGTCCTGCGCGGCATCAGGCTTCACGTTTTCTTCCTACGCCATGTCCTGGGTGCGCCA |
| ScFv domain | | GGCCCCTGGAAAGGGACTGGAATGGGTGTCCGCGATTTCGGGGTCCGGCGGGAGCACC |
| | | TACTACGCCGATTCCGTGAAGGGCCGCTTCACTATCTCGCGGGACAACTCCAAGAACA |
| | | CCCTCTACCTCCAAATGAATAGCCTGCGGGCCGAGGATACCGCCGTCTACTATTGCGC |
| | | TAAGGTCGTGCGCGACGGAATGGACGTGTGGGGACAGGGTACCACCGTGACAGTGTCC |
| | | TCGGGGGGAGGCGGTAGCGGCGGAGGAGGAAGCGGTGGTGGAGGTTCCGAGATTGTGC |
| | | TGACTCAATCACCCGCGACCCTGAGCCTGTCCCCCGGCGAAAGGGCCACTCTGTCCTG |
| | | TCGGGCCAGCCAATCAGTCTCCTCCTCGTACCTGGCCTGGTACCAGCAGAAGCCAGGA |
| | | CAGGCTCCGAGACTCCTTATCTATGGCGCATCCTCCCGCGCCACCGGAATCCCGGATA |
| | | GGTTCTCGGGAAACGGATCGGGGACCGACTTCACTCTCACCATCTCCCGGCTGGAACC |
| | | GGAGGACTTCGCCGTGTACTACTGCCAGCAGTACGGCAGCCCGCCTAGATTCACTTTC |
| | | GGCCCCGGCACCAAAGTGGACATCAAG |
| BCMA_EBB- | 185 | EVQLVESGGGLVQPGGSLRLSCAASGFTFSSYAMSWVRQAPGKGLEWVSAISGSGGST |
| C1980-G4-aa | | YYADSVKGRFTISRDNSKNTLYLQMNSLRAEDTAVYYCAKVVRDGMDVWGQGTTVTVS |
| VH | | S |
| BCMA_EBB- | 206 | EIVLTQSPATLSLSPGERATLSCRASQSVSSSYLAWYQQKPGQAPRLLIYGASSRATG |
| C1980-G4-aa | | IPDRFSGNGSGTDFTLTISRLEPEDFAVYYCQQYGSPPRFTFGPGTKVDIK |
| VL | | |
| BCMA_EBB- | 227 | MALPVTALLLPLALLLHAARPEVQLVESGGGLVQPGGSLRLSCAASGFTFSSY |
| C1980-G4-aa | | AMSWVRQAPGKGLEWVSAISGSGGSTYYADSVKGRFTISRDNSKNTLYLQMN |
| Full CART | | SLRAEDTAVYYCAKVVRDGMDVWGQGTTVTVSSGGGGSGGGGSGGGGSEIV |
| | | LTQSPATLSLSPGERATLSCRASQSVSSSYLAWYQQKPGQAPRLLIYGASSRAT |
| | | GIPDRFSGNGSGTDFTLTISRLEPEDFAVYYCQQYGSPPRFTFGPGTKVDIKTTT |
| | | PAPRPPTPAPTIASQPLSLRPEACRPAAGGAVHTRGLDFACDIYIWAPLAGTCG |
| | | VLLLSLVITLYCKRGRKKLLYIFKQPFMRPVQTTQLEDGCSCRFPEELEGGCEL |
| | | RVKFSRSADAPAYKQGQNQLYNELNLGRREEYDVLDKRRGRDPEMGGKPRR |
| | | KNPQEGLYNELQKDKMALAYSEIGMKGERRRGKGHDGLYQGLSTATKDTYD |
| | | ALHMQALPPR |
| BCMA_EBB- | 248 | ATGGCCCTCCCTGTCACCGCCCTGCTGCTTCCGCTGGCTCTTCTGCTCCACGCCGCTC |
| C1980-G4-nt | | GGCCCGAGGTGCAGTTGGTCGAAAGCGGGGGCGGGCTTGTGCAGCCTGGCGGATCACT |
| Full CART | | GCGGCTGTCCTGCGCGGCATCAGGCTTCACGTTTTCTTCCTACGCCATGTCCTGGGTG |
| | | CGCCAGGCCCCTGGAAAGGGACTGGAATGGGTGTCCGCGATTTCGGGGTCCGGCGGGA |
| | | GCACCTACTACGCCGATTCCGTGAAGGGCCGCTTCACTATCTCGCGGGACAACTCCAA |
| | | GAACACCCTCTACCTCCAAATGAATAGCCTGCGGGCCGAGGATACCGCCGTCTACTAT |
| | | TGCGCTAAGGTCGTGCGCGACGGAATGGACGTGTGGGGACAGGGTACCACCGTGACAG |
| | | TGTCCTCGGGGGGAGGCGGTAGCGGCGGAGGAGGAAGCGGTGGTGGAGGTTCCGAGAT |
| | | TGTGCTGACTCAATCACCCGCGACCCTGAGCCTGTCCCCCGGCGAAAGGGCCACTCTG |
| | | TCCTGTCGGGCCAGCCAATCAGTCTCCTCCTCGTACCTGGCCTGGTACCAGCAGAAGC |
| | | CAGGACAGGCTCCGAGACTCCTTATCTATGGCGCATCCTCCCGCGCCACCGGAATCCC |
| | | GGATAGGTTCTCGGGAAACGGATCGGGGACCGACTTCACTCTCACCATCTCCCGGCTG |
| | | GAACCGGAGGACTTCGCCGTGTACTACTGCCAGCAGTACGGCAGCCCGCCTAGATTCA |
| | | CTTTCGGCCCCGGCACCAAAGTGGACATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |

BCMA_EBB-C1980-D2

| | | |
|---|---|---|
| BCMA_EBB- | 144 | EVQLLESGGGLVQPGGSLRLSCAASGFTFSSYAMSWVRQAPGKGLEWVSAISGSGGST |
| C1980-D2-aa | | YYADSVKGRFTISRDNSKNTLYLQMNSLRAEDTAVYYCAKIPQTGTFDYWGQGTLVTV |
| ScFv domain | | SSGGGGSGGGGSGGGGSEIVLTQSPGTLSLSPGERATLSCRASQSVSSSYLAWYQQRP |
| | | GQAPRLLIYGASSRATGIPDRFSGSGSGTDFTLTISRLEPEDFAVYYCQHYGSSPSWT |
| | | FGQGTRLEIK |
| BCMA_EBB- | 165 | GAAGTGCAGCTGCTGGAGTCCGGCGGTGGATTGGTGCAACCGGGGGGATCGCTCAGAC |
| C1980-D2-nt | | TGTCCTGTGCGGCGTCAGGCTTCACCTTCTCGAGCTACGCCATGTCATGGGTCAGACA |
| ScFv domain | | GGCCCCTGGAAAGGGTCTGGAATGGGTGTCCGCCATTTCCGGGAGCGGGGGATCTACA |
| | | TACTACGCCGATAGCGTGAAGGGCCGCTTCACCATTTCCCGGGACAACTCCAAGAACA |
| | | CTCTCTATCTGCAAATGAACTCCCTCCGCGCTGAGGACACTGCCGTGTACTACTGCGC |
| | | CAAAATCCCTCAGACCGGCACCTTCGACTACTGGGGACAGGGGACTCTGGTCACCGTC |
| | | AGCAGCGGTGGCGGAGGTTCGGGGGGAGGAGGAAGCGGCGGCGGAGGGTCCGAGATTG |
| | | TGCTGACCCAGTCACCCGGCACTTTGTCCCTGTCGCCTGGAGAAAGGGCCACCCTTTC |
| | | CTGCCGGGCATCCCAATCCGTGTCCTCCTCGTACCTGGCCTGGTACCAGCAGAGGCCC |
| | | GGACAGGCCCCACGGCTTCTGATCTACGGAGCAAGCAGCCGCGCGACCGGTATCCCGG |
| | | ACCGGTTTTCGGGCTCGGGCTCAGGAACTGACTTCACCCTCACCATCTCCCGCCTGGA |
| | | ACCCGAAGATTTCGCTGTGTATTACTGCCAGCACTACGGCAGCTCCCCGTCCTGGACG |
| | | TTCGGCCAGGGAACTCGGCTGGAGATCAAG |
| BCMA_EBB- | 186 | EVQLLESGGGLVQPGGSLRLSCAASGFTFSSYAMSWVRQAPGKGLEWVSAISGSGGST |
| C1980-D2-aa | | YYADSVKGRFTISRDNSKNTLYLQMNSLRAEDTAVYYCAKIPQTGTFDYWGQGTLVTV |
| VH | | SS |
| BCMA_EBB- | 207 | EIVLTQSPGTLSLSPGERATLSCRASQSVSSSYLAWYQQRPGQAPRLLIYGASSRATG |
| C1980-D2-aa | | IPDRFSGSGSGTDFTLTISRLEPEDFAVYYCQHYGSSPSWTFGQGTRLEIK |
| VL | | |
| BCMA_EBB- | 228 | MALPVTALLLPLALLLHAARPEVQLLESGGGLVQPGGSLRLSCAASGFTFSSYAMSWV |
| C1980-D2-aa | | RQAPGKGLEWVSAISGSGGSTYYADSVKGRFTISRDNSKNTLYLQMNSLRAEDTAVYY |
| Full CART | | CAKIPQTGTFDYWGQGTLVTVSSGGGGSGGGGSGGGGSEIVLTQSPGTLSLSPGERAT |
| | | LSCRASQSVSSSYLAWYQQRPGQAPRLLIYGASSRATGIPDRFSGSGSGTDFTLTISR |
| | | LEPEDFAVYYCQHYGSSPSWTFGQGTRLEIKTTTPAPRPPTPAPTIASQPLSLRPEAC |
| | | RPAAGGAVHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNELNLGRREEY |
| | | DVLDKRRGRDPEMGGKPRRKNPQEGLYNELQKDKMAEAYSEIGMKGERRRGKGHDGLY |
| | | QGLSTATKDTYDALHMQALPPR |
| BCMA_EBB- | 249 | ATGGCCCTCCCTGTCACCGCCCTGCTGCTTCCGCTGGCTCTTCTGCTCCACGCCGCTC |
| C1980-D2-nt | | GGCCCGAAGTGCAGCTGCTGGAGTCCGGCGGTGGATTGGTGCAACCGGGGGGATCGCT |
| Full CART | | CAGACTGTCCTGTGCGGCGTCAGGCTTCACCTTCTCGAGCTACGCCATGTCATGGGTC |
| | | AGACAGGCCCCTGGAAAGGGTCTGGAATGGGTGTCCGCCATTTCCGGGAGCGGGGGAT |
| | | CTACATACTACGCCGATAGCGTGAAGGGCCGCTTCACCATTTCCCGGGACAACTCCAA |
| | | GAACACTCTCTATCTGCAAATGAACTCCCTCCGCGCTGAGGACACTGCCGTGTACTAC |
| | | TGCGCCAAAATCCCTCAGACCGGCACCTTCGACTACTGGGGACAGGGGACTCTGGTCA |
| | | CCGTCAGCAGCGGTGGCGGAGGTTCGGGGGGAGGAGGAAGCGGCGGCGGAGGGTCCGA |
| | | GATTGTGCTGACCCAGTCACCCGGCACTTTGTCCCTGTCGCCTGGAGAAAGGGCCACC |
| | | CTTTCCTGCCGGGCATCCCAATCCGTGTCCTCCTCGTACCTGGCCTGGTACCAGCAGA |
| | | GGCCCGGACAGGCCCCACGGCTTCTGATCTACGGAGCAAGCAGCCGCGCGACCGGTAT |
| | | CCCGGACCGGTTTTCGGGCTCGGGCTCAGGAACTGACTTCACCCTCACCATCTCCCGC |
| | | CTGGAACCCGAAGATTTCGCTGTGTATTACTGCCAGCACTACGGCAGCTCCCCGTCCT |
| | | GGACGTTCGGCCAGGGAACTCGGCTGGAGATCAAGACCACTACCCCAGCACCGAGGCC |
| | | ACCCACCCCGGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCTGCGATATCT |
| | | ACATTTGGGCCCCTCTGGCTGGTACTTGCGGGGTCCTGCTGCTTTCACTCGTGATCAC |
| | | TCTTTACTGTAAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTCCCAGAGGAGG |
| | | AGGAAGGCGGCTGCGAACTGCGCGTGAAATTCAGCCGCAGCGCAGATGCTCCAGCCTA |
| | | CAAGCAGGGGCAGAACCAGCTCTACAACGAACTCAATCTTGGTCGGAGAGAGGAGTAC |
| | | GACGTGCTGGACAAGCGGAGAGGACGGGACCCAGAAATGGGCGGGAAGCCGCGCAGAA |
| | | AGAATCCCCAAGAGGGCCTGTACAACGAGCTCCAAAAGGATAAGATGGCAGAAGCCTA |
| | | TAGCGAGATTGGTATGAAAGGGGAACGCAGAAGAGGCAAAGGCCACGACGGACTGTAC |
| | | CAGGGACTCAGCACCGCCACCAAGGACACCTATGACGCTCTTCACATGCAGGCCCTGC |
| | | CGCCTCGG |

BCMA_EBB-C1978-A10

| | | |
|---|---|---|
| BCMA_EBB- | 145 | EVQLVETGGGLVQPGGSLRLSCAASGFTFSSYAMSWVRQAPGKGLEWVSAIS |
| C1978-A10-aa | | GSGGSTYYADSVKGRFTMSRENDKNSVFLQMNSLRVEDTGVYYCARANYKR |
| ScFv domain | | ELRYYYGMDVWGQGTMVTVSSGGGGSGGGGSGGGGSEIVMTQSPGTLSLSP |
| | | GESATLSCRASQRVASNYLAWYQHKPGQAPSLLISGASSRATGVPDRFSGSGS |
| | | GTDFTLAISRLEPEDSAVYYCQHYDSSPSWTFGQGTKVEIK |
| BCMA_EBB- | 166 | GAAGTGCAACTGGTGGAAACCGGTGGAGGACTCGTGCAGCCTGGCGGCAGCCTCCGGC |
| C1978-A10-nt | | TGAGCTGCGCCGCTTCGGGATTCACCTTTTCCTCCTACGCGATGTCTTGGGTCAGACA |
| ScFv domain | | GGCCCCCGGAAAGGGGCTGGAATGGGTGTCAGCCATCTCCGGCTCCGGCGGATCAACG |
| | | TACTACGCCGACTCCGTGAAAGGCCGGTTCACCATGTCGCGCGAGAATGACAAGAACT |
| | | CCGTGTTCCTGCAAATGAACTCCCTGAGGGTGGAGGACACCGGAGTGTACTATTGTGC |
| | | GCGCGCCAACTACAAGAGAGAGCTGCGGTACTACTACGGAATGGACGTCTGGGGACAG |
| | | GGAACTATGGTGACCGTGTCATCCGGTGGAGGGGGAAGCGGCGGTGGAGGCAGCGGGG |
| | | GCGGGGGTTCAGAAATTGTCATGACCCAGTCCCCGGGAACTCTTTCCCTCTCCCCCGG |
| | | GGAATCCGCGACTTTGTCCTGCCGGGCCAGCCAGCGCGTGGCCTCGAACTACCTCGCA |
| | | TGGTACCAGCATAAGCCAGGCCAAGCCCCTTCCCTGCTGATTTCCGGGGCTAGCAGCC |
| | | GCGCCACTGGCGTGCCGGATAGGTTCTCGGGAAGCGGCTCGGGTACCGATTTCACCCT |
| | | GGCAATCTCGCGGCTGGAACCGGAGGATTCGGCCGTGTACTACTGCCAGCACTATGAC |
| | | TCATCCCCCTCCTGGACATTCGGACAGGGCACCAAGGTCGAGATCAAG |
| BCMA_EBB- | 187 | EVQLVETGGGLVQPGGSLRLSCAASGFTESSYAMSWVRQAPGKGLEWVSAISGSGGST |
| C1978-A10-aa | | YYADSVKGRFTMSRENDKNSVFLQMNSLRVEDTGVYYCARANYKRELRYYYGMDVWGQ |
| VH | | GTMVTVSS |
| BCMA_EBB- | 208 | EIVMTQSPGTLSLSPGESATLSCRASQRVASNYLAWYQHKPGQAPSLLISGASSRATG |
| C1978-A10-aa | | VPDRFSGSGSGTDFTLAISRLEPEDSAVYYCQHYDSSPSWTFGQGTKVEIK |
| VL | | |
| BCMA_EBB- | 229 | MALPVTALLLPLALLLHAARPEVQLVETGGGLVQPGGSLRLSCAASGFTFSSY |
| C1978-A10-aa | | AMSWVRQAPGKGLEWVSAISGSGGSTYYADSVKGRFTMSRENDKNSVFLQM |
| Full CART | | NSLRVEDTGVYYCARANYKRELRYYYGMDVWGQGTMVTVSSGGGGSGGGG |
| | | SGGGGSEIVMTQSPGTLSLSPGESATLSCRASQRVASNYLAWYQHKPGQAPSL |
| | | LISGASSRATGVPDRFSGSGSGTDFTLAISRLEPEDSAVYYCQHYDSSPSWTFG |
| | | QGTKVEIKTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSCRF |
| | | PEEEEGGCELRVKFSRSADAPAYKQGQNQLYNELNLGRREEYDVLDKRRGRD |
| | | PEMGGKPRRKNPQEGLYNELQKDKMAEAYSEIGMKGERRRGKGHDGLYQGL |
| | | STATKDTYDALHMQALPPR |
| BCMA_EBB- | 250 | ATGGCCCTCCCTGTCACCGCCCTGCTGCTTCCGCTGGCTCTTCTGCTCCACGCCGCTC |
| C1978-A10-nt | | GGCCCGAAGTGCAACTGGTGGAAACCGGTGGAGGACTCGTGCAGCCTGGCGGCAGCCT |
| Full CART | | CCGGCTGAGCTGCGCCGCTTCGGGATTCACCTTTTCCTCCTACGCGATGTCTTGGGTC |
| | | AGACAGGCCCCCGGAAAGGGGCTGGAATGGGTGTCAGCCATCTCCGGCTCCGGCGGAT |
| | | CAACGTACTACGCCGACTCCGTGAAAGGCCGGTTCACCATGTCGCGCGAGAATGACAA |
| | | GAACTCCGTGTTCCTGCAAATGAACTCCCTGAGGGTGGAGGACACCGGAGTGTACTAT |
| | | TGTGCGCGCGCCAACTACAAGAGAGAGCTGCGGTACTACTACGGAATGGACGTCTGGG |
| | | GACAGGGAACTATGGTGACCGTGTCATCCGGTGGAGGGGGAAGCGGCGGTGGAGGCAG |
| | | CGGGGGCGGGGGTTCAGAAATTGTCATGACCCAGTCCCCGGGAACTCTTTCCCTCTCC |
| | | CCCGGGGAATCCGCGACTTTGTCCTGCCGGGCCAGCCAGCGCGTGGCCTCGAACTACC |
| | | TCGCATGGTACCAGCATAAGCCAGGCCAAGCCCCTTCCCTGCTGATTTCCGGGGCTAG |
| | | CAGCCGCGCCACTGGCGTGCCGGATAGGTTCTCGGGAAGCGGCTCGGGTACCGATTTC |
| | | ACCCTGGCAATCTCGCGGCTGGAACCGGAGGATTCGGCCGTGTACTACTGCCAGCACT |
| | | ATGACTCATCCCCCTCCTGGACATTCGGACAGGGCACCAAGGTCGAGATCAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |

BCMA_EBB-C1978-D4

| | | |
|---|---|---|
| BCMA_EBB- | 146 | EVQLLETGGGLVQPGGSLRLSCAASGFSFSSYAMSWVRQAPGKGLEWVSAISGSGGST |
| C1978-D4-aa | | YYADSVKGRFTISRDNSKNTLYLQMNSLRAEDTAVYYCAKALVGATGAFDIWGQGTLV |
| ScFv domain | | TVSSGGGGSGGGGSGGGGSEIVLTQSPGTLSLSPGERATLSCRASQSLSSNFLAWYQQ |
| | | KPGQAPGLLIYGASNWATGTPDRFSGSGSGTDFTLTITRLEPEDFAVYYCQYYGTSPM |
| | | YTFGQGTKVEIK |
| BCMA_EBB- | 167 | GAAGTGCAGCTGCTCGAAACCGGTGGAGGGCTGGTGCAGCCAGGGGGCTCCCTGAGGC |
| C1978-D4-nt | | TTTCATGCGCCGCTAGCGGATTCTCCTTCTCCTCTTACGCCATGTCGTGGGTCCGCCA |
| ScFv domain | | AGCCCCTGGAAAAGGCCTGGAATGGGTGTCCGCGATTTCCGGGAGCGGAGGTTCGACC |
| | | TATTACGCCGACTCCGTGAAGGGCCGCTTTACCATCTCCCGGGATAACTCCAAGAACA |
| | | CTCTGTACCTCCAAATGAACTCGCTGAGAGCCGAGGACACCGCCGTGTATTACTGCGC |
| | | GAAGGCGCTGGTCGGCGCGACTGGGGCATTCGACATCTGGGGACAGGGAACTCTTGTG |
| | | ACCGTGTCGAGCGGAGGCGGCGGCTCCGGCGGAGGAGGGAGCGGGGGCGGTGGTTCCG |
| | | AAATCGTGTTGACTCAGTCCCCGGGAACCCTGAGCTTGTCACCCGGGGAGCGGGCCAC |
| | | TCTCTCCTGTCGCGCCTCCCAATCGCTCTCATCCAATTTCCTGGCCTGGTACCAGCAG |
| | | AAGCCCGGACAGGCCCCGGGCCTGCTCATCTACGGCGCTTCAAACTGGGCAACGGGAA |
| | | CCCCTGATCGGTTCAGCGGAAGCGGATCGGGTACTGACTTTACCCTGACCATCACCAG |
| | | ACTGGAACCGGAGGACTTCGCCGTGTACTACTGCCAGTACTACGGCACCTCCCCCATG |
| | | TACACATTCGGACAGGGTACCAAGGTCGAGATTAAG |
| BCMA_EBB- | 188 | EVQLLETGGGLVQPGGSLRLSCAASGFSFSSYAMSWVRQAPGKGLEWVSAISGSGGST |
| C1978-D4-aa | | YYADSVKGRFTISRDNSKNTLYLQMNSLRAEDTAVYYCAKALVGATGAFDIWGQGTLV |
| VH | | TVSS |
| BCMA_EBB- | 209 | EIVLTQSPGTLSLSPGERATLSCRASQSLSSNFLAWYQQKPGQAPGLLIYGASNWATG |
| C1978-D4-aa | | TPDRFSGSGSGTDFTLTITRLEPEDFAVYYCQYYGTSPMYTFGQGTKVEIK |
| VL | | |
| BCMA_EBB- | 230 | MALPVTALLLPLALLLHAARPEVQLLETGGGLVQPGGSLRLSCAASGFSFSSYAMSWV |
| C1978-D4-aa | | RQAPGKGLEWVSAISGSGGSTYYADSVKGRFTISRDNSKNTLYLQMNSLRAEDTAVYY |
| Full CART | | CAKALVGATGAFDIWGQGTLVTVSSGGGGSGGGGSGGGGSEIVLTQSPGTLSLSPGER |
| | | ATLSCRASQSLSSNFLAWYQQKPGQAPGLLIYGASNWATGTPDRFSGSGSGTDFTLTI |
| | | TRLEPEDFAVYYCQYYGTSPMYTFGQGTKVEIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| BCMA_EBB- | 251 | ATGGCCCTCCCTGTCACCGCCCTGCTGCTTCCGCTGGCTCTTCTGCTCCACGCCGCTC |
| C1978-D4-nt | | GGCCCGAAGTGCAGCTGCTCGAAACCGGTGGAGGGCTGGTGCAGCCAGGGGGCTCCCT |
| Full CART | | GAGGCTTTCATGCGCCGCTAGCGGATTCTCCTTCTCCTCTTACGCCATGTCGTGGGTC |
| | | CGCCAAGCCCCTGGAAAAGGCCTGGAATGGGTGTCCGCGATTTCCGGGAGCGGAGGTT |
| | | CGACCTATTACGCCGACTCCGTGAAGGGCCGCTTTACCATCTCCCGGGATAACTCCAA |
| | | GAACACTCTGTACCTCCAAATGAACTCGCTGAGAGCCGAGGACACCGCCGTGTATTAC |
| | | TGCGCGAAGGCGCTGGTCGGCGCGACTGGGGCATTCGACATCTGGGGACAGGGAACTC |
| | | TTGTGACCGTGTCGAGCGGAGGCGGCGGCTCCGGCGGAGGAGGGAGCGGGGGCGGTGG |
| | | TTCCGAAATCGTGTTGACTCAGTCCCCGGGAACCCTGAGCTTGTCACCCGGGGAGCGG |
| | | GCCACTCTCTCCTGTCGCGCCTCCCAATCGCTCTCATCCAATTTCCTGGCCTGGTACC |
| | | AGCAGAAGCCCGGACAGGCCCCGGGCCTGCTCATCTACGGCGCTTCAAACTGGGCAAC |
| | | GGGAACCCCTGATCGGTTCAGCGGAAGCGGATCGGGTACTGACTTTACCCTGACCATC |
| | | ACCAGACTGGAACCGGAGGACTTCGCCGTGTACTACTGCCAGTACTACGGCACCTCCC |
| | | CCATGTACACATTCGGACAGGGTACCAAGGTCGAGATTAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |

BCMA_EBB-C1980-A2

| | | |
|---|---|---|
| BCMA_EBB- | 147 | EVQLLESGGGLVQPGGSLRLSCAASGFTFSSYAMSWVRQAPGKGLEWVSAISGSGGST |
| C1980-A2-aa | | YYADSVKGRFTISRDNSKNTLYLQMNSLRAEDTAVYYCVLWFGEGFDPWGQGTLVTVS |
| ScFv domain | | SGGGGSGGGGSGGGGSDIVLTQSPLSLPVTPGEPASISCRSSQSLLHSNGYNYLDWYL |
| | | QKPGQSPQLLIYLGSNRASGVPDRFSGSGSGTDFTLKISRVEAEDVGVYYCMQALQTP |
| | | LTFGGGTKVDIK |
| BCMA_EBB- | 168 | GAAGTGCAGCTGCTTGAGAGCGGTGGAGGTCTGGTGCAGCCCGGGGGATCACTGCGCC |
| C1980-A2-nt | | TGTCCTGTGCCGCGTCCGGTTTCACTTTCTCCTCGTACGCCATGTCGTGGGTCAGACA |
| ScFv domain | | GGCACCGGGAAAGGGACTGGAATGGGTGTCAGCCATTTCGGGTTCGGGGGGCAGCACC |
| | | TACTACGCTGACTCCGTGAAGGGCCGGTTCACCATTTCCCGCGACAACTCCAAGAACA |
| | | CCTTGTACCTCCAAATGAACTCCCTGCGGGCCGAAGATACCGCCGTGTATTACTGCGT |
| | | GCTGTGGTTCGGAGAGGGATTCGACCCGTGGGGACAAGGAACACTCGTGACTGTGTCA |
| | | TCCGGCGGAGGCGGCAGCGGTGGCGGCGGTTCCGGCGGCGGCGGATCTGACATCGTGT |
| | | TGACCCAGTCCCCTCTGAGCCTGCCGGTCACTCCTGGCGAACCAGCCAGCATCTCCTG |
| | | CCGGTCGAGCCAGTCCCTCCTGCACTCCAATGGGTACAACTACCTCGATTGGTATCTG |
| | | CAAAAGCCGGGCCAGAGCCCCCAGCTGCTGATCTACCTTGGGTCAAACCGCGCTTCCG |
| | | GGGTGCCTGATAGATTCTCCGGGTCCGGGAGCGGAACCGACTTTACCCTGAAAATCTC |
| | | GAGGGTGGAGGCCGAGGACGTCGGAGTGTACTACTGCATGCAGGCGCTCCAGACTCCC |
| | | CTGACCTTCGGAGGAGGAACGAAGGTCGACATCAAGA |
| BCMA_EBB- | 189 | EVQLLESGGGLVQPGGSLRLSCAASGFTFSSYAMSWVRQAPGKGLEWVSAISGSGGST |
| C1980-A2-aa | | YYADSVKGRFTISRDNSKNTLYLQMNSLRAEDTAVYYCVLWFGEGFDPWGQGTLVTVS |
| VH | | S |
| BCMA_EBB- | 210 | DIVLTQSPLSLPVTPGEPASISCRSSQSLLHSNGYNYLDWYLQKPGQSPQLLIYLGSN |
| C1980-A2-aa | | RASGVPDRFSGSGSGTDFTLKISRVEAEDVGVYYCMQALQTPLTFGGGTKVDIK |
| VL | | |
| BCMA_EBB- | 231 | MALPVTALLLPLALLLHAARPEVQLLESGGGLVQPGGSLRLSCAASGFTFSSYAMSWV |
| C1980-A2-aa | | RQAPGKGLEWVSAISGSGGSTYYADSVKGRFTISRDNSKNTLYLQMNSLRAEDTAVYY |
| Full CART | | CVLWFGEGFDPWGQGTLVTVSSGGGGSGGGGSGGGGSDIVLTQSPLSLPVTPGEPASI |
| | | SCRSSQSLLHSNGYNYLDWYLQKPGQSPQLLIYLGSNRASGVPDRFSGSGSGTDFTLK |
| | | ISRVEAEDVGVYYCMQALQTPLTFGGGTKVDIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| BCMA_EBB- | 252 | ATGGCCCTCCCTGTCACCGCCCTGCTGCTTCCGCTGGCTCTTCTGCTCCACGCCGCTC |
| C1980-A2-nt | | GGCCCGAAGTGCAGCTGCTTGAGAGCGGTGGAGGTCTGGTGCAGCCCGGGGGATCACT |
| Full CART | | GCGCCTGTCCTGTGCCGCGTCCGGTTTCACTTTCTCCTCGTACGCCATGTCGTGGGTC |
| | | AGACAGGCACCGGGAAAGGGACTGGAATGGGTGTCAGCCATTTCGGGTTCGGGGGGCA |
| | | GCACCTACTACGCTGACTCCGTGAAGGGCCGGTTCACCATTTCCCGCGACAACTCCAA |
| | | GAACACCTTGTACCTCCAAATGAACTCCCTGCGGGCCGAAGATACCGCCGTGTATTAC |
| | | TGCGTGCTGTGGTTCGGAGAGGGATTCGACCCGTGGGGACAAGGAACACTCGTGACTG |
| | | TGTCATCCGGCGGAGGCGGCAGCGGTGGCGGCGGTTCCGGCGGCGGCGGATCTGACAT |
| | | CGTGTTGACCCAGTCCCCTCTGAGCCTGCCGGTCACTCCTGGCGAACCAGCCAGCATC |
| | | TCCTGCCGGTCGAGCCAGTCCCTCCTGCACTCCAATGGGTACAACTACCTCGATTGGT |
| | | ATCTGCAAAAGCCGGGCCAGAGCCCCCAGCTGCTGATCTACCTTGGGTCAAACCGCGC |
| | | TTCCGGGGTGCCTGATAGATTCTCCGGGTCCGGGAGCGGAACCGACTTTACCCTGAAA |
| | | ATCTCGAGGGTGGAGGCCGAGGACGTCGGAGTGTACTACTGCATGCAGGCGCTCCAGA |
| | | CTCCCCTGACCTTCGGAGGAGGAACGAAGGTCGACATCAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |

BCMA_EBB-C1981-C3

| | | |
|---|---|---|
| BCMA_EBB- | 148 | QVQLVESGGGLVQPGGSLRLSCAASGFTFSSYAMSWVRQAPGKGLEWVSAISGSGGST |
| C1981-C3-aa | | YYADSVKGRFTISRDNSKNTLYLQMNSLRAEDTAVYYCAKVGYDSSGYYRDYYGMDVW |
| ScFv domain | | GQGTTVTVSSGGGGSGGGGSGGGGSEIVLTQSPGTLSLSPGERATLSCRASQSVSSSY |
| | | LAWYQQKPGQAPRLLIYGTSSRATGISDRFSGSGSGTDFTLTISRLEPEDFAVYYCQH |
| | | YGNSPPKFTFGPGTKLEIK |
| BCMA_EBB- | 169 | CAAGTGCAGCTCGTGGAGTCAGGCGGAGGACTGGTGCAGCCCGGGGGCTCCCTGAGAC |
| C1981-C3-nt | | TTTCCTGCGCGGCATCGGGTTTTACCTTCTCCTCCTATGCTATGTCCTGGGTGCGCCA |
| ScFv domain | | GGCCCCGGGAAAGGGACTGGAATGGGTGTCCGCAATCAGCGGTAGCGGGGGCTCAACA |
| | | TACTACGCCGACTCCGTCAAGGGTCGCTTCACTATTTCCCGGGACAACTCCAAGAATA |
| | | CCCTGTACCTCCAAATGAACAGCCTCAGGGCCGAGGATACTGCCGTGTACTACTGCGC |
| | | CAAAGTCGGATACGATAGCTCCGGTTACTACCGGGACTACTACGGAATGGACGTGTGG |
| | | GGACAGGGCACCACCGTGACCGTGTCAAGCGGCGGAGGCGGTTCAGGAGGGGGAGGCT |
| | | CCGGCGGTGGAGGGTCCGAAATCGTCCTGACTCAGTCGCCTGGCACTCTGTCGTTGTC |
| | | CCCGGGGGAGCGCGCTACCCTGTCGTGTCGGGCGTCGCAGTCCGTGTCGAGCTCCTAC |
| | | CTCGCGTGGTACCAGCAGAAGCCCGGACAGGCCCCTAGACTTCTGATCTACGGCACTT |
| | | CTTCACGCGCCACCGGGATCAGCGACAGGTTCAGCGGCTCCGGCTCCGGGACCGACTT |
| | | CACCCTGACCATTAGCCGGCTGGAGCCTGAAGATTTCGCCGTGTATTACTGCCAACAC |
| | | TACGGAAACTCGCCGCCAAAGTTCACGTTCGGACCCGGAACCAAGCTGGAAATCAAG |
| BCMA_EBB- | 190 | QVQLVESGGGLVQPGGSLRLSCAASGFTFSSYAMSWVRQAPGKGLEWVSAISGSGGST |
| C1981-C3-aa | | YYADSVKGRFTISRDNSKNTLYLQMNSLRAEDTAVYYCAKVGYDSSGYYRDYYGMDVW |
| VH | | GQGTTVTVSS |
| BCMA_EBB- | 211 | EIVLTQSPGTLSLSPGERATLSCRASQSVSSSYLAWYQQKPGQAPRLLIYGTSSRATG |
| C1981-C3-aa | | ISDRFSGSGSGTDFTLTISRLEPEDFAVYYCQHYGNSPPKFTFGPGTKLEIK |
| VL | | |
| BCMA_EBB- | 232 | MALPVTALLLPLALLLHAARPQVQLVESGGGLVQPGGSLRLSCAASGFTFSSYAMSWV |
| C1981-C3-aa | | RQAPGKGLEWVSAISGSGGSTYYADSVKGRFTISRDNSKNTLYLQMNSLRAEDTAVYY |
| Full CART | | CAKVGYDSSGYYRDYYGMDVWGQGTTVTVSSGGGGSGGGGSGGGGSEIVLTQSPGTLS |
| | | LSPGERATLSCRASQSVSSSYLAWYQQKPGQAPRLLIYGTSSRATGISDRFSGSGSGT |
| | | DFTLTISRLEPEDFAVYYCQHYGNSPPKFTFGPGTKLEIKTTTPAPRPPTPAPTIASQ |
| | | PLSLRPEACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEAYSEIGMKGERR |
| | | RGKGHDGLYQGLSTATKDTYDALHMQALPPR |
| BCMA_EBB- | 253 | ATGGCCCTCCCTGTCACCGCCCTGCTGCTTCCGCTGGCTCTTCTGCTCCACGCCGCTC |
| C1981-C3-nt | | GGCCCCAAGTGCAGCTCGTGGAGTCAGGCGGAGGACTGGTGCAGCCCGGGGGCTCCCT |
| Full CART | | GAGACTTTCCTGCGCGGCATCGGGTTTTACCTTCTCCTCCTATGCTATGTCCTGGGTG |
| | | CGCCAGGCCCCGGGAAAGGGACTGGAATGGGTGTCCGCAATCAGCGGTAGCGGGGGCT |
| | | CAACATACTACGCCGACTCCGTCAAGGGTCGCTTCACTATTTCCCGGGACAACTCCAA |
| | | GAATACCCTGTACCTCCAAATGAACAGCCTCAGGGCCGAGGATACTGCCGTGTACTAC |
| | | TGCGCCAAAGTCGGATACGATAGCTCCGGTTACTACCGGGACTACTACGGAATGGACG |
| | | TGTGGGGACAGGGCACCACCGTGACCGTGTCAAGCGGCGGAGGCGGTTCAGGAGGGGG |
| | | AGGCTCCGGCGGTGGAGGGTCCGAAATCGTCCTGACTCAGTCGCCTGGCACTCTGTCG |
| | | TTGTCCCCGGGGGAGCGCGCTACCCTGTCGTGTCGGGCGTCGCAGTCCGTGTCGAGCT |
| | | CCTACCTCGCGTGGTACCAGCAGAAGCCCGGACAGGCCCCTAGACTTCTGATCTACGG |
| | | CACTTCTTCACGCGCCACCGGGATCAGCGACAGGTTCAGCGGCTCCGGCTCCGGGACC |
| | | GACTTCACCCTGACCATTAGCCGGCTGGAGCCTGAAGATTTCGCCGTGTATTACTGCC |
| | | AACACTACGGAAACTCGCCGCCAAAGTTCACGTTCGGACCCGGAACCAAGCTGGAAAT |
| | | CAAGACCACTACCCCAGCACCGAGGCCACCCACCCCGGCTCCTACCATCGCCTCCCAG |
| | | CCTCTGTCCCTGCGTCCGGAGGCATGTAGACCCGCAGCTGGTGGGGCCGTGCATACCC |
| | | GGGGTCTTGACTTCGCCTGCGATATCTACATTTGGGCCCCTCTGGCTGGTACTTGCGG |
| | | GGTCCTGCTGCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAGAGGAGGACG |
| | | GCTGTTCATGCCGGTTCCCAGAGGAGGAGGAAGGCGGCTGCGAACTGCGCGTGAAATT |
| | | CAGCCGCAGCGCAGATGCTCCAGCCTACAAGCAGGGGCAGAACCAGCTCTACAACGAA |
| | | CTCAATCTTGGTCGGAGAGAGGAGTACGACGTGCTGGACAAGCGGAGAGGACGGGACC |
| | | CAGAAATGGGCGGGAAGCCGCGCAGAAAGAATCCCCAAGAGGGCCTGTACAACGAGCT |
| | | CCAAAAGGATAAGATGGCAGAAGCCTATAGCGAGATTGGTATGAAAGGGGAACGCAGA |
| | | AGAGGCAAAGGCCACGACGGACTGTACCAGGGACTCAGCACCGCCACCAAGGACACCT |
| | | ATGACGCTCTTCACATGCAGGCCCTGCCGCCTCGG |

BCMA_EBB-C1978-G4

| | | |
|---|---|---|
| BCMA_EBB- | 149 | EVQLVESGGGLVQPGGSLRLSCAASGFTFSSYAMSWVRQAPGKGLEWVSAISGSGGST |
| C1978-G4-aa | | YYADSVKGRFTISRDNSKNTLYLQMNSLRAEDTAVYYCAKMGWSSGYLGAFDIWGQGT |
| ScFv domain | | TVTVSSGGGGSGGGGSGGGGSEIVLTQSPGTLSLSPGERATLSCRASQSVASSFLAWY |
| | | QQKPGQAPRLLIYGASGRATGIPDRFSGSGSGTDFTLTISRLEPEDFAVYYCQHYGGS |
| | | PRLTFGGGTKVDIK |
| BCMA_EBB- | 170 | GAAGTCCAACTGGTGGAGTCCGGGGGAGGGCTCGTGCAGCCCGGAGGCAGCCTTCGGC |
| C1978-G4-nt | | TGTCGTGCGCCGCCTCCGGGTTCACGTTCTCATCCTACGCGATGTCGTGGGTCAGACA |
| ScFv domain | | GGCACCAGGAAAGGGACTGGAATGGGTGTCCGCCATTAGCGGCTCCGGCGGTAGCACC |
| | | TACTATGCCGACTCAGTGAAGGGAAGGTTCACTATCTCCCGCGACAACAGCAAGAACA |
| | | CCCTGTACCTCCAAATGAACTCTCTGCGGGCCGAGGATACCGCGGTGTACTATTGCGC |
| | | CAAGATGGGTTGGTCCAGCGGATACTTGGGAGCCTTCGACATTTGGGGACAGGGCACT |
| | | ACTGTGACCGTGTCCTCCGGGGGTGGCGGATCGGGAGGCGGCGGCTCGGGTGGAGGGG |
| | | GTTCCGAAATCGTGTTGACCCAGTCACCGGGAACCCTCTCGCTGTCCCCGGGAGAACG |
| | | GGCTACACTGTCATGTAGAGCGTCCCAGTCCGTGGCTTCCTCGTTCCTGGCCTGGTAC |
| | | CAGCAGAAGCCGGGACAGGCACCCCGCCTGCTCATCTACGGAGCCAGCGGCCGGGCGA |
| | | CCGGCATCCCTGACCGCTTCTCCGGTTCCGGCTCGGGCACCGACTTTACTCTGACCAT |
| | | TAGCAGGCTTGAGCCCGAGGATTTTGCCGTGTACTACTGCCAACACTACGGGGGGAGC |
| | | CCTCGCCTGACCTTCGGAGGCGGAACTAAGGTCGATATCAAAA |
| BCMA_EBB- | 191 | EVQLVESGGGLVQPGGSLRLSCAASGFTFSSYAMSWVRQAPGKGLEWVSAISGSGGST |
| C1978-G4-aa | | YYADSVKGRFTISRDNSKNTLYLQMNSLRAEDTAVYYCAKMGWSSGYLGAFDIWGQGT |
| VH | | TVTVSS |
| BCMA_EBB- | 212 | EIVLTQSPGTLSLSPGERATLSCRASQSVASSFLAWYQQKPGQAPRLLIYGASGRATG |
| C1978-G4-aa | | IPDRFSGSGSGTDFTLTISRLEPEDFAVYYCQHYGGSPRLTFGGGTKVDIK |
| VL | | |
| BCMA_EBB- | 233 | MALPVTALLLPLALLLHAARPEVQLVESGGGLVQPGGSLRLSCAASGFTFSSYAMSWV |
| C1978-G4-aa | | RQAPGKGLEWVSAISGSGGSTYYADSVKGRFTISRDNSKNTLYLQMNSLRAEDTAVYY |
| Full CART | | CAKMGWSSGYLGAFDIWGQGTTVTVSSGGGGSGGGGSGGGGSEIVLTQSPGTLSLSPG |
| | | ERATLSCRASQSVASSFLAWYQQKPGQAPRLLIYGASGRATGIPDRFSGSGSGTDFTL |
| | | TISRLEPEDFAVYYCQHYGGSPRLTFGGGTKVDIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 254 | ATGGCCCTCCCTGTCACCGCCCTGCTGCTTCCGCTGGCTCTTCTGCTCCACGCCGCTC |
| C1978-G4-nt | | GGCCCGAAGTCCAACTGGTGGAGTCCGGGGGAGGGCTCGTGCAGCCCGGAGGCAGCCT |
| Full CART | | TCGGCTGTCGTGCGCCGCCTCCGGGTTCACGTTCTCATCCTACGCGATGTCGTGGGTC |
| | | AGACAGGCACCAGGAAAGGGACTGGAATGGGTGTCCGCCATTAGCGGCTCCGGCGGTA |
| | | GCACCTACTATGCCGACTCAGTGAAGGGAAGGTTCACTATCTCCCGCGACAACAGCAA |
| | | GAACACCCTGTACCTCCAAATGAACTCTCTGCGGGCCGAGGATACCGCGGTGTACTAT |
| | | TGCGCCAAGATGGGTTGGTCCAGCGGATACTTGGGAGCCTTCGACATTTGGGGACAGG |
| | | GCACTACTGTGACCGTGTCCTCCGGGGGTGGCGGATCGGGAGGCGGCGGCTCGGGTGG |
| | | AGGGGGTTCCGAAATCGTGTTGACCCAGTCACCGGGAACCCTCTCGCTGTCCCCGGGA |
| | | GAACGGGCTACACTGTCATGTAGAGCGTCCCAGTCCGTGGCTTCCTCGTTCCTGGCCT |
| | | GGTACCAGCAGAAGCCGGGACAGGCACCCCGCCTGCTCATCTACGGAGCCAGCGGCCG |
| | | GGCGACCGGCATCCCTGACCGCTTCTCCGGTTCCGGCTCGGGCACCGACTTTACTCTG |
| | | ACCATTAGCAGGCTTGAGCCCGAGGATTTTGCCGTGTACTACTGCCAACACTACGGGG |
| | | GGAGCCCTCGCCTGACCTTCGGAGGCGGAACTAAGGTCGATATCAAAACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

TABLE 3
Additional exemplary BCMA CAR sequences

| | | SEQ |
|---|---|---|
| | | ID |
| Name | Sequence | NO: |
| A7D12.2 | QIQLVQSGPDLKKPGETVKLSCKASGYTFTNFGMNWVKQAPGKGFKWMAWINT | 255 |
| VH | YTGESYFADDFKGRFAFSVETSATTAYLQINNLKTEDTATYFCARGEIYYGYDGGF | |
| | AYWGQGTLVTVSA | |
| A7D12.2 | DVVMTQSHRFMSTSVGDRVSITCRASQDVNTAVSWYQQKPGQSPKLLIFSASYRY | 259 |
| VL | TGVPDRFTGSGSGADFTLTISSVQAEDLAVYYCQQHYSTPWTFGGGTKLDIK | |
| A7D12.2 | QIQLVQSGPDLKKPGETVKLSCKASGYTFTNFGMNWVKQAPGKGFKWMAWINT | 263 |
| scFv | YTGESYFADDFKGRFAFSVETSATTAYLQINNLKTEDTATYFCARGEIYYGYDGGF | |
| domain | AYWGQGTLVTVSAGGGGSGGGGSGGGGSDVVMTQSHRFMSTSVGDRVSITCRAS | |
| | QDVNTAVSWYQQKPGQSPKLLIFSASYRYTGVPDRFTGSGSGADFTLTISSVQAED | |
| | LAVYYCQQHYSTPWTFGGGTKLDIK | |
| A7D12.2 | QIQLVQSGPDLKKPGETVKLSCKASGYTFTNFGMNWVKQAPGKGFKWMAWINT | 267 |
| Full CART | YTGESYFADDFKGRFAFSVETSATTAYLQINNLKTEDTATYFCARGEIYYGYDGGF | |
| | AYWGQGTLVTVSAGGGGSGGGGSGGGGSDVVMTQSHRFMSTSVGDRVSITCRAS | |
| | QDVNTAVSWYQQKPGQSPKLLIFSASYRYTGVPDRFTGSGSGADFTLTISSVQAED | |
| | LAVYYCQQHYSTPWTFGGGTKLDIKTTTPAPRPPTPAPTIASQPLSLRPEACRPAAG | |
| | GAVHTRGLDFACDIYIWAPLAGTCGVLLLSLVITLYCKRGRKKLLYIFKQPFMRPV | |
| | QTTQEEDGCSCRFPEEEEGGCELRVKFSRSADAPAYKQGQNQLYNELNLGRREEY | |
| | DVLDKRRGRDPEMGGKPRRKNPQEGLYNELQKDKMAEAYSEIGMKGERRRGKG | |
| | HDGLYQGLSTATKDTYDALHMQALPPR | |
| C11D5.3 | QIQLVQSGPELKKPGETVKISCKASGYTFTDYSINWVKRAPGKGLKWMGWINTET | 256 |
| VH | REPAYAYDFRGRFAFSLETSASTAYLQINNLKYEDTATYFCALDYSYAMDYWGQ | |
| | GTSVTVSS | |
| C11D5.3 | DIVLTQSPASLAMSLGKRATISCRASESVSVIGAHLIHWYQQKPGQPPKLLIYLASN | 260 |
| VL | LETGVPARFSGSGSGTDFTLTIDPVEEDDVAIYSCLQSRIFPRTFGGGTKLEIK | |
| C11D5.3 | QIQLVQSGPELKKPGETVKISCKASGYTFTDYSINWVKRAPGKGLKWMGWINTET | 264 |
| scFv | REPAYAYDFRGRFAFSLETSASTAYLQINNLKYEDTATYFCALDYSYAMDYWGQ | |
| domain | GTSVTVSSGGGGSGGGGSGGGGSQIQLVQSGPELKKPGETVKISCKASGYTFTDYS | |
| | INWVKRAPGKGLKWMGWINTETREPAYAYDFRGRFAFSLETSASTAYLQINNLKY | |
| | EDTATYFCALDYSYAMDYWGQGTSVTVSS | |
| C11D5.3 | QIQLVQSGPELKKPGETVKISCKASGYTFTDYSINWVKRAPGKGLKWMGWINTET | 268 |
| Full CART | REPAYAYDFRGRFAFSLETSASTAYLQINNLKYEDTATYFCALDYSYAMDYWGQ | |
| | GTSVTVSSGGGGSGGGGSGGGGSQIQLVQSGPELKKPGETVKISCKASGYTFTDYS | |
| | INWVKRAPGKGLKWMGWINTETREPAYAYDFRGRFAFSLETSASTAYLQINNLKY | |
| | EDTATYFCALDYSYAMDYWGQGTSVTVSSTTTPAPRPPTPAPTIASQPLSLRPEAC | |
| | RPAAGGAVHTRGLDFACDIYIWAPLAGTCGVLLLSLVITLYCKRGRKKLLYIFKQP | |
| | FMRPVQTTQEEDGCSCRFPEEEEGGCELRVKFSRSADAPAYKQGQNQLYNELNLG | |
| | RREEYDVLDKRRGRDPEMGGKPRRKNPQEGLYNELQKDKMAEAYSEIGMKGER | |
| | RRGKGHDGLYQGLSTATKDTYDALHMQALPPR | |
| C12A3.2 | QIQLVQSGPELKKPGETVKISCKASGYTFRHYSMNWVKQAPGKGLKWMGRINTES | 257 |
| VH | GVPIYADDFKGRFAFSVETSASTAYLVINNLKDEDTASYFCSNDYLYSLDFWGQGT | |
| | ALTVSS | |
| C12A3.2 | DIVLTQSPPSLAMSLGKRATISCRASESVTILGSHLIYWYQQKPGQPPTLLIQLASNV | 261 |
| VL | QTGVPARFSGSGSRTDFTLTIDPVEEDDVAVYYCLQSRTIPRTFGGGTKLEIK | |
| C12A3.2 | QIQLVQSGPELKKPGETVKISCKASGYTFRHYSMNWVKQAPGKGLKWMGRINTES | 265 |
| scFv | GVPIYADDFKGRFAFSVETSASTAYLVINNLKDEDTASYFCSNDYLYSLDFWGQGT | |
| domain | ALTVSSGGGGSGGGGSGGGGSDIVLTQSPPSLAMSLGKRATISCRASESVTILGSHL | |
| | IYWYQQKPGQPPTLLIQLASNVQTGVPARFSGSGSRTDFTLTIDPVEEDDVAVYYC | |
| | LQSRTIPRTFGGGTKLEIK | |
| C12A3.2 | QIQLVQSGPELKKPGETVKISCKASGYTFRHYSMNWVKQAPGKGLKWMGRINTES | 269 |
| Full CART | GVPIYADDFKGRFAFSVETSASTAYLVINNLKDEDTASYFCSNDYLYSLDFWGQGT | |
| | ALTVSSGGGGSGGGGSGGGGSDIVLTQSPPSLAMSLGKRATISCRASESVTILGSHL | |
| | IYWYQQKPGQPPTLLIQLASNVQTGVPARFSGSGSRTDFTLTIDPVEEDDVAVYYC | |
| | LQSRTIPRTFGGGTKLEIKTTTPAPRPPTPAPTIASQPLSLRPEACRPAAGGAVHTRG | |
| | LDFACDIYIWAPLAGTCGVLLLSLVITLYCKRGRKKLLYIFKQPFMRPVQTTQEED | |
| | GCSCRFPEEEEGGCELRVKFSRSADAPAYKQGQNQLYNELNLGRREEYDVLDKRR | |
| | GRDPEMGGKPRRKNPQEGLYNELQKDKMAEAYSEIGMKGERRRGKGHDGLYQG | |
| | LSTATKDTYDALHMQALPPR | |
| C13F12.1 | QIQLVQSGPELKKPGETVKISCKASGYTFTHYSMNWVKQAPGKGLKWMGRINTET | 258 |
| VH | GEPLYADDFKGRFAFSLETSASTAYLVINNLKNEDTATFFCSNDYLYSCDYWGQG | |
| | TTLTVSS | |
| C13F12.1 | DIVLTQSPPSLAMSLGKRATISCRASESVTILGSHLIYWYQQKPGQPPTLLIQLASNV | 262 |
| VL | QTGVPARFSGSGSRTDFTLTIDPVEEDDVAVYYCLQSRTIPRTFGGGTKLEIK | |
| C13F12.1 | QIQLVQSGPELKKPGETVKISCKASGYTFTHYSMNWVKQAPGKGLKWMGRINTET | 266 |
| scFv | GEPLYADDFKGRFAFSLETSASTAYLVINNLKNEDTATFFCSNDYLYSCDYWGQG | |
| domain | TTLTVSSGGGGSGGGGSGGGGSDIVLTQSPPSLAMSLGKRATISCRASESVTILGSH | |
| | LIYWYQQKPGQPPTLLIQLASNVQTGVPARFSGSGSRTDFTLTIDPVEEDDVAVYY | |
| | CLQSRTIPRTFGGGTKLEIK | |
| C13F12.1 | QIQLVQSGPELKKPGETVKISCKASGYTFTHYSMNWVKQAPGKGLKWMGRINTET | 270 |
| Full CART | GEPLYADDFKGRFAFSLETSASTAYLVINNLKNEDTATFFCSNDYLYSCDYWGQG | |
| | TTLTVSSGGGGSGGGGSGGGGSDIVLTQSPPSLAMSLGKRATISCRASESVTILGSH | |
| | LIYWYQQKPGQPPTLLIQLASNVQTGVPARFSGSGSRTDFTLTIDPVEEDDVAVYY | |
| | CLQSRTIPRTFGGGTKLEIKTTTPAPRPPTPAPTIASQPLSLRPEACRPAAGGAVHTR | |
| | GLDFACDIYIWAPLAGTCGVLLLSLVITLYCKRGRKKLLYIFKQPFMRPVQTTQEE | |
| | DGCSCRFPEEEEGGCELRVKFSRSADAPAYKQGQNQLYNELNLGRREEYDVLDKR | |
| | RGRDPEMGGKPRRKNPQEGLYNELQKDKMAEAYSEIGMKGERRRGKGHDGLYQ | |
| | GLSTATKDTYDALHMQALPPR | |

### RNA Transfection

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases (SEQ ID NO: 276) in length. RNA so produced can efficiently transfect different kinds of cells. In one aspect, the template includes sequences for the CAR.

In one aspect the anti-BCMA CAR is encoded by a messenger RNA (mRNA). In one aspect the mRNA encoding the anti-BCMA CAR is introduced into an immune effector cell, e.g., a T cell or a NK cell, for production of a CAR-expressing cell (e.g., CART cell or CAR-expressing NK cell).

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR of the present invention. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 277) (size can be 50-5000 T (SEQ ID NO: 278)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (SEQ ID NO: 279).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 (SEQ ID NO: 280) nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Nucleic Acid Constructs Encoding a CAR

The present invention also provides nucleic acid molecules encoding one or more CAR constructs described herein. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), wherein the CAR comprises an antigen binding domain, a transmembrane domain, and an intracellular signaling domain comprising a stimulatory domain, e.g., a costimulatory signaling domain and/or a primary signaling domain, e.g., zeta chain.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See below June et al. 2009Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

WT PGK Promoter
(SEQ ID NO: 281)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT
Exemplary truncated PGK Promoters:
PGK100:
(SEQ ID NO: 282)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 283)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 284)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 285)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to a target expressed on acute myeloid leukemia cells, such as, e.g., CD123, CD34, CLL-1, folate receptor beta, or FLT3; or a target expressed on a B cell, e.g., CD10, CD19, CD20, CD22, CD34, CD123, FLT-3, ROR1, CD79b, CD179b, or CD79a. In one embodiment, the vector comprises a nucleic acid sequence encoding a first CAR that specifically binds a first antigen and includes an intracellular signaling domain having a costimulatory signaling domain but not a primary signaling domain, and a nucleic acid encoding a second CAR that specifically binds a second, different, antigen and includes an intracellular signaling domain having a primary signaling domain but not a costimulatory signaling domain. In one embodiment, the vector comprises a nucleic acid encoding a first BCMA CAR that includes a BCMA binding domain, a transmembrane domain and a costimulatory domain and a nucleic acid encoding a second CAR that targets an antigen other than BCMA (e.g., an antigen expressed on AML cells, e.g., CD123, CD34, CLL-1, folate receptor beta, or FLT3; or an antigen expressed on a B cell, e.g., CD10, CD19, CD20, CD22, CD34, CD123, FLT-3, ROR1, CD79b, CD179b, or CD79a) and includes an antigen binding domain, a transmembrane domain and a primary signaling domain. In another embodiment, the vector comprises a nucleic acid encoding a first BCMA CAR that includes a BCMA binding domain, a transmembrane domain and a primary signaling domain and a nucleic acid encoding a second CAR that specifically binds an antigen other than BCMA (e.g., an antigen expressed on AML cells, e.g., CD123, CD34, CLL-1, folate receptor beta, or FLT3; or an antigen expressed on a B cell, e.g., CD10, CD19, CD20, CD22, CD34, CD123, FLT-3, ROR1, CD79b, CD179b, or CD79a) and includes an antigen binding domain to the antigen, a transmembrane domain and a costimulatory signaling domain.

In one embodiment, the vector comprises a nucleic acid encoding a BCMA CAR described herein and a nucleic acid encoding an inhibitory CAR. In one embodiment, the inhibitory CAR comprises an antigen binding domain that binds an antigen found on normal cells but not cancer cells, e.g., normal cells that also express BCMA. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta.

In embodiments, the vector may comprise two or more nucleic acid sequences encoding a CAR, e.g., a BCMA CAR described herein and a second CAR, e.g., an inhibitory CAR or a CAR that specifically binds to an antigen other than BCMA (e.g., an antigen expressed on AML cells, e.g., CD123, CLL-1, CD34, FLT3, or folate receptor beta; or antigen expression B cells, e.g., CD10, CD19, CD20, CD22, CD34, CD123, FLT-3, ROR1, CD79b, CD179b, or CD79a). In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:
(SEQ ID NO: 286)
(GSG)EGRGSLLTCGDVEENPGP
P2A:
(SEQ ID NO: 287)
(GSG)ATNFSLLKQAGDVEENPGP
E2A:
(SEQ ID NO: 288)
(GSG)QCTNYALLKLAGDVESNPGP
F2A:
(SEQ ID NO: 289)
(GSG)VKQTLNFDLLKLAGDVESNPGP

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells or NK cells. In one aspect, the mammalian T cell is a human T cell. In one aspect, the mammalian NK cell is a human NK cell.

### Sources of Cells

Prior to expansion and genetic modification, a source of cells, e.g., immune effector cells (e.g., T cells or NK cells), is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present invention, any number of immune effector cell (e.g., T cell or NK cell) lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD4+, CD8+, CD45RA+, and/or CD45RO+T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. In certain aspects, it may be desirable to enrich for cells that are CD127low. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11 b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta. In embodiments, the checkpoint inhibitor is PD1 or PD-L1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/m1 to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as immune effector cells, e.g., T cells or NK cells, isolated and frozen for later use in cell therapy, e.g., T cell therapy, for any number of diseases or conditions that would benefit from cell therapy, e.g., T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the immune effector cells (e.g., T cells or NK cells) may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Modifications of CAR Cells, Including Allogeneic CAR Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II, and/or beta-2 microglobulin (β₂m). Compositions of allogeneic CAR and methods thereof have been described in, e.g., pages 227-237 of WO 2016/014565, incorporated herein by reference in its entirety.

In some embodiments, a cell, e.g., a T cell or a NK cell, is modified to reduce the expression of a TCR, and/or HLA, and/or β₂m, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta), using, e.g., a method described herein, e.g., siRNA, shRNA, clustered regularly interspaced short palindromic repeats (CRISPR) transcription-activator like effector nuclease (TALEN), or zinc finger endonuclease (ZFN).

In some embodiments, a cell, e.g., a T cell or a NK cell is engineered to express a telomerase subunit, e.g., the catalytic subunit of telomerase, e.g., TERT, e.g., hTERT. In one embodiment, such modification improves persistence of the cell in a patient.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al, J Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a BCMA CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a BCMA CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a BCMA CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a BCMA CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence of IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population. Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a BCMA CAR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a BCMA CAR are described in further detail below

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ T cells following antigen stimulation can be measured by flow cytometry. For example, a mixture of CD4⁺ and CD8⁺ T cells are stimulated with aCD3/aCD28 aAPCs followed by transduction with lentiviral vectors expressing GFP under the control of the promoters to be analyzed. Exemplary promoters include the CMV IE gene, EF-1 a, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with aCD3/aCD28 coated magnetic beads on day 0, and transduced with CAR on day 1 using a bicistronic lentiviral vector expressing CAR along with eGFP using a 2A ribosomal skipping sequence. Cultures are re-stimulated with BCMA-expressing cells, such as multiple myeloma cell lines or K562-BCMA, following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with aCD3/aCD28 coated magnetic beads on day 0, and transduction with the indicated CAR on day 1.

Animal models can also be used to measure a CART activity. For example, xenograft model using human BCMA-specific CAR⁺ T cells to treat a primary human multiple myeloma in immunodeficient mice can be used. See, e.g., Milone et al., Molecular Therapy 17(8): 1453-1464 (2009). Very briefly, after establishment of MM, mice are randomized as to treatment groups. Different numbers of BCMA CART cells can be injected into immunodeficient mice bearing MM. Animals are assessed for disease progression and tumor burden at weekly intervals. Survival curves for the groups are compared using the log-rank test. In addition, absolute peripheral blood CD4⁺ and CD8⁺ T cell counts 4 weeks following T cell injection in the immunodeficient mice can also be analyzed. Mice are injected with multiple myeloma cells and 3 weeks later are injected with T cells engineered to express BCMA CAR, e.g., by a bicistronic lentiviral vector that encodes the CAR linked to eGFP. T cells are normalized to 45-50% input GFP⁺ T cells by mixing with mock-transduced cells prior to injection, and confirmed by flow cytometry. Animals are assessed for leukemia at 1-week intervals. Survival curves for the CAR⁺ T cell groups are compared using the log-rank test.

Assessment of cell proliferation and cytokine production has been previously described, e.g., at Milone et al., Molecular Therapy 17(8): 1453-1464 (2009). Briefly, assessment of CAR-mediated proliferation is performed in microtiter plates by mixing washed T cells with K562 cells expressing BCMA or other BCMA-expressing myeloma cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, Calif.) and flow cytometry as described by the manufacturer. CAR⁺ T cells are identified by GFP expression using T cells that are engineered with eGFP-2A linked CAR-expressing lentiviral vectors. For CAR+ T cells not expressing GFP, the CAR+ T cells are detected with biotinylated recombinant BCMA protein and a secondary avidin-PE conjugate. CD4+ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, Calif.) according the manufacturer's instructions. Fluorescence is assessed using a FACScalibur flow cytometer, and data is analyzed according to the manufacturer's instructions.

Cytotoxicity can be assessed by a standard 51Cr-release assay. See, e.g., Milone et al., Molecular Therapy 17(8): 1453-1464 (2009). Briefly, target cells (e.g., K562 lines expressing BCMA and primary multiple myeloma cells) are loaded with 51Cr (as NaCrO4, New England Nuclear, Boston, Mass.) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released 51Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average 51Cr released for each experimental condition. Alternatively, cytotoxicity can also be assessed using a Bright-Glo™ Luciferase Assay.

Imaging technologies can be used to evaluate specific trafficking and proliferation of CARs in tumor-bearing animal models. Such assays have been described, for example, in Barrett et al., Human Gene Therapy 22:1575-1586 (2011). Briefly, NOD/SCID/γc^{-/-} (NSG) mice or other immunodeficient are injected IV with multiple myeloma cells followed 7 days later with BCMA CART cells 4 hour after electroporation with the CAR constructs. The T cells are stably transfected with a lentiviral construct to express firefly luciferase, and mice are imaged for bioluminescence. Alternatively, therapeutic efficacy and specificity of a single injection of CAR⁺ T cells in a multiple myeloma xenograft model can be measured as the following: NSG mice are injected with multiple myeloma cells transduced to stably express firefly luciferase, followed by a single tail-vein injection of T cells electroporated with BCMA CAR construct days later. Animals are imaged at various time points post injection. For example, photon-density heat maps of firefly luciferasepositive tumors in representative mice at day 5 (2 days before treatment) and day 8 (24 hr post CAR⁺ PBLs) can be generated.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In one embodiment, the anti-idiotypic antibody molecule recognizes an anti-CD19 antibody molecule, e.g., an anti-CD19 scFv. For instance, the anti-idiotypic antibody molecule can compete for binding with the CD19-specific CAR mAb clone no. 136.20.1 described in Jena et al., PLOS March 2013 8:3 e57838; may have the same CDRs (e.g., one or more of, e.g., all of, VH CDR1, VH CDR2, CH CDR3, VL CDR1, VL CDR2, and VL CDR3, using the Kabat definition, the Chothia definition, or a combination of the Kabat and Chothia definitions) as the CD19-specific CAR mAb clone no. 136.20.1; may have one or more (e.g., 2) variable regions as the CD19-specific CAR mAb clone no. 136.20.1, or may comprise the CD19-specific CAR mAb clone no. 136.20.1. In some embodiments, the anti-idiotypic antibody was made according to a method described in Jena et al. In another embodiment, the anti-idiotypic antibody molecule is an anti-idiotypic antibody molecule described in WO 2014/190273. In some embodiments, the anti-idiotypic antibody molecule has the same CDRs (e.g., one or more of, e.g., all of, VH CDR1, VH CDR2, CH CDR3, VL CDR1, VL CDR2, and VL CDR3) as an antibody molecule of WO 2014/190273 such as 136.20.1; may have one or more (e.g., 2) variable regions of an antibody molecule of WO 2014/190273, or may comprise an antibody molecule of WO 2014/190273 such as 136.20.1. In other embodiments, the anti-CAR antibody binds to a constant region of the extracellular binding domain of the CAR molecule, e.g., as described in WO 2014/190273. In some embodiments, the anti-CAR antibody binds to a constant region of the extracellular binding domain of the CAR molecule, e.g., a heavy chain constant region (e.g., a CH2-CH3 hinge region) or light chain constant region. For instance, in some embodiments the anti-CAR antibody competes for binding with the 2D3 monoclonal antibody described in WO 2014/190273, has the same CDRs (e.g., one or more of, e.g., all of, VH CDR1, VH CDR2, CH CDR3, VL CDR1, VL CDR2, and VL CDR3) as 2D3, or has one or more (e.g., 2) variable regions of 2D3, or comprises 2D3 as described in WO 2014/190273.

In some aspects and embodiments, the compositions and methods herein are optimized for a specific subset of T cells, e.g., as described in U.S. Ser. No. 62/031,699 filed Jul. 31, 2014, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR described herein, which CAR comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR described herein, which CAR comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain that targets BCMA).

In an aspect, described herein is a method of treating a subject, e.g., a subject having cancer. The method includes administering to said subject, an effective amount of:

1) a CD4⁺ T cell comprising a CAR (the CAR^{CD4+})

comprising:

an antigen binding domain, e.g., an antigen binding domain described herein, e.g., an antigen binding domain that targets BCMA;

a transmembrane domain; and

an intracellular signaling domain, e.g., a first costimulatory domain, e.g., an ICOS domain; and

2) a CD8⁺ T cell comprising a CAR (the CAR^{CD8+}) comprising:

an antigen binding domain, e.g., an antigen binding domain described herein, e.g., an antigen binding domain that targets BCMA;

a transmembrane domain; and

an intracellular signaling domain, e.g., a second costimulatory domain, e.g., a 4-1BB domain, a CD28 domain, or another costimulatory domain other than an ICOS domain;

wherein the CAR^{CD4+} and the CAR^{CD8+} differ from one another.

Optionally, the method further includes administering:

3) a second CD8+ T cell comprising a CAR (the second CAR^{CD8+}) comprising:

an antigen binding domain, e.g., an antigen binding domain described herein, e.g., an antigen binding domain that specifically binds BCMA;

a transmembrane domain; and

an intracellular signaling domain, wherein the second CAR^{CD8+} comprises an intracellular signaling domain, e.g., a costimulatory signaling domain, not present on the CAR^{CD8+}, and, optionally, does not comprise an ICOS signaling domain.

Other assays, including those that are known in the art can also be used to evaluate the BCMA CAR constructs of the invention.

### Therapeutic Application

BCMA Associated Diseases and/or Disorders

In one aspect, the invention provides methods for treating a disease associated with BCMA expression. In one aspect, the invention provides methods for treating a disease wherein part of the tumor is negative for BCMA and part of the tumor is positive for BCMA For example, the CAR of the invention is useful for treating subjects that have undergone treatment for a disease associated with elevated expression of BCMA, wherein the subject that has undergone treatment for elevated levels of BCMA exhibits a disease associated with elevated levels of BCMA. In embodiments, the CAR of the invention is useful for treating subjects that have undergone treatment for a disease associated with expression of BCMA, wherein the subject that has undergone treatment related to expression of BCMA exhibits a disease associated with expression of BCMA.

In one embodiment, the invention provides methods for treating a disease wherein BCMA is expressed on both normal cells and cancers cells, but is expressed at lower levels on normal cells. In one embodiment, the method further comprises selecting a CAR that binds of the invention with an affinity that allows the BCMA CAR to bind and kill the cancer cells expressing BCMA but less than 30%, 25%, 20%, 15%, 10%, 5% or less of the normal cells expressing BCMA are killed, e.g., as determined by an assay described herein. For example, a killing assay such as flow cytometry based on Cr51 CTL can be used. In one embodiment, the BCMA CAR has an antigen binding domain that has a binding affinity KD of 10⁻⁴ M to 10⁻⁸ M, e.g., 10⁻⁵ M to 10 M, e.g., 10⁻⁶ M or 10 M, for the target antigen. In one embodiment, the BCMA antigen binding domain has a binding affinity that is at least five-fold, 10-fold, 20-fold, 30-fold, 50-fold, 100-fold or 1,000-fold less than a reference antibody, e.g., an antibody described herein.

In one aspect, the invention pertains to a vector comprising BCMA CAR operably linked to promoter for expression in mammalian immune effector cells, e.g., T cells or NK cells. In one aspect, the invention provides a recombinant immune effector cell, e.g., T cell or NK cell, expressing the BCMA CAR for use in treating BCMA-expressing tumors, wherein the recombinant immune effector cell (e.g., T cell or NK cell) expressing the BCMA CAR is termed a BCMA CAR-expressing cell (e.g., BCMA CART or BCMA CAR-expressing NK cell). In one aspect, the BCMA CAR-expressing cell (e.g., BCMA CART or BCMA CAR-expressing NK cell) of the invention is capable of contacting a tumor cell with at least one BCMA CAR of the invention expressed on its surface such that the BCMA CAR-expressing cell (e.g., BCMA CART or BCMA CAR-expressing NK cell)targets the tumor cell and growth of the tumor is inhibited.

In one aspect, the invention pertains to a method of inhibiting growth of a BCMA-expressing tumor cell, comprising contacting the tumor cell with a BCMA CAR-expressing cell (e.g., BCMA CART or BCMA CAR-expressing NK cell) of the present invention such that the BCMA CAR-expressing cell (e.g., BCMA CART or BCMA CAR-expressing NK cell) is activated in response to the antigen and targets the cancer cell, wherein the growth of the tumor is inhibited.

In one aspect, the invention pertains to a method of treating cancer in a subject. The method comprises administering to the subject a BCMA CAR-expressing cell (e.g., BCMA CART or BCMA CAR-expressing NK cell) of the present invention such that the cancer is treated in the subject. An example of a cancer that is treatable by the BCMA CAR-expressing cell (e.g., BCMA CART or BCMA CAR-expressing NK cell) of the invention is a cancer associated with expression of BCMA.

The invention includes a type of cellular therapy where immune effector cells (e.g., T cells or NK cells) are genetically modified to express a chimeric antigen receptor (CAR) and the BCMA CAR-expressing cell (e.g., BCMA CART or BCMA CAR-expressing NK cell) is infused to a recipient in need thereof. The infused cell is able to kill tumor cells in the recipient. Unlike antibody therapies, CAR-modified cells, e.g., T cells or NK cells, are able to replicate in vivo resulting in long-term persistence that can lead to sustained tumor control. In various aspects, the cells (e.g., T cells or NK cells) administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cell (e.g., T cell or NK cell) to the patient.

The invention also includes a type of cellular therapy where immune effector cells (e.g., T cells or NK cells) are modified, e.g., by in vitro transcribed RNA, to transiently express a chimeric antigen receptor (CAR) and the immune effector cell (e.g., T cell or NK cell) is infused to a recipient in need thereof. The infused cell is able to kill tumor cells in the recipient. Thus, in various aspects, the immune effector cells (e.g., T cells or NK cells) administered to the patient, is present for less than one month, e.g., three weeks, two weeks, one week, after administration of the immune effector cell (e.g., T cell or NK cell) to the patient.

Without wishing to be bound by any particular theory, the anti-tumor immunity response elicited by the CAR-modified immune effector cells (e.g., T cells or NK cells) may be an active or a passive immune response, or alternatively may be due to a direct vs indirect immune response. In one aspect, the CAR transduced immune effector cells (e.g., T cells or NK cells) exhibit specific proinflammatory cytokine secretion and potent cytolytic activity in response to human cancer cells expressing the BCMA, resist soluble BCMA inhibition, mediate bystander killing and mediate regression of an established human tumor. For example, antigen-less tumor cells within a heterogeneous field of BCMA-expressing tumor may be susceptible to indirect destruction by BCMA-redirected immune effector cells (e.g., T cells or NK cells) that has previously reacted against adjacent antigen-positive cancer cells.

In one aspect, the fully-human CAR-modified immune effector cells (e.g., T cells or NK cells) of the invention may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the CAR-modified immune effector cells (e.g., T cells or NK cells) of the invention are used in the treatment of diseases, disorders and conditions associated with expression of BCMA. In certain aspects, the cells of the invention are used in the treatment of patients at risk for developing diseases, disorders and conditions associated with expression of BCMA. Thus, the present invention provides methods for the treatment or prevention of diseases, disorders and conditions associated with expression of BCMA comprising administering to a subject in need thereof, a therapeutically effective amount of the CAR-modified immune effector cells (e.g., T cells or NK cells) of the invention.

In one aspect the CAR-expressing cells (e.g., CART cells or CAR-expressing NK cells) of the inventions may be used to treat a proliferative disease such as a cancer or malignancy or is a precancerous condition such as a myelodysplasia, a myelodysplastic syndrome or a preleukemia. In one aspect, the cancer is a hematolical cancer. Hematological cancer conditions are the types of cancer such as leukemia and malignant lymphoproliferative conditions that affect blood, bone marrow and the lymphatic system. In one aspect, the hematological cancer is a leukemia or a hematological. An example of a disease or disorder associated with BCMA is multiple myeloma (also known as MM) (See Claudio et al., Blood. 2002, 100(6):2175-86; and Novak et al., Blood. 2004, 103(2):689-94). Multiple myeloma, also known as plasma cell myeloma or Kahler's disease, is a cancer characterized by an accumulation of abnormal or malignant plasma B-cells in the bone marrow. Frequently, the cancer cells invade adjacent bone, destroying skeletal structures and resulting in bone pain and fractures. Most cases of myeloma also features the production of a paraprotein (also known as M proteins or myeloma proteins), which is an abnormal immunoglobulin produced in excess by the clonal proliferation of the malignant plasma cells. Blood serum paraprotein levels of more than 30 g/L is diagnostic of multiple myeloma, according to the diagnostic criteria of the International Myeloma Working Group (IMWG) (See Kyle et al. (2009), Leukemia. 23:3-9). Other symptoms or signs of multiple myeloma include reduced kidney function or renal failure, bone lesions, anemia, hypercalcemia, and neurological symptoms.

Criteria for distinguishing multiple myeloma from other plasma cell proliferative disorders have been established by the International Myeloma Working Group (See Kyle et al. (2009), Leukemia. 23:3-9). All three of the following criteria must be met:
Clonal bone marrow plasma cells ≥10%
Present of serum and/or urinary monoclonal protein (except in patients with true non-secretory multiple myeloma)
Evidence of end-organ damage attributable to the underlying plasma cell proliferative disorder, specifically:
   Hypercalcemia: serum calcium ≥11.5 mg/100 ml
   Renal insufficienty: serum creatinine >1.73 mmol/1
   Anemia: normochromic, normocytic with a hemoglobin value of ≥2 g/100 ml below the lower limit of normal, or a hemoglobin value <10 g/100 ml
   Bone lesions: lytic lesions, severe osteopenia, or pathologic fractures.

Other plasma cell proliferative disorders that can be treated by the compositions and methods described herein include, but are not limited to, asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

Two staging systems are used in the staging of multiple myeloma: the International Staging System (ISS) (See Greipp et al. (2005), J. Clin. Oncol. 23 (15):3412-3420, herein incorporated by reference in its entirety) and the Durie-Salmon Staging system (DSS) (See Durie et al. (1975), Cancer 36 (3): 842-854, herein incorporated by reference in its entirety). The two staging systems are summarized in the table below:

TABLE 4
Staging systems for the staging of multiple myeloma

| | International Staging System | | Dune-Salmon Staging System | |
|---|---|---|---|---|
| | | Median | | Median |
| Stage | Criteria | survival | Criteria | survival* |
| I | β₂M < 3.5 mg/l and serum | 62 months | All of the following: | IA: 62 |
| | albumin ≥ 3.5 g/dL | | Hemoglobin level > 10 g/dL | months |
| | | | Serum calcium, normal or | IB: 22 |
| | | | <12 mg/dL | months |
| | | | Bone x-ray, normal or 1 | |
| | | | plasmacytoma only | |
| | | | Low monoclonal protein production | |
| | | | (IgG < 5 g/dL, IgA < 3 g/dL, Bence | |
| | | | Jones protein < 4 g/dL per 24 hours | |
| II | Neither stage I or stage III | 44 months | Neither stage I or stage III | IIA: 58 |
| | | | | months |
| | | | | IIB: 354 |
| | | | | months |
| III | β₂M ≥ 5.5 mg/l | 29 months | One or more of the following: | IIIA: 45 |
| | | | Hemogloblin level < 8.5g/dL | months |
| | | | Serum calcium, normal or | IIIB: 24 |
| | | | >12 mg/dL | months |
| | | | Advanced osteolytic lesions | |
| | | | High monoclonal protein | |
| | | | production (IgG > 7g/dL, | |
| | | | IgA > 5g/dL, Bence Jones | |
| | | | protein > 12g/dL per 24 hours | |

*The Durie-Salmon Staging system also includes a subclassification that designates the status of renal function. The designation of ″A″ or ″B″ is added after the stage number, wherein ″A″ indicates relatively normal renal function (serum creatinine value <2.0 mg/dL), and B indicates abnormal renal function (serum creatinine value >2.0 mg/dL).

A third staging system for multiple myeloma is referred to as Revised International Staging System (R-ISS) (see Palumbo A, Avet-Loiseau H, Oliva S, et al. Journal of clinical oncology: official journal of the American Society of Clinical Oncology 2015; 33:2863-9, herein incorporated by reference in its entirety). R-ISS stage I includes ISS stage I (serum β2-microglobulin level <3.5 mg/L and serum albumin level ≥3.5 g/dL), no high-risk CA [del(17p) and/or t(4;14) and/or t(14;16)], and normal LDH level (less than the upper limit of normal range). R-ISS stage III includes ISS stage III (serum β2-microglobulin level >5.5 mg/L) and high-risk CA or high LDH level. R-ISS stage II includes all the other possible combinations.

The response of patients can be determined based on IMWG 2016 criteria, as disclosed in Kumar S, et al. International Myeloma Working Group consensus criteria for response and minimal residual disease assessment in multiple myeloma. The Lancet Oncology; 2016; 17(8):e328-e346, herein incorporated by reference in its entirety. Table 5 provides IMWG 2016 criteria for response assessment.

TABLE 5
IMWG criteria for response assessment including criteria for minimal residual disease (MRD)

Response criteria*

IMWG MRD criteria (requires a complete response as defined below)

| | |
|---|---|
| Sustained MRD-negative | MRD negativity in the marrow (NGF or NGS, or both) and by imaging as |
| | defined below, confirmed minimum of 1 year apart. Subsequent evaluations can |
| | be used to further specify the duration of negativity (e.g., MRD-negative at 5 |
| | years)† |
| Flow MRD-negative | Absence of phenotypically aberrant clonal plasma cells by NGF‡on bone |
| | marrow aspirates using the EuroFlow standard operation procedure for MRD |
| | detection in multiple myeloma (or validated equivalent method) with a minimum |
| | sensitivity of 1 in 10₅ nucleated cells or higher |
| Sequencing MRD-negative | Absence of clonal plasma cells by NGS on bone marrow aspirate in which |
| | presence of a clone is defined as less than two identical sequencing reads |
| | obtained after DNA sequencing of bone marrow aspirates using the |
| | LymphoSIGHT platform (or validated equivalent method) with a minimum |
| | sensitivity of 1 in 10₅ nucleated cells§ or higher |
| Imaging plus MRD- | MRD negativity as defined by NGF or NGS plus disappearance of every area of |
| negative | increased tracer uptake found at baseline or a preceding PET/CT or decrease to |
| | less mediastinal blood pool SUV or decrease to less than that of surrounding |
| | normal tissue¶ |

Standard IMWG response criteria||

| | |
|---|---|
| Stringent complete response | Complete response as defined below plus normal FLC ratio** and absence of |
| | clonal cells in bone marrow biopsy by immunohistochemistry (κ/λ ratio ≤4:1 or |
| | ≥1:2 for |
| | κ and λ patients, respectively, after counting ≥100 plasma cells)†† |
| Complete response | Negative immunofixation on the serum and urine and disappearance of any soft |
| | tissue plasmacytomas and <5% plasma cells in bone marrow aspirates |
| Very good partial response | Serum and urine M-protein detectable by immunofixation but not on |
| | electrophoresis or >90% reduction in serum M-protein plus urine M-protein |
| | level <100 mg per 24 h |
| Partial response | ≥50% reduction of serum M-protein plus reduction in 24 h urinary M-protein by |
| | ≥90% or to <200 mg per 24 h; |
| | If the serum and urine M-protein are unmeasurable, a ≥50% decrease in the |
| | difference between involved and uninvolved FLC levels is required in place of |
| | the M-protein criteria; |
| | If serum and urine M-protein are unmeasurable, and serum-free light assay is |
| | also unmeasurable, >50% reduction in plasma cells is required in place of M- |
| | protein, provided baseline bone marrow plasma-cell percentage was ≥30%. In |
| | addition to these criteria, if present at baseline, a >50% reduction in the size |
| | (SPD)§§ of soft tissue plasmacytomas is also required |
| Minimal response | ≥25% but ≤49% reduction of serum M-protein and reduction in 24-h urine M- |
| | protein by 50-89%. In addition to the above listed criteria, if present at baseline, |
| | a >50% reduction in the size (SPD)§§ of soft tissue plasmacytomas is also |
| | required |
| Stable disease | Not recommended for use as an indicator of response; stability of disease is best |
| | described by providing the time-to-progression estimates. Not meeting criteria |
| | for complete response, very good partial response, partial response, minimal |
| | response, or progressive disease |
| Progressive disease ¶¶, \|\|\|\| | Any one or more of the following criteria: |
| | Increase of 25% from lowest confirmed response value in one or more of the |
| | following criteria: |
| | Serum M-protein (absolute increase must be ≥0.5 g/dL); |
| | Serum M-protein increase ≥1 g/dL, if the lowest M component was ≥5 g/dL; |
| | Urine M-protein (absolute increase must be ≥200 mg/24 h); |
| | In patients without measurable serum and urine M-protein levels, the difference |
| | between involved and uninvolved FLC levels (absolute increase must be >10 |
| | mg/dL); |
| | In patients without measurable serum and urine M-protein levels and without |
| | measurable involved FLC levels, bone marrow plasma-cell percentage |
| | irrespective of baseline status (absolute increase must be >10%); |
| | Appearance of a new lesion(s), ≥50% increase from nadir in SPD§§ of >1 lesion, |
| | or ≥50% increase in the longest diameter of a previous lesion >1 cm in short |
| | axis; |
| | ≥50% increase in circulating plasma cells (minimum of 200 cells per μL) if this |
| | is the only measure of disease |
| Clinical relapse | Clinical relapse requires one or more of the following criteria: |
| | Direct indicators of increasing disease and/or end organ dysfunction (CRAB |
| | features) related to the underlying clonal plasma-cell proliferative disorder. It is |
| | not used in calculation of time to progression or progression-free survival but is |
| | listed as something that can be reported optionally or for use in clinical practice; |
| | Development of new soft tissue plasmacytomas or bone lesions (osteoporotic |
| | fractures do not constitute progression); |
| | Definite increase in the size of existing plasmacytomas or bone lesions. A |
| | definite increase is defined as a 50% (and ≥1 cm) increase as measured serially |
| | by the SPD§§ of the measurable lesion; |
| | Hypercalcaemia (>11 mg/dL); |
| | Decrease in haemoglobin of ≥2 g/dL not related to therapy or other non- |
| | myeloma-related conditions; |
| | Rise in serum creatinine by 2 mg/dL or more from the start of the therapy and |
| | attributable to myeloma; |
| | Hyperviscosity related to serum paraprotein |
| Relapse from complete | Any one or more of the following criteria: |
| response (to be used only if | Reappearance of serum or urine M-protein by immunofixation or |
| the end point is disease-free | electrophoresis; |
| survival) | Development of ≥5% plasma cells in the bone marrow; |
| | Appearance of any other sign of progression (i.e., new plasmacytoma, lytic bone |
| | lesion, or hypercalcaemia see above) |
| Relapse from MRD | Any one or more of the following criteria: |
| negative (to be used only if | Loss of MRD negative state (evidence of clonal plasma cells on NGF or NGS, or |
| the end point is disease-free | positive imaging study for recurrence of myeloma); |
| | survival) Reappearance of serum or urine M-protein by immunofixation or |
| | electrophoresis; |
| | Development of ≥5% clonal plasma cells in the bone marrow; |
| | Appearance of any other sign of progression (i.e., new plasmacytoma, lytic bone |
| | lesion, or hypercalcaemia) |

For MRD assessment, the first bone marrow aspirate should be sent to MRD (not for morphology) and this sample should be taken in one draw with a volume of minimally 2 mL (to obtain sufficient cells), but maximally 4-5 mL to avoid haemodilution.
IMWG = International Myeloma Working Group.
MRD = minimal residual disease.
NGF = next-generation flow.
NGS = next-generation sequencing.
FLC = free light chain.
M-protein = myeloma protein.
SPD = sum of the products of the maximal perpendicular diameters of measured lesions.
CRAB features = calcium elevation, renal failure, anaemia, lytic bone lesions.
FCM = flow cytometry.
SUVₘₐₓ = maximum standardised uptake value.
MFC = multiparameter flow cytometry.
¹^{⁸}F-FDG PET = ¹^{⁸}F-fluorodeoxyglucose PET.
ASCT = autologous stem cell transplantation.
*All response categories require two consecutive assessments made any time before starting any new therapy; for MRD there is no need for two consecutive assessments, but information on MRD after each treatment stage is recommended (eg, after induction, high-dose therapy/ASCT, consolidation, maintenance). MRD tests should be initiated only at the time of suspected complete response.
All categories of response and MRD require no known evidence of progressive or new bone lesions if radiographic studies were performed. However, radiographic studies are not required to satisfy these response requirements except for the requirement of FDG PET if imaging MRD-negative status is reported.
†Sustained MRD negativity when reported should also annotate the method used (eg, sustained flow MRD-negative, sustained sequencing MRD-negative).
‡Bone marrow MFC should follow NGF guidelines (Paiva B, Gutierrez NC, Rosinol L, et al, Blood 2012; 119: 687-91). The reference NGF method is an eight-colour two-tube approach, which has been extensively validated. The two-tube approach improves reliability, consistency, and sensitivity because of the acquisition of a greater number of cells. The eight-colour technology is widely available globally and the NGF method has already been adopted in many flow laboratories worldwide. The complete eight-colour method is most efficient using a lyophilised mixture of antibodies which reduces errors, time, and costs. 5 million cells should be assessed. The FCM method employed should have a sensitivity of detection of at least 1 in 10⁵ plasma cells.
§DNA sequencing assay on bone marrow aspirate should use a validated assay such as LymphoSIGHT (Sequenta).
¶Criteria used by Zamagni and colleagues (Zamagni E, Nanni C, Mancuso K, et al. Clin Cancer Res 2015; 21: 4384-90), and expert panel (IMPetUs; Italian Myeloma criteria for PET Use) (Usmani SZ, Mitchell A, Waheed S, et al. Blood 2013; 121: 1819-23; Nanni C, Zamagni E, Versari A, et al. Eur J Nucl Med Mol Imaging 2015; 43: 414-21.). Baseline positive lesions were identified by presence of focal areas of increased uptake within bones, with or without any underlying lesion identified by CT and present on at least two consecutive slices. Alternatively, an SUVₘₐₓ = 2.5 within osteolytic CT areas >1 cm in size, or SUVₘₐₓ = 1.5 within osteolytic CT areas ≤1 cm in size were considered positive. Imaging should be performed once MRD negativity is determined by MFC or NGS.
||Derived from international uniform response criteria for multiple myeloma (Dune BG, Harousseau JL, Miguel JS, et al, Leukemia 2006; 20: 1467-73). Minor response definition and clarifications derived from Rajkumar and colleagues (Rajkumar SV, Harousseau JL, Dune B, et al, Blood 2011; 117: 4691-95). When the only method to measure disease is by serum FLC levels: complete response can be defined as a normal FLC ratio of 0.26 to 1.65 in addition to the complete response criteria listed previously. Very good partial response in such patients requires a ≥90% decrease in the difference between involved and uninvolved FLC levels. All response categories require two consecutive assessments made at any time before the institution of any new therapy; all categories also require no known evidence of progressive or new bone lesions or extramedullary plasmacytomas if radiographic studies were performed. Radiographic studies are not required to satisfy these response requirements. Bone marrow assessments do not need to be confirmed. Each category, except for stable disease, will be considered unconfirmed until the confirmatory test is performed. The date of the initial test is considered as the date of response for evaluation of time dependent outcomes such as duration of response.
**All recommendations regarding clinical uses relating to serum FLC levels or FLC ratio are based on results obtained with the validated Freelite test (Binding Site, Birmingham, UK).
††Presence/absence of clonal cells on immunohistochemistry is based upon the κ/λ/L ratio. An abnormal κ/λ ratio by immunohistochemistry requires a minimum of 100 plasma cells for analysis. An abnormal ratio reflecting presence of an abnormal clone is κ/λ of >4:1 or <1:2.
‡‡Special attention should be given to the emergence of a different monoclonal protein following treatment, especially in the setting of patients having achieved a conventional complete response, often related to oligoclonal reconstitution of the immune system. These bands typically disappear over time and in some studies have been associated with a better outcome. Also, appearance of monoclonal IgG κ in patients receiving monoclonal antibodies should be diff erentiated from the therapeutic antibody.
§§Plasmacytoma measurements should be taken from the CT portion of the PET/CT, or MRI scans, or dedicated CT scans where applicable. For patients with only skin involvement, skin lesions should be measured with a ruler. Measurement of tumour size will be determined by the SPD.
¶¶Positive immunofixation alone in a patient previously classified as achieving a complete response will not be considered progression. For purposes of calculating time to progression and progression-free survival, patients who have achieved a complete response and are MRD-negative should be evaluated using criteria listed for progressive disease. Criteria for relapse from a complete response or relapse from MRD should be used only when calculating disease-free survival.
||||In the case where a value is felt to be a spurious result per physician discretion (eg, a possible laboratory error), that value will not be considered when determining the lowest value.

Standard treatment for multiple myeloma and associated diseases includes chemotherapy, stem cell transplant (autologous or allogeneic), radiation therapy, and other drug therapies. Frequently used anti-myeloma drugs include alkylating agents (e.g., bendamustine, cyclophosphamide and melphalan), proteasome inhibitors (e.g., bortezomib), corticosteroids (e.g., dexamethasone and prednisone), and immunomodulators (e.g., thalidomide and lenalidomide or Revlimid®), or any combination thereof. Biphosphonate drugs are also frequently administered in combination with the standard anti-MM treamtents to prevent bone loss. Patients older than 65-70 years of age are unlikely candidates for stem cell transplant. In some cases, double-autologous stem cell transplants are options for patients less than 60 years of age with suboptimal response to the first transplant. The compositions and methods of the present invention may be administered in combination with any of the currently prescribed treatments for multiple myeloma.

The first phase of treatment for multiple myeloma is induction therapy. The goal of induction therapy is to reduce the number of plasma cells in the bone marrow and the molecules (e.g., proteins) produced by the plasma cells. Induction therapy usually comprises a combination of 2 or 3 of the following types of drugs: targeted therapy, chemotherapy, or corticosteroids.

Induction therapy for patients who can have a stem cell transplant

Patients for a stem cell transplant are usually 70 years of age or younger and in generally good health. Patients can have induction therapy followed by high-dose chemotherapy and a stem cell transplant. Induction therapy is usually given for several cycles and may include one or more of the following drugs: CyBorD regimen—cyclophosphamide (Cytoxan, Procytox), bortezomib (Velcade) and dexamethasone (Decadron, Dexasone); VRD regimen—bortezomib, lenalidomide (Revlimid) and dexamethasone; thalidomide (Thalomid) and dexamethasone; lenalidomide and low-dose dexamethasone; bortezomib and dexamethasone; VTD regimen—bortezomib, thalidomide and dexamethasone; bortezomib, cyclophosphamide and prednisone; bortezomib, doxorubicin (Adriamycin) and dexamethasone; dexamethasone; or liposomal doxorubicin (Caelyx, Doxil), vincristine (Oncovin) and dexamethasone

Induction Therapy for Patients Who Cannot have a Stem Cell Transplant

Patients who cannot have a stem cell transplant may have induction therapy using one or more of the following drugs: CyBorD regimen—cyclophosphamide, bortezomib and dexamethasone; lenalidomide (Revlimid) and low-dose dexamethasone; MPT regimen—melphalan, prednisone and thalidomide; VMP regimen—bortezomib, melphalan and prednisone; MPL regimen—melphalan, prednisone and lenalidomide; melphalan and prednisone; bortezomib and dexamethasone; dexamethasone; liposomal doxorubicin, vincristine and dexamethasone; thalidomide and dexamethasone; VAD regimen—vincristine, doxorubicin and dexamethasone; or VRD regimen—bortezomib, lenalidomide and dexamethasone.

Another example of a disease or disorder associated with BCMA is Hodgkin's lymphoma and non-Hodgkin's lymphoma (See Chiu et al., Blood. 2007, 109(2):729-39; He et al., J Immunol. 2004, 172(5):3268-79).

Hodgkin's lymphoma (HL), also known as Hodgkin's disease, is a cancer of the lymphatic system that originates from white blood cells, or lymphocytes. The abnormal cells that comprise the lymphoma are called Reed-Sternberg cells. In Hodgkin's lymphoma, the cancer spreads from one lymph node group to another. Hodgkin's lymphoma can be subclassified into four pathologic subtypes based upon Reed-Sternberg cell morphology and the cell composition around the Reed-Sternberg cells (as determined through lymph node biopsy): nodular sclerosing HL, mixed-cellularity subtype, lymphocyte-rich or lymphocytic predominance, lymphocyte depleted. Some Hodgkin's lymphoma can also be nodular lymphocyte predominant Hodgkin's lymphoma, or can be unspecified. Symptoms and signs of Hodgkin's lymphoma include painless swelling in the lymph nodes in the neck, armpits, or groin, fever, night sweats, weight loss, fatigue, itching, or abdominal pain.

Non-Hodgkin's lymphoma (NHL) comprises a diverse group of blood cancers that include any kind of lymphoma other than Hodgkin's lymphoma. Subtypes of non-Hodgkin's lymphoma are classified primarily by cell morphology, chromosomal aberrations, and surface markers. NHL subtypes (or NHL-associated cancers) include B cell lymphomas such as, but not limited to, Burkitt's lymphoma, B-cell chronic lymphocytic leukemia (B-CLL), B-cell prolymphocytic leukemia (B-PLL), chronic lymphocytic leukemia (CLL), diffuse large B-cell lymphoma (DLBCL) (e.g., intravascular large B-cell lymphoma and primary mediastinal B-cell lymphoma), follicular lymphoma (e.g., follicle center lymphoma, follicular small cleaved cell), hair cell leukemia, high grade B-cell lymphoma (Burkitt's like), lymphoplasmacytic lymphoma (Waldenstrom's macroglublinemia), mantle cell lymphoma, marginal zone B-cell lymphomas (e.g., extranodal marginal zone B-cell lymphoma or mucosa-associated lymphoid tissue (MALT) lymphoma, nodal marginal zone B-cell lymphoma, and splenic marginal zone B-cell lymphoma), plasmacytoma/myeloma, precursor B-lymphoblastic leukemia/lymphoma (PB-LBL/L), primary central nervous system (CNS) lymphoma, primary intraocular lymphoma, small lymphocytic lymphoma (SLL); and T cell lymphomas, such as, but not limited to, anaplastic large cell lymphoma (ALCL), adult T-cell lymphoma/leukemia (e.g., smoldering, chronic, acute and lymphomatous), angiocentric lymphoma, angioimmunoblastic T-cell lymphoma, cutaneous T-cell lymphomas (e.g., mycosis fungoides, Sezary syndrome, etc.), extranodal natural killer/T-cell lymphoma (nasal-type), enteropathy type intestinal T-cell lymphoma, large granular lymphocyte leukemia, precursor T-lymphoblastic lymphoma/leukemia (T-LBL/L), T-cell chronic lymphocytic leukemia/prolymphocytic leukemia (T-CLL/PLL), and unspecified peripheral T-cell lymphoma. Symptoms and signs of Hodgkin's lymphoma include painless swelling in the lymph nodes in the neck, armpits, or groin, fever, night sweats, weight loss, fatigue, itching, abdominal pain, coughing, or chest pain.

The staging is the same for both Hodgkin's and non-Hodgkin's lymphoma, and refers to the extent of spread of the cancer cells within the body. In stage I, the lymphoma cells are in one lymph node group. In stage II, lymphoma cells are present in at least two lymph node groups, but both groups are on the same side of the diaphragm, or in one part of a tissue or organ and the lymph nodes near that organ on the same side of the diaphragm. In stage III, lymphoma cells are in lymph nodes on both sides of the diaphragm, or in one part of a tissue or organ near these lymph node groups or in the spleen. In stage IV, lymphoma cells are found in several parts of at least one organ or tissue, or lymphoma cells are in an organ and in lymph nodes on the other side of the diaphragm. In addition to the Roman numeral staging designation, the stages of can also be described by letters A, B, E, and S, wherein A refers to patients without symptoms, B refers to patients with symptoms, E refers to patients in which lymphoma is found in tissues outside the lymph system, and S refers to patients in which lymphoma is found in the spleen.

Hodgkin's lymphoma is commonly treated with radiation therapy, chemotherapy, or hematopoietic stem cell transplantation. The most common therapy for non-Hodgkin's lymphoma is R-CHOP, which consists of four different chemotherapies (cyclophosphamide, doxorubicin, vincristine, and prenisolone) and rituximab (Rituxan®). Other therapies commonly used to treat NHL include other chemotherapeutic agents, radiation therapy, stem cell transplantation (autologous or allogeneic bone marrow transplantation), or biological therapy, such as immunotherapy. Other examples of biological therapeutic agents include, but are not limited to, rituximab (Rituxan®), tositumomab (Bexxar®), epratuzumab (LymphoCide®), and alemtuzumab (MabCampath®). The compositions and methods of the present invention may be administered in combination with any of the currently prescribed treatments for Hodgkin's lymphoma or non-Hodgkin's lymphoma.

BCMA expression has also been associated Waldenstrom's macroglobulinemia (WM), also known as lymphoplasmacytic lymphoma (LPL). (See Elsawa et al., Blood. 2006, 107(7):2882-8). Waldenstrom's macroglobulinemia was previously considered to be related to multiple myeloma, but has more recently been classified as a subtype of non-Hodgkin's lymphoma. WM is characterized by uncontrolled B-cell lymphocyte proliferation, resulting in anemia and production of excess amounts of paraprotein, or immunoglobulin M (IgM), which thickens the blood and results in hyperviscosity syndrome. Other symptoms or signs of WM include fever, night sweats, fatigue, anemia, weight loss, lymphadenopathy or splenomegaly, blurred vision, dizziness, nose bleeds, bleeding gums, unusual bruises, renal impairment or failure, amyloidosis, or peripheral neuropathy.

Standard treatment for WM consists of chemotherapy, specifically with rituximab (Rituxan®). Other chemotherapeutic drugs can be used in combination, such as chlorambucil (Leukeran®), cyclophosphamide (Neosar®), fludarabine (Fludara®), cladribine (Leustatin®), vincristine, and/or thalidomide. Corticosteriods, such as prednisone, can also be administered in combination with the chemotherapy. Plasmapheresis, or plasma exchange, is commonly used throughout treatment of the patient to alleviate some symptoms by removing the paraprotein from the blood. In some cases, stem cell transplantation is an option for some patients.

Another example of a disease or disorder associated with BCMA is brain cancer. Specifically, expression of BCMA has been associated with astrocytoma or glioblastoma (See Deshayes et al, Oncogene. 2004, 23(17):3005-12, Pelekanou et al., PLoS One. 2013, 8(12):e83250). Astrocytomas are tumors that arise from astrocytes, which are a type of glial cell in the brain. Glioblastoma (also known as glioblastoma multiforme or GBM) is the most malignant form of astrocytoma, and is considered the most advanced stage of brain cancer (stage IV). There are two variants of glioblastoma: giant cell glioblastoma and gliosarcoma. Other astrocytomas include juvenile pilocytic astrocytoma (JPA), fibrillary astrocytoma, pleomorphic xantroastrocytoma (PXA), desembryoplastic neuroepithelial tumor (DNET), and anaplastic astrocytoma (AA).

Symptoms or signs associated with glioblastoma or astrocytoma include increased pressure in the brain, headaches, seizures, memory loss, changes in behavior, loss in movement or sensation on one side of the body, language dysfunction, cognitive impairments, visual impairment, nausea, vomiting, and weakness in the arms or legs.

Surgical removal of the tumor (or resection) is the standard treatment for removal of as much of the glioma as possible without damaging or with minimal damage to the normal, surrounding brain. Radiation therapy and/or chemotherapy are often used after surgery to suppress and slow recurrent disease from any remaining cancer cells or satellite lesions. Radiation therapy includes whole brain radiotherapy (conventional external beam radiation), targeted three-dimensional conformal radiotherapy, and targeted radionuclides. Chemotherapeutic agents commonly used to treat glioblastoma include temozolomide, gefitinib or erlotinib, and cisplatin. Angiogenesis inhibitors, such as Bevacizumab (Avastin®), are also commonly used in combination with chemotherapy and/or radiotherapy.

Supportive treatment is also frequently used to relieve neurological symptoms and improve neurologic function, and is administered in combination any of the cancer therapies described herein. The primary supportive agents include anticonvulsants and corticosteroids. Thus, the compositions and methods of the present invention may be used in combination with any of the standard or supportive treatments to treat a glioblastoma or astrocytoma.

Non-cancer related diseases and disorders associated with BCMA expression can also be treated by the compositions and methods disclosed herein. Examples of non-cancer related diseases and disorders associated with BCMA expression include, but are not limited to: viral infections; e.g., HIV, fungal invections, e.g., C. neoformans; irritable bowel disease; ulcerative colitis, and disorders related to mucosal immunity.

The CAR-modified immune effector cells (e.g., T cells or NK cells) of the present invention may be administered either alone, or as a pharmaceutical composition in combination with diluents and/or with other components such as IL-2 or other cytokines or cell populations.

The present invention provides for compositions and methods for treating cancer. In one aspect, the cancer is a hematologic cancer including but is not limited to hematolical cancer is a leukemia or a lymphoma. In one aspect, the CAR-expressing cells (e.g., CART cells or CAR-expressing NK cells) of the invention may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with BCMA expression includes, but not limited to, e.g., atypical and/or non-classical cancers, malignancies, precancerous conditions or proliferative diseases expressing BCMA.

In embodiments, a composition described herein can be used to treat a disease including but not limited to a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

In embodiments, a composition described herein can be used to treat a disease including but not limited to a cancer, e.g., a cancer described herein, e.g., a prostate cancer (e.g., castrate-resistant or therapy-resistant prostate cancer, or metastatic prostate cancer), pancreatic cancer, or lung cancer.

The present invention also provides methods for inhibiting the proliferation or reducing a BCMA-expressing cell population, the methods comprising contacting a population of cells comprising a BMCA-expressing cell with an anti-BCMA CAR-expressing cell (e.g., BCMA CART cell or BCMA CAR-expressing NK cell) of the invention that binds to the BCMA-expressing cell. In a specific aspect, the present invention provides methods for inhibiting the proliferation or reducing the population of cancer cells expressing BCMA, the methods comprising contacting the BCMA-expressing cancer cell population with an anti-BCMA CAR-expressing cell (e.g., BCMA CART cell or BCMA CAR-expressing NK cell) of the invention that binds to the BCMA-expressing cell. In one aspect, the present invention provides methods for inhibiting the proliferation or reducing the population of cancer cells expressing BCMA, the methods comprising contacting the BMCA-expressing cancer cell population with an anti-BCMA CAR-expressing cell (e.g., BCMA CART cell or BCMA CAR-expressing NK cell) of the invention that binds to the BCMA-expressing cell. In certain aspects, the anti-BCMA CAR-expressing cell (e.g., BCMA CART cell or BCMA CAR-expressing NK cell) of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with BCMA-expressing cells relative to a negative control. In one aspect, the subject is a human.

The present invention also provides methods for preventing, treating and/or managing a disease associated with BCMA-expressing cells (e.g., a hematologic cancer or atypical cancer expessing BCMA), the methods comprising administering to a subject in need an anti-BCMA CAR-expressing cell (e.g., BCMA CART cell or BCMA CAR-expressing NK cell) of the invention that binds to the BCMA-expressing cell. In one aspect, the subject is a human. Non-limiting examples of disorders associated with BCMA-expressing cells include viral or fungal infections, and disorders related to mucosal immunity.

The present invention also provides methods for preventing, treating and/or managing a disease associated with BCMA-expressing cells, the methods comprising administering to a subject in need an anti-BCMA CAR-expressing cell (e.g., BCMA CART cell or BCMA CAR-expressing NK cell) of the invention that binds to the BCMA-expressing cell. In one aspect, the subject is a human.

The present invention provides methods for preventing relapse of cancer associated with BCMA-expressing cells, the methods comprising administering to a subject in need thereof an anti-BCMA CAR-expressing cell (e.g., BCMA CART cell or BCMA CAR-expressing NK cell) of the invention that binds to the BCMA-expressing cell. In one aspect, the methods comprise administering to the subject in need thereof an effective amount of an anti-BCMA CAR-expressing cell (e.g., BCMA CART cell or BCMA CAR-expressing NK cell)described herein that binds to the BCMA-expressing cell in combination with an effective amount of another therapy.

### Methods Using Biomarkers for Evaluating CAR-Effectiveness, Subject Suitability, or Sample Suitability

In another aspect, the invention features a method of evaluating or monitoring the effectiveness of a CAR-expressing cell therapy (e.g., a BCMA CAR therapy), in a subject (e.g., a subject having a cancer, e.g., a hematological cancer), or the suitability of a sample (e.g., an apheresis sample) for a CAR therapy (e.g., a BCMA CAR therapy). The method includes acquiring a value of effectiveness to the CAR therapy, subject suitability, or sample suitability, wherein said value is indicative of the effectiveness or suitability of the CAR-expressing cell therapy.

In some embodiments of any of the methods disclosed herein, the CAR-expressing cell therapy comprises a plurality (e.g., a population) of CAR-expressing immune effector cells, e.g., a plurality (e.g., a population) of T cells or NK cells, or a combination thereof. In one embodiment, the CAR-expressing cell therapy is a BCMACAR therapy.

In some embodiments of any of the methods disclosed herein, the subject is evaluated prior to receiving, during, or after receiving, the CAR-expressing cell therapy.

In some embodiments of any of the methods disclosed herein, a responder (e.g., a complete responder) has, or is identified as having, a greater level or activity of one, two, or more (all) of GZMK, PPF1BP2, or naïve T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In an embodiment, a relapser is a patient having, or who is identified as having, an increased level of expression of one or more of (e.g., 2, 3, 4, or all of) the following genes, compared to non relapsers: MIR199A1, MIR1203, uc021ovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDMSD, USP9Y, PRKY, RPS4Y2, RPS4Y1, NCRNA00185, SULT1E1, and EIF1AY.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In one embodiment, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In one embodiment, a non-responder has, or is identified as having, a greater percentage of immune cells having an exhausted phenotype, e g, immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population (e.g., a BCMACAR+ cell population) compared to a responder (e.g., a complete responder) to the CAR-expressing cell therapy.

In some embodiments of any of the methods disclosed herein, a partial responder has, or is identified as having, a higher percentages of PD-1/PD-L1+/LAG-3+ cells, than a responder, in the CAR-expressing cell population (e.g., a BCMACAR+ cell population).

In some embodiments of any of the methods disclosed herein, a non-responder has, or is identified as having, an exhausted phenotype of PD1/PD-L1+ CAR+ and co-expression of LAG3 in the CAR-expressing cell population (e.g., a BCMACAR+ cell population).

In some embodiments of any of the methods disclosed herein, a non-responder has, or is identified as having, a greater percentage of PD-1/PD-L1+/TIM-3+ cells in the CAR-expressing cell population (e.g., a BCMACAR+ cell population) compared to the responder (e.g., a complete responder).

In some embodiments of any of the methods disclosed herein, a partial responders has, or is identified as having, a higher percentage of PD-1/PD-L1+/TIM-3+ cells, than responders, in the CAR-expressing cell population (e.g., a BCMACAR+ cell population).

In some embodiments of any of the methods disclosed herein, the presence of CD8+CD27+CD45RO− T cells in an apheresis sample is a positive predictor of the subject response to a CAR-expressing cell therapy (e.g., a BCMACAR therapy).

In some embodiments of any of the methods disclosed herein, a high percentage of PD1+ CAR+ and LAG3+ or TIM3+ T cells in an apheresis sample is a poor prognostic predictor of the subject response to a CAR-expressing cell therapy (e.g., a BCMACAR therapy).

In some embodiments of any of the methods disclosed herein, the responder (e.g., the complete or partial responder) has one, two, three or more (or all) of the following profile:

(i) has a greater number of CD27+ immune effector cells compared to a reference value, e.g., a non-responder number of CD27+ immune effector cells;

(ii) (i) has a greater number of CD8+ T cells compared to a reference value, e.g., a non-responder number of CD8+ T cells;

(iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or

(iv) has a greater number of one, two, three, four or more (all) of resting T_{EFF} cells, resting T_{REG} cells, naïve CD4 cells, unstimulated memory cells or early memory T cells, or a combination thereof, compared to a reference value, e.g., a non-responder number of resting T_{EFF} cells, resting T_{REG} cells, naïve CD4 cells, unstimulated memory cells or early memory T cells.

In some embodiments of any of the methods disclosed herein, the cytokine level or activity of (vi) is chosen from one, two, three, four, five, six, seven, eight, or more (or all) of cytokine CCL20/MIP3a, IL17A, IL6, GM-CSF, IFN-γ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFa. In one embodiment, an increased level or activity of a cytokine is chosen from one or both of IL-17a and CCL20, is indicative of increased responsiveness or decreased relapse.

In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, or Cheson criteria as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

Alternatively, or in combination with the methods disclosed herein, responsive to said value, performing one, two, three four or more of:

administering e.g., to a responder or a non-relapser, a CAR-expressing cell therapy;

administered an altered dosing of a CAR-expressing cell therapy;

altering the schedule or time course of a CAR-expressing cell therapy;

administering, e.g., to a non-responder or a partial responder, an additional agent in combination with a CAR-expressing cell therapy, e.g., a checkpoint inhibitor, e.g., a checkpoint inhibitor described herein;

administering to a non-responder or partial responder a therapy that increases the number of younger T cells in the subject prior to treatment with a CAR-expressing cell therapy;

modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder;

administering an alternative therapy, e.g., for a non-responder or partial responder or relapser; or

if the subject is, or is identified as, a non-responder or a relapser, decreasing the T_{REG} cell population and/or T_{REG} gene signature, e.g., by one or more of CD25 depletion, administration of cyclophosphamide, anti-GITR antibody, or a combination thereof.

In certain embodiments, the subject is pre-treated with an anti-GITR antibody. In certain embodiment, the subject is treated with an anti-GITR antibody prior to infusion or re-infusion.

### Combination Therapies

A CAR-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

### CD19 CAR

In some embodiments, the BCMA CAR-expressing cell therapy is administered in combination with a CD19 CAR-expressing cell therapy.

In one embodiment, the antigen binding domain of the CD19 CAR has the same or a similar binding specificity as the FMC63 scFv fragment described in Nicholson et al. Mol. Immun 34 (16-17): 1157-1165 (1997). In one embodiment, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (e.g., a humanized antigen binding domain) according to Table 3 of WO2014/153270, incorporated herein by reference. WO2014/153270 also describes methods of assaying the binding and efficacy of various CAR constructs.

In one aspect, the parental murine scFv sequence is the CAR19 construct provided in PCT publication WO2012/079000 (incorporated herein by reference). In one embodiment, the anti-CD19 binding domain is a scFv described in WO2012/079000.

In one embodiment, the CAR molecule comprises the fusion polypeptide sequence provided as SEQ ID NO: 12 in PCT publication WO2012/079000, which provides an scFv fragment of murine origin that specifically binds to human CD19.

In one embodiment, the CD19 CAR comprises an amino acid sequence provided as SEQ ID NO: 12 in PCT publication WO2012/079000. In embodiment, the amino acid sequence is

(MALPVTALLLPLALLLHAARP)diqmtqttsslsaslgdrvtiscrasqdiskylnwyqqkpdgtvklliyhtsrlhsg vpsrfsgsgsgtdysltisnleqediatyfcqqgntlpytfgggtkleitggggsggggsggggsevklqesgpglvapsqslsvtctvsgvslpdyg vswirqpprkglewlgviwgsettyynsalksrltiikdnsksqvflkmnslqtddtaiyycakhyyyggsyamdywgqgtsvtvsstapaprp ptpaptiasqplslrpeacrpaaggavhtrgldfacdiyiwaplagtcgvlllslvitlyckrgrkkllyiflcqpfmrpvqttqeedgcscrfpeeeegg celrvkfsrsadapaykqgqnqlynelnlgrreeydvldlargrdpemggkprrknpqeglynelqkdkmaeayseigmkgeragkghdgl yqglstatkdtydalhmqalppr (SEQ ID NO: 290), or a sequence substantially homologous thereto. The optional sequence of the signal peptide is shown in capital letters and parenthesis.

In one embodiment, the amino acid sequence is:

Diqmtqttsslsaslgdrvtiscrasqdiskylnwyqqkpdgtvklliyhtsrlhsgvpsrfsgsgsgtdysltisnleqediatyfcqqgn tlpytfgggtkleitggggsggggsggggsevklqesgpglvapsqslsvtctvsgvslpdygvswirqpprkglewlgviwgsettyynsalksr ltiikdnsksqvflkmnslqtddtaiyycakhyyyggsyamdywgqgtsvtvsstapaprpptpaptiasqplslrpeacrpaaggavhtrgldfa cdiyiwaplagtcgvlllslvitlyckrgrkkllyiflcqpfmrpvqttqeedgcscrfpeeeeggcelrvkfsrsadapaykqgqnqlynelnlgrre eydvldkagrdpemggkprrknpqeglynelqkdkmaeayseigmkgeragkghdglyqglstatkdtydalhmqalppr (SEQ ID NO: 291), or a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, CD19 CAR constructs are described in PCT publication WO 2012/079000, incorporated herein by reference, and the amino acid sequence of the murine CD19 CAR and scFv constructs are shown in Table 6 below, or a sequence substantially identical to any of the aforesaid sequences (e.g., at least 85%, 90%, 95% or more identical to any of the sequences described herein).

TABLE 6
CD19 CAR Constructs

| SEQ ID NO | Region | Sequence |
|---|---|---|
| CTL019 | | |
| SEQ ID NO: | CTL019 | MALPVTALLLPLALLLHAARPDIQMTQTTSSLSASLGDRVTISCRASQDIS |
| 292 | Full amino | KYLNWYQQKPDGTVKLLIYHTSRLHSGVPSRFSGSGSGTDYSLTISNLEQE |
| | acid | DIATYFCQQGNTLPYTFGGGTKLEITGGGGSGGGGSGGGGSEVKLQESGP |
| | sequence | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMD |
| | | YWGQGTSVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| SEQ ID NO: | CTL019 | ATGGCCTTACCAGTGACCGCCTTGCTCCTGCCGCTGGCCTTGCTGCTCC |
| 293 | Full | ACGCCGCCAGGCCGGACATCCAGATGACACAGACTACATCCTCCCTGT |
| | nucleotide | CTGCCTCTCTGGGAGACAGAGTCACCATCAGTTGCAGGGCAAGTCAGG |
| | sequence | ACATTAGTAAATATTTAAATTGGTATCAGCAGAAACCAGATGGAACTG |
| | | TTAAACTCCTGATCTACCATACATCAAGATTACACTCAGGAGTCCCATC |
| | | AAGGTTCAGTGGCAGTGGGTCTGGAACAGATTATTCTCTCACCATTAG |
| | | CAACCTGGAGCAAGAAGATATTGCCACTTACTTTTGCCAACAGGGTAA |
| | | TACGCTTCCGTACACGTTCGGAGGGGGGACCAAGCTGGAGATCACAGG |
| | | TGGCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGCCT |
| | | GTCCGTCACATGCACTGTCTCAGGGGTCTCATTACCCGACTATGGTGTA |
| | | AGCTGGATTCGCCAGCCTCCACGAAAGGGTCTGGAGTGGCTGGGAGTA |
| | | ATATGGGGTAGTGAAACCACATACTATAATTCAGCTCTCAAATCCAGA |
| | | CTGACCATCATCAAGGACAACTCCAAGAGCCAAGTTTTCTTAAAAATG |
| | | AACAGTCTGCAAACTGATGACACAGCCATTTACTACTGTGCCAAACAT |
| | | TATTACTACGGTGGTAGCTATGCTATGGACTACTGGGGCCAAGGAACC |
| | | TCAGTCACCGTCTCCTCAACCACGACGCCAGCGCCGCGACCACCAACA |
| | | CCGGCGCCCACCATCGCGTCGCAGCCCCTGTCCCTGCGCCCAGAGGCG |
| | | TGCCGGCCAGCGGCGGGGGGCGCAGTGCACACGAGGGGGCTGGACTT |
| | | CGCCTGTGATATCTACATCTGGGCGCCCTTGGCCGGGACTTGTGGGGT |
| | | CCTTCTCCTGTCACTGGTTATCACCCTTTACTGCAAACGGGGCAGAAAG |
| | | AAACTCCTGTATATATTCAAACAACCATTTATGAGACCAGTACAAACT |
| | | ACTCAAGAGGAAGATGGCTGTAGCTGCCGATTTCCAGAAGAAGAAGA |
| | | AGGAGGATGTGAACTGAGAGTGAAGTTCAGCAGGAGCGCAGACGCCC |
| | | CCGCGTACAAGCAGGGCCAGAACCAGCTCTATAACGAGCTCAATCTAG |
| | | GACGAAGAGAGGAGTACGATGTTTTGGACAAGAGACGTGGCCGGGAC |
| | | CCTGAGATGGGGGGAAAGCCGAGAAGGAAGAACCCTCAGGAAGGCCT |
| | | GTACAATGAACTGCAGAAAGATAAGATGGCGGAGGCCTACAGTGAGA |
| | | TTGGGATGAAAGGCGAGCGCCGGAGGGGCAAGGGGCACGATGGCCTT |
| | | TACCAGGGTCTCAGTACAGCCACCAAGGACACCTACGACGCCCTTCAC |
| | | ATGCAGGCCCTGCCCCCTCGC |
| SEQ ID NO: | CTL019 | DIQMTQTTSSLSASLGDRVTISCRASQDISKYLNWYQQKPDGTVKLLIYHT |
| 294 | scFv domain | SRLHSGVPSRFSGSGSGTDYSLTISNLEQEDIATYFCQQGNTLPYTFGGGTK |
| | | LEITGGGGSGGGGSGGGGSEVKLQESGPGLVAPSQSLSVTCTVSGVSLPD |
| | | YGVSWIRQPPRKGLEWLGVIWGSETTYYNSALKSRLTIIKDNSKSQVFLK |
| | | MNSLQTDDTAIYYCAKHYYYGGSYAMDYWGQGTSVTVSS |
| mCAR1 | | |
| SEQ ID NO: | mCAR1 | QVQLLESGAELVRPGSSVKISCKASGYAFSSYWMNWVKQRPGQGLEWIG |
| 295 | scFv | QIYPGDGDTNYNGKFKGQATLTADKSSSTAYMQLSGLTSEDSAVYSCAR |
| | | KTISSVVDFYFDYWGQGTTVTGGGSGGGSGGGSGGGSELVLTQSPKFMST |
| | | SVGDRVSVTCKASQNVGTNVAWYQQKPGQSPKPLIYSATYRNSGVPDRF |
| | | TGSGSGTDFTLTITNVQSKDLADYFCQYNRYPYTSFFFTKLEIKRRS |
| SEQ ID NO: | mCAR1 Full | QVQLLESGAELVRPGSSVKISCKASGYAFSSYWMNWVKQRPGQGLEWIG |
| 296 | amino acid | QIYPGDGDTNYNGKFKGQATLTADKSSSTAYMQLSGLTSEDSAVYSCAR |
| | sequence | KTISSVVDFYFDYWGQGTTVTGGGSGGGSGGGSGGGSELVLTQSPKFMST |
| | | SVGDRVSVTCKASQNVGTNVAWYQQKPGQSPKPLIYSATYRNSGVPDRF |
| | | TGSGSGTDFTLTITNVQSKDLADYFCQYNRYPYTSFFFTKLEIKRRSKIEV |
| | | MYPPPYLDNEKSNGTIIHVKGKHLCPSPLFPGPSKPFWVLVVVGGVLACY |
| | | SLLVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| mCAR2 | | |
| SEQ ID NO: | mCAR2 | DIQMTQTTSSLSASLGDRVTISCRASQDISKYLNWYQQKPDGTVKLLIYHT |
| 297 | scFv | SRLHSGVPSRFSGSGSGTDYSLTISNLEQEDIATYFCQQGNTLPYTFGGGTK |
| | | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSE |
| SEQ ID NO: | mCAR2 | DIQMTQTTSSLSASLGDRVTISCRASQDISKYLNWYQQKPDGTVKLLIYHT |
| 298 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSESKYGPPC |
| | | PPCPMFWVLVVVGGVLACYSLLVTVAFIIFWVKRGRKKLLYIFKQPFMRP |
| | | VQTTQEEDGCSCRFEEEEGGCELRVKFSRSADAPAYQQGQNQLYNELNL |
| | | GRREEYDVLDKRRGRDPEMGGKPRRKNPQEGLYNELQKDKMAEAYSEI |
| | | GMKGERRRGKGHDGLYQGLSTATKDTYDALHMQALPPRL |
| SEQ ID NO: | mCAR2 full | DIQMTQTTSSLSASLGDRVTISCRASQDISKYLNWYQQKPDGTVKLLIYHT |
| 299 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSESKYGPPC |
| | | PPCPMFWVLVVVGGVLACYSLLVTVAFIIFWVKRGRKKLLYIFKQPFMRP |
| | | VQTTQEEDGCSCRFEEEEGGCELRVKFSRSADAPAYQQGQNQLYNELNL |
| | | GRREEYDVLDKRRGRDPEMGGKPRRKNPQEGLYNELQKDKMAEAYSEI |
| | | GMKGERRRGKGHDGLYQGLSTATKDTYDALHMQALPPRLEGGGEGRGS |
| | | LLTCGDVEENPGPRMLLLVTSLLLCELPHPAFLLIPRKVCNGIGIGEFKDSL |
| | | SINATNIKHFKNCTSISGDLHILPV AFRGDSFTHTPPLDPQELDILKTVKEIT |
| | | GFLLIQAWPENRTDLHAFENLEIIRGRTKQHGQFSLAVVSLNITSLGLRSLK |
| | | EISDGDVIISGNKNLCYANTINWKKLFGTSGQKTKIISNRGENSCKATGQV |
| | | CHALCSPEGCWGPEPRDCVSCRNVSRGRECVDKCNLLEGEPREFVENSEC |
| | | IQCHPECLPQAMNITCTGRGPDNCIQCAHYIDGPHCVKTCPAGVMGENNT |
| | | LVWKYADAGHVCHLCHPNCTYGCTGPGLEGCPTNGPKIPSIATGMVGAL |
| | | LLLLVVALGIGLFM |
| mCAR3 | | |
| SEQ ID NO: | mCAR3 | DIQMTQTTSSLSASLGDRVTISCRASQDISKYLNWYQQKPDGTVKLLIYHT |
| 300 | scFv | SRLHSGVPSRFSGSGSGTDYSLTISNLEQEDIATYFCQQGNTLPYTFGGGTK |
| | | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSS |
| SEQ ID NO: | mCAR3 full | DIQMTQTTSSLSASLGDRVTISCRASQDISKYLNWYQQKPDGTVKLLIYHT |
| 301 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KAMNSLQTDDTAIYYCAKHYYYGGSYAMDYWGQGTSVTVSSAAAIEVMY |
| | | PPPYLDNEKSNGTIIHVKGKHLCPSPLFPGPSKPFWVLVVVGGVLACYSLL |
| | | VTVAFIIFWVRSKRSRLLHSDYMNMTPRRPGPTRKHYQPYAPPRDFAAYR |
| | | SRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLST |
| | | ATKDTYDALHMQALPPR |
| SSJ25-C1 | | |
| SEQ ID NO: | SSJ25-C1 | QVQLLESGAELVRPGSSVKISCKASGYAFSSYWMNWVKQRPGQGLEWIG |
| 302 | VH | QIYPGDGDTNYNGKFKGQATLTADKSSSTAYMQLSGLTSEDSAVYSCAR |
| | sequence | KTISSVVDFYFDYWGQGTTVT |
| SEQ ID NO: | SSJ25-C1 | ELVLTQSPKFMSTSVGDRVSVTCKASQNVGTNVAWYQQKPGQSPKPLIYS |
| 303 | VL | ATYRNSGVPDRFTGSGSGTDFTLTITNVQSKDLADYFYFCQYNRYPYTSG |
| | | GGTKLEIKRRS |
| Humanized | | |
| CAR1 | | |
| SEQ ID NO: | CAR1 scFv | EIVMTQSPATLSLSPGERATLSCRASQDISKYLNWYQQKPGQAPRLLIYHT |
| 304 | domain | SRLHSGIPARFSGSGSGTDYTLTISSLQPEDFAVYFCQQGNTLPYTFGQGTK |
| | | LEIKGGGGSGGGGSGGGGSQVQLQESGPGLVKPSETLSLTCTVSGVSLPD |
| | | YGVSWIRQPPGKGLEWIGVIWGSETTYYSSSLKSRVTISKDNSKNQVSLKL |
| | | SSVTAADTAVYYCAKHYYYGGSYAMDYWGQGTLVTVSS |
| SEQ ID NO: | CAR 1 - | MALPVTALLLPLALLLHAARPEIVMTQSPATLSLSPGERATLSCRASQDIS |
| 305 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | SSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Humanized | | |
| CAR2 | | |
| SEQ ID NO: | CAR2 scFv | EIVMTQSPATLSLSPGERATLSCRASQDISKYLNWYQQKPGQAPRLLIYHT |
| 306 | domain - aa | SRLHSGIPARFSGSGSGTDYTLTISSLQPEDFAVYFCQQGNTLPYTFGQGTK |
| | (Linker is | LEIKGGGGSGGGGSGGGGSQVQLQESGPGLVKPSETLSLTCTVSGVSLPD |
| | underlined) | YGVSWIRQPPGKGLEWIGVIWGSETTYYQSSLKSRVTISKDNSKNQVSLK |
| | | LSSVTAADTAVYYCAKHYYYGGSYAMDYWGQGTLVTVSS |
| SEQ ID NO: | CAR2 scFv | atggccctccctgtcaccgccctgctgcttccgctggctcttctgctcca |
| 307 | domain - nt | cgccgctcggcccgaaattgtgatgacccagtcacccgccactcttagcc |
| | | tttcacccggtgagcgcgcaaccctgtcttgcagagcctcccaagacatct |
| | | caaaataccttaattggtatcaacagaagcccggacaggctcctcgccttc |
| | | tgatctaccacaccagccggctccattctggaatccctgccaggttcagcg |
| | | gtagcggatctgggaccgactacaccctcactatcagctcactgcagccag |
| | | aggacttcgctgtctatttctgtcagcaagggaacaccctgccctacacct |
| | | ttggacagggcaccaagctcgagattaaaggtggaggtggcagcggaggag |
| | | gtgggtccggcggtggaggaagccaggtccaactccaagaaagcggaccgg |
| | | gtcttgtgaagccatcagaaactctttcactgacttgtactgtgagcggag |
| | | tgtctctccccgattacggggtgtcttggatcagacagccaccggggaagg |
| | | gtctggaatggattggagtgatttggggctctgagactacttactaccaat |
| | | catccctcaagtcacgcgtcaccatctcaaaggacaactctaagaatcagg |
| | | tgtcactgaaactgtcatctgtgaccgcagccgacaccgccgtgtactatt |
| | | gcgctaagcattactattatggcgggagctacgcaatggattactggggac |
| | | agggtactctggtcaccgtgtccagccaccaccatcatcaccatcaccat |
| SEQ ID NO: | CAR 2 - | MALPVTALLLPLALLLHAARPEIVMTQSPATLSLSPGERATLSCRASQDIS |
| 308 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | QSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| SEQ ID NO: | CAR 2 - | atggccctccctgtcaccgccctgctgcttccgctggctcttctgctccac |
| 309 | Full - nt | gccgctcggcccgaaattgtgatgacccagtcacccgccactcttagcctt |
| | | tcacccggtgagcgcgcaaccctgtcttgcagagcctcccaagacatctca |
| | | aaataccttaattggtatcaacagaagcccggacaggctcctcgccttctg |
| | | atctaccacaccagccggctccattctggaatccctgccaggttcagcggt |
| | | agcggatctgggaccgactacaccctcactatcagctcactgcagccagag |
| | | gacttcgctgtctatttctgtcagcaagggaacaccctgccctacaccttt |
| | | ggacagggcaccaagctcgagattaaaggtggaggtggcagcggaggaggt |
| | | gggtccggcggtggaggaagccaggtccaactccaagaaagcggaccgggt |
| | | cttgtgaagccatcagaaactctttcactgacttgtactgtgagcggagtg |
| | | tctctccccgattacggggtgtcttggatcagacagccaccggggaagggt |
| | | ctggaatggattggagtgatttggggctctgagactacttactaccaatca |
| | | tccctcaagtcacgcgtcaccatctcaaaggacaactctaagaatcaggtg |
| | | tcactgaaactgtcatctgtgaccgcagccgacaccgccgtgtactattgc |
| | | gctaagcattactattatggcgggagctacgcaatggattactggggacag |
| | | ggtactctggtcaccgtgtccagcaccactaccccagcaccgaggccaccc |
| | | accccggctcctaccatcgcctcccagcctctgtccctgcgtccggaggca |
| | | tgtagacccgcagctggtggggccgtgcatacccggggtcttgacttcgcc |
| | | tgcgatatctacatttgggcccctctggctggtacttgcggggtcctgctg |
| | | ctttcactcgtgatcactctttactgtaagcgcggtcggaagaagctgctg |
| | | tacatctttaagcaacccttcatgaggcctgtgcagactactcaagaggag |
| | | gacggctgttcatgccggttcccagaggaggaggaaggcggctgcgaactg |
| | | cgcgtgaaattcagccgcagcgcagatgctccagcctacaagcaggggcag |
| | | aaccagctctacaacgaactcaatcttggtcggagagaggagtacgacgtg |
| | | ctggacaagcggagaggacgggacccagaaatgggcgggaagccgcgcaga |
| | | aagaatccccaagagggcctgtacaacgagctccaaaaggataagatggca |
| | | gaagcctatagcgagattggtatgaaaggggaacgcagaagaggcaaaggc |
| | | cacgacggactgtaccagggactcagcaccgccaccaaggacacctatgac |
| | | gctcttcacatgcaggccctgccgcctcgg |
| SEQ ID NO: | CAR2 - | MALPVTALLLPLALLLHAARPeivmtqspat1s1spgeratlscrasqdis |
| 310 | Soluble | kylnwyqqkpgqaprlliyhtsrlhsgiparfsgsgsgtdytltisslqpe |
| | scFv - aa | dfavyfcqqgntlpytfgqgtkleikggggsggggsggggsqvqlqesgpg |
| | | lvkpsetlsltctvsgvslpdygvswirqppgkglewigviwgsettyyqs |
| | | slksrvtiskdnsknqvslklssvtaadtavyycakhyyyggsyamdywgq |
| | | gtivtvsshhhhhhhh |
| Humanized | | |
| CAR3 | | |
| SEQ ID NO: | CAR3 scFv | QVQLQESGPGLVKPSETLSLTCTVSGVSLPDYGVSWIRQPPGKGLEWIGVI |
| 311 | domain | WGSETTYYSSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR - | MALPVTALLLPLALLLHAARPQVQLQESGPGLVKPSETLSLTCTVSGVSLP |
| 312 | Full - aa | DYGVSWIRQPPGKGLEWIGVIWGSETTYYSSSLKSRVTISKDNSKNQVSLK |
| | | LSSVTAADTAVYYCAKHYYYGGSYAMDYWGQGTLVTVSSGGGGSGGG |
| | | GSGGGGSEIVMTQSPATLSLSPGERATLSCRASQDISKYLNWYQQKPGQA |
| | | PRLLIYHTSRLHSGIPARFSGSGSGTDYTLTISSLQPEDFAVYFCQQGNTLP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKG |
| | | ERRRGKGHDGLYQGLSTATKDTYDALHMQALPPR |
| Humanized | | |
| CAR4 | | |
| SEQ ID NO: | CAR4 scFv | QVQLQESGPGLVKPSETLSLTCTVSGVSLPDYGVSWIRQPPGKGLEWIGVI |
| 313 | domain | WGSETTYYQSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR - | MALPVTALLLPLALLLHAARPQVQLQESGPGLVKPSETLSLTCTVSGVSLP |
| 314 | Full - aa | DYGVSWIRQPPGKGLEWIGVIWGSETTYYQSSLKSRVTISKDNSKNQVSL |
| | | KLSSVTAADTAVYYCAKHYYYGGSYAMDYWGQGTLVTVSSGGGGSGG |
| | | GGSGGGGSEIVMTQSPATLSLSPGERATLSCRASQDISKYLNWYQQKPGQ |
| | | APRLLIYHTSRLHSGIPARFSGSGSGTDYTLTISSLQPEDFAVYFCQQGNTL |
| | | PYTFGQGTKLEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Humanized | | |
| CAR5 | | |
| SEQ ID NO: | CAR5 scFv | EIVMTQSPATLSLSPGERATLSCRASQDISKYLNWYQQKPGQAPRLLIYHT |
| 315 | domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYSSSLKSRVTISKDNSKNQ |
| | | VSLKLSSVTAADTAVYYCAKHYYYGGSYAMDYWGQGTLVTVSS |
| SEQ ID NO: | CAR - | MALPVTALLLPLALLLHAARPEIVMTQSPATLSLSPGERATLSCRASQDIS |
| 316 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYSSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR6 | | |
| SEQ ID NO: | CAR6 | EIVMTQSPATLSLSPGERATLSCRASQDISKYLNWYQQKPGQAPRLLIYHT |
| 317 | scFv domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYQSSLKSRVTISKDNSKNQ |
| | | VSLKLSSVTAADTAVYYCAKHYYYGGSYAMDYWGQGTLVTVSS |
| SEQ ID NO: | CAR6 - | MALPVTALLLPLALLLHAARPEIVMTQSPATLSLSPGERATLSCRASQDIS |
| 318 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYQSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR7 | | |
| SEQ ID NO: | CAR7 scFv | QVQLQESGPGLVKPSETLSLTCTVSGVSLPDYGVSWIRQPPGKGLEWIGVI |
| 319 | domain | WGSETTYYSSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 7 | MALPVTALLLPLALLLHAARPQVQLQESGPGLVKPSETLSLTCTVSGVSLP |
| 320 | Full - aa | DYGVSWIRQPPGKGLEWIGVIWGSETTYYSSSLKSRVTISKDNSKNQVSLK |
| | | LSSVTAADTAVYYCAKHYYYGGSYAMDYWGQGTLVTVSSGGGGSGGG |
| | | GSGGGGSGGGGSEIVMTQSPATLSLSPGERATLSCRASQDISKYLNWYQQ |
| | | KPGQAPRLLIYHTSRLHSGIPARFSGSGSGTDYTLTISSLQPEDFAVYFCQQ |
| | | GNTLPYTFGQGTKLEIKTTTPAPRPPTPAPTIASQPLSLRPEACRPAAGGAV |
| | | HTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPR |
| Humanized | | |
| CAR8 | | |
| SEQ ID NO: | CAR8 scFv | QVQLQESGPGLVKPSETLSLTCTVSGVSLPDYGVSWIRQPPGKGLEWIGVI |
| 321 | domain | WGSETTYYQSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 8 - | MALPVTALLLPLALLLHAARPQVQLQESGPGLVKPSETLSLTCTVSGVSLP |
| 322 | Full - aa | DYGVSWIRQPPGKGLEWIGVIWGSETTYYQSSLKSRVTISKDNSKNQVSL |
| | | KLSSVTAADTAVYYCAKHYYYGGSYAMDYWGQGTLVTVSSGGGGSGG |
| | | GGSGGGGSGGGGSEIVMTQSPATLSLSPGERATLSCRASQDISKYLNWYQ |
| | | QKPGQAPRLLIYHTSRLHSGIPARFSGSGSGTDYTLTISSLQPEDFAVYFCQ |
| | | QGNTLPYTFGQGTKLEIKTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR9 | | |
| SEQ ID NO: | CAR9 scFv | EIVMTQSPATLSLSPGERATLSCRASQDISKYLNWYQQKPGQAPRLLIYHT |
| 323 | domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYNSSLKSRVTISKDNSKNQ |
| | | VSLKLSSVTAADTAVYYCAKHYYYGGSYAMDYWGQGTLVTVSS |
| SEQ ID NO: | CAR 9 - | MALPVTALLLPLALLLHAARPEIVMTQSPATLSLSPGERATLSCRASQDIS |
| 324 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYNSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR10 | | |
| SEQ ID NO: | CAR10 scFv | QVQLQESGPGLVKPSETLSLTCTVSGVSLPDYGVSWIRQPPGKGLEWIGVI |
| 325 | domain | WGSETTYYNSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 10 | MALPVTALLLPLALLLHAARPEIVMTQSPATLSLSPGERATLSCRASQDIS |
| 326 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYNSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR11 | | |
| SEQ ID NO: | CAR11 | EIVMTQSPATLSLSPGERATLSCRASQDISKYLNWYQQKPGQAPRLLIYHT |
| 327 | scFv domain | SRLHSGIPARFSGSGSGTDYTLTISSLQPEDFAVYFCQQGNTLPYTFGQGTK |
| | | LEIKGGGGSGGGGSGGGGSQVQLQESGPGLVKPSETLSLTCTVSGVSLPD |
| | | YGVSWIRQPPGKGLEWIGVIWGSETTYYNSSLKSRVTISKDNSKNQVSLK |
| | | LSSVTAADTAVYYCAKHYYYGGSYAMDYWGQGTLVTVSS |
| SEQ ID NO: | CAR 11 | MALPVTALLLPLALLLHAARPQVQLQESGPGLVKPSETLSLTCTVSGVSLP |
| 328 | Full - aa | DYGVSWIRQPPGKGLEWIGVIWGSETTYYNSSLKSRVTISKDNSKNQVSL |
| | | KLSSVTAADTAVYYCAKHYYYGGSYAMDYWGQGTLVTVSSGGGGSGG |
| | | GGSGGGGSGGGGSEIVMTQSPATLSLSPGERATLSCRASQDISKYLNWYQ |
| | | QKPGQAPRLLIYHTSRLHSGIPARFSGSGSGTDYTLTISSLQPEDFAVYFCQ |
| | | QGNTLPYTFGQGTKLEIKTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR12 | | |
| SEQ ID NO: | CAR12 | QVQLQESGPGLVKPSETLSLTCTVSGVSLPDYGVSWIRQPPGKGLEWIGVI |
| 329 | scFv domain | WGSETTYYNSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR 12 - | MALPVTALLLPLALLLHAARPEIVMTQSPATLSLSPGERATLSCRASQDIS |
| 330 | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | NSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Murine | | |
| CART19 | | |
| SEQ ID NO: | HCDR1 | DYGVS |
| 331 | (Kabat) | |
| SEQ ID NO: | HCDR2 | VIWGSETTYYNSALKS |
| 332 | (Kabat) | |
| SEQ ID NO: | HCDR3 | HYYYGGSYAMDY |
| 333 | (Kabat) | |
| SEQ ID NO: | LCDR1 | RASQDISKYLN |
| 334 | (Kabat) | |
| SEQ ID NO: | LCDR2 | HTSRLHS |
| 335 | (Kabat) | |
| SEQ ID NO: | LCDR3 | QQGNTLPYT |
| 336 | (Kabat) | |
| Humanized | | |
| CART19 a | | |
| SEQ ID NO: | HCDR1 | DYGVS |
| 337 | (Kabat) | |
| SEQ ID NO: | HCDR2 | VIWGSETTYYSSSLKS |
| 338 | (Kabat) | |
| SEQ ID NO: | HCDR3 | HYYYGGSYAMDY |
| 339 | (Kabat) | |
| SEQ ID NO: | LCDR1 | RASQDISKYLN |
| 340 | (Kabat) | |
| SEQ ID NO: | LCDR2 | HTSRLHS |
| 341 | (Kabat) | |
| SEQ ID NO: | LCDR3 | QQGNTLPYT |
| 342 | (Kabat) | |
| Humanized | | |
| CART19 b | | |
| SEQ ID NO: | HCDR1 | DYGVS |
| 343 | (Kabat) | |
| SEQ ID NO: | HCDR2 | VIWGSETTYYQSSLKS |
| 344 | (Kabat) | |
| SEQ ID NO: | HCDR3 | HYYYGGSYAMDY |
| 345 | (Kabat) | |
| SEQ ID NO: | LCDR1 | RASQDISKYLN |
| 346 | (Kabat) | |
| SEQ ID NO: | LCDR2 | HTSRLHS |
| 347 | (Kabat) | |
| SEQ ID NO: | LCDR3 | QQGNTLPYT |
| 348 | (Kabat) | |
| Humanized | | |
| CART19 c | | |
| SEQ ID NO: | HCDR1 | DYGVS |
| 349 | (Kabat) | |
| SEQ ID NO: | HCDR2 | VIWGSETTYYNSSLKS |
| 350 | (Kabat) | |
| SEQ ID NO: | HCDR3 | HYYYGGSYAMDY |
| 351 | (Kabat) | |
| SEQ ID NO: | LCDR1 | RASQDISKYLN |
| 352 | (Kabat) | |
| SEQ ID NO: | LCDR2 | HTSRLHS |
| 353 | (Kabat) | |
| SEQ ID NO: | LCDR3 | QQGNTLPYT |
| 354 | (Kabat) | |

CD19 CAR constructs containing humanized anti-CD19 scFv domains are described in PCT publication WO 2014/153270, incorporated herein by reference.

The sequences of murine and humanized CDR sequences of the anti-CD19 scFv domains are shown in Table 7 for the heavy chain variable domains and in Table 8 for the light chain variable domains. The SEQ ID NOs refer to those found in Table 6.

TABLE 7
Heavy Chain Variable Domain CDR (Kabat) SEQ ID NO′s of CD19 Antibodies

| Candidate | HCDR1 | HCDR2 | HCDR3 |
|---|---|---|---|
| murine_CART19 | SEQ ID NO: 331 | SEQ ID NO: 332 | SEQ ID NO: 333 |
| humanized_CART19 a | SEQ ID NO: 337 | SEQ ID NO: 338 | SEQ ID NO: 339 |
| humanized_CART19 b | SEQ ID NO: 343 | SEQ ID NO: 344 | SEQ ID NO: 345 |
| humanized_CART19 c | SEQ ID NO: 349 | SEQ ID NO: 350 | SEQ ID NO: 351 |

TABLE 8
Light Chain Variable Domain CDR (Kabat) SEQ ID NO′s of CD19 Antibodies

| Candidate | LCDR1 | LCDR2 | LCDR3 |
|---|---|---|---|
| murine_CART19 | SEQ ID NO: 334 | SEQ ID NO: 335 | SEQ ID NO: 336 |
| humanized_CART19 a | SEQ ID NO: 340 | SEQ ID NO: 341 | SEQ ID NO: 342 |
| humanized_CART19 b | SEQ ID NO: 346 | SEQ ID NO: 347 | SEQ ID NO: 348 |
| humanized_CART19 c | SEQ ID NO: 352 | SEQ ID NO: 353 | SEQ ID NO: 354 |

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### Chemotherapeutic Agents

In some embodiments, the BCMA CAR-expressing cell therapy is administered in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 355), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### Pharmaceutical Compositions and Treatments

Pharmaceutical compositions of the present invention may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10cc to 400cc. In certain aspects, T cells are activated from blood draws of 20cc, 30cc, 40cc, 50cc, 60cc, 70cc, 80cc, 90cc, or 100cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the CAR-expressing cell (e.g., T cell or NK cell) compositions of the present invention are administered by i.v. injection. The compositions of CAR-expressing cells (e.g., T cells or NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the invention may be introduced, thereby creating a CAR-expressing cell (e.g., CAR T cell or CAR-expressing NK cell) of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells (e.g., CAR T cells or NK cells) of the present invention. In an additional aspect, expanded cells are administered before or following surgery.

In embodiments, lymphodepletion is performed on a subject, e.g., prior to administering one or more cells that express a CAR described herein, e.g., a BCMA-binding CAR described herein. In embodiments, the lymphodepletion comprises administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the CAR is introduced into immune effector cells (e.g., T cells or NK cells), e.g., using in vitro transcription, and the subject (e.g., human) receives an initial administration of CAR immune effector cells (e.g., T cells or NK cells) of the invention, and one or more subsequent administrations of the CAR immune effector cells (e.g., T cells or NK cells) of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR immune effector cells (e.g., T cells or NK cells) of the invention are administered to the subject (e.g., human) per week, e.g., 2, 3, or 4 administrations of the CAR immune effector cells (e.g., T cells or NK cells) of the invention are administered per week. In one embodiment, the subject (e.g., human subject) receives more than one administration of the CAR immune effector cells (e.g., T cells or NK cells) per week (e.g., 2, 3 or 4 administrations per week) (also referred to herein as a cycle), followed by a week of no CAR immune effector cells (e.g., T cells or NK cells) administrations, and then one or more additional administration of the CAR immune effector cells (e.g., T cells or NK cells) (e.g., more than one administration of the CAR immune effector cells (e.g., T cells or NK cells) per week) is administered to the subject. In another embodiment, the subject (e.g., human subject) receives more than one cycle of CAR immune effector cells (e.g., T cells or NK cells), and the time between each cycle is less than 10, 9, 8, 7, 6, 5, 4, or 3 days. In one embodiment, the CAR immune effector cells (e.g., T cells or NK cells) are administered every other day for 3 administrations per week. In one embodiment, the CAR immune effector cells (e.g., T cells or NK cells) of the invention are administered for at least two, three, four, five, six, seven, eight or more weeks.

In one aspect, BCMA CAR-expressing cells (e.g., BCMA CARTs or BCMA CAR-expressing NK cells) are generated using lentiviral viral vectors, such as lentivirus. CAR-expressing cells (e.g., CARTs or CAR-expressing NK cells) generated that way will have stable CAR expression.

In one aspect, CAR-expressing cells, e.g., CARTs, are generated using a viral vector such as a gammaretroviral vector, e.g., a gammaretroviral vector described herein. CARTs generated using these vectors can have stable CAR expression.

In one aspect, CAR-expressing cells (e.g., CARTs or CAR-expressing NK cells) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., T cell or NK cell, by electroporation.

A potential issue that can arise in patients being treated using transiently expressing CAR-expressing cells (e.g., CARTs or CAR-expressing NK cells) (particularly with murine scFv bearing CAR-expressing cells (e.g., CARTs or CAR-expressing NK cells)) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., CART or CAR-expressing NK cell) infusion breaks should not last more than ten to fourteen days.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples specifically point out various aspects of the present invention, and are not to be construed as limiting in any way the remainder of the disclosure.

### Example 1: Phase 1 Study of CART-BCMA with or without huCART19 as Consolidation of Standard First or Second-Line Therapy for High-Risk Multiple Myeloma

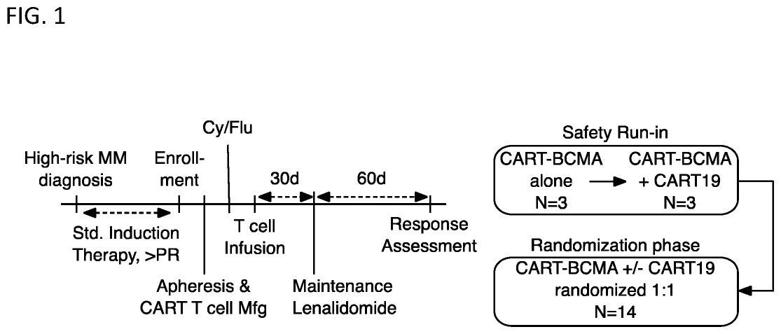

Experimental design: FIG. 1 depicts the overall design of the phase 1 study combining anti-CD19 CART cells and anti-BCMA CART cells as consolidation of first-line therapy in high-risk multiple myeloma (MM) patients. In this combination CAR T cell study, CART-BCMA will be co-administered with CART19 (also known as CTL119) after first-line therapy for high-risk MM; this study replaces a previously opened study administering CART19 alone after first-line therapy for MM that was closed early in anticipation of a CART-BCMA-based combination (NCT 02794246).

For this phase 1 study of CART-BCMA+CART19, the target population is patients with high-risk multiple myeloma defined as stage 3 in the Revised International Staging System (Palumbo A, Avet-Loiseau H, Oliva S, et al. Journal of clinical oncology: official journal of the American Society of Clinical Oncology 2015; 33:2863-9, herein incorporated by reference in its entirety) who are responding to first-line therapy. It is hypothesized that CAR T cells in this setting (compared to the relapsed/refractory setting) will be more effective because the T cells used for manufacturing will be less functionally compromised by high disease burden and many lines of prior MM therapy; it is also hypothesized that CAR T cells in this setting will be safer due to a lower disease burden, which is expected to lead to less pronounced initial CAR T cell proliferation in vivo. Compared to our phase 1 study, this study also adds to the pre-infusion conditioning regimen fludarabine, which has been used in most other CAR T cell studies and likely promotes prolonged survival of CAR T cells in vivo (Turtle C J, Hanafi L-A, Berger C, et al. Science translational medicine 2016; 8:355ra116-355ra116), and administers CART-BCMA as a single infusion rather than a split-dose infusion. Finally, subjects without excess toxicity at 30 days post-infusion will be administered standard-of-care lenalidomide maintenance therapy. Given these new aspects of the infusion regimen, the study will begin with a 3-patient safety run-in with CART-BCMA alone at a dose of 5×10⁸ CAR T cells, the established safe dose from our phase 1 study. If no excess toxicity is observed, an additional 3 patients will be enrolled receiving CART-BCMA and CART19, also at cell dose of 5×10⁸ cells. The CART19 dose on this study will be 10-fold higher than that used in our pilot CART19+ASCT study, where cell dose was low due to concerns about toxicity of CART19 with ASCT; this is designed to increase the likelihood of benefit from addition of CART19. If no excess toxicity is observed with the combination regimen, the study will proceed to a randomization phase where subjects will receive either CART-BCMA alone or CART-BCMA+CART19 until a total of 20 subjects have been treated, 10 each with the CART-BCMA monotherapy and CART-BCMA+CART19 combination therapy. The primary endpoint of the study is safety of this approach. Though it is hypothesized that addition of CART19 will improve progression-free survival, the study is not powered for this comparison. Rather, the randomized portion will allow comparison of correlative endpoints between the monotherapy and combined therapy arms to evaluate for evidence that CART19 eliminated de-differentiated BCMA^{dim} MM cells that resist CART-BCMA (to be analyzed by flow cytometry on bone marrow (BM) cells obtained post-treatment) and whether CART19 is targeting MM stem cells (MMSC).

Whether CART19 is targeting MMSC can be analyzed by (1) determining whether CART19 induces immune response (e.g., antibody response and/or T cell response) against the stem cell antigen Sox2; and/or (2) evaluating the expression of Sox2 as a clinical biomarker of MMSC in patient samples.

### Example 2: Phase 1 Study of CART-BCMA with or without huCART19 as Consolidation of Standard First or Second-Line Therapy for High-Risk Multiple Myeloma

### Study Summary

### Study Design

This is an open-label, phase 1 study to assess the safety and pharmacodynamics of autologous T cells expressing BCMA (B-cell maturation antigen)-specific chimeric antigen receptors with tandem TCRζ and 4-1BB (TCRζ/4-1BB) costimulatory domains (referred to as “CART-BCMA”), with or without huCART19 (also known as CTL119), as consolidation in patients responding to first- or second-line therapy for high-risk multiple myeloma.

The regimen evaluated in this study is based on established safety of CART-BCMA demonstrated in UPCC 14415/IRB #822756 at dose of 5×10⁸ cells, administered as split infusions, following cyclophosphamide 1.5 g/m² in patients with relapsed/refractory myeloma. This study tests CART-BCMA (1) as consolidation of early therapy for multiple myeloma, (2) as a single rather than split-dose infusion, (3) with addition of fludarabine to the lymphodepleting chemotherapy regimen, (4) in combination with huCART19, and (5) with planned re-infusion in subjects who progress or fail to achieve stringent complete response (Kumar S, Paiva B, Anderson K C, et al. International Myeloma Working Group consensus criteria for response and minimal residual disease assessment in multiple myeloma. The Lancet Oncology; 17(8):e328-e346).

Study enrollment will consist of two study phases: a). Safety Run-In Phase, and b).

Randomization Phase. Within each study phase, subjects will be enrolled into one of two cohorts:

1. Cohort 1: CART-BCMA monotherapy-administered as a single infusion of 5×10⁸ CART-BCMA cells, 3 days (+/−1 day) after cyclophosphamide+fludarabine chemotherapy 2. Cohort 2: CART-BCMA+huCART19-administered as a single infusion of 5×10⁸ CART-BCMA cells +a separate single infusion of 5×10⁸ huCART19 cells, 3 days (+/−1 day) after cyclophosphamide+fludarabine chemotherapy

### Randomization Phase

In the randomization phase, subjects will be randomized (1:1 ratio) to receive either CART-BCMA alone (Cohort 1) or CART-BCMA+huCART19 (Cohort 2). Following CAR T cell therapy, subjects will be eligible to receive standard-of-care maintenance therapy at the discretion of the treating investigator following the first formal response assessment at 28 days post-infusion or upon resolution to grade ≤2 of regimen-related toxicity, whichever is later.

Standard clinical assessments will be performed for toxicity and myeloma response. Correlative analyses will be conducted to assess pharmacokinetics/pharmacodynamics of CART-BCMA and huCART19 expansion/persistence and depletion of cells expressing target antigen, effects on clonogenic multiple myeloma subsets, and anti-myeloma immunity (including immunity against myeloma stem-cell antigens).

### Additional CAR T Cell Infusions

Subjects will be eligible for additional CAR T cell doses after the initial infusion if the following conditions are met:

1. Subject did not experience a dose-limiting toxicity (DLT) to any prior CAR T cell infusion.

2. Adverse events that are probably/definitely related to CAR T cells and/or lymphodepleting chemotherapy have recovered to baseline or grade ≤2.

3. Either 90 days have elapsed since prior CAR T cell infusion or subject's multiple myeloma has progressed after last CAR T cell infusion.

4. In the judgment of the investigator, subject has objective evidence of residual multiple myeloma. Determination of whether a subject has objective evidence of residual multiple myeloma will be made by the investigator.

5. In the judgment of the investigator, risks and benefits of additional CAR T cell infusion are balanced.

6. A minimum acceptable dose of CAR T cells remains from initial manufacturing.

7. Cohort 2 subjects only: >3% peripheral blood lymphocytes are CD19+. If <3% peripheral blood lymphocytes are CD19+ and the subject meets all other criteria above, the subject may receive an additional infusion of CART-BCMA alone at the investigator's discretion.

Subjects may only receive up to two additional CAR T cell infusions if the above criteria are satisfied and as long as the study remains open.

### Objectives

### Primary Objectives:

1) Evaluate the safety of CART-BCMA as a single dose of 5×10⁸ CAR+ cells as consolidation of standard first- or second-line multiple myeloma therapy following lymphodepletion with cyclophosphamide+fludarabine.

2) Evaluate the safety of huCART19 administered with CART-BCMA in this clinical setting. Secondary Objectives:

1) Assess clinical outcomes after each CAR T cell regimen (CART-BCMA monotherapy or combination CART-BCMA+huCART19).

a. Assess conventional response criteria, attainment of minimal residual disease (MRD)-negativity (according to IMWG 2016 criteria (Kumar S, et al. The Lancet Oncology; 2016; 17(8):e328-e346)), and attainment of PET-negative response (absence of detectable FDG-avid disease by PET/CT).

i. Response to first dose

ii. Response to subsequent doses

b. Duration of response, progression-free survival, and overall survival

c. Molecular MRD by igH sequencing

2) Evaluate CART-BCMA and huCART19 expansion and persistence kinetics in this clinical setting. Degree of expansion and persistence, and bioactivity after initial infusion will be compared to subsequent infusions.

3) Evaluate effects of huCART19 on correlative parameters of CART-BCMA resistance and clonogenic multiple myeloma cells, such as the following:

a. Persistence of clonal BCMA^{dim/neg} or CD19⁺ plasma cells as measured by flow cytometry and immunohistochemistry

b. Depletion of multiple myeloma clonogenicity as measured using in vitro colony formation assays on bone marrow samples

c. Induction of anti-Sox2 and other anti-myeloma immune responses

d. Depletion of clonal CD19+B cells

4) Evaluate cellular composition of apheresis product and CART-BCMA/huCART19 cells.

5) Evaluate effects of post-infusion maintenance therapy on CAR T cell persistence and phenotype, and CAR-related adverse events.

6) Characterize the phenotype (cell-surface immunophenotype, gene expression profile) of multiple myeloma cells that persist after CART-BCMA+/−huCART19.

### Diagnosis and Main Inclusion Criteria

Adult patients with high-risk multiple myeloma who have not achieved a stringent complete response after first- or second-line therapy.

### Investigational Agents, Dose, and Regimen

### Investigational Agent(s):

CART-BCMA Cells: Autologous T cells expressing BCMA (B-cell maturation antigen)-specific chimeric antigen receptors with tandem TCRζ and 4-1BB (TCRζ/4-1BB) costimulatory domains.
huCART19 Cells: Autologous T cells transduced with lentiviral vector to express anti-CD19 scFV TCrζ:4-1BB. Also known as CTL119 cells.
Cyclophosphamide/Fludarabine: Cytotoxic chemotherapy agents use for lymphodepletion prior to CAR T-cell product administration.

### Dose and Route of Administration:

CART-BCMA Cells: 5×10⁸ cells by intravenous infusion; Minimum acceptable dose for infusion is 1×10⁸.
huCART19 Cells: 5×10⁸ cells by intravenous infusion; Minimum acceptable dose for infusion is 1×10⁸.
Cyclophosphamide and Fludarabine: Cyclophosphamide 300 mg/m² and Fludarabine 30 mg/m² by intravenous infusion.

### Regimen:

CART-BCMA Cells: Single infusion on Day 0.
huCART19 Cells: Single infusion on Day 0 after completion of CART-BCMA infusion (in applicable subjects).
Cyclophosphamide/Fludarabine: Given over 3 days; Scheduled so that the last day of chemotherapy falls 3 days (+/−1 day) prior to the first CAR T-cell infusion (Day 0).

### Additional CAR T Cell Infusions:

Additional CAR T cell doses may be optionally infused at intervals of at least three months or upon disease progression for subjects that meet required eligibility criteria.

For second and subsequent CAR T cell infusions, the default regimen (CART-BCMA alone vs CART-BCMA+huCART19) will be the regimen the subject received with his/her initial infusion. However, CART-BCMA may be infused alone to a subject who previously received both CART-BCMA and huCART19 if insufficient huCART19 cells remain to formulate an acceptable dose, and/or <3% peripheral blood lymphocytes are CD19+. Cohort 2 subjects for whom no sufficient CART-BCMA dose remains, however, will not be eligible for additional infusions with huCART19 alone.

### INTRODUCTION

### Rationale for CAR T Cells as Consolidation Therapy for Multiple Myeloma

“Consolidation therapy” refers to treatment after response to prior therapy to prolong the response and/or reduce risk of relapse/progression. In multiple myeloma, standard first-line therapy with regimens such as lenalidomide, bortezomib, and dexamethasone is often consolidated with high-dose melphalan and autologous stem cell transplantation (ASCT). This study involves use of CAR T cell therapy rather than ASCT as consolidation of first-line therapy.

This study will enroll subjects who have responded to first- or second-line therapy and harvest T cells for CAR T cell manufacturing before extensive exposure to cytotoxic chemotherapy. It is expected that T cells harvested in this clinical setting will lead to a more clinically active CAR T cell product compared to cells harvested in the relapsed/refractory setting. This expectation is rooted in findings from CLL patients treated with CART19 showing that clinical outcome correlated strongly with presence of early memory T cell phenotypes (Fraietta J A, Lacey S F, Wilcox N S, et al. Biomarkers of Response to Anti-CD19 Chimeric Antigen Receptor (CAR) T-Cell Therapy in Patients with Chronic Lymphocytic Leukemia. Blood. 2016;128(22):57). Multiple myeloma progression is accompanied by development of exhausted T cell phenotypes (Chung D J, Pronschinske K B, Shyer J A, et al. T-cell Exhaustion in Multiple Myeloma Relapse after Autotransplant: Optimal Timing of Immunotherapy. Cancer Immunol Res. 2016; 4(1):61-71). As patients progress through multiple lines of therapy, the T cell repertoire likely becomes progressively impaired as disease burden increases and patients receive increasingly aggressive therapies, often with broadly cytotoxic mechanisms of action.

A challenge in evaluating efficacy of consolidation therapies is how to distinguish response to the investigational therapy from response to the preceding therapy. To avoid this limitation, this protocol restricts enrollment to subjects who have achieved at least a minor response but not a complete response to prior therapy despite having received at least three cycles, at which point responses typically “level-off” (i.e., fail to appreciably improve with further therapy). Moreover, subjects will defer standard consolidation with high-dose melphalan and ASCT to a later line of therapy and receive cyclophosphamide and fludarabine, which are not themselves expected to effect a significant anti-myeloma response in this population, as lymphodepleting chemotherapy. Therefore, with this study design, we expect to be able to attribute any multiple myeloma responses observed to clinical activity of the CAR T cells.

### Combination CAR T Cell Therapy

Selection and outgrowth of rare target-negative subsets of the neoplastic clone is a documented mechanism of resistance to CAR T cell therapy. Targeting two antigens simultaneously may circumvent this resistance mechanism and increase the likelihood of eliminating all immunophenotypic subsets of the clone with a single dose. In some subjects who received CART-BCMA, there was enrichment for BCMA-dim and CD19-expressing multiple myeloma cells after initial response, suggesting that co-administration of huCART19 may prevent progression after CART-BCMA.

### Serial CAR T Cell Infusions

Residual disease may persist after an initial CAR T cell infusion due to loss of in vivo functional capacity of infused CAR T cells before all disease is eradicated. Many subjects who initially responded to CART-BCMA later progressed despite low-level in vivo persistence of CART-BCMA cells. This finding suggests loss of CAR T cell potency after the initial wave of in vivo expansion. Since BCMA expression was still present at relapse in all patients analyzed, residual disease may be sensitive to re-infusion of additional CART-BCMA doses. This protocol therefore includes provision for serial infusions of CAR T cells as long as sufficient doses remain from the initial manufactured product. This provision is expected to generate safety, pharmacokinetic, and pharmacodynamic data, along with preliminary clinical response data, on serial CART-BCMA and huCART19 infusions in this clinical setting. This provision is also driven by the increasing recognition that very low levels of residual myeloma are clinically important and should likely be targeted. Even subjects in conventionally defined complete responses differ in their prognosis based on the persistence of minimal residual disease (MRD) (Munshi N C, Avet-Loiseau H, Rawstron A C, et al. Association of Minimal Residual Disease With Superior Survival Outcomes in Patients With Multiple Myeloma: A Meta-analysis. JAMA Oncol. 2017; 3(1):28-35).

### Subject Selection

### Inclusion Criteria

1. Subjects must have a diagnosis of multiple myeloma according to IMWG 2014 criteria (Rajkumar S V, Dimopoulos M A, Palumbo A, et al. International Myeloma Working Group updated criteria for the diagnosis of multiple myeloma. The lancet oncology. 2014; 15(12):e538-548) with any of the following high-risk features:
a. Beta-2-microglobulin ≥5.5 mg/L and LDH greater than upper limit of normal. Note:subjects in whom Beta-2-microglobulin were not measured prior to initiation of systemic therapy may qualify based on measurements obtained after initiation of systemic therapy.
b. High-risk FISH features: deletion 17p, t(14;16), t(14;20), t(4;14) in conjunction with Beta-2-microglobulin ≥5.5 mg/L (revised ISS stage 3). Note: subjects in whom Beta-2-microglobulin was not measured prior to initiation of systemic therapy may qualify based on measurements obtained after initiation of systemic therapy.
c. Metaphase karyotype with >3 structural abnormalities except hyperdiploidy.
d. Plasma cell leukemia (>20% plasma cells in peripheral blood) at any time prior to enrollment.
e. Failure to achieve partial response or better (by IMWG 2016 criteria (Kumar S, et al. The Lancet Oncology; 2016; 17(8):e328-e346)) to an “imid/PI” combination (thalidomide, lenalidomide, or pomalidomide in combination with bortezomib, ixazomib, or carfilzomib).
f. Progression (according to IMWG 2016 criteria (Kumar S, et al. The Lancet Oncology; 2016; 17(8):e328-e346)) on first-line therapy with an “imid/PI” combination within six months of starting therapy.

2. Subjects must meet the following criteria with respect to prior myeloma therapy:
a. Subjects must be in their first line of multiple myeloma therapy, with the following exception: subjects who have advanced to second-line therapy due to disease progression during first-line therapy are eligible if such progression occurred within six months of beginning first-line therapy. Lines of therapy are defined by IMWG 2016 criteria (Kumar S, et al. The Lancet Oncology; 2016; 17(8):e328-e346).
b. Subjects must not have undergone autologous or allogeneic stem cell transplantation.
c. Subjects must have initiated systemic therapy for multiple myeloma ≤1 year prior to enrollment.
d. Subjects must have received at least 3 complete cycles of their current regimen and have achieved at least a minimal response (as defined by IMWG 2016 criteria (Kumar S, et al. The Lancet Oncology; 2016; 17(8):e328-e346)) overall to prior therapy.
e. Subjects must not have received cytotoxic chemotherapy (e.g., doxorubicin, cyclophosphamide, etoposide, cisplatin) with the following exceptions:
   i. Low-dose weekly cyclophosphamide (≤500 mg/m²/week)
   ii. Continuous infusion cyclophosphamide, if limited to a single cycle.

3. Subjects must not have achieved a complete or stringent complete response according to IMWG 2016 criteria (Kumar S, et al. The Lancet Oncology; 2016; 17(8):e328-e346) at time of enrollment unless clonal plasma cells are detectable in bone marrow by flow cytometry. (I.e., subjects in complete or stringent complete response are eligible if minimal residual disease can be documented by bone marrow flow cytometry).

4. Subjects must have signed written, informed consent.

5. Subjects must be ≥18 years of age.

6. Subjects must have adequate vital organ function:
a. Serum creatinine ≤2.5 or creatinine clearance ≥30 ml/min (measured or estimated according to CKD-EPI) and not dialysis-dependent.
b. Absolute neutrophil count ≥1000/μl and platelet count ≥50,000/μl (≥30,000/μl if bone marrow plasma cells are ≥50% of cellularity).
c. SGOT ≤3× the upper limit of normal and total bilirubin ≤2.0 mg/dl (except for patients in whom hyperbilirubinemia is attributed to Gilbert's syndrome).
d. Left ventricular ejection fraction (LVEF) ≥45%. LVEF assessment must have been performed within 8 weeks of enrollment.

7. Toxicities from prior/ongoing therapies, with the exception of peripheral neuropathy attributable to multiple myeloma therapy, must have recovered to grade ≤2 according to the CTCAE 4.03 criteria or to the subject's prior baseline.

8. Subjects must have an ECOG performance status of 0-2.

9. Subjects must be willing to forego first-line ASCT.

10. Subjects of reproductive potential must agree to use acceptable birth control methods.

### Exclusion Criteria

1. Pregnant or lactating women

2. Inadequate venous access for or contraindications to leukapheresis.

3. Active hepatitis B, hepatitis C, or HIV infection, or other active, uncontrolled infection.

4. Any uncontrolled medical or psychiatric disorder that would preclude participation as outlined.

5. NYHA Class III or IV heart failure, unstable angina, or a history of recent (within 6 months) myocardial infarction or sustained (≥30 seconds) ventricular tachyarrhythmias.

6. Have active auto-immune disease, including connective tissue disease, uveitis, sarcoidosis, inflammatory bowel disease, or multiple sclerosis, or have a history of severe (as judged by the investigator) autoimmune disease requiring prolonged immunosuppressive therapy.

7. Have prior or active central nervous system (CNS) involvement (e.g. leptomeningeal disease, parenchymal masses) with myeloma. Screening for this (e.g. with lumbar puncture) is not required unless suspicious symptoms or radiographic findings are present. Subjects with calvarial disease that extends intracranially and involves the dura will be excluded, even if CSF is negative for myeloma.

### Study Drugs

### CART-BCMA Cells

CART-BCMA cells are autologous T cells that have been engineered to express an extracellular single chain antibody (scFv) with specificity for BCMA linked to an intracellular signaling molecule consisting of tandem signaling domains comprised of the TCRζ signaling module linked to the 4-1BB costimulatory domain. The CART-BCMA cells are cryopreserved in infusible cryomedia, and dispensed in an infusion bag. CART-BCMA cells will be administered as a single infusion. The target CART-BCMA dose will be 5×10⁸ transduced cells, with a minimum acceptable infusion dose of 1×10⁸ transduced cells. Doses will be formulated to achieve the maximum number of doses containing 5×10⁸ CAR T cells; i.e., doses will not be reduced below the target of 5×10⁸ CAR T cells for the purpose of increasing the quantity of available doses.

huCART19 Cells

huCART19 cells are autologous T cells that have been engineered to express an extracellular single chain antibody (scFv) with specificity for CD19 linked to an intracellular signaling molecule consisting of a tandem signaling domains comprised of the TCRζ signaling module linked to the 4-1BB costimulatory domain. The CTL119 cells are cryopreserved in infusible cryomedia, and dispensed in an infusion bag. huCART19 cells will be administered as a single infusion. The target huCART19 dose will be 5×10⁸ transduced cells, with a minimum acceptable infusion dose of 1×10⁸ transduced cells. Doses will be formulated to achieve the maximum number of doses containing 5×10⁸ CAR T cells; i.e., doses will not be reduced below the target of 5×10⁸ CAR T cells for the purpose of increasing the quantity of available doses.

CART-BCMA and huCART19 Infusion

The products will be administered in a sequential fashion, with the CART-BCMA cells thawed and infused first, followed by the huCART19 cell thaw and infusion, which must occur at least one hour after the completion of the CART-BCMA infusion. The huCART19 product must remain on dry ice during this time.

### Lymphodepleting Chemotherapy Before Initial CAR T Cell Infusion

Prior to the initial CAR T cell infusion(s), lymphodepleting chemotherapy will be administered as a regimen of cyclophosphamide 300 mg/m²+fludarabine 30 mg/m² daily for three days. Lymphodepleting chemotherapy must be scheduled so that the last day of therapy falls 3 days (+/−1 day) prior to the CAR T cell infusion.

While administered for research purposes as part of this study, both cyclophosphamide and fludarabine are FDA-approved agents and will be prepared and infused in accordance with their FDA-approved labels and standard institutional practice. The preferred anti-emetic pre-medication for this regimen is ondansetron 16 mg and dexamethasone 12 mg, each administered daily prior to each chemotherapy infusion; this regimen may be altered at the discretion of the treating investigator. Additional standard home anti-emetics may be prescribed by the investigator for as-needed use (e.g., ondansetron, prochlorperazine, lorazepam). Fludarabine dose may be reduced for subjects with estimated GFR <80 mL/min at investigator discretion.

### Lymphodepleting Chemotherapy Before Subsequent CAR T Cell Infusions

Prior to subsequent CAR T cell infusions, lymphodepleting chemotherapy will be administered as either the cyclophosphamide+fludarabine regimen (as described above), or a single infusion of cyclophosphamide 1.5 g/m². The choice between these two options for lymphodepleting chemotherapy will be at the discretion of the investigators guided by tolerance of lymphodepleting chemotherapy administered prior to first CAR T cell infusion and overall clinical condition. Lymphodepleting chemotherapy will be scheduled so that the last day of therapy falls 3 days (+/−1 day) prior to the CAR T cell infusion.

For cyclophosphamide 1.5 g/m², the preferred anti-emetic pre-medication is ondansetron 24 mg and dexamethasone 12 mg, followed by ondansetron 8 mg twice daily on the two days following cyclophosphamide. Subjects receiving cyclophosphamide 1.5 g/m² will also receive intravenous pre-hydration with 1L normal saline.

### Study Procedures

The study consists of (1) a screening phase, (2) a manufacturing phase consisting of apheresis and preparation of the CAR T cell product(s), (3) a treatment phase consisting of lymphodepleting chemotherapy and infusion of CAR T cells, and (4) follow-up.

### Pre-Entry Evaluations/Screening

During the pre-entry evaluation period, subjects will be asked to provide informed consent and a bone marrow aspirate and core biopsy for research correlative studies. This biopsy may be performed while the subject is still receiving first-line therapy. Samples may also be sent for standard anatomic pathology or genetic analyses (FISH, cytogenetics, next-generation sequencing, etc) at investigator discretion.

### Apheresis

A large volume apheresis procedure is carried out at the Hospital of the University of Pennsylvania apheresis center. PBMC are obtained for CAR T cells during this procedure.

### Autologous Stem Cell Mobilization and Collection

Subjects for whom high-dose melphalan and autologous stem cell transplantation would be considered in a future line of therapy may undergo autologous stem cell mobilization and collection after enrollment but before CAR T cell infusion as long as restrictions on pre-apheresis chemotherapy and myeloid growth factor usage are respected. It is anticipated (but not required) that autologous stem cell mobilization and collection would occur after leukapheresis for CAR T cell manufacturing.

### Pre-Infusion Evaluation

Subjects will undergo evaluations within 7 days prior to CAR T cell infusion and prior to lymphodepleting chemotherapy, to obtain pre-infusion baseline clinical and myeloma status and assess eligibility to proceed with CAR T cell infusion.

### Lymphodepleting Chemotherapy

Lymphodepleting chemotherapy will be scheduled so that the last day of lymphodepleting chemotherapy falls 3 days+/−1 day prior to CAR T cell infusion.

### CAR T Cell Infusion

CAR T cell infusion will begin 3 days (+/−1 day) after completion of chemotherapy. Each CAR T cell product (CART-BCMA and huCART19) will be administered as separate, single infusions. For subjects receiving both CART-BCMA and huCART19, CART-BCMA will be administered first, and huCART19 infusion will begin immediately after completion of CART-BCMA infusion.

In the unlikely event that an acute infusion reaction develops to CART-BCMA prior to infusion of huCART19, huCART19 infusion may be delayed up to 48 hours at the discretion of the investigator. If >4 hours have passed between pre-medication and huCART19 infusion, pre-medication will be re-administered. If the huCART19 infusion is delayed and the subject has not stabilized to permit huCART19 infusion within 48 hours of the initially scheduled infusion time, the huCART19 dose will be canceled unless further delay is approved by the Sponsor.

### Post-Infusion Evaluations

Subjects will return to the clinic at days+2, +4, +7, +10, +14 and +21 (+/−1 day) and +28 (+/−3 days) for evaluations.

### Maintenance Therapy

Maintenance therapy may be administered beginning after the day 28 evaluation or once adverse events that are probably/definitely related to CAR T cells and/or lymphodepleting chemotherapy have recovered to baseline or grade ≤2, whichever is later. Beyond these constraints, the decision on whether and when to initiate maintenance therapy will be based on the investigator's discretion. Choice of maintenance regimen is also at the discretion of the investigator. Lenalidomide monotherapy is the preferred maintenance therapy, and steroid-containing regimens should be avoided if possible.

### Monthly (+/−7 Days) Evaluations and Quarterly Evaluations

Subjects will return to the clinic for monthly (+/−7 days) evaluations until six months after CAR T cell infusion for evaluations. After the six-month evaluation, subjects will undergo quarterly evaluations (+/−14 days) for up to one year post-infusion.

### Research Correlative Studies Assessment

Standard research peripheral blood draws will consist of the following:

1. ˜25 mL peripheral blood in EDTA tubes (i.e., purple-top tubes) for PBMC and nucleic acid isolation

2. ˜5 mL peripheral blood in plain plastic tubes (i.e., red-top tubes) for serum isolation

Target collection volume for bone marrow aspirates will be 10-20 mL. For bone marrow aspirate samples designated for both standard anatomic pathology and correlative studies, ˜2-5 mL will be allocated to standard anatomic pathology and the remainder for correlative studies. Aspirate available for correlative studies will be divided as follows: ˜1-3 mL to plastic tube (i.e., red-top tube) for serum isolation and the remainder to EDTA tubes (i.e., purple-top tubes) for PBMC and nucleic acid isolation.

Target length of bone marrow core biopsies is 1 cm each for standard anatomic pathology (where applicable) and correlative studies. This length may be obtained from separate biopsies or division of a single biopsy at the discretion of the clinician performing the procedure.

The following are correlative assays that will be planned for peripheral blood and (where applicable) bone marrow aspirates:

1. Flow cytometry and qPCR for detection of huCART19 and CART-BCMA cells.

2. Flow cytometry for target antigen expression on multiple myeloma cells (bone marrow aspirates) and detection/characterization of minimal residual disease.

3. Phenotyping of T cell subsets by flow cytometry and/or molecular methods.

4. Molecular detection/characterization of minimal residual disease by immunoglobulin heavy chain sequencing.

5 Immunohistochemical analysis on bone marrow core biopsies for target antigen expression and characterization of T cell and other cellular components of the bone marrow.

6. sBCMA/BAFF/APRIL ELISA for soluble markers.

### Example 3: Phase 1 Study of CART-BCMA with or without huCART19 as Consolidation of Standard First or Second-Line Therapy for High-Risk Multiple Myeloma

### Study Summary

### Study Design

This is an open-label phase 1 study to assess the safety, pharmacodynamics, and anti-myeloma effects of CART-BCMA, with or without huCART19, in patients responding to first- or second-line therapy for high-risk multiple myeloma and in relapsed/refractory multiple myeloma patients responding to salvage therapy.

The regimen evaluated in this study is based on established safety of CART-BCMA demonstrated in UPCC 14415/IRB #822756 at dose of 5×10⁸ cells, administered as split infusions, following cyclophosphamide 1.5 g/m² in patients with relapsed/refractory myeloma. This study tests CART-BCMA (1) as consolidation of early therapy for multiple myeloma and as consolidation of standard salvage therapy for relapsed/refractory multiple myeloma, (2) with addition of fludarabine to the lymphodepleting chemotherapy regimen, (3) in combination with huCART19, and (4) as a single rather than split-dose infusion.

The study will introduce these changes to the CART-BCMA regimen in stepwise fashion over three enrollment phases:
Phase A: Safety Run-in to test the safety of CART-BCMA+huCART19 as split-dose infusions after lymphodepleting chemotherapy with cyclophosphamide+fludarabine in patients who have relapsed/refractory myeloma after two prior regimens but who are responding to their current therapy.
Phase A Expansion: Expansion of the Phase A cohort to increase understanding of safety, pharmacodynamics, and anti-myeloma effects of the combined CART-BCMA/huCART19 regimen as a consolidation approach in the relapsed/refractory setting. Enrollment into the Phase A Expansion may occur concurrently with Phase B.
Phase B: Randomization Phase in which patients responding to first- or second-line therapy will receive either CART-BCMA alone (Cohort 1) or CART-BCMA+huCART19 (Cohort 2) as split-dose infusions after lymphodepleting chemotherapy with cyclophosphamide+fludarabine.
Phase C: Single-dose infusion phase to test the safety of single-dose infusion of CART-BCMA alone (Cohort 1) and CART-BCMA+huCART19 (Cohort 2) as single-dose infusions after lymphodepleting chemotherapy with cyclophosphamide+fludarabine in patients responding to first- or second-line therapy.

Throughout the study, the target dose for CART-BCMA and huCART19 will be 5×10⁸ CAR-expressing cell for each product. “Cohort 1” refers to the group of subjects assigned to receive CART-BCMA alone; “Cohort 2” refers to the group of subjects assigned to receive CART-BCMA+huCART19. Split-dose infusions will consist of a 10% dose (of one or both products) on the first infusion day, 30% dose (of one or both products) on the second infusion day, or 60% dose (of one or both products) on the third infusion day. Infusion days may be spread over 7 calendar days due to scheduling constraints or to allow observation of suspected early cytokine release syndrome or other toxicity. Infusions will begin 3 days (+/−1 day) after completion of lymphodepleting chemotherapy with cyclophosphamide+fludarabine.

### Safety Run-in Phase (Phase A)

To test the safety of this new combination CAR T cell approach, the safety run-in will enroll subjects with high-risk multiple myeloma that is relapsed/refractory to two prior lines of therapy but responding to current therapy. Three subjects will receive CART-BCMA+huCART19 beginning 3 days (+/−1 day) after cyclophosphamide+fludarabine lymphodepleting chemotherapy. A 21-day waiting period will be instituted between the start of each subject's regimen (i.e. the first day of lymphodepleting chemotherapy). A safety pause will occur until all three subjects have completed 28 days of follow-up and a formal DLT assessment is performed by the Medical Director and Principal Investigator. If a DLT occurs among the first three subjects infused, further enrollment/infusions will be delayed until a formal safety review is performed by the Sponsor Medical Director and Principal Investigator, and any recommended protocol changes have been implemented and approved by appropriate oversight bodies. The safety run-in will then be further expanded to enroll up to six evaluable subjects.

If a DLT was not identified per Medical Director and Principal Investigator assessment, the cumulative safety data will be submitted to the FDA for review and confirmation that the study may proceed to the Randomization Phase (Phase B).

If a DLT was identified per Medical Director and Principal Investigator assessment and the Safety Run-In phase was expanded to enroll up to six evaluable subjects, a Data and Safety Monitoring Board (DSMB) meeting will occur at the end of the Safety Run-In Phase (after all planned subjects have reached the Day +28 timepoint) to evaluate whether the study may progress to the Randomization Phase (Phase B). Concurrently, the cumulative safety data will be submitted to the FDA for review and confirmation of cohort advancement.

### Phase a Expansion Phase

Once safety of CART-BCMA/huCART19 combination therapy is established in the Phase A Safety Run-in Phase (i.e. no DLTs identified per Medical Director and Principal Investigator assessment), an expansion of Phase A will occur in which the Phase A target population (patients with relapsed/refractory multiple myeloma responding to a standard salvage therapy regimen) will receive both CART-BCMA and huCART19. Enrollment into the Phase A Expansion may occur concurrently with Phase B once opened.

### Randomization Phase (Phase B)

Following completion of the safety run-in phase, subjects with high-risk myeloma who are responding to first- or second-line therapy will be randomized (1:1) to receive either CART-BCMA alone (Cohort 1) or CART-BCMA+huCART19 (Cohort 2) beginning 3 days (+/−1 day) after cyclophosphamide+fludarabine chemotherapy. Enrollment will continue until a total of 20 subjects are infused, with a target of 10 evaluable subjects per cohort (1:1 randomization). During the randomization phase, there will be no required waiting periods between subjects.

A DSMB meeting will occur at the end of the Randomization Phase (after all subjects have reached the Day +28 timepoint) to evaluate whether the study may progress to the Single-dose Infusion Phase (Phase C). Concurrently, the cumulative safety data will be submitted to the FDA for review and confirmation of cohort advancement.

### Single-Dose Infusion Phase (Phase C)

This phase will assess the safety of single-dose infusion of the CART-BCMA monotherapy and CART-BCMA+huCART19 combination. This Phase will enroll subjects with high-risk multiple myeloma responding to either first- or second-line therapy. First, three subjects will receive CART-BCMA monotherapy as a single-dose infusion (Cohort 1), 3 days (+/−1 day) after cyclophosphamide+fludarabine chemotherapy. A safety pause will occur until all 3 subjects have completed 28 days of follow-up. Next, three subjects will receive CART-BCMA+huCART19 as single-dose, sequential infusions on the same day (Cohort 2), 3 days (+/−1 day) after cyclophosphamide+fludarabine chemotherapy. A 21-day waiting period will be instituted between the start of each subject's regimen (i.e. the first day of lymphodepleting chemotherapy). In the event of a DLT, further enrollment/infusions will be delayed until a formal safety review is performed by the Sponsor Medical Director and Principal Investigator, and any recommended protocol changes have been implemented and approved by appropriate oversight bodies. Up to 6 evaluable subjects will participate in Phase C (3 in each cohort).

Following CAR T cell therapy, subjects in all Phases will be eligible to receive standard-of-care maintenance therapy at the discretion of the treating investigator following the first formal response assessment at 28 days post-infusion or upon resolution to grade ≤2 of regimen-related toxicity, whichever is later.

Standard clinical assessments will be performed for toxicity and myeloma response. Correlative analyses will be conducted to assess pharmacokinetics/pharmacodynamics of CART-BCMA and huCART19 expansion/persistence and depletion of cells expressing target antigen, effects on clonogenic multiple myeloma subsets, and anti-myeloma immunity (including immunity against myeloma stem-cell antigens).

### Objectives

### Primary Objectives

1) Evaluate the safety of CART-BCMA as consolidation of standard first- or second-line multiple myeloma therapy following lymphodepletion with cyclophosphamide+fludarabine.

2) Evaluate the safety of huCART19 administered with CART-BCMA as consolidation of first- or second-line therapy in patients with recently diagnosed multiple myeloma and as consolidation of salvage therapy in patients with relapsed/refractory multiple myeloma.

### Secondary Objectives

1) Assess clinical outcomes after each CAR T cell regimen (CART-BCMA monotherapy or combination CART-BCMA+huCART19).

a. Assess conventional response criteria, attainment of MRD-negativity (according to IMWG 2016 criteria (Kumar S, et al. The Lancet Oncology; 2016; 17(8):e328-e346))), and attainment of PET-negative response (absence of detectable FDG-avid disease by PET/CT).

b. Duration of response, progression-free survival, and overall survival

c. Molecular MRD by IgH sequencing

2) Evaluate CART-BCMA and huCART19 expansion and persistence kinetics in this clinical setting.

3) Evaluate effects of huCART19 on correlative parameters of CART-BCMA resistance and clonogenic multiple myeloma cells, such as the following:

a. Persistence of clonal BCMAdim/neg or CD19+plasma cells as measured by flow cytometry and immunohistochemistry

b. Depletion of multiple myeloma clonogenicity as measured using in vitro colony formation assays on bone marrow samples

c. Induction of anti-Sox2 and other anti-myeloma immune responses

d. Depletion of clonal CD19+B cells

4) Evaluate cellular composition of apheresis product and CART-BCMA/huCART19 cells.

5) Evaluate effects of post-infusion maintenance therapy on CAR T cell persistence and phenotype, and CAR-related adverse events.

6) Characterize the phenotype (cell-surface immunophenotype, gene expression profile) of multiple myeloma cells that persist after CART-BCMA+/−huCART19.

### Diagnosis and Main Inclusion Criteria

Phase A (Safety Run-In): Adult patients high-risk multiple myeloma that is relapsed/refractory to two prior lines of therapy but responding to current therapy.

Phase A Expansion: Adults with multiple myeloma that is relapsed/refractory to two prior lines of therapy but responding to current therapy.

Phase B (Randomization Phase) and Phase C (Single-dose Infusion Phase): Adult patients with high-risk multiple myeloma who have not achieved a stringent complete response after first- or second-line therapy.

### Investigational Agents, Dose, and Regimen

### Investigational Agent(s):

CART-BCMA Cells: Autologous T cells expressing BCMA (B-cell maturation antigen)-specific chimeric antigen receptors with tandem TCRζ and 4-1BB (TCRζ/4-1BB) costimulatory domains.
huCART19 Cells: Autologous T cells transduced with lentiviral vector to express anti-CD19 scFV TCRζ:4-1BB. Also known as CTL119 cells.
Cyclophosphamide and Fludarabine: Cytotoxic chemotherapy agents use for lymphodepletion prior to CAR T-cell product administration.

### Dose and Route of Administration:

CART-BCMA Cells: 5×10⁸ cells by intravenous infusion; Minimum acceptable dose for infusion is 0.5×10⁸.
huCART19 Cells: 5×10⁸ cells by intravenous infusion; Minimum acceptable dose for infusion is 0.5×10⁸.
Cyclophosphamide and Fludarabine: Cyclophosphamide 300 mg/m² and Fludarabine 30 mg/m² by intravenous infusion

### Regimen:

CART-BCMA Cells: Split-dose and single-dose infusion
huCART19 Cells: Split-dose and single-dose infusion.
Cyclophosphamide/Fludarabine: Given over 3 days; scheduled so that the last day of chemotherapy falls 3 days (+/−1 day) prior to the first CAR T-cell infusion (Day 0).

### Duration of Administration

Based on the total volume to be infused and the recommended infusion rate of 10-20 mL per minute.

### Summary of Study Phases

Phase A: target population (N=3-6) includes patients who (1) have high-risk multiple myeloma, (2) relapsed after or refractory to 2 prior regimens, and (3) show minimal response or better to current regimen. CART-BCMA and huCART19 are administered in split-dose infusions:
Dose 1: 0.5×10⁸ CART-BCMA+0.5×10⁸ huCART19
Dose 2: 1.5×10⁸ CART-BCMA+1.5×10⁸ huCART19
Dose 3: 3.0×10⁸ CART-BCMA+3.0×10⁸ huCART19

Phase A Expansion: target population (N=7) includes patients who (1) relapsed after or refractory to 2 prior regimens and (2) show minimal response or better to current regimen. CART-BCMA and huCART19 are administered in split-dose infusions:
Dose 1: 0.5×10⁸ CART-BCMA+0.5×10⁸ huCART19
Dose 2: 1.5×10⁸ CART-BCMA+1.5×10⁸ huCART19
Dose 3: 3.0×10⁸ CART-BCMA+3.0×10⁸ huCART19

Phase B: target population (N=20) includes patients who (1) have high-risk multiple myeloma, (2) are responding to first or second line therapy, and (3) show minimal response or better to current regimen. Patients are randomized into two cohorts.

In Cohort 1, patients receive CART-BCMA alone in split-dose infusions:
Dose 1: 0.5×10⁸ CART-BCMA
Dose 2: 1.5×10⁸ CART-BCMA
Dose 3: 3.0×10⁸ CART-BCMA

In Cohort 2, CART-BCMA and huCART19 are administered in split-dose infusions:
Dose 1: 0.5×10⁸ CART-BCMA+0.5×10⁸ huCART19
Dose 2: 1.5×10⁸ CART-BCMA+1.5×10⁸ huCART19
Dose 3: 3.0×10⁸ CART-BCMA+3.0×10⁸ huCART19

Phase C: target population (N=3-6) includes patients who (1) have high-risk multiple myeloma, (2) are responding to first or second line therapy, and (3) show minimal response or better to current regimen. Patients are randomized into two cohorts. Patients in Cohort 1 (N=3) receive 5×10⁸ CART-BCMA cells in a single-dose infusion. Patients in Cohort 2 (N=3) receive 5×10⁸ CART-BCMA cells and 5×10⁸ huCART19 cells in a single-dose infusion.

### INTRODUCTION

This is a phase 1 study to evaluate CART-BCMA with or without huCART19 as consolidation therapy in patients with multiple myeloma. This study builds on results of the Penn-sponsored phase 1 study of CART-BCMA in patients with relapsed/refractory multiple myeloma (UPCC #14415/IRB #822756). Compared to UPCC #14415/Penn IRB #822756, this study is designed to evaluate the safety of the following modifications to CART-BCMA administration that investigators hypothesize will increase the response rate and response duration to CART-BCMA and simplify its administration:
Administration of CART-BCMA early in patients' disease course: The rationale for this modification is that clinical efficacy of CART-BCMA in subjects with relapsed/refractory disease is likely limited by functional deficiencies of the pre-treatment T cell repertoire imposed by high disease burden and extensive prior therapy. Subjects responding to first- or second-line therapy will have a lower disease burden and will have been exposed to less cytotoxic chemotherapy.
Administration of CAR T cells as a consolidation therapy: In patients with advanced multiple myeloma, initiating CAR T cell manufacturing while patients are responding to, rather than progressing on, their most recent therapy may increase the potency of CAR T cell products and decrease the likelihood that that disease-related complications develop while awaiting CAR T cell manufacturing.
Use of fludarabine in addition to cyclophosphamide in the lymphodepleting chemotherapy regimen: Addition of fludarabine is expected to increase the likelihood of in vivo CAR T cell expansion and persistence, which are thought to be required for sustained efficacy.
Administration as a single rather than split-dose infusion: This is intended to simplify the infusion schedule and enable co-administration of huCART19 (see below).
Co-administer huCART19: The rationale for addition of huCART19 is evidence that CD19-expressing multiple myeloma cells are clonogenic and mediate resistance to CART-BCMA.

The Phase A and Phase A expansion cohorts target a relapsed/refractory patient population in whom standard therapies enable only short-term disease control. Though Phases B & C of this study entail a high-risk intervention in an early line of therapy, the study population is patients with high-risk multiple myeloma who have failed to achieve a complete response to first-line therapy; the poor prognosis with standard therapy in this population justifies the risks associated with this novel approach.

### Rationale for CAR T Cells as Consolidation Therapy for Multiple Myeloma

“Consolidation therapy” refers to treatment after response to prior therapy to prolong the response and/or reduce risk of relapse/progression. This study will evaluate CAR T cell as consolidation in two different clinical settings: (1) in patients with relapsed/refractory multiple myeloma who are responding to standard salvage therapy (Phase A and Phase A expansion), and (2) in patients with recently diagnosed high-risk multiple myeloma who are responding to standard first- or second-line therapy (Phase B and Phase C).

In the relapsed/refractory setting, some patients progressed with multiple myeloma complications during CAR T cell manufacturing that precluded administration of CART-BCMA. To reduce the likelihood of morbid disease progression during CAR T cell manufacturing and potentially increase the likelihood of durable response, Phase A and the Phase A expansion of this study will focus on relapsed/refractory multiple myeloma patients who are responding to standard salvage therapy. Patients responding to standard salvage therapy, but who still have detectable residual disease, still have a poor prognosis and are appropriate for clinical trials of investigational agents.

In multiple myeloma, standard first-line therapy with regimens such as lenalidomide, bortezomib, and dexamethasone is often consolidated with high-dose melphalan and autologous stem cell transplantation (ASCT). Consolidation with ASCT prolongs overall survival for multiple myeloma patients60-62. The optimal timing of ASCT in the course of modern multiple myeloma therapy is uncertain. Historically, ASCT has been undertaken after response to first-line therapy. This study involves use of CAR T cell therapy rather than ASCT as consolidation of first-line therapy. Two ongoing, large, randomized studies are comparing ASCT after first-line therapy versus ASCT after a later line of therapy. Initial results indicate no overall survival difference between the two arms3, indicating that deferred ASCT yields long-term outcomes comparable to ASCT as consolidation of first-line therapy. Thus, deferring ASCT in favor of investigational consolidation of first-line therapy with CAR T cell therapy is not expected to jeopardize outcomes for subjects.

Phases B and C of this study will enroll subjects who have responded to first- or second-line therapy and harvest T cells for CAR T cell manufacturing before extensive exposure to cytotoxic chemotherapy. It is expected that T cells harvested in this clinical setting will lead to a more clinically active CAR T cell product compared to cells harvested in the relapsed/refractory setting. This expectation is rooted in findings from CLL patients treated with CART19 showing that clinical outcome correlated strongly with presence of early memory T cell phenotypes63. Multiple myeloma progression is accompanied by development of exhausted T cell phenotypes64. As patients progress through multiple lines of therapy, the T cell repertoire likely becomes progressively impaired as disease burden increases and patients receive increasingly aggressive therapies, often with broadly cytotoxic mechanisms of action. These factors—increasing disease burden and immunosuppressive therapy—likely limit clinical efficacy of CAR T cell therapy in patients with relapsed/refractory disease.

There is precedent in prior studies conducted at Penn for evaluation of novel cellular therapies as consolidation of early therapy for multiple myeloma. Several studies have investigated infusion of autologous T cells after standard-of-care ASCT following ex vivo activation and expansion using the same technology that underlies CAR T cell manufacturing. In these studies, T cells were harvested for manufacturing after response to first line therapy but prior to ASCT. The initial studies of this approach demonstrated that post-ASCT immune reconstitution could be augmented by combining vaccination with autologous T cell infusion65-70. In UPCC 01411, genetically modified T cells were utilized for the first time in this setting. In this study, T cells were transduced with affinity-enhanced T cell receptors against cancer-testis antigens (either NY-ESO1 or MAGE-A3, depending on the subject's HLA type). In the cohort who received T cells targeting NY-ESO1, 9/10 subjects exhibited persistence of engineered cells in peripheral blood ≥2 years after infusion, indicating that engineered T cells manufactured from this patient population can achieve long-term in vivo persistence71.

A challenge in evaluating efficacy of consolidation therapies is how to distinguish response to the investigational therapy from response to the preceding therapy. For example, in UPCC 01411, T cells were administered immediately after high-dose melphalan and ASCT, making it was difficult to distinguish the clinical response to melphalan from the clinical response to the engineered T cells. To avoid this limitation, this protocol restricts enrollment to subjects who have achieved at least a minor response but not a complete response to prior therapy despite having received at least three cycles, at which point responses typically “level-off” (i.e., fail to appreciably improve with further therapy). Moreover, subjects will defer standard consolidation with high-dose melphalan and ASCT to a later line of therapy and receive cyclophosphamide and fludarabine, which are not themselves expected to effect a significant anti-myeloma response in this population, as lymphodepleting chemotherapy. Therefore, with this study design, we expect to be able to attribute any multiple myeloma responses observed to clinical activity of the CAR T cells.

### Subject Selection

### Inclusion Criteria

1. Subjects must have a diagnosis of multiple myeloma according to IMWG 2014 criteria (Rajkumar S V, et al. The lancet oncology. 2014; 15(12):e538-548) with any of the following high-risk features. Subjects in the Phase A Expansion are not required to have any high-risk features:
   a. Beta-2-microglobulin ≥5.5 mg/L and LDH greater than upper limit of normal. Note: subjects in whom LDH and/or Beta-2-microglobulin were not measured prior to initiation of systemic therapy may qualify based on measurements obtained after initiation of systemic therapy.
   b. High-risk FISH features: deletion 17p, t(14;16), t(14;20), t(4;14) in conjunction with Beta-2-microglobulin ≥5.5 mg/L (i.e., revised ISS stage 3). Note: subjects in whom Beta-2-microglobulin was not measured prior to initiation of systemic therapy may qualify based on measurements obtained after initiation of systemic therapy.
   c. Metaphase karyotype with >3 structural abnormalities except hyperdiploidy
   d. Plasma cell leukemia (>20% plasma cells in peripheral blood) at any time prior to enrollment
   e. Failure to achieve partial response or better (by IMWG 2016 criteria (Kumar S, et al. The Lancet Oncology; 2016; 17(8):e328-e346)) to initial therapy with an “imid/PI” combination (thalidomide, lenalidomide, or pomalidomide in combination with bortezomib, ixazomib, or carfilzomib).
   f. Early progression on first-line therapy, defined as progression (according to IMWG 2016 criteria (Kumar S, et al. The Lancet Oncology; 2016; 17(8):e328-e346))
      i. Within one year of starting first-line therapy with an “imid/PI” combination
      ii. Within six months of completing first line therapy with an “imid/PI” combination (i.e. a patient who receives an “imid/PI” combination, transitions to observation or maintenance therapy, and progresses within six months of this transition)
      iii. Within one year of a high-dose melphalan and autologous stem cell transplantation (Phase A subjects only)
2. Subjects must meet the following criteria with respect to prior myeloma therapy:
   a. Phase A and Phase A expansion:
      a. Subjects must have disease that has relapsed after or has been refractory to at least two regimens, including a proteasome inhibitor and thalidomide analog (thalidomide, lenalidomide, pomalidomide). Refractoriness is defined as disease progression on-therapy or within 60 days of stopping therapy. b. Subjects must have achieved at least a minimal response (as defined by IMWG 2016 criteria (Kumar S, et al. The Lancet Oncology; 2016; 17(8):e328-e346)) to their current regimen.
      c. Subjects must not have received prior treatment with anti-BCMA cellular therapy. Subjects may have received treatment with other BCMA-directed agents (e.g., anti-BCMA antibody-drug conjugates or bispecific antibodies).
   b. Phases B and C:
      a. Subjects must be in their first line of multiple myeloma therapy, with the following exception: subjects who have advanced to second-line therapy due to disease progression during first-line therapy are eligible if such progression occurred within six months of beginning first-line therapy. Lines of therapy are defined by IMWG 2016 criteria (Kumar S, et al. The Lancet Oncology; 2016; 17(8):e328-e346).
      b. Subjects must not have received cytotoxic chemotherapy (e.g., doxorubicin, cyclophosphamide, etoposide, cisplatin) with the following exceptions:
         i. Low-dose weekly cyclophosphamide (≤500 mg/m²/week)
         ii. Continuous infusion cyclophosphamide, if limited to a single cycle.
      c. Subjects must not have undergone autologous or allogeneic stem cell transplantation.
      d. Subjects must have initiated systemic therapy for multiple myeloma ≤1 year prior to enrollment.
      e. Subjects must have received at least 3 complete cycles of their current regimen and have achieved at least a minimal response (as defined by IMWG 2016 criteria (Kumar S, et al. The Lancet Oncology; 2016; 17(8):e328-e346)) to the most recent line of therapy.
3. Subjects must not have achieved a complete or stringent complete response according to IMWG 2016 criteria (Kumar S, et al. The Lancet Oncology; 2016; 17(8):e328-e346) at time of enrollment unless clonal plasma cells are detectable in bone marrow by flow cytometry. (I.e., subjects in complete or stringent complete response are eligible if minimal residual disease can be documented by bone marrow flow cytometry).
4. Subjects must have signed written, informed consent.
5. Subjects must be ≥18 years of age.
6. Subjects must have adequate vital organ function:
   a. Serum creatinine ≤2.5 or creatinine clearance ≥30 ml/min (measured or estimated according to CKD-EPI) and not dialysis-dependent.
   b. Absolute neutrophil count ≥1000/μl and platelet count ≥50,000/μl (≥30,000/μl if bone marrow plasma cells are ≥50% of cellularity).
   c. SGOT ≤3× the upper limit of normal and total bilirubin ≤2.0 mg/dl (except for patients in whom hyperbilirubinemia is attributed to Gilbert's syndrome).
   d. Left ventricular ejection fraction (LVEF) ≥45%. LVEF assessment must have been performed within 8 weeks of enrollment.
7. Toxicities from prior/ongoing therapies, with the exception of peripheral neuropathy attributable to multiple myeloma therapy, must have recovered to grade ≤2 according to the CTCAE 5.0 criteria or to the subject's prior baseline.
8. Subjects must have an ECOG performance status of 0-2.
9. Subjects must be willing to forego first-line ASCT.
10. Subjects of reproductive potential must agree to use acceptable birth control methods, as described in protocol Section 4.3.

### Exclusion Criteria

1. Pregnant or lactating women
2. Inadequate venous access for or contraindications to leukapheresis.
3. Active hepatitis B, hepatitis C, or HIV infection, or other active, uncontrolled infection.
4. Any uncontrolled medical or psychiatric disorder that would preclude participation as outlined.
5. NYHA Class III or IV heart failure (see Appendix 2), unstable angina, or a history of recent (within 6 months) myocardial infarction or sustained (>30 seconds) ventricular tachyarrhythmias.
6. Have active auto-immune disease, including connective tissue disease, uveitis, sarcoidosis, inflammatory bowel disease, or multiple sclerosis, or have a history of severe (as judged by the investigator) autoimmune disease requiring prolonged immunosuppressive therapy.
7. Have prior or active central nervous system (CNS) involvement (e.g. leptomeningeal disease, parenchymal masses) with myeloma. Screening for this (e.g. with lumbar puncture) is not required unless suspicious symptoms or radiographic findings are present. Subjects with calvarial disease that extends intracranially and involves the dura will be excluded, even if CSF is negative for myeloma.

### Study Drugs

### CART-BCMA Cells

CART-BCMA cells are autologous T cells that have been engineered to express an extracellular single chain antibody (scFv) with specificity for BCMA linked to an intracellular signaling molecule consisting of tandem signaling domains comprised of the TCRζ signaling module linked to the 4-1BB costimulatory domain. The CART-BCMA cells are cryopreserved in infusible cryomedia, and dispensed in an infusion bag. CART-BCMA cells will be administered as split-dose infusions in Phases A and B (10% dose on day 0, 30% dose on day 1, and 60% dose on day 2) or in Phase C (as a single infusion on day 0). The target CART-BCMA dose will be 5×10⁸ transduced cells, with a minimum acceptable infusion dose of 0.5×108 transduced cells. Doses will be formulated to achieve at least the minimum acceptable dose for the 10% dose. Subsequent doses will be formulated dependent on how many cells are available.

huCART19 Cells

huCART19 cells are autologous T cells that have been engineered to express an extracellular single chain antibody (scFv) with specificity for CD19 linked to an intracellular signaling molecule consisting of a tandem signaling domains comprised of the TCRζ signaling module linked to the 4-1BB costimulatory domain. The huCART19 cells are cryopreserved in infusible cryomedia, and dispensed in an infusion bag. huCART19 cells will be administered as split-dose infusions in Phases A and B (10% dose on day 0, 30% dose on day 1, and 60% dose on day 2) or as a single infusion on Day 0 (Phase C). The target huCART19 dose will be 5×10⁸ transduced cells, with a minimum acceptable infusion dose of 0.5×10⁸ transduced cells. Doses will be formulated to achieve at least the minimum acceptable dose for the 10% dose. Subsequent doses will be formulated dependent on how many cells are available.

### Phase a, Phase a Expansion, and Phase B Only: Eligibility to Receive Subsequent CAR T Cell Infusions (30%+60% Doses):

1. Patients should not experience a significant change in performance or clinical status compared to their previous study visit that would, in the opinion of the treating physician or PI, increase the risk of experimental cell infusion.
2. Patients experiencing new laboratory abnormalities that in the opinion of the treating investigator or PI may adversely affect subject safety or the subject's ability to receive further infusions of CAR T cells may have their infusion delayed until both the treating investigator and PI determine it is clinically appropriate to proceed with the CART-BCMA cell infusion.
3. If the treating physician and/or PI feels the patient may be experiencing signs/symptoms of cytokine release syndrome (CRS) or other severe CART-related toxicities, the 30% and 60% infusions may be preemptively delayed to allow for longer observation and monitoring prior to each subsequent infusion. A single isolated temperature elevation does not in itself define CRS, but the investigator should consider delaying the infusion for 24 hours to observe the subject in such instances. If a pre-emptive delay occurs, all infusions must be completed by the end of day +7.

### Lymphodepleting Chemotherapy

Prior to the initial CAR T cell infusion(s), lymphodepleting chemotherapy will be administered as a regimen of cyclophosphamide 300 mg/m²+fludarabine 30 mg/m² daily for three days. Lymphodepleting chemotherapy must be scheduled so that the last day of therapy falls 3 days (+/−1 day) prior to the first CAR T cell infusion.

While administered for research purposes as part of this study, both cyclophosphamide and fludarabine are FDA-approved agents and will be prepared and infused in accordance with their FDA-approved labels and standard institutional practice. The preferred anti-emetic pre-medication for this regimen is ondansetron 16 mg and dexamethasone 12 mg, each administered daily prior to each chemotherapy infusion; this regimen may be altered at the discretion of the treating investigator. Additional standard home anti-emetics may be prescribed by the investigator for as-needed use (e.g., ondansetron, prochlorperazine, lorazepam). Fludarabine dose may be reduced for subjects with estimated GFR <80 mL/min at investigator discretion.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of treating a subject having a disease associated with the expression of BCMA, comprising administering to the subject a first BCMA CAR-expressing cell therapy, wherein:
(i) the subject has high-risk multiple myeloma, e.g., stage III high-risk multiple myeloma based on Revised International Staging System;
(ii) the subject is receiving or has received a first-line therapy (e.g., induction therapy, e.g., induction therapy comprising one, two, or all of: lenalidomide, bortezomib, or dexamethasone) or a second-line therapy, e.g., at least three cycles of the first-line therapy or second-line therapy, e.g., based on IMWG 2016 criteria, e.g., as described in Table 5, and the subject has not progressed from the first-line or second-line therapy; and
(iii) the subject has shown at least a minimal response, e.g., the subject has shown a very good partial response, a partial response, or a minimal response, to a most recent therapy received by the subject (e.g., the first-line therapy or second-line therapy), e.g., based on IMWG 2016 criteria, e.g., as described in Table 5,
thereby treating the subject.

2. The method of claim 1, wherein the subject is receiving or has received a first-line therapy and has not received a second-line therapy.

3. The method of claim 1, wherein the subject is receiving or has received a second-line therapy and has not received a third-line therapy, wherein the subject advanced to the second-line therapy due to disease progression during or after receiving a first-line therapy, wherein the disease progression occurred within one year of beginning the first-line therapy or within six months of completing the first-line therapy.

4. The method of any one of claims 1-3, wherein the subject has not shown or is not showing a complete response or a stringent complete response to the most recent therapy received by the subject (e.g., the first-line therapy or second-line therapy), e.g., based on IMWG 2016 criteria, e.g., as described in Table 5.

5. The method of any one of claims 1-4, wherein:
(i) the subject has not received cytotoxic chemotherapy (e.g., doxorubicin, cyclophosphamide, etoposide, or cisplatin) with the following exceptions:
(a) the subject has received low-dose weekly cyclophosphamide (e.g., ≤500 mg/m²/week), or
(b) the subject has received a single cycle of continuous infusion of cyclophosphamide; or
(ii) T cells are isolated from the subject to manufacture the first BCMA CAR-expressing cell therapy before the subject receives cytotoxic chemotherapy.

6. The method of any one of claims 1-5, wherein the subject has not received autologous or allogeneic stem cell transplantation.

7. The method of any one of claims 1-6, wherein the subject has initiated systemic therapy for multiple myeloma within one year.

8. The method of any one of claims 1-7, wherein:
(i) the subject shows beta-2-microglobulin ≥5.5 mg/L and high-risk FISH features: deletion 17p, t(14;16), t(14;20), t(4;14);
(ii) the subject shows beta-2-microglobulin ≥5.5 mg/L and LDH greater than upper limit of normal;
(iii) the subject shows metaphase karyotype with >3 structural abnormalities except hyperdiploidy;
(iv) the subject has plasma cell leukemia, e.g., the subject shows >20% plasma cells in peripheral blood;
(v) the subject fails to achieve a partial response or better (e.g., based on IMWG 2016 criteria, e.g., as described in Table 5) to an Imid/PI combination (thalidomide, lenalidomide, or pomalidomide in combination with bortezomib, ixazomib, or carfilzomib); or
(vi) the subject progresses on a first-line therapy with an Imid/PI combination within one year (e.g., within six months) of starting the first-line therapy; or within six months of completing the first-line therapy.

9. A method of treating a subject having a disease associated with the expression of BCMA, comprising administering to the subject a first BCMA CAR-expressing cell therapy, wherein:
(i) the subject has high-risk multiple myeloma,
(ii) the subject's multiple myeloma has relapsed after or has been refractory to at least two regimens, e.g., a proteasome inhibitor and/or thalidomide or its analog (e.g., thalidomide, lenalidomide, or pomalidomide), and
(iii) the subject has shown at least a minimal response, e.g., the subject has shown a very good partial response, a partial response, or a minimal response, to a most recent therapy received by the subject (e.g., a third-line therapy, e.g., a salvage therapy, e.g., a standard salvage therapy), e.g., based on IMWG 2016 criteria, e.g., as described in Table 5,
thereby treating the subject.

10. The method of claim 9, wherein the subject has not shown or is not showing a complete response or a stringent complete response to the most recent therapy received by the subject (e.g., a third-line therapy, e.g., a salvage therapy, e.g., a standard salvage therapy), e.g., based on IMWG 2016 criteria, e.g., as described in Table 5.

11. The method of claim 9 or 10, wherein the subject shows detectable residual disease after receiving the most recent therapy (e.g., a third-line therapy, e.g., a salvage therapy, e.g., a standard salvage therapy).

12. The method of any one of claims 9-11, wherein the subject has not received an anti-BCMA cell therapy.

13. The method of any one of claims 9-12, wherein the subject progressed within one year of receiving melphalan and stem cell transplantation (e.g., autologous stem cell transplantation).

14. The method of any one of claims 1-13, further comprising administering to the subject a first CD19 CAR-expressing cell therapy.

15. The method of claim 14, wherein the first CD19 CAR-expressing cell therapy is administered prior to, concurrently with, or after the administration of the first BCMA CAR-expressing cell therapy.

16. The method of claim 14, wherein the first CD19 CAR-expressing cell therapy is administered on the same day as the first BCMA CAR-expressing cell therapy, optionally wherein the first CD19 CAR-expressing cell therapy is administered at least one hour after the completion of the administration of the first BCMA CAR-expressing cell therapy.

17. The method of claim 14, wherein the first CD19 CAR-expressing cell therapy is administered after the first BCMA CAR-expressing cell therapy, wherein if the subject develops acute infusion reaction after the administration of the first BCMA CAR-expressing cell therapy, the first CD19 CAR-expressing cell therapy is administered up to 48 hours (e.g., 24, 36, or 48 hours) after the administration of the first BCMA CAR-expressing cell therapy.

18. The method of any one of claims 1-17, wherein the first BCMA CAR-expressing cell therapy is administered in a single infusion or a split-dose infusion.

19. The method of claim 18, wherein the first BCMA CAR-expressing cell therapy is administered in a single infusion.

20. The method of claim 18, wherein the first BCMA CAR-expressing cell therapy is administered in a split-dose infusion, e.g., wherein the subject receives about 10% of a total dose on a first infusion date, about 30% of a total dose on a second infusion date, and about 60% of a total dose on a third infusion date.

21. The method of any one of claims 1-20, wherein the first BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells, e.g., in a single infusion, e.g., intravenously.

22. The method of any one of claims 14-21, wherein the first CD19 CAR-expressing cell therapy is administered in a single infusion or a split-dose infusion.

23. The method of claim 22, wherein the first CD19 CAR-expressing cell therapy is administered in a single infusion.

24. The method of claim 22, wherein the first CD19 CAR-expressing cell therapy is administered in a split-dose infusion, e.g., wherein the subject receives about 10% of a total dose on a first infusion date, about 30% of a total dose on a second infusion date, and about 60% of a total dose on a third infusion date.

25. The method of any one of claims 14-24, wherein the first CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells, e.g., in a single infusion, e.g., intravenously.

26. The method of any one of claims 1-25, further comprising administering to the subject a first conditioning agent (e.g., a lymphodepletion agent, e.g., a lymphodepleting chemotherapy, e.g., cyclophosphamide and/or fludarabine) before administering the first BCMA CAR-expressing cell therapy and/or the first CD19 CAR-expressing cell therapy.

27. The method of claim 26, comprising administering to the subject cyclophosphamide and fludarabine before administering the first BCMA CAR-expressing cell therapy and/or the first CD19 CAR-expressing cell therapy, optionally wherein:
(i) cyclophosphamide is administered at 300 mg/m² intravenously daily for three days; and
(ii) fludarabine is administered at 30 mg/m² intravenously daily for three days.

28. The method of claim 26 or 27, wherein the first BCMA CAR-expressing cell therapy and/or the first CD19 CAR-expressing cell therapy are administered 2, 3, or 4 days, e.g., 3 days, after the administration of the first conditioning agent is completed (e.g., after the administration of a last dose of the lymphodepletion agent, e.g., a last dose of the lymphodepleting chemotherapy, e.g., a last dose of cyclophosphamide and/or fludarabine).

29. The method of any one of claims 26-28, further comprising, prior to the administration of the first conditioning agent (e.g., the lymphodepletion agent, e.g., the lymphodepleting chemotherapy, e.g., cyclophosphamide and/or fludarabine), obtaining a first sample (e.g., an apheresis sample) from the subject and manufacturing the first BCMA CAR-expressing cell therapy and/or the first CD19 CAR-expressing cell therapy using the sample.

30. The method of claim 29, further comprising, prior to the administration of the first conditioning agent (e.g., the lymphodepletion agent, e.g., the lymphodepleting chemotherapy, e.g., cyclophosphamide and/or fludarabine) and after obtaining the first sample, obtaining a second sample (e.g., stem cells) from the subject for preparing autologous stem cell transplantation.

31. The method of any one of claims 1-30, further comprising administering to the subject a maintenance agent (e.g., lenalidomide) after the administration of the first BCMA CAR-expressing cell therapy and/or the first CD19 CAR-expressing cell therapy, e.g., at the later of:
(i) 26, 27, 28, 29, 30, 31, or 32 days, e.g., 28 days, after the administration of the first BCMA CAR-expressing cell therapy and/or the first CD19 CAR-expressing cell therapy; or
(ii) resolution of grade ≤2 of treatment-related toxicity.

32. The method of claim 31, further comprising administering to the subject a second BCMA CAR-expressing cell therapy after the administration of the maintenance agent, wherein:
(i) 80-100 days (e.g., 90 days) have elapsed since the administration of the first BCMA CAR-expressing cell therapy;
(ii) the subject's multiple myeloma has progressed after the administration of the first BCMA CAR-expressing cell therapy; or
(iii) the subject has exhibited or is exhibiting objective evidence of residual multiple myeloma after the administration of the first BCMA CAR-expressing cell therapy.

33. The method of claim 32, further comprising administering to the subject a second CD19 CAR-expressing cell therapy after the administration of the maintenance agent, wherein >3% peripheral blood lymphocytes of the subject are CD19+ after the administration of the first CD19 CAR-expressing cell therapy, e.g., 7-28 days after the administration of the first CD19 CAR-expressing cell therapy.

34. The method of claim 33, wherein the second CD19 CAR-expressing cell therapy is administered prior to, concurrently with, or after the administration of the second BCMA CAR-expressing cell therapy.

35. The method of claim 33, wherein the second CD19 CAR-expressing cell therapy is administered on the same day as the second BCMA CAR-expressing cell therapy, optionally wherein the second CD19 CAR-expressing cell therapy is administered at least one hour after the completion of the administration of the second BCMA CAR-expressing cell therapy.

36. The method of claim 33, wherein the second CD19 CAR-expressing cell therapy is administered after the second BCMA CAR-expressing cell therapy, wherein if the subject develops acute infusion reaction after the administration of the second BCMA CAR-expressing cell therapy, the second CD19 CAR-expressing cell therapy is administered up to 48 hours (e.g., 24, 36, or 48 hours) after the administration of the second BCMA CAR-expressing cell therapy.

37. The method of any one of claims 32-36, wherein the second BCMA CAR-expressing cell therapy is administered in a single infusion or a split-dose infusion.

38. The method of claim 37, wherein the second BCMA CAR-expressing cell therapy is administered in a single infusion.

39. The method of claim 37, wherein the second BCMA CAR-expressing cell therapy is administered in a split-dose infusion, e.g., wherein the subject receives about 10% of a total dose on a first infusion date, about 30% of a total dose on a second infusion date, and about 60% of a total dose on a third infusion date.

40. The method of any one of claims 32-39, wherein the second BCMA CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

41. The method of any one of claims 33-40, wherein the second CD19 CAR-expressing cell therapy is administered in a single infusion or a split-dose infusion.

42. The method of claim 41, wherein the second CD19 CAR-expressing cell therapy is administered in a single infusion.

43. The method of claim 41, wherein the second CD19 CAR-expressing cell therapy is administered in a split-dose infusion, e.g., wherein the subject receives about 10% of a total dose on a first infusion date, about 30% of a total dose on a second infusion date, and about 60% of a total dose on a third infusion date

44. The method of any one of claims 33-43, wherein the second CD19 CAR-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ viable CAR-expressing cells, e.g., about 5×10⁸ viable CAR-expressing cells, e.g., about 5×10⁸ viable CAR-expressing cells in a single infusion, e.g., intravenously.

45. The method of any one of claims 32-44, wherein the second BCMA CAR-expressing cell therapy is the same as the first BCMA CAR-expressing cell therapy.

46. The method of any one of claims 33-45, wherein the second CD19 CAR-expressing cell therapy is the same as the first CD19 CAR-expressing cell therapy.

47. The method of any one of claims 32-46, further comprising administering to the subject a second conditioning agent (e.g., a lymphodepletion agent, e.g., a lymphodepleting chemotherapy, e.g., cyclophosphamide and/or fludarabine) before administering the second BCMA CAR-expressing cell therapy and/or the second CD19 CAR-expressing cell therapy.

48. The method of claim 47, comprising administering to the subject cyclophosphamide and fludarabine before administering the second BCMA CAR-expressing cell therapy and/or the second CD19 CAR-expressing cell therapy, optionally wherein:
(i) cyclophosphamide is administered at 300 mg/m² intravenously daily for three days; and
(ii) fludarabine is administered at 30 mg/m² intravenously daily for three days.

49. The method of claim 47, comprising administering to the subject cyclophosphamide, e.g., at 1.5 g/m², before administering the second BCMA CAR-expressing cell therapy and/or the second CD19 CAR-expressing cell therapy.

50. The method of any one of claims 1-49, wherein the first or second BCMA CAR-expressing cell therapy comprises a cell (e.g., a population of cells) expressing a BCAM CAR, wherein:
(i) the BCMA CAR comprises one or more of (e.g., all three of) heavy chain complementary determining region 1 (HCDR1), HCDR2, and HCDR3 of any BCMA scFv domain amino acid sequence listed in Table 2 or 3 and/or one or more of (e.g., all three of) light chain complementary determining region 1 (LCDR1), LCDR2, and LCDR3 of any BCMA scFv domain amino acid sequence listed in Table 2 or 3, or a sequence with 95-99% identity thereof;
(ii) the BCMA CAR comprises a heavy chain variable region (VH) listed in Table 2 or 3 and/or a light chain variable region (VL) listed in Table 2 or 3, or a sequence with 95-99% identity thereof;
(iii) the BCMA CAR comprises a BCMA scFv domain amino acid sequence listed in Table 2 or 3 (e.g., SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, and SEQ ID NO: 149), or a sequence with 95-99% identity thereof;
(iv) the BCMA CAR comprises a full-length BCMA CAR amino acid sequence listed in Table 2 or 3(e.g., the amino acid sequence of the immature BCMA CAR comprises the amino acid sequence of SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, and SEQ ID NO: 233), or a sequence with 95-99% identity thereof; or
(v) the BCMA CAR is encoded by a nucleic acid sequence listed in Table 2 or 3 (e.g., SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170), or a sequence with 95-99% identity thereof.

51. The method of any one of claims 14-50, wherein the first or second CD19 CAR-expressing cell therapy comprises a cell (e.g., a population of cells) expressing a CD19 CAR, wherein:
(i) the CD19 CAR comprises one or more of (e.g., all three of) heavy chain complementary determining region 1 (HCDR1), HCDR2, and HCDR3 listed in Table 6 or 7 and/or one or more of (e.g., all three of) light chain complementary determining region 1 (LCDR1), LCDR2, and LCDR3 listed in Table 6 or 8, or a sequence with 95-99% identity thereof;
(ii) the CD19 CAR comprises a heavy chain variable region (VH) of any CD19 scFv domain amino acid sequence listed in Table 6 and/or a light chain variable region (VL) of any CD19 scFv domain amino acid sequence listed in Table 6, or a sequence with 95-99% identity thereof;
(iii) the CD19 CAR comprises a CD19 scFv domain amino acid sequence listed in Table 6, or a sequence with 95-99% identity thereof;
(iv) the CD19 CAR comprises a full-length CD19 CAR amino acid sequence listed in Table 6, or a sequence with 95-99% identity thereof; or
(v) the CD19 CAR is encoded by a nucleic acid sequence listed in Table 6, or a sequence with 95-99% identity thereof.

52. The method of any one of claims 1-51, wherein the subject is a human patient.
